(19) United States
(12) Patent Application Publication
(10) Pub. No.: US 2025/0109165 A1
(43) Pub. Date: Apr. 3, 2025

(54) CYCLOPHILIN INHIBITORS AND USES THEREOF

(71) Applicants: The Broad Institute, Inc., Cambridge, MA (US); President and Fellows of Harvard College, Cambridge, MA (US)

(72) Inventors: David R. Liu, Cambridge, MA (US); Alexander A. Peterson, Cambridge, MA (US)

(73) Assignees: The Broad Institute, Inc., Cambridge, MA (US); President and Fellows of Harvard College, Cambridge, MA (US)

(21) Appl. No.: 18/974,042

(22) Filed: Dec. 9, 2024

Related U.S. Application Data
(63) Continuation of application No. PCT/US2023/024965, filed on Jun. 9, 2023.
(60) Provisional application No. 63/351,133, filed on Jun. 10, 2022.

(51) Int. Cl. C07K 5/02

(52) U.S. Cl. CPC C07K 5/0202

(57) ABSTRACT

Provided herein are compounds of Formula (I-A), (I-B), or (I-C), and pharmaceutically acceptable salts, solvates, hydrates, polymorphs, co-crystals, tautomers, stereoisomers, isotopically enriched forms, prodrugs, or mixtures thereof, and compositions thereof. Also provided are methods and kits involving the inventive compounds or compositions for treating and/or preventing diseases and/or conditions (e.g., neurological disease (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), conditions associated with autophagy (e.g., neurodegenerative disease, infection, cancer, conditions associated with aging, heart disease), conditions associated with aging, conditions associated with modulating the mPTP, cardiovascular conditions (e.g., ischemia-reperfusion injury), stroke, heart attack, conditions associated with oxidative stress, mitochondrial diseases, or other diseases associated with cyclophilins) in a subject, as well as for reducing oxidative stress. Provided are methods of inhibiting a cyclophilin in a subject, cell, tissue, and/or biological sample. Provided are methods of selectively inhibiting a cyclophilin (e.g., CypD, CypE) in a subject, cell, tissue, and/or biological sample.

### RELATED APPLICATIONS

This application claims priority under 35 U.S.C. § 119(e) to U.S. Provisional Application, U.S. Ser. No. 63/351,133, filed Jun. 10, 2022, which is incorporated herein by reference in its entirety.

### GOVERNMENT SUPPORT

This invention was made with government support under grant numbers R35GM118062 and R01EB022376 awarded by the National Institutes of Health. The government has certain rights in the invention.

### BACKGROUND OF THE INVENTION

The human cyclophilin family consists of seventeen proteins containing a structurally conserved peptidyl-prolyl-isomerase (PPIase) domain of around 180 residues (˜20 kDa)¹. Twelve members of this family are reported to catalyze the cis-trans isomerization of peptidyl-proline bonds^{1,2}, a rate-limiting step in the folding of many proteins^{3,4}. Cyclophilin D (CypD) is unique as a mitochondrial cyclophilin^{1,3,5} and is a key regulator of the mitochondrial permeability transition pore (mPTP), a transient channel on the inner mitochondrial membrane that opens under oxidative stress or high mitochondrial matrix calcium levels⁶⁻⁹. Inhibition^{6,7,9,10} or knockout⁶⁻⁹ of CypD causes the mPTP to be more resistant to pore opening events. Prolonged opening of the mPTP results in equilibration of molecules<1.5 kDa between the matrix and intennembrane space, osmotic imbalance, mitochondrial swelling and rupture, and cell death^{8,9,11}. This pathway has been implicated in a variety of diseases associated with oxidative stress, including ischemia-reperfusion injury (IRI)^{9,12,13}, a large number of neurodegenerative disorders¹⁴⁻²¹, liver diseases²², ageing and autophagy^{23,24}, diabetes^{25,26}, and mitochondrial dysfunction diseases²⁷. Inhibition of CypD thus has been recognized as a potential therapeutic strategy for treating IRI^{8,9}, Alzheimer's disease^{15,16}, Huntington's disease¹⁷, multiple sclerosis (MS)¹⁹, Parkinson's disease²¹, amyotrophic lateral sclerosis (ALS)¹⁴, X-linked adrenoleukodystrophy (X-ALD)²⁰, liver cirrhosis²², and diabetes-related diseases^{25,26}. The exact structure²⁸⁻³⁰, function^{6,11,23} and regulatory pathways^{6,11} of the mPTP are still under investigation.

The 16 other known cyclophilin isoforms play diverse and important biological roles^{3,4,31-36}. Therefore, cyclophilin subtype-selectivity is beneficial for an inhibitor used either as a biological probe or as a potential therapeutic to minimize off-target cyclophilin perturbation and unwanted side effects. Thus far, no subtype-selective cyclophilin inhibitor has been described for any of the 17 cyclophilin isoforms^{1,3}. Efforts to develop selective cyclophilin inhibitors are stymied by the high sequence identity (61-86%) and highly conserved structures of the human PPIase domains¹.

All cyclophilin isoforms share a shallow peptidyl-prolyl isomerization (PPI) active site^{1,36,37}. These active site amino acid residues are highly conserved, with modest variation among disparate isoforms. Classic cyclophilin inhibitors such as cyclosporine A (CsA) bind to this active site^{1,37} and thus inhibit most or all cyclophilins. Adjacent to the active site is the S2 pocket, which forms a long substrate binding groove for peptides. S2 pocket residues are more diverse among cyclophilins than active site residues, providing an opportunity for isoform-selective binding¹. In particular, three gatekeeper residues (amino acids 123, 124, and 145 in CypD) and one far S2 pocket residue (amino acid 118 in CypD) are highly diverse among cyclophilins. Small-molecule cyclophilin inhibitors reported thus far show poor isoform selectivity and are not known to exploit non-conserved S2 pocket residues^{3,13,37-43}. To date, neither isoform-selective inhibition of cyclophilin proteins nor complete family-wide screens for selectivity have been reported. Previously described inhibitors either were tested against one cyclophilin, or when counter-screened against other cyclophilins showed preference only among a small subset of cyclophilins^{1,3,13,16,38-45}. There is a need for selective inhibitors of one specific cyclophilin over other cyclophilins, and in particular, for selective inhibitors of CypD over other cyclophilins.

### SUMMARY OF THE INVENTION

It was hypothesized that a CypD-selective inhibitor may be developed through engagement of gatekeeper residues and other S2 pocket amino acids. In this study, the development of novel cyclophilin-binding compounds from a DNA-templated macrocycle library, iterative structural biology, and small-molecule engineering was reported, which resulted in potent and isoform-selective CypD and cyclophilin E (CypE) inhibitors. Prior work has been performed to access compounds that inhibit cyclophilins. See, e.g., International PCT Application Publication No. WO 2021/119249, published Jun. 17, 2021, which is incorporated herein by reference.

The present disclosure provides compounds of Formulae (I-A), (I-B), and (I-C), and pharmaceutically acceptable salts, solvates, hydrates, polymorphs, co-crystals, tautomers, stereoisomers, isotopically enriched forms, prodrugs, and mixtures thereof. The compounds of Formulae (I-A), (I-B), and (I-C), and pharmaceutically acceptable salts, solvates, hydrates, polymorphs, co-crystals, tautomers, stereoisomers, isotopically enriched forms, prodrugs, or mixtures thereof, may bind a cyclophilin (e.g., CypB, CypC, CypD, CypE, CypG, CypH, Cyp40, PPWD1, PPIL1, NKTR) in a subject, cell, tissue, or biological sample. The compounds of Formulae (I-A), (I-B), and (I-C), and pharmaceutically acceptable salts, solvates, hydrates, polymorphs, co-crystals, tautomers, stereoisomers, isotopically enriched forms, prodrugs, or mixtures thereof, may inhibit the activity of a cyclophilin (e.g., CypB, CypC, CypD, CypE, CypG, CypH, Cyp40, PPWD1, PPIL1, NKTR) in a subject, cell, tissue, or biological sample. In certain embodiments, a compound of Formula (I-A), (I-B), or (I-C) selectively inhibits one or more cyclophilins. In certain embodiments, the cyclophilin inhibited by a compound described herein is cyclophilin D (CypD). In certain embodiments, the cyclophilin inhibited by a compound described herein is cyclophilin E (CypE). In certain embodiments, the cyclophilin inhibited by a compound described herein is cyclophilin B (CypB), cyclophilin C (CypC), cyclophilin G (CypG), cyclophilin H (CypH), cyclophilin 40 (Cyp40), PPWD1, PPIL1, or NKTR. In certain embodiments, the compounds of Formulae (I-A), (I-B), are (I-C) are selective for cyclophilin D compared to other cyclophilins (e.g., at least 2-fold, 5-fold, 10-fold, or more selective for cyclophilin D than another cyclophilin). In certain embodiments, the compounds of Formulae (I-A) and (I-B) are selective for cyclophilin D compared to other cyclophilins (e.g., at least 2-fold, 5-fold, 10-fold, or more selective for cyclophilin D than another cyclophilin). In certain embodiments, the compounds of Formula (I-C) are selective for cyclophilin E compared to other cyclophilins (e.g., at least 2-fold, 5-fold, 10-fold, or more selective for cyclophilin E than another cyclophilin). Described herein are methods of using the compounds described herein, and pharmaceutically acceptable salts, solvates, hydrates, polymorphs, co-crystals, tautomers, stereoisomers, isotopically enriched forms, prodrugs, or mixtures thereof, to study the inhibition of a cyclophilin (e.g., CypB, CypC, CypD, CypE, CypG, CypH, Cyp40, PPWD1, PPIL1, NKTR), or as therapeutics for the prevention and/or treatment of diseases associated with the overexpression and/or aberrant (e.g., increased or unwanted) activity of a cyclophilin (e.g., CypB, CypC, CypD, CypE, CypG, CypH, Cyp40, PPWD1, PPIL1, NKTR). The compounds described herein may be useful in treating and/or preventing a disease and/or condition, e.g., in treating and/or preventing a disease and/or condition (e.g., neurodegenerative disease (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases, or other diseases associated with cyclophilins (e.g., CypD, CypE)), in a subject in need thereof. In certain embodiments, the compounds described herein may be useful in treating and/or preventing a disease and/or condition associated with CypD (e.g., ischemia-reperfusion injury (IRI), Alzheimer's disease, Huntington's disease, multiple sclerosis, Parkinson's disease, amyotrophic lateral sclerosis, X-linked adrenoleukodystrophy, liver cirrhosis, or diabetes). Also provided are uses, pharmaceutical compositions, and kits including a compound described herein, or a pharmaceutically acceptable salt, solvate, hydrate, polymorph, co-crystal, tautomer, stereoisomer, isotopically enriched form, prodrug, or mixture thereof.

In one aspect, the present disclosure provides compounds of Formula (I-A):

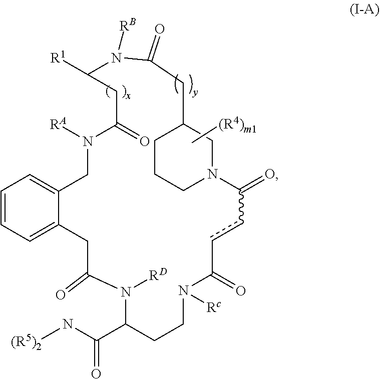

or a pharmaceutically acceptable salt, solvate, hydrate, polymorph, co-crystal, tautomer, stereoisomer, isotopically enriched form, prodrug, or mixture thereof, wherein:
each instance of [symbol] is independently a single or double C—C bond, as valency permits, wherein when [symbol] is a double C—C bond adjacent to [symbol] , then [symbol] indicates that the adjacent C—C double bond may be in a cis or trans configuration;
R¹ is

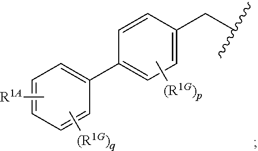

R^{1A} is

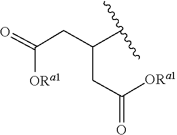

or —(CH₂)ₙN(R^{a1})₂;
each instance of R^{a1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a1} are joined together with the intervening atoms to form a substituted or unsubstituted heterocyclyl ring;
each instance of R^{1G} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{g1}, —NO₂, —N(R^{g2})₂, —SR^{g1}, —SO₂R^{g1}, —CN, or —SCN; R^{g1} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —N(R^{g2})₂, or —O(R^{g3});
each instance of R^{g2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two instances of R^{g2} are taken together to form a ring when attached to nitrogen;
each instance of R^{g3} is independently hydrogen, substituted or unsubstituted alkyl, substituted or unsubstituted acyl, or an oxygen protecting group when attached to an oxygen atom;
R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl;
each instance of R⁵ is independently hydrogen, substituted or unsubstituted alkyl, substituted or unsubstituted alkenyl, substituted or unsubstituted alkynyl, substituted or unsubstituted acyl, or a nitrogen protecting group;
each of R^{A}, R^{B}, R^{C}, and R^{D} is independently hydrogen, substituted or unsubstituted alkyl, substituted or unsubstituted acyl, or a nitrogen protecting group;
x is 0 or 1;
y is 0 or 1;
m1 is 0, 1, 2, 3, 4, 5, or 6;
n is 3, 4, 5, 6, 7, 8, 9, or 10;
p is 0, 1, 2, 3, or 4; and
q is 0, 1, 2, 3, or 4.

In one aspect, the present disclosure provides compounds of Formula (I-B):

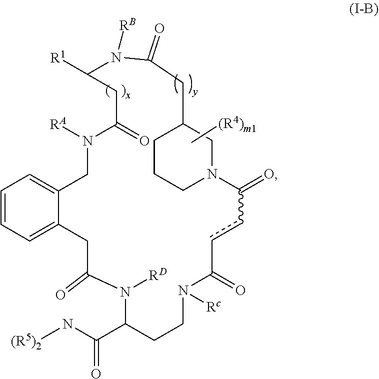

or a pharmaceutically acceptable salt, solvate, hydrate, polymorph, co-crystal, tautomer, stereoisomer, isotopically enriched form, prodrug, or mixture thereof, wherein:
each instance of [symbol] is independently a single or double C—C bond, as valency permits, wherein when [symbol] is a double C—C bond adjacent to [symbol] , then [symbol] indicates that the adjacent C—C double bond may be in a cis or trans configuration;
R¹ is

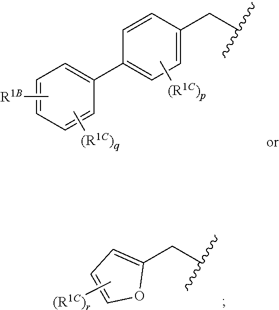

R^{1B} is

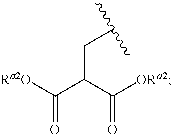

each instance of R^{a2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a2} are joined together with the intervening atoms to form a substituted or unsubstituted heterocyclyl ring;
each instance of R^{1C} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —SO₂R^{c1}, —CN, or —SCN;
R^{c1} is halogen, hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —N(R^{c2})₂, or —O(R^{c3});
each instance of R^{c2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two instances of R^{c2} are taken together to form a ring when attached to nitrogen;
each instance of R^{c3} is independently hydrogen, substituted or unsubstituted alkyl, substituted or unsubstituted acyl, or an oxygen protecting group when attached to an oxygen atom;
R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl;
each instance of R⁵ is independently hydrogen or

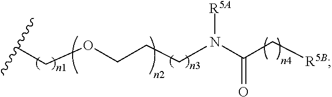

R^{5A} is independently hydrogen, substituted or unsubstituted alkyl, substituted or unsubstituted acyl, or a nitrogen protecting group;
R^{5B} is substituted or unsubstituted carbocyclyl, substituted or unsubstituted aryl, substituted or unsubstituted heterocyclyl, or substituted or unsubstituted heteroaryl;
each of R^{A}, R^{B}, R^{C}, and R^{D} is independently hydrogen, substituted or unsubstituted alkyl, substituted or unsubstituted acyl, or a nitrogen protecting group;
x is 0 or 1;
y is 0 or 1;
m1 is 0, 1, 2, 3, 4, 5, or 6;
p is 0, 1, 2, 3, or 4;
q is 0, 1, 2, 3, or 4;
r is 0, 1, 2, or 3;
n1 is 0, 1, 2, 3, 4, 5, 6, 7, 8, 9, or 10;
n2 is 0, 1, 2, 3, 4, 5, 6, 7, 8, 9, or 10;
n3 is 0, 1, 2, 3, 4, 5, 6, 7, 8, 9, or 10; and
n4 is 0, 1, 2, 3, 4, 5, 6, 7, 8, 9, or 10.

In one aspect, the present disclosure provides compounds of Formula (I-C):

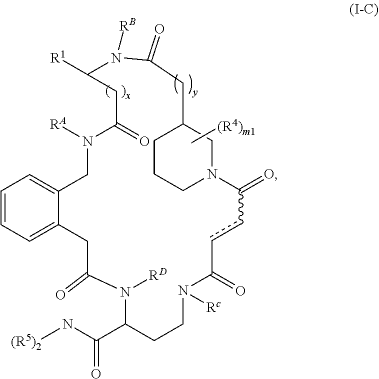

or a pharmaceutically acceptable salt, solvate, hydrate, polymorph, co-crystal, tautomer, stereoisomer, isotopically enriched form, prodrug, or mixture thereof, wherein:
each instance of [symbol] is independently a single or double C—C bond, as valency permits, wherein when [symbol] is a double C—C bond adjacent to [symbol] , then [symbol] indicates that the adjacent C—C double bond may be in a cis or trans configuration;
R¹ is

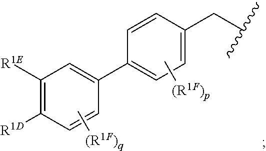

R^{1D} is hydrogen, —B(OR^{a3})₂, or —C(O)R^{a3};
R^{1E} is hydrogen, —B(OR^{a3})₂, or —C(O)R^{a3};
each instance of R^{a3} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a3} are joined together with the intervening atoms to form a substituted or unsubstituted heterocyclyl or heteroaryl ring;
each instance of R^{1F} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{f1}, —NO₂, —N(R^{f2})₂, —SR^{f1}, —SO₂R^{f1}, —CN, or —SCN;
R^{f1} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —N(R^{f2})₂, or —O(R^{f3});
each instance of R^{f2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two instances of R^{f2} are taken together to form a ring when attached to nitrogen;
each instance of R^{f3} is independently hydrogen, substituted or unsubstituted alkyl, substituted or unsubstituted acyl, or an oxygen protecting group when attached to an oxygen atom;
R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl;
each instance of R⁵ is independently hydrogen, substituted or unsubstituted alkyl, substituted or unsubstituted alkenyl, substituted or unsubstituted alkynyl, substituted or unsubstituted acyl, or a nitrogen protecting group;
each of R^{A}, R^{B}, R^{C}, and R^{D} is independently hydrogen, substituted or unsubstituted alkyl, substituted or unsubstituted acyl, or a nitrogen protecting group;
x is 0 or 1;
y is 0 or 1;
m1 is 0, 1, 2, 3, 4, 5, or 6;
p is 0, 1, 2, 3, or 4; and
q is 0, 1,2, or 3;
provided that R¹ is not

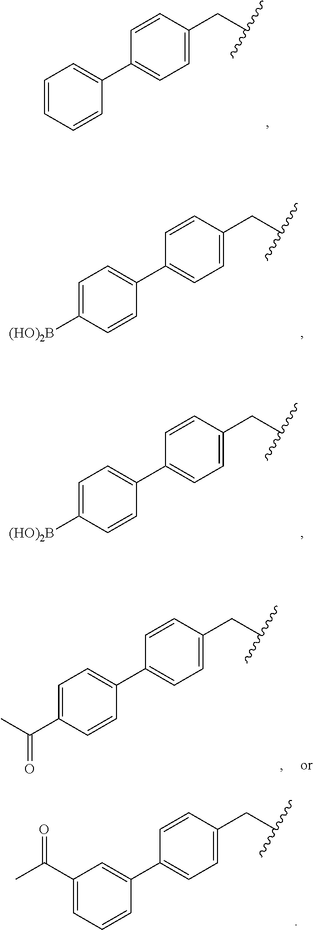 [SMILES: *Cc1ccc(-c2ccccc2)cc1 ; *Cc1ccc(-c2ccc(B(O)O)cc2)cc1 ; *Cc1ccc(-c2ccc(B(O)O)cc2)cc1 ; *Cc1ccc(-c2ccc(C(C)=O)cc2)cc1 ; *Cc1ccc(-c2cccc(C(C)=O)c2)cc1]

Exemplary compounds of Formula (I-A) include, but are not limited to:

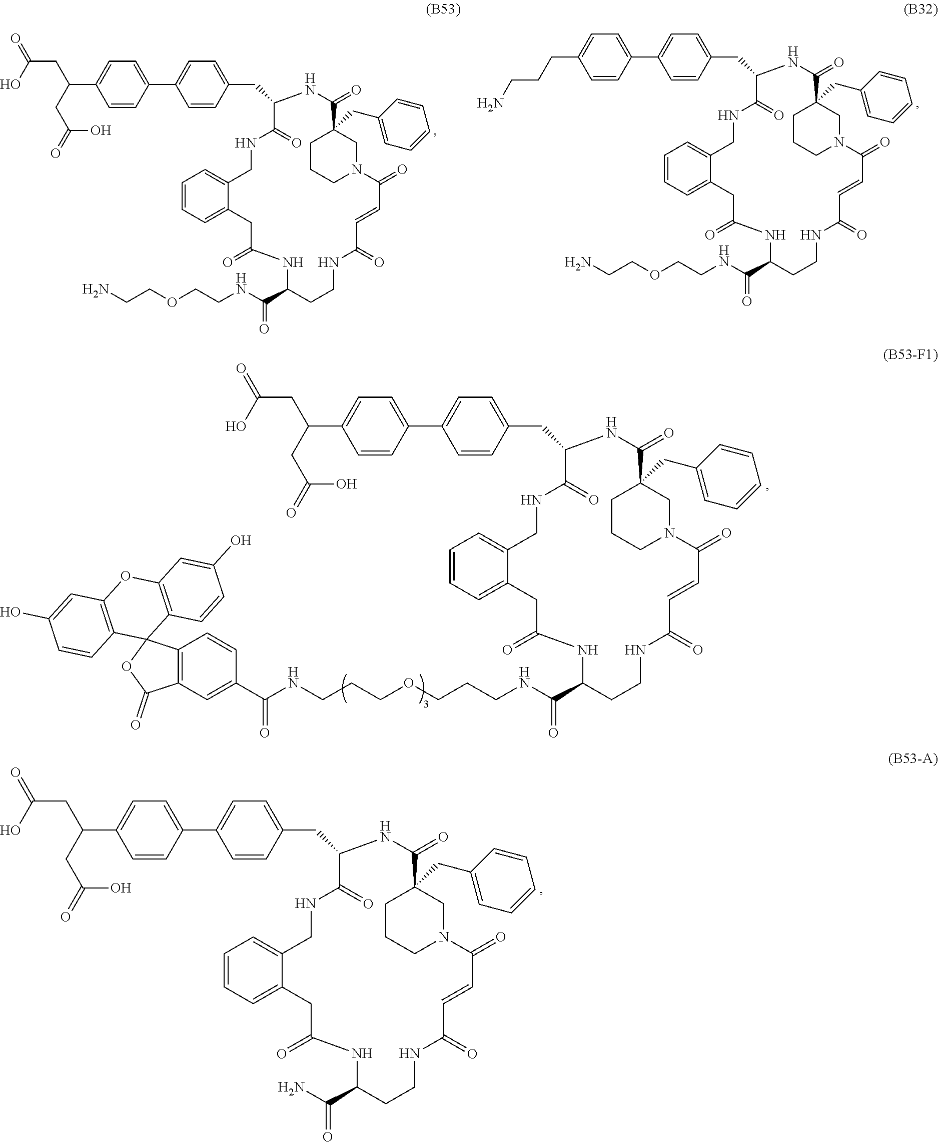

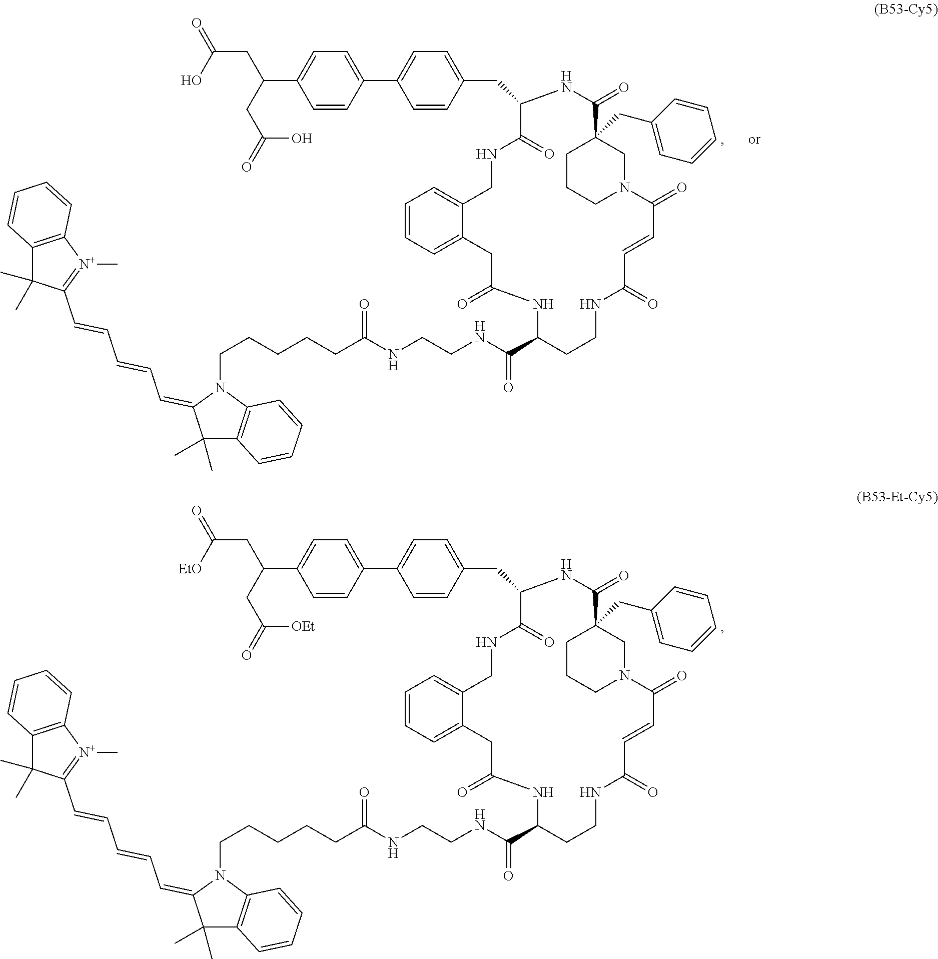 [SMILES: C[N+]1=C(/C=C/C=C/C=C2\N(CCCCCC(=O)NCCNC(=O)[C@@H]3CCNC(=O)/C=C/C(=O)N4CCC[C@](Cc5ccccc5)(C4)C(=O)N[C@@H](Cc4ccc(-c5ccc(C(CC(=O)O)CC(=O)O)cc5)cc4)C(=O)NCc4ccccc4CC(=O)N3)c3ccccc3C2(C)C)C(C)(C)c2ccccc21 ; CCOC(=O)CC(CC(=O)OCC)c1ccc(-c2ccc(C[C@@H]3NC(=O)[C@]4(Cc5ccccc5)CCCN(C4)C(=O)/C=C/C(=O)NCC[C@@H](C(=O)NCCNC(=O)CCCCCN4\C(=C/C=C/C=C/C5=[N+](C)c6ccccc6C5(C)C)C(C)(C)c5ccccc54)NC(=O)Cc4ccccc4CNC3=O)cc2)cc1]

or a pharmaceutically acceptable salt, solvate, hydrate, polymorph, co-crystal, tautomer, stereoisomer, isotopically enriched form, prodrug, or mixture thereof.

Exemplary compounds of Formula (I-B) include, but are not limited to:

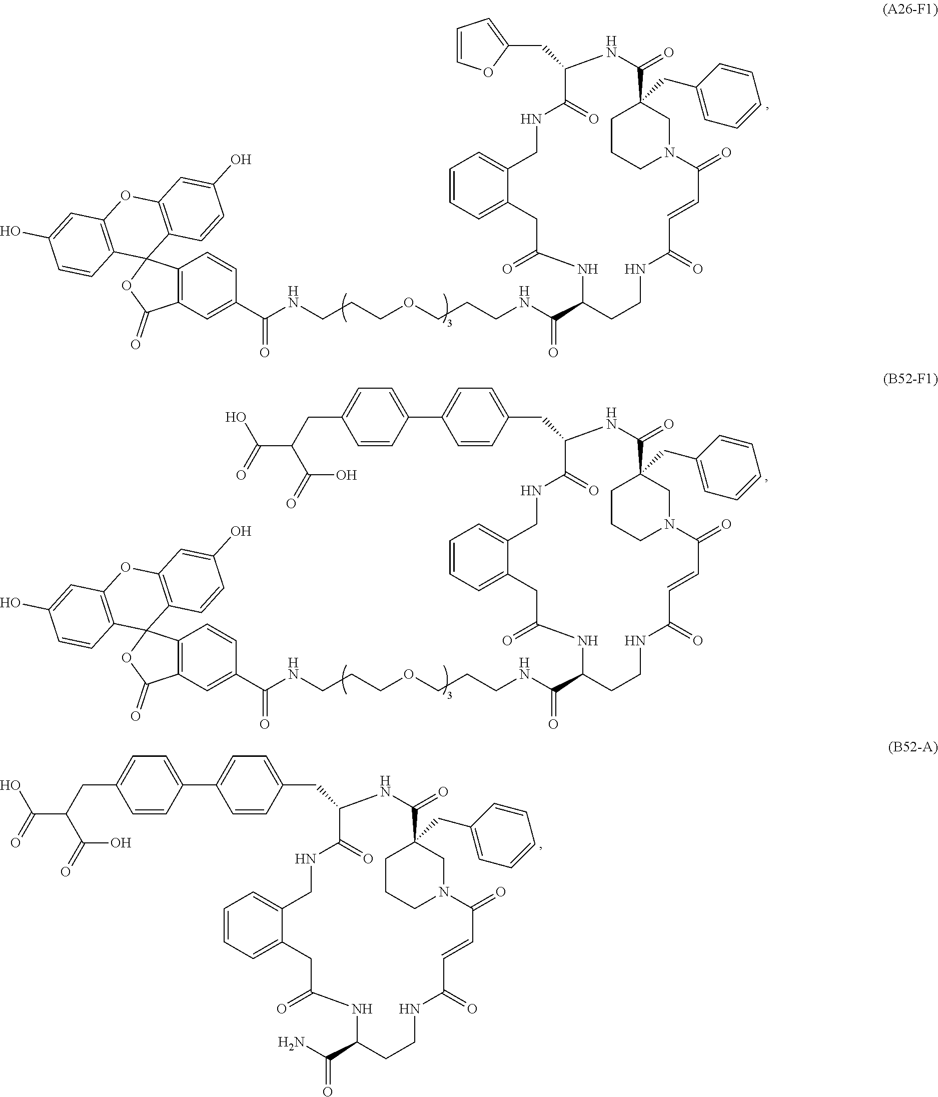

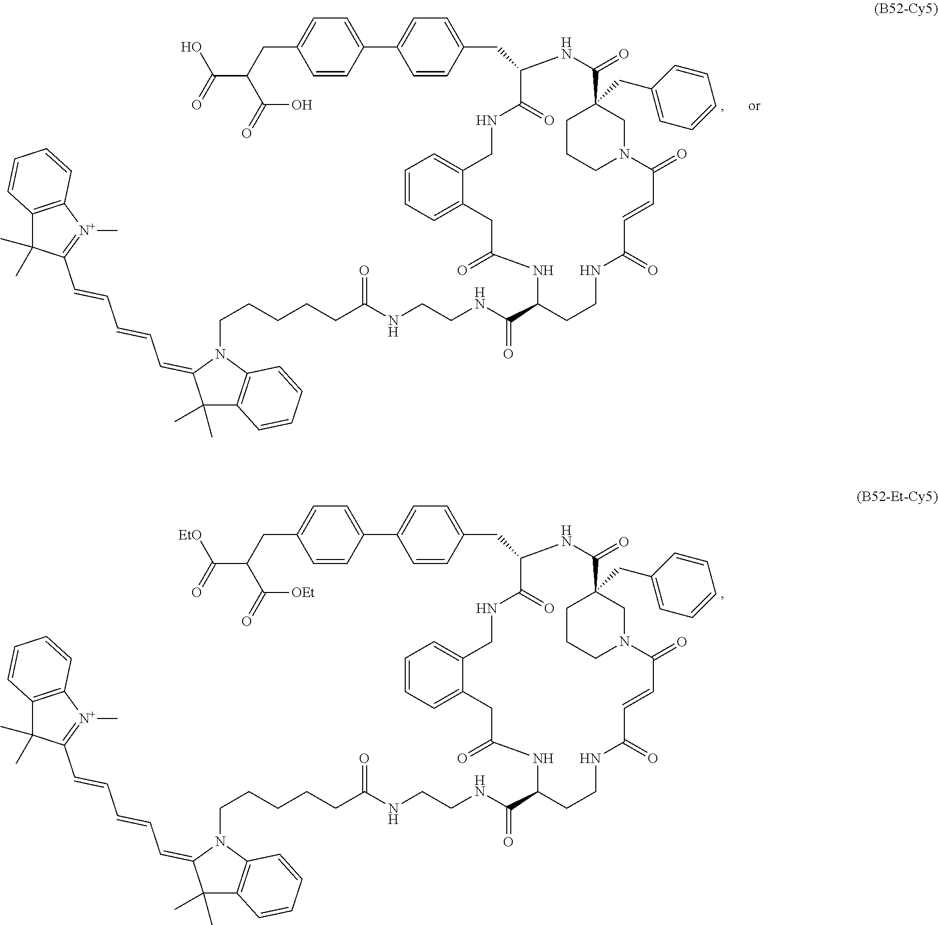 [SMILES: C[N+]1=C(/C=C/C=C/C=C2\N(CCCCCC(=O)NCCNC(=O)[C@@H]3CCNC(=O)/C=C/C(=O)N4CCC[C@](Cc5ccccc5)(C4)C(=O)N[C@@H](Cc4ccc(-c5ccc(CC(C(=O)O)C(=O)O)cc5)cc4)C(=O)NCc4ccccc4CC(=O)N3)c3ccccc3C2(C)C)C(C)(C)c2ccccc21 ; CCOC(=O)C(Cc1ccc(-c2ccc(C[C@@H]3NC(=O)[C@]4(Cc5ccccc5)CCCN(C4)C(=O)/C=C/C(=O)NCC[C@@H](C(=O)NCCNC(=O)CCCCCN4\C(=C/C=C/C=C/C5=[N+](C)c6ccccc6C5(C)C)C(C)(C)c5ccccc54)NC(=O)Cc4ccccc4CNC3=O)cc2)cc1)C(=O)OCC]

pharmaceutically acceptable salt, solvate, hydrate, polymorph, co-crystal, tautomer, stereoisomer, isotopically enriched form, prodrug, or mixture thereof.

Exemplary compounds of Formula (I-C) include, but are not limited to:

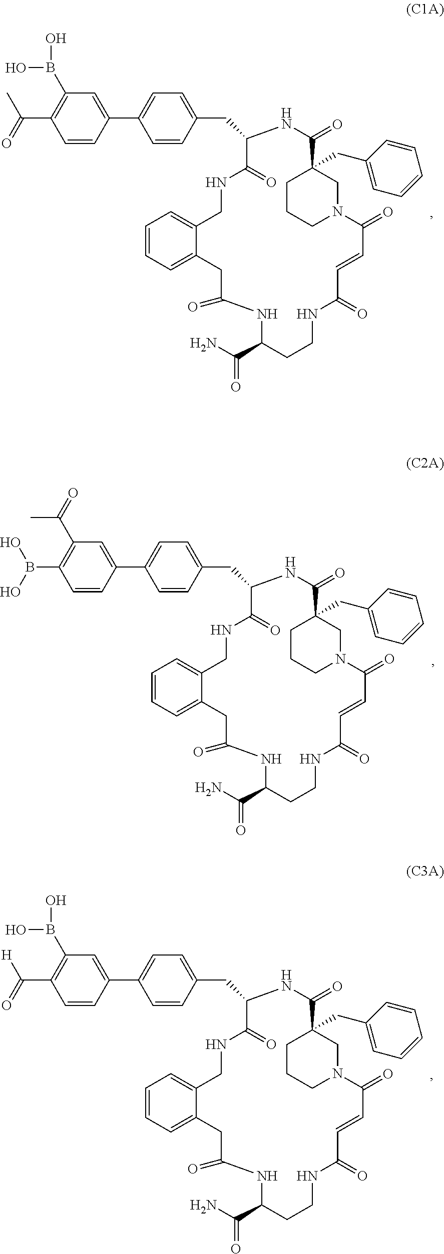 [SMILES: CC(=O)c1ccc(-c2ccc(C[C@@H]3NC(=O)[C@]4(Cc5ccccc5)CCCN(C4)C(=O)/C=C/C(=O)NCC[C@@H](C(N)=O)NC(=O)Cc4ccccc4CNC3=O)cc2)cc1B(O)O ; CC(=O)c1cc(-c2ccc(C[C@@H]3NC(=O)[C@]4(Cc5ccccc5)CCCN(C4)C(=O)/C=C/C(=O)NCC[C@@H](C(N)=O)NC(=O)Cc4ccccc4CNC3=O)cc2)ccc1B(O)O ; NC(=O)[C@@H]1CCNC(=O)/C=C/C(=O)N2CCC[C@](Cc3ccccc3)(C2)C(=O)N[C@@H](Cc2ccc(-c3ccc(C=O)c(B(O)O)c3)cc2)C(=O)NCc2ccccc2CC(=O)N1]

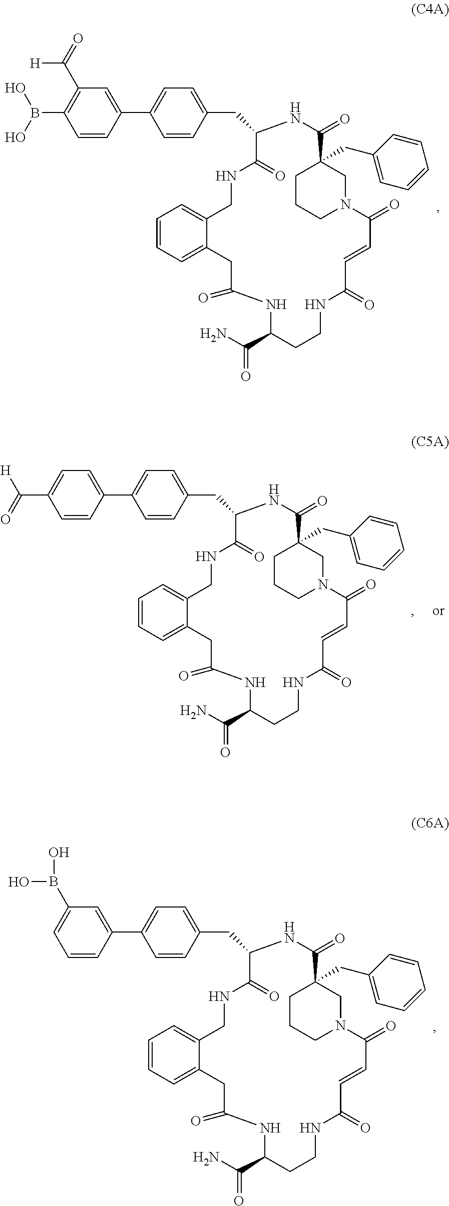 [SMILES: NC(=O)[C@@H]1CCNC(=O)/C=C/C(=O)N2CCC[C@](Cc3ccccc3)(C2)C(=O)N[C@@H](Cc2ccc(-c3ccc(B(O)O)c(C=O)c3)cc2)C(=O)NCc2ccccc2CC(=O)N1 ; NC(=O)[C@@H]1CCNC(=O)/C=C/C(=O)N2CCC[C@](Cc3ccccc3)(C2)C(=O)N[C@@H](Cc2ccc(-c3ccc(C=O)cc3)cc2)C(=O)NCc2ccccc2CC(=O)N1 ; NC(=O)[C@@H]1CCNC(=O)/C=C/C(=O)N2CCC[C@](Cc3ccccc3)(C2)C(=O)N[C@@H](Cc2ccc(-c3cccc(B(O)O)c3)cc2)C(=O)NCc2ccccc2CC(=O)N1]

pharmaceutically acceptable salt, solvate, hydrate, polymorph, co-crystal, tautomer, stereoisomer, isotopically enriched form, prodrug, or mixture thereof.

Exemplary compounds of Formula (I-A) include, but are not limited to, B53, B32, B53-Fl, B53-A, B53-Cy5, B53-Et-Cy5, or a pharmaceutically acceptable salt thereof.

Exemplary compounds of Formula (I-B) include, but are not limited to, A26-Fl, B52-Fl, B52-A, B52-Cy5, B52-Et-Cy5, or a pharmaceutically acceptable salt thereof.

Exemplary compounds of Formula (I-C) include, but are not limited to, C1A, C2A, C3A, C4A, C5A, or C6A, or a pharmaceutically acceptable salt thereof.

Exemplary compounds of Formula (I-A), (I-B), or (I-C) include, but are not limited to, compounds disclosed in Examples 1, 2, 3, 4, and 5, or pharmaceutically acceptable salts, solvates, hydrates, polymorphs, co-crystals, tautomers, stereoisomers, isotopically enriched forms, prodrugs, or mixtures thereof.

In another aspect, the present disclosure provides pharmaceutical compositions comprising a compound described herein (e.g., a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof) and a pharmaceutically acceptable excipient. In certain embodiments, a pharmaceutical composition described herein includes a therapeutically or prophylactically effective amount of a compound described herein. The pharmaceutical compositions may be useful in reducing oxidative stress in a subject, cell, tissue, or biological sample, inhibiting a cyclophilin (e.g., CypB, CypC, CypD, CypE, CypG, CypH, Cyp40, PPWD1, PPIL1, NKTR) in a subject, cell, tissue, or biological sample, in treating and/or preventing a disease in a subject in need thereof. In certain embodiments, the compound being administered or used inhibits a cyclophilin (e.g., CypB, CypC, CypD, CypE, CypG, CypH, Cyp40, PPWD1, PPIL1, NKTR) in a subject, cell, tissue, or biological sample, treats and/or prevents a disease, in a subject in need thereof.

In still another aspect, described herein are kits including a container with a compound or pharmaceutical composition described herein. A kit described herein may include a single dose or multiple doses of the compound or pharmaceutical composition. The described kits may be useful in reducing oxidative stress in a subject, cell, tissue, or biological sample. The described kits may be useful in inhibiting a cyclophilin (e.g., CypB, CypC, CypD, CypE, CypG, CypH, Cyp40, PPWD1, PPIL1, NKTR) in a subject, cell, tissue, or biological sample. The described kits may be useful in modulating (e.g., regulating) the mPTP and/or reducing oxidating stress. The described kits may be useful in inhibiting a cyclophilin (e.g., CypD, CypE). The described kits (e.g., including compounds of Formula (I-A), (I-B), or (I-C)) may be useful in treating and/or preventing a disease in a subject in need thereof. In certain embodiments, a kit described herein further includes instructions for using the compound or pharmaceutical composition included in the kit. A kit described herein may also include information (e.g., prescribing information) as required by a regulatory agency, such as the U.S. Food and Drug Administration (FDA).

In certain embodiments, the compound being administered or used inhibits a cyclophilin (e.g., CypB, CypC, CypD, CypE, CypG, CypH, Cyp40, PPWD1, PPIL1, NKTR) in a subject, cell, tissue, or biological sample. In certain embodiments, the compound being administered or used inhibits a cyclophilin (e.g., CypD, CypE). In certain embodiments, the compound being administered or used inhibits CypD selectively. In certain embodiments, the compound being administered or used inhibits CypE selectively.

Another aspect of the present disclosure relates to methods of treating a disease in a subject in need thereof comprising administering to the subject a therapeutically effective amount of a compound or pharmaceutical composition described herein. In another aspect, the present disclosure provides methods of preventing a disease in a subject in need thereof comprising administering to the subject a prophylactically effective amount of a compound or pharmaceutical composition described herein.

In yet another aspect, the present disclosure provides compounds and pharmaceutical compositions described herein for use in a method of the disclosure (e.g., a method of inhibiting a cyclophilin (e.g., CypB, CypC, CypD, CypE, CypG, CypH, Cyp40, PPWD1, PPIL1, NKTR) in a subject, cell, tissue, or biological sample, a method of reducing oxidative stress in a subject, cell, tissue, or biological sample, and a method of treating and/or preventing a disease in a subject in need thereof. In yet another aspect, the present disclosure provides compounds (e.g., including compounds of Formula (I-A), (I-B), or (I-C)) and pharmaceutical compositions described herein for use in a method of the disclosure (e.g., a method of inhibiting a cyclophilin (e.g., CypB, CypC, CypD, CypE, CypG, CypH, Cyp40, PPWD1, PPIL1, NKTR) in a subject, cell, tissue, or biological sample, a method of reducing oxidative stress in a subject, cell, tissue, or biological sample, and a method of treating and/or preventing a disease in a subject in need thereof. In another aspect, the present disclosure provides compounds and pharmaceutical compositions described herein for use in a method of the disclosure (e.g., a method of inhibiting a cyclophilin (e.g., CypB, CypC, CypD, CypE, CypG, CypH, Cyp40, PPWD1, PPIL1, NKTR) in a subject, cell, tissue, or biological sample, a method of reducing oxidative stress in a subject, cell, tissue, or biological sample, and a method of treating and/or preventing a disease in a subject in need thereof. In another aspect, the present disclosure provides compounds (e.g., including compounds of Formula (I-A), (I-B), or (I-C)) and pharmaceutical compositions described herein for use in a method of the disclosure (e.g., a method of inhibiting a cyclophilin (e.g., CypB, CypC, CypD, CypE, CypG, CypH, Cyp40, PPWD1, PPIL1, NKTR) in a subject, cell, tissue, or biological sample, a method of reducing oxidative stress in a subject, cell, tissue, or biological sample, and a method of treating and/or preventing a disease in a subject in need thereof.

The present application refers to various issued patents, published patent applications, journal articles, and other publications, all of which are incorporated herein by reference. The details of one or more embodiments of the invention are set forth herein. Other features, objects, and advantages of the invention will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

When a range of values is listed, it is intended to encompass each value and subrange within the range. For example, “C₁₋₆” is intended to encompass C₁, C₂, C₃, C₄, C₅, C₆, C₁-6, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

“Hydrocarbon chain” refers to a substituted or unsubstituted divalent alkyl, alkenyl, or alkynyl group. A hydrocarbon chain includes at least one chain, each node (“carbon unit”) of which including at least one carbon atom, between the two radicals of the hydrocarbon chain. For example, hydrocarbon chain —C^{A}H(C^{B}H₂C^{C}H₃)— includes only one carbon unit C^{A}. The term “Cₓ hydrocarbon chain,” wherein x is a positive integer, refers to a hydrocarbon chain that includes x number of carbon unit(s) between the two radicals of the hydrocarbon chain. If there is more than one possible value of x, the smallest possible value of x is used for the definition of the hydrocarbon chain. For example, —CH(C₂H₅)— is a C₁ hydrocarbon chain, and

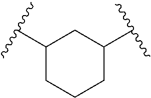 [SMILES: *C1CCCC(*)C1]

is a C₃ hydrocarbon chain. When a range of values is used, e.g., a C₁₋₆ hydrocarbon chain, the meaning of the range is as described herein. A hydrocarbon chain may be saturated (e.g., —(CH₂)₄—). A hydrocarbon chain may also be unsaturated and include one or more C═C and/or C≡C bonds anywhere in the hydrocarbon chain. For instance, —CH═CH—(CH₂)₂—, —CH₂—C≡C—CH₂—, and —C≡C—CH═CH— are all examples of a unsubstituted and unsaturated hydrocarbon chain. In certain embodiments, the hydrocarbon chain is unsubstituted (e.g., —(CH₂)₄—). In certain embodiments, the hydrocarbon chain is substituted (e.g., —CH(C₂H₅)— and —CF₂—). Any two substituents on the hydrocarbon chain may be joined to form a substituted or unsubstituted carbocyclyl, substituted or unsubstituted heterocyclyl, substituted or unsubstituted aryl, or substituted or unsubstituted heteroaryl ring. For instance,

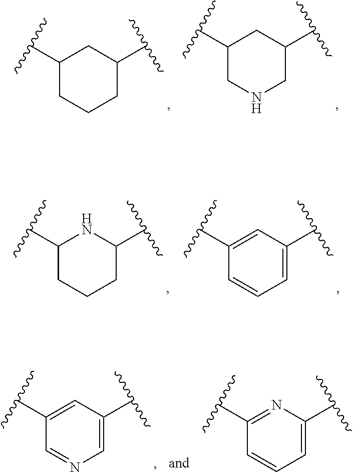 [SMILES: *C1CCCC(*)C1 ; *C1CNCC(*)C1 ; *C1CCCC(*)N1 ; *c1cccc(*)c1 ; *c1cncc(*)c1 ; *c1cccc(*)n1]

are all examples of a hydrocarbon chain. In contrast, in certain embodiments

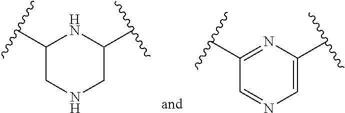 [SMILES: *C1CNCC(*)N1 ; *c1cncc(*)n1]

are not within the scope of the hydrocarbon chains described herein.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

“Alkenyl” refers to a radical of a straight-chain or branched hydrocarbon group having from 1 to 20 carbon atoms one or more carbon-carbon double bonds (e.g., 1, 2, 3, or 4 double bonds), and no triple bonds (“C₁₋₂₀ alkenyl”). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. In certain embodiments, the alkynyl group is an optionally substituted C₂₋₂₀ alkenyl. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

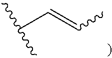

may be in the (E)- or (Z)-configuration.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl. In certain embodiments, the alkynyl group is an optionally substituted C₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅); and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

“Heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl and refers to an optionally substituted alkyl group substituted by an optionally substituted heteroaryl group.

“Partially unsaturated” refers to a group that includes at least one double or triple bond. A “partially unsaturated” ring system is further intended to encompass rings having multiple sites of unsaturation but is not intended to include aromatic groups (e.g., aryl or heteroaryl groups) as defined herein. Likewise, “saturated” refers to a group that does not contain a double or triple bond, i.e., contains all single bonds.

Alkyl, alkenyl, alkynyl, carbocyclyl, heterocyclyl, aryl, and heteroaryl groups, which are divalent bridging groups are further referred to using the suffix -ene, e.g., alkylene, alkenylene, alkynylene, carbocyclylene, heterocyclylene, arylene, and heteroarylene.

A group is optionally substituted unless expressly provided otherwise. The term “optionally substituted” refers to being substituted or unsubstituted.

In certain embodiments, alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}—, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{cc})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{cc}, —Si(R^{cc})₃, —OSi(R^{cc})₃, —C(═S)N(R^{ff})₂, —C(═O)SR^{cc}, —C(═S)SR^{cc}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}—N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R—, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄, BPh₄, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, P₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

“Alkoxy” or “alkoxyl” refers to a radical of the formula: —O-alkyl.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂—CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR—, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pme), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR—, —C(═NR^{bb})N(R^{bb})₂, —S(═O)R^{aa}, —SO₂R^{aa}, —Si(R^{cc})₃, —P(R^{cc})₂, —P(R^{cc})₃⁺X⁻, —P(OR^{cc})₂, —P(OR⁾₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR—, —C(═NR^{bb})N(R^{bb})₂, —S(═O)R^{aa}, —SO₂R^{aa}, —Si(R^{aa})₃, —P(R^{cc})₂, —P(R)₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{cc})₂, —OP(═O)(OR⁾₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, -ONf), or trifluoromethanesulfonate (triflate, -OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “isotopes” refers to variants of a particular chemical element such that, while all isotopes of a given element share the same number of protons in each atom of the element, those isotopes differ in the number of neutrons.

The term “prodrugs” refer to compounds, including derivatives of the compounds of Formula (I-A), (I-B), or (I-C), which have cleavable groups and become by solvolysis or under physiological conditions the compounds of Formula (I-A), (I-B), or (I-C) which are pharmaceutically active in vivo. Such examples include, but are not limited to, ester derivatives and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

The terms “administer,” “administering,” or “administration” refer to implanting, absorbing, ingesting, injecting, inhaling, or otherwise introducing an inventive compound, or a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The terms “condition,” “disease,” and “disorder” are used interchangeably.

An “effective amount” of a compound of Formula (I-A), (I-B), or (I-C) refers to an amount sufficient to elicit the desired biological response, i.e., treating the condition. As will be appreciated by those of ordinary skill in this art, the effective amount of a compound of Formula (I-A), (I-B), or (I-C) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I-A), (I-B), or (I-C) is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms or causes of the condition, or enhances the therapeutic efficacy of another therapeutic agent.

The term “angiogenesis” refers to the formation and the growth of new blood vessels. Normal angiogenesis occurs in the healthy body of a subject for healing wounds and for restoring blood flow to tissues after injury. The healthy body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal or pathological angiogenesis refers to angiogenesis greater than that in a normal body, especially angiogenesis in an adult not related to normal angiogenesis (e.g., menstruation or wound healing). Abnormal angiogenesis can provide new blood vessels that feed diseased tissues and/or destroy normal tissues, and in the case of cancer, the new vessels can allow tumor cells to escape into the circulation and lodge in other organs (tumor metastases). In certain embodiments, the angiogenesis is pathological angiogenesis.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as a transgenic oocyte, sperm cell, blastocyst, embryo, fetus, donor cell, or cell nucleus, or cells or cell lines derived from biological samples.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent. In certain embodiments, a therapeutically effective amount is an amount sufficient for treating a disease and/or condition (e.g., neurodegenerative disease (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases, or other diseases associated with a cyclophilin). In certain embodiments, a therapeutically effective amount is an amount sufficient for treating a disease and/or condition associated with CypD (e.g., ischemia-reperfusion injury (IRI), Alzheimer's disease, Huntington's disease, multiple sclerosis, Parkinson's disease, amyotrophic lateral sclerosis, X-linked adrenoleukodystrophy, liver cirrhosis, or diabetes). In certain embodiments, a therapeutically effective amount is an amount sufficient for binding and/or inhibiting a cyclophilin (e.g., CypB, CypC, CypD, CypE, CypG, CypH, Cyp40, PPWD1, PPIL1, NKTR). In certain embodiments, a therapeutically effective amount is an amount sufficient for binding and/or inhibiting a cyclophilin (e.g., CypD, CypE). In certain embodiments, a therapeutically effective amount is an amount sufficient for binding and/or inhibiting CypD. In certain embodiments, a therapeutically effective amount is an amount sufficient for binding and/or inhibiting CypE. In certain embodiments, a therapeutically effective amount is an amount sufficient for binding and/or inhibiting CypC.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for binding a cyclophilin (e.g., CypB, CypC, CypD, CypE, CypG, CypH, Cyp40, PPWD1, PPIL1, NKTR) and/or inhibiting the cyclophilin (e.g., CypB, CypC, CypD, CypE, CypG, CypH, Cyp40, PPWD1, PPIL1, NKTR). In certain embodiments, a prophylactically effective amount is an amount sufficient for binding a cyclophilin (e.g., CypD, CypE) and/or inhibiting the cyclophilin (e.g., CypD, CypE). In certain embodiments, a prophylactically effective amount is an amount sufficient for binding and/or inhibiting CypD. In certain embodiments, a prophylactically effective amount is an amount sufficient for binding and/or inhibiting CypE. In certain embodiments, a prophylactically effective amount is an amount sufficient for binding and/or inhibiting CypC. In certain embodiments, a prophylactically effective amount is an amount sufficient for treating a disease and/or condition (e.g., neurodegenerative disease (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases, or other diseases associated with cyclophilins (e.g., CypD, CypE)). In certain embodiments, a prophylactically effective amount is an amount sufficient for treating a disease and/or condition associated with CypD (e.g., ischemia-reperfusion injury (IRI), Alzheimer's disease, Huntington's disease, multiple sclerosis, Parkinson's disease, amyotrophic lateral sclerosis, X-linked adrenoleukodystrophy, liver cirrhosis, or diabetes).

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). The term “neurodegenerative disease” refers to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), X-linked adrenoleukodystrophy, hyperglycemia, hyperinsulinemia, insulin resistance, and obesity.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, lymphoma, non-Hodgkin's lymphoma, Waldenstrom macroglobulinemia, MYD88-mutated Waldenstrom macroglobulinemia, activated B-cell diffuse large B-cell lymphoma, leukemia. Exemplary proliferative diseases include cancers (i.e., “malignant neoplasms”), benign neoplasms, angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary original tumor to another organ or tissue and is typically identifiable by the presence of a “secondary tumor” or “secondary cell mass” of the tissue type of the primary original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), MYD88-mutated Waldenstrom macroglobulinemia, activated B-cell (ABC) diffuse large B-cell lymphoma, mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

The “mitochondrial permeability transition pore” (mPTP) is a protein within the inner membrane of the mitochondria that is permeable to molecules less than 1.5 kDa. The mPTP is usually closed, but may be opened under certain conditions including mitochondrial matrix Ca²⁺ accumulation, adenine nucleotide depletion, increased phosphate concentration, or oxidative stress. The opening of the mPTP pore is associated with apoptosis. Cyclophilins (e.g., CypD) can regulate the opening and closing of the mPTP.

“Autophagy” relates to a self-degradation maintenance process in a cell where the cell breaks down and destroys old, damaged, or abnormal proteins and/or other substances in its cytoplasm, to keep the cell functioning properly. Three exemplary types of autophagy include: pexophagy, autophagy selective for degradation of peroxisomes; mitophagy, autophagy selective for degradation of mitochondria; and xenophagy, autophagy selective for degradation of intracellular bacteria and viruses. Exemplary diseases and/or conditions associated with autophagy include, but are not limited to, neurodegenerative disease (e.g., Alzheimer's disease, multiple sclerosis, Parkinson's disease, Huntington's disease, amyotrophic lateral sclerosis), infection (e.g., infection by bacteria, viruses, microbes), cancer, aging, and heart disease.

“Aging” is a phenomenon characterized by progressive accumulation of dysfunctional proteins and damaged organelles at the cellular level. Increased malfunction in the cellular regulatory processes for maintenance, repair, and turnover of defective protein structures is thought to be associated with aging. With age, autophagy activity appears to decline and contributes to the accumulation of cellular components associated with aging.

“Cardiovascular disease” refers any disease or disorder relating to the heart and blood vessels, including, but not limited to hypertension (high blood pressure), coronary heart disease (heart attack), cerebrovascular disease (stroke), peripheral vascular disease, heart failure, rheumatic heart disease, congenital heart disease, and cardiomyopathies. In certain embodiments, cardiovascular disease is caused by “oxidative stress” (e.g., increased production of reactive oxygen species (ROS)). In certain embodiments, a “cardiovascular” condition is an ischemia-reperfusion injury.

“Ischemia-reperfusion injury” refers to the injury characterized by cellular dysfunction and death, after restoration of blood flow to ischemic tissues. “Ischemia” refers to a state where the tissues have a lower than normal blood supply (e.g., resulting in a deficiency of oxygen, glucose, and other materials required for metabolism). “Reperfusion injury” refers to the restoration of blood flow to damaged tissues (e.g., damaged myocardium) which triggers additional ischemic cellular damage.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

A “cyclophilin” is a protein from the cyclophilin family, a group of 17 proteins characterized by a highly conserved peptidyl-prolyl-isomerase domain. A majority of the cyclophilin family members possess enzymatic activity to convert between cis and trans proline-peptide bonds. Cyclophilin D (CypD) acts as a regulator of the mitochondrial permeability transition pore (mPTP), a channel across the inner mitochondrial membrane where prolonged opening results in cell necrosis. For CypD, an exemplary sequence from GenBank is: P30405.1 (Homo sapiens). For CypE, an exemplary sequence from GenBank is: Q9UNP9.1 (Homo sapiens). Other exemplary cyclophilins include, but are not limited to, CypB, CypC, CypD, CypH, and Cyp40.

### BRIEF DESCRIPTION OF THE DRAWINGS

FIGS. 1A-1D show biphenyl dicarboxylates achieve strong CypD selectivity. FIG. 1A shows the structure of B52 and B53. FIG. 1B shows the prolyl-isomerase cyclophilin inhibition profile for B52 and B53. FIG. 1C shows the co-crystal structures of B52 (PDB ID 7THD, 1.16 Å resolution) and B53 (PDB ID 7THF, 1.10 Å resolution) bound to CypD, viewing the S2 pocket. Dashes indicate predicted hydrogen bonds. FIG. 1D is a list of residues on the far side of the S2 pocket of cyclophilins that are proximal to the ligand carboxylates. Both compounds retain potency similar to that of mono-carboxylate B23, while enhancing selectivity for CypD over CypA, CypB, CypE, and PPIL1. The malonic and glutaric acids of B52 and B53, respectively, position the carboxylate in a similar pose as B23 (FIG. 7B), while presenting a second carboxylate to the S123 residue. B52 forms a predicted hydrogen bond with the peptide backbone of S123-R124. R124 is pushed out of the S2 pocket, consistent with other macrocycles containing large S2-binding groups such as B1. B52 and B53 achieve selectivity over CypA and CypB through charge repulsion with a glutamate at the analogous 123 position, while creating a steric clash with CypE's lysine at this same position. Values and error bars reflect mean±SEM of three technical replicates.

FIGS. 2A-2E show inhibition potency is dependent on favorable interactions with S2 pocket residues. FIG. 2A shows dose response curves for B52 against CypD, CypB, and CypB E121S mutant, with residue tables for dicarboxylate proximal residues. FIG. 2B shows dose response curves for B52 against CypD, CypA, and CypA E81S/K82R double mutant, with residue tables for dicarboxylate proximal residues. Mutated residues are underlined. B52 inhibits both CypB and CypA mutants that have the appropriate ‘CypD’ gatekeeper residues at similar potency values compared wild-type CypD. FIG. 2C shows the dose response curves for B32 against wild-type CypD and CypD mutants shown in the table. Two mutations to remove positively charged residues K118 and R124 restore inhibition potency of amine derivative B32.

FIG. 2D is the structure of B32. FIG. 2E shows that inserting an amine with a net positive charge results in >1,000-fold reduction in CypD potency for B32, presumably due to charge-charge repulsion at the K118 residue. Values and error bars reflect mean±SEM of three technical replicates.

FIG. 3A-3F shows Cy5-conjugated cyclophilin D inhibitors delay calcium induced opening of the PTP in isolated mouse liver mitochondria. The calcium retention capacity of mitochondria was determined in isolated mouse liver mitochondria (0.5 μg/ml) in response to pulses of 60 μM CaCl₂) in the presence of the indicated CypD inhibitors (or inactive enantiomers). Concentrations used were 2 μM CsA, 10 μM B52-Cy5, 10 μM*B52-Cy5, 20 μM B53-Cy5, 20 μM*B53-Cy5. Mitochondrial uptake of extramitochondrial Ca²⁺ was assessed by monitoring the fluorescence of Calcium-green 5n, depicted in arbitrary units (A.U.). The rapid increase in fluorescence after several pulses of Ca²⁺ are taken up corresponds to mitochondrial Ca²⁺ release through mPTP opening. FIGS. 3A-3C are traces from a representative experiment, with all assays performed on the same mitochondrial preparation and day. FIG. 3D shows the quantitation of calcium retention capacity (CRC) reported as the ratio of the number of Ca²⁺ pulses required to induce mPTP opening in the listed condition relative to DMSO control conditions on the same mitochondrial preparation and day. Data are from three independent experiments/mitochondrial isolations. Error bars represent SD. *P<0.05, **P<0.005 by Student's t test, one-sided. FIG. 3E shows the structures of Cy5 conjugated CypD selective inhibitors and prodrugs. FIG. 3F shows fluorescence microscopy of HeLa cells co-incubated with ester prodrugs B52-Et-Cy5 and B53-Et-Cy5 (red), co-stained with mitochondrial (green) and nuclear (blue) dyes (Mitotracker Green and Hoechst 33342, respectively). Both prodrugs show good plasma membrane permeability and co-localization with Mitotracker Green.

FIGS. 4A-4E show aryl-carbonyl boronic acid C3A achieves selective inhibition of CypE. FIG. 4A shows the structure of C3A. FIG. 4B shows the C3A fluorescence polarization (FP) competition with A26-Fl against cyclophilins with S2 pocket lysines. FIG. 4C depicts the prolyl-isomerase screen of C3A, showcasing potency and selectivity for CypE. FIG. 4D shows the mass spectrometry trace of CypE incubated with C3A and reduced with sodium cyanoborohydride. C3A shows an adduct consistent with CypE+amine-H₂O (+806 Da), the result of iminoboronate formation followed by reductive amination. The mass of CypE is 20,708 Da. The CypE preparation also included N-terminal gluconoylation. FIG. 4E shows prolyl-isomerase inhibition by C3A against CypE S2 pocket lysine to alanine mutants. For the FP assay, the y-axis is normalized to internal control wells containing A26-Fl only (100%) and A26-Fl with cyclophilin (0%). Values reflect mean of three technical replicates and error bars reflect SD of individual assays at one doseFor the prolyl-isomerase assay in FIG. 4C and the CypE wild-type dose response curve in FIG. 4E, IC50 values reflect mean±SD of four independent replicates (each comprising three technical replicates). Graph shows a representative single independent replicate (Independent replicate 3 is shown, containing three technical replicates) with data points and error bars reflecting mean±SD of individual assays at one dose. For the prolyl-isomerase assay in FIG. 4E, IC50 values for the CypE mutants reflect mean±SEM of three technical replicates, while data points and error bars reflect mean±SD of individual assays at one dose.

FIGS. 5A-5E show the residue K118 is important for potent CypD inhibition with carboxylate-containing inhibitors. Dose response curves of CypD, CypD K118A, and CypD K118E with FIG. 5A, CsA; FIG. 5B, B23; FIG. 5C, B25; FIG. 5D, B52; and FIG. 5E, B53. CsA as an active site ligand does not show appreciable changes in potency for CypD mutants compared to wild-type CypD. A salt bridge was observed in each crystal structure between a carboxylate on B23, B25, B52, and B53, and residue K118 (FIG. 1C, FIG. 7B). Mutation to a neutral alanine results in a 6- to 20-fold decrease in potency compared to wild-type CypD. Mutation to a negatively charged glutamate results in a 20- to 100-fold decrease in potency compared to wild-type CypD. Values and error bars reflect mean±SEM of three technical replicates. Graphs show a representative single independent replicate (Independent replicate 1 is shown, containing three technical replicates) with data points and error bars reflecting mean±SD of individual assays at one dose. All other IC50 values reflect mean±SEM of three technical replicates, with data points and error bars reflecting mean±SD of individual assays at one dose.

FIGS. 6A-6E show that the S123 gatekeeper position dictates inhibitor potency. Dose response curves of CypD, CypB, and CypD S123E, shown for FIG. 6A, CsA; FIG. 6B, B23; FIG. 6C, B25. FIG. 6D, B52; and FIG. 6E, B53. CsA as an active site ligand does not show appreciable differences in potency between wild-type CypD, wild-type CypB, and CypD S123E. Wild-type CypB and wild-type CypD share identical S2 pockets with the exception of the analogous CypD S123 residue, which is a glutamate in CypB. CypD S123E thus provides the same S2 pocket as CypB WT, resulting in almost identical potencies for these two proteins for all inhibitors shown. For CypD and CypB wild-type IC50 data with B52 and B53, values reflect mean±SD of four independent replicates (each comprising three technical replicates). Graphs show a representative single independent replicate (Independent replicate 1 is shown, containing three technical replicates) with data points and error bars reflecting mean±SD of individual assays at one dose. All other IC50 values reflect mean±SEM of three technical replicates, with data points and error bars reflecting mean±SD of individual assays at one dose.

FIGS. 7A-7B show carboxylate-containing S2 binding moieties induce K118 side-chain and S123 backbone migration. FIG. 7A shows the co-crystal structure of B2 (PDB ID 7TGV, 1.46 Å resolution) bound to CypD, viewing the S2 pocket. FIG. 7B shows the co-crystal structure of B23 (PDB ID 7TH7, 1.18 Å resolution) and B25 (PDB ID 7THC, 1.57 Å resolution) bound to CypD, viewing the S2 pocket. The side-chain of K118 typically is oriented away from the S2 pocket, as shown in co-crystal structures that do not contain carboxylate ligands, such as that of B2. A properly placed carboxylate group (B23) migrates K118's side-chain into the S2 pocket, forming a salt bridge along with a hydrogen bond with S119's peptide backbone (dashes). An S123 loop migration occurs when carboxylate-containing biphenyl groups bind deep into the S2 pocket. All properly placed carboxylate-containing ligands (including B52 and B53) induce the same K118 and S123 conformational change. Dicarboxylates such as B52 and B53 also present the second carboxylate towards the S123 gatekeeper residue, the former exhibiting a hydrogen bond with the S123-R124 peptide backbone.

FIGS. 8A-8E show prolyl isomerase inhibition activity on CypD gatekeeper mutants. Dose response curves of CypD and CypD S123E, CypD R124A, and CypD R124K mutants, shown for FIG. 8A, CsA; FIG. 8B, B23; FIG. 8C, B25; FIG. 8D, B52; and FIG. 8E, B53. CsA as an active site ligand does not show appreciable differences in potency between wild-type CypD and CypD mutants. Each carboxylate-containing ligand shows a modest drop in potency for CypD S123E compared to wild-type CypD, indicating a transient interaction with this residue. Little to no drop-in potency for R124A or R124K mutants suggest that the carboxylates for each compound do not interact with this residue directly. For CypD wild-type IC50 data with B52 and B53, values reflect mean±SD of four independent replicates (each comprising three technical replicates). Graphs show a representative single independent replicate (Independent replicate 1 is shown, containing three technical replicates) with data points and error bars reflecting mean±SD of individual assays at one dose. All other IC50 values reflect mean±SEM of three technical replicates, with data points and error bars reflecting mean±SD of individual assays at one dose.

FIGS. 9A-9C show B23 derivatives that can simultaneously interact with CypD residues K118 and S119 and present a carboxylate near gatekeeper residues. Structure and cyclophilin inhibition dose response data for FIG. 9A, B51; FIG. 9B, B52; and FIG. 9C, B53. Combination of a nitrile group with the carboxylate resulted in loss of potency for B51, but a similar selectivity profile as B23. A large improvement in selectivity was observed when dicarboxylate groups were used. For both B52 and B53, one carboxylate is oriented to interact with K118 and S119, while the second carboxylate is directed towards the gatekeeper residue S123 of CypD (FIG. 1C). For B52 and B53, four independent replicates (each comprising three technical replicates) are shown, with their respective fitted values that reflect mean±SEM of three technical replicates. Data points and error bars reflect mean±SD of individual assays at one dose. Tables show final reported IC50 values that reflect mean±SD of four independent replicates, who's values are used for the entirety of this work. Independent replicate 1 was collected with a separately synthesized compound batch than independent replicates 2, 3, and 4. For B51, IC50 values reflect mean±SEM of three technical replicates, with data points and error bars reflecting mean±SD of individual assays at one dose.

FIGS. 10A-10E show the CypB E121S gatekeeper mutant is inhibited more potently compared to wild-type CypB by carboxylate-containing inhibitors. Dose response curves of CypD, CypB, and CypB E121S, shown for FIG. 10A, CsA; FIG. 10B, B23; FIG. 10C, B25; FIG. 10D, B52; and FIG. 10E, B53. CsA as an active site ligand does not show appreciable differences in potency between wild-type cyclophilins and the tested cyclophilin mutants. For carboxylate-containing inhibitors, wild-type CypB shows attenuated potency compared to wild-type CypD. Carboxylate-containing compounds inhibit wild-type CypD and CypB E121S equipotently, as this CypB mutant contains S2 pocket residues that mimic those in CypD's S2 pocket. For CypD and CypB wild-type IC50 data with B52 and B53, values reflect mean±SD of four independent replicates (each comprising three technical replicates). Graphs show a representative single independent replicate (Independent replicate 1 is shown, containing three technical replicates) with data points and error bars reflecting mean±SD of individual assays at one dose. All other IC50 values reflect mean±SEM of three technical replicates, with data points and error bars reflecting mean±SD of individual assays at one dose.

FIGS. 11A-11E show the CypA E81S/K82R gatekeeper mutant is inhibited more potently compared to wild-type CypA with carboxylate-containing inhibitors. Dose response curves of CypD, CypA, and CypA E81S/K82R, shown for FIG. 11A, CsA; FIG. 11B, B23; FIG. 11C, B25; FIG. 11D, B52; FIG. 11E, B53. CsA as an active site ligand does not show appreciable changes in potency between the wild-type cyclophilins and mutants. For carboxylate-containing inhibitors, wild-type CypA shows attenuated potency compared to wild-type CypD. Carboxylate-containing compounds inhibit wild-type CypD and CypA E81S/K82R equipotently, as this CypA mutant contains S2 pocket residues that mimic those in CypD's S2 pocket. For CypD and CypA wild-type IC50 data with B52 and B53, values reflect mean±SD of four independent replicates (each comprising three technical replicates). Graphs show a representative single independent replicate (Independent replicate 1 is shown, containing three technical replicates) with data points and error bars reflecting mean±SD of individual assays at one dose. All other IC50 values reflect mean±SEM of three technical replicates, with data points and error bars reflecting mean±SD of individual assays at one dose.

FIGS. 12A-12C show the cyclophilin binding profiles using fluorescence polarization of fluorescein-labeled macrocycles. Each cyclophilin was titrated against 0.5 nM fluorescein-labeled macrocycle FIG. 12A, A26-Fl; FIG. 12B, B52-Fl; or FIG. 12C, B53-Fl. Trends for selectivity follow those observed in the prolyl isomerase assay. Cyclophilins in the legend below the dashed line are either prolyl-isomerase inactive, or require much higher concentrations to observe prolyl-isomerization in vitro. K_{d} values and error bars reflect mean±SEM of three technical replicates. Data points and error bars reflect mean±SD of individual assays at one dose.

FIGS. 13A-13D show the prolyl isomerase inhibition of other CypD selective inhibitors. Structure and cyclophilin inhibition dose response data for FIG. 13A, B52-A; FIG. 13B, B53-A; FIG. 13C, *B52-A; and FIG. 13D, *B53-A. B52-A and B53-A retain the same selectivity profile as their associated analogs B52 and B53, respectively, with a 2- to 3-fold decrease in potency. Enantiomers *B52-A and *B53-A show no substantial inhibition activity on any cyclophilins tested. IC₅₀ values reflect mean±SEM of three technical replicates. Data points and error bars reflect mean±SD of individual assays at one dose.

FIGS. 14A-14D show the prolyl isomerase inhibition by CypD-selective inhibitors used in mitochondrial models of mPTP. Structure and cyclophilin inhibition dose response data for FIG. 14A, B52-Cy5; FIG. 14B, B53-Cy5; FIG. 14C, *B52-Cy5; and FIG. 14D, *B53-Cy5. B52-Cy5 and B53-Cy5 retain the same selectivity profile as their associated analogs, B52 and B53, respectively. Enantiomers *B52-Cy5 and *B53-Cy5 show no substantial inhibition profile on any cyclophilins tested. IC₅₀ values reflect mean±SEM of three technical replicates. Data points and error bars reflect mean±SD of individual assays at one dose.

FIGS. 15A-15B represent additional replicates for calcium retention assay in mitochondria. The calcium retention capacity was determined in additional preparations of isolated mouse liver mitochondria (0.5 μg/mL) in response to pulses of 60 μM CaCl₂) in the presence of the indicated CypD inhibitors (or inactive enantiomers). Concentrations used were 2 μM CsA, 10 μM B52-Cy5, 10 μM*B52-Cy5, 20 μM B53-Cy5, and 20 μM*B53-Cy5. Mitochondrial uptake of extra-mitochondrial Ca²⁺ was assessed by monitoring the fluorescence of Calcium-green 5n, depicted in arbitrary units (A.U.). The rapid increase in fluorescence after several pulses of Ca²⁺ are taken up corresponds to mitochondrial Ca²⁺ release via mPTP. FIG. 15A shows all assays performed on the same mitochondrial preparation and day. FIG. 4B shows all assays performed on a different mitochondrial preparation and day.

FIGS. 16A-16D show the fluorescence polarization competition with A26-Fl against lysine-containing cyclophilins. Structure and FP competition with 0.5 nM A26-Fl dose response data for FIG. 16A, C1A; FIG. 16B, C2A; FIG. 16C, C3A; and FIG. 16D, C4A. C3A shows selectivity for CypE, while regiosiomer C4A and acetyl derivatives C1A and C2A show attenuated binding to CypE. Y-axes are normalized to internal control wells containing A26-Fl only (100%) and A26-Fl with cyclophilin (0%). Data points and error bars reflect mean±SEM of three technical replicates. Kᵢ values reflect the mean of three technical replicates.

FIGS. 17A-17C show the fluorescence polarization competition with A26-Fl against CypE with control compounds C5A and C6A. Structures of compounds FIG. 17A, C5A; and FIG. 17B, C6A. FIG. 17C, Dose response of C3A, C5A, and C6A against CypE. Removal of either the aldehyde or boronic acid from C3A results in attenuated inhibition of CypE. Y-axes are normalized to internal control wells containing A26-Fl only (100%) and A26-Fl with cyclophilin (0%). Data points and error bars reflect mean±SEM of three technical replicates. Kᵢ values reflect mean of three technical replicates.

FIGS. 18A-18E show the prolyl Isomerase inhibition by C1A, C3A, C5A, and C6A. Structure and cyclophilin inhibition dose response data for FIG. 18A, C1A; FIG. 18B, C3A; FIG. 18C, C5A; and FIG. 18D, C6A. C3A shows good selectivity and potency for CypE, while retaining only the aldehyde in C4A or the boronic acid in C5A results in loss in CypE potency and promiscuous cyclophilin inhibition. Replacing the aldehyde with an acetyl group in C1A also results in loss of potency and selectivity for CypE. FIG. 18E depicts the dose response curves of C3A, C4A, and C5A against CypE, showing the importance of both parts of the covalent warhead for CypE potency. For C3A, four independent replicates (each comprising three technical replicates) are shown, with their respective fitted values that reflect mean±SEM of three technical replicates. Data points and error bars reflect mean±SD of individual assays at one dose. Table shows final reported IC₅₀ values that reflect mean±SD of four independent replicates for CypD, CypA, CypB, CypE, Cyp40 and PPIL1, and mean±SD of three independent replicates (Independent replicates 2-4) for CypC, CypG, CypH, NKTR, and PPWD1. The Table IC₅₀ values are used for the entirety of this work. Independent replicate 1 was collected with a separately synthesized compound batch than was used in independent replicates 2, 3, and 4.

FIGS. 19A-19B show the mass spectrometry analysis of C3A covalent modification on 13 cyclophilins. FIG. 19A shows the mass spectrometry analysis of lysine covalent modification. FIG. 19B shows the same analysis as described in FIG. 19A, but after treatment with NaCNBH₃ to trap iminoboronate as lysine-modified secondary amine, highlighting relevant m/z regions. Primary protein peaks are shown along with expected region of C3A modification (+824: C3A modification non-reductive, represented as a dashed line for the most abundant ion peak(s) in panel a, +806: C3A —H₂O reductive amination covalent adduct, represented as a dashed line on cyclophilins with no observed covalent modification for the most abundant ion peak in FIG. 19B). Small amounts of covalent modification were observed for PPWD1, however with a diminished peak intensity compared to CypE's+806 covalent adduct, suggesting minimal adduct formation in solution. Some C-terminal truncation for CypB of residues that are not anticipated to affect active site or S2 pocket integrity, prolylisomerase catalytic activity, or inhibitor binding was observed. A mixture of CypH, CypG and PPIL2 constructs that include various cleaved and non-cleaved His-tag forms was also observed, with representative mass spectra shown for each primary peak. N-terminal His-tag or cleaved cyclophilin constructs are not anticipated to affect active site or S2 pocket integrity, prolylisomerase catalytic activity, or inhibitor binding. Various recombinantly expressed constructs show N-terminal gluconoylation. Some proteins when co-treated with NaCNBH₃ showed +14 mass off the primary peak not present in non-reductive conditions (see CypC, CypH, NKTR, Cyp40, and PPIL1) suggesting various methylation events via reductive amination.

FIGS. 20A-20B show mass spectroscopy analysis of compounds co-incubated with CypE and CypE mutants. FIG. 20A shows mass spectroscopy analysis of lysine covalent modification of CypE with C3A, C5A, and C6A. FIG. 20B shows mass spectroscopy analysis of lysine covalent modification after treatment with NaCNBH₃ to trap the iminoboronate as a reduced lysine-linked secondary amine, highlighting relevant m/z regions. C3A and C5A show covalent modification (+779 and +806, respectively) of CypE after reductive amination with NaCNBH₃.

FIG. 21 shows the cyclophilin S2 pocket residues occupying the S2 pockets of all 17 human cyclophilin isoforms with accompanying protein and gene identifier. Primary gatekeeper residues where residues are the least conserved between cyclophilin isoforms are 123, 124, and 145. Residue 118 is another site of high diversity. Residue numbering is in reference to CypD.

FIG. 22 shows high-resolution mass spectrometry results for intermediates reported in this work. Experiments and formula confirmation were performed by Harvard University's Center for Mass Spectroscopy.

FIG. 23 shows high-resolution mass spectrometry results for macrocycles reported in this work. Experiments and formula confirmation were performed by Harvard University's Center for Mass Spectroscopy.

FIG. 24 shows plasmids and primers used for USER cloning of CypD mutant expression constructs.

FIG. 25 shows plasmids and primers used for cloning of CypA, CypB, and CypE mutant expression constructs.

FIG. 26 shows plasmids used for recombinant expression of cyclophilin proteins.

FIG. 27 shows concentrations of cyclophilins used in competition anisotropy binding assay with A26-Fl.

FIG. 28 shows crystal diffraction statistics for all CypD-inhibitor co-crystal structures reported in this work. Statistics for the highest-resolution shell are shown in parentheses. *Data was collected to 1.18 Å resolution but cut off for refinement to 1.3 Å resolution to improve completeness.

FIG. 29 shows an analysis of CypD-selective prolyl isomerase inhibition based on S2 pocket containing residues. IC₅₀ values for each cyclophilin are accompanied by fold difference normalized to IC₅₀^{CypD}. Residues listed next to each cyclophilin are proximal residues near the carboxylate-containing ligands. CsA does not bind the S2 pocket and shows almost no cyclophilin isoform selectivity, while B2's large biphenyl group exhibits selectivity over cyclophilins with sterically occluded S2 pockets. Further selectivity over CypC, Cyp40, and PPIL1 is achieved through interactions between CypD's K118 residue and carboxylate-containing ligands such as B23. Installation of dicarboxylates in B52 and B53 result in CypD selectivity by presenting a second carboxylate near the analogous S123 position on CypD. Potency for CsA, B2, and B23 values reflect mean of three technical replicates. Potency for B52 and B53 values reflect mean of four independent replicates.

FIG. 30 shows the calculated abundance of each human cyclophilin family member from paxdb^{4.1}. CypD is the third most abundant cyclophilin, while CypB and CypA are the second and first, respectively⁴⁷. CypA is one of the most abundantly expressed proteins in human cells. Therefore, selectivity over CypA is useful for CypD selective inhibition in biological models. Compounds B52/B53, B32, and C3A in vitro show >100-fold selectivity for CypD, CypD K118E/R124A, and CypE, respectively, over CypA.

FIG. 31 shows a general scheme of the solid-phase peptide synthesis of macrocycle inhibitors.

FIG. 32 shows an overview of cyclophilin-selective inhibitors. Inhibitor IC₅₀ values from prolyl-isomerase inhibition on the targeted cyclophilin are highlighted in dashed boxes. The inhibition potencies against all other cyclophilins are normalized to this IC₅₀ value and are shown as a fold-difference. Boxes are shaded according to the fold-difference value. Each cyclophilin is inhibited selectively due to the identity of its unique S2 pocket residues and the S2 pocket binding moiety of the inhibitor.

FIGS. 33A-33I show quantification of mitochondrial localization in HeLa cells by fluorescence microscopy of Cy5-conjugated compounds. HeLa cells were treated with Cy5-conjugated compounds and analyzed for total identifiable Cy5 spots (FIG. 33A); Cy5 spots per cell (FIG. 33B); mean fluorescence intensity of identified Cy5 spots (FIG. 33C); sum of fluorescence intensity of identified Cy5 spots (FIG. 33D); mean Cy5 fluorescence intensity per cell (FIG. 33E); sum of Cy5 fluorescence in all measured cells (FIG. 33F); percent of Cy5 spots that overlap >70% with Mitotracker Green co-stain (FIG. 33G); fluorescence intensity of Cy5 spots that overlap >70% with Mitotracker Green (FIG. 33H); and values of data shown in FIGS. 33A-33H (FIG. 33I). Values and error bars reflect mean±SD of three technical replicates.

FIGS. 34A-34H show hydrolysis of ester prodrug CypD inhibitors. Compounds were evaluated for their ability to be hydrolyzed from di-ester to mono-ester, or to di-acid CypD inhibitors. Each reaction was analyzed by LC-MS, and ion abundances for each is shown as a percent of the total sum. These were conducted under conditions of: FIG. 34A: Tris-HCl buffer only; FIG. 34B: 250 nM carboxylesterase 1 (CES1); FIG. 34C: 250 nM carboxylesterase 2 (CES2); FIG. 34D: incubated with A549 cells for 48 hours and intracellular fraction isolated;

FIG. 34E: incubated with HeLa cells for 48 hours and intracellular fraction isolated; FIG. 34F: incubated with HEK293T cells for 48 hours and intracellular fraction isolated; FIG. 34G: incubated with MEFs for 48 hours and intracellular fraction isolated; FIG. 34H: incubated with HepG2 cells for 36 hours and intracellular fraction isolated. Esters show good stability in buffer and are only cleaved under esterase conditions, or intracellularly, with B52-Et-Cy5 showing the most rapidly hydrolyzed esters. Values and error bars reflect mean±SD of three technical replicates.

FIGS. 35A-35F show representative images of Cy5-conjugated CypD inhibitor localization in HeLa cells. HeLa cells were co-treated with Cy5 conjugated compounds shown in FIG. 35A, and co-stained with Mitotracker Green, Hoechst 33342 and imaged by fluorescence microscopy. Images include the following channels; FIG. 35B: Hoechst 33342 nuclear stain; FIG. 35C: Mitotracker Green mitochondrial stain; FIG. 35D: Deep red channel showing Cy5-conjugated compound; FIG. 35E: overlay of Mitotracker Green and Cy5 channels; FIG. 35F: overlay of Hoechst 33342, Mitotracker Green, and Cy5 channels. Cy5-enAc (127) and ester derivatives B52-Et-Cy5, B53-Et-Cy5, *B52-Et-Cy5, and *B53-Et-Cy5 show both good mitochondrial localization via overlap with Mitotracker Green and sufficient mitochondrial fluorescence. In contrast, dicarboxylate derivatives B52-Cy5, B53-Cy5, *B52-Cy5, and *B53-Cy5 show poor mitochondrial localization and fluorescence. Scale bars, 200 m.

FIG. 36 shows CypD inhibition of Cy5-conjugated CypD inhibitors. CypD prolyl-isomerase inhibition of Cy5-conjugated compounds is shown. Compounds with correct stereochemistry and exposed dicarboxylate moieties are potent CypD inhibitors. Cy5-enAc alone does not inhibit CypD. IC50 values reflect mean±SEM of three technical replicates. Data points and error bars reflect mean±SD of individual assays at one dose.

FIGS. 37A-37B show CypE mutant FP analysis. FIG. 37A shows protein titration against 0.5 nM A26-Fl. K_{d} values and error bars reflect mean±SEM of three technical replicates. Data points and error bars reflect SD of individual assays at one dose. FIG. 37A shows FP competition with 0.5 nM A26-Fl dose response data C3A CypE proteins. C3A has significantly attenuated binding for only the K217A mutant compared to wild-type. Y-axes are normalized to internal control wells containing A26-Fl only (100%) and A26-Fl with cyclophilin (0%). Data points and error bars reflect mean±SD of three technical replicates. Kᵢ values reflect the mean of three technical replicates.

FIGS. 38A-38B show that prolyl isomerase inhibition screening of CypE lysine mutants reveals K217 residue as site of C3A covalent modification. Dose response curves of CypD, CypE K212A, CypE K217A, and CypE K218A shown for CsA (FIG. 38A) and C3A (FIG. 38B). CsA as an active site ligand does not show appreciable differences in potency between wild-type CypE and the tested CypE mutants. Loss of C3A inhibition potency for only the K217A mutant compared to wild-type or K212A and K218A mutants indicates that K217 is being covalently modified by C3A. For wild-type CypE IC₅₀ data with C3A, values reflect mean±SD of four independent replicates (each comprising three technical replicates). Graphs show a representative single independent replicate (Independent replicate 3 is shown, containing three technical replicates) with data points and error bars reflecting mean±SD of individual assays at one dose. All other IC₅₀ values reflect mean±SEM of three technical replicates, with data points and error bars reflecting mean±SD of individual assays at one dose.

FIG. 39 shows electron densities of ligands from CypD-inhibitor co-crystal structures.

FIGS. 40A-40B show SDS-PAGE of recombinantly expressed cyclophilin proteins. FIG. 40A shows representative samples of the following: Lane 1-Blank; Lane-2: Protein Ladder; Lane 3-GSSHHHHHHSSGLVPRGS-NKTR(7-179); Lane 4-Mixture of GSSHHHHHHSSGLVPRGS-CypG(1-179), S-CypG(1-179), CypG(2-179), CypG(3-179); Lane 5-GSSHHHHHHSSGLVPRG-CypC(24-212); Lane 6-GSSHHHHHHSSGLVPRGS-PPWD1(473-646); Lane 7-Mixture of KSSHHHHHHENLYFQSNA-CypH(1-177), A-CypH(1-176), and SNA-CypH(1-176); Lane 8-KSSHHHHHHENLYFQSNA-Cyp40(1-183); Lane 9-KSSHHHHHHENLYFQSNA-PPIL1(1-166); Lane 10-Mixture of GS-PPIL2(280-457), GSSHHHHHHSSGLVPRGS-PPIL2(280-457), GSPPIL2(280-457)+G, and GSSHHHHHHSSGLVPRGS-PPIL2(280-457)+G; Lane 11-KSSHHHHHHENLYFQSNA-CypA (1-165); Lane 12-SNA-CypA E81S/K82R (1-165); Lane 13-Mixture of SHHHHHHENLYFQSNA-CypB (34-198) and HENLYFQSNA-CypB (34-216); Lane 14-SNA-CypB E121S (34-216); Lane 15-blank. FIG. 40B shows representative samples of the following: Lane 1-Blank; Lane-2: Protein Ladder; Lane 3-GSSHHHHHHSSGLVPRGS-CypE(131-301); Lane 4-GS-CypE K212A (131-301); Lane 5-GS-CypE K217A (131-301); Lane 6-GS-CypE K218A (131-301); Lane 7-SNA-PPIL3(1-161); Lane 8-MKSSHHHHHHENLYFQSNA-CypD(45-207); Lane 9-MKSSHHHHHHENLYFQSNA-CypD K118A (45-207); Lane 10-MKSSHHHHHHENLYFQSNA-CypD K118E (45-207); Lane 11-MKSSHHHHHHENLYFQSNA-CypD S123E (45-207); Lane 12-MKSSHHHHHHENLYFQSNA-CypD R124A (45-207); Lane 13-MKSSHHHHHHENLYFQSNA-CypD R124K (45-207); Lane 14-MKSSHHHHHHENLYFQSNA-CypD K118E/R124A (45-207); Lane 15-blank. All wild-type recombinant proteins were also verified by LC-MS.

### DETAILED DESCRIPTION OF CERTAIN EMBODIMENTS OF THE INVENTION

The present disclosure provides inhibitors (e.g., selective inhibitors) of cyclophilins (e.g., CypB, CypC, CypD, CypE, CypG, CypH, Cyp40, PPWD1, PPIL1, NKTR). In certain embodiments, the inventive compounds inhibit the activity of CypD. In certain embodiments, the inventive compounds inhibit the activity of CypE. The present disclosure further provides methods of using the compounds described herein, e.g., as biological probes to study the inhibition of the activity of a cyclophilin (e.g., CypB, CypC, CypD, CypE, CypG, CypH, Cyp40, PPWD1, PPIL1, NKTR), and as therapeutics, e.g., in the treatment and/or prevention of diseases associated with the overexpression and/or aberrant activity of the cyclophilin (e.g., CypB, CypC, CypD, CypE, CypG, CypH, Cyp40, PPWD1, PPIL1, NKTR). In certain embodiments, the compounds covalently modify a cyclophilin (e.g., CypD, CypE). In certain embodiments, the diseases treated and/or prevented with a compound described herein are associated with CypD. In certain embodiments, the diseases treated and/or prevented with a compound described herein are associated with CypE. In certain embodiments, the diseases treated and/or prevented include, but are not limited to, neurodegenerative disease (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases, or other diseases associated with cyclophilins (e.g., CypD, CypE)). The neurodegenerative diseases include, but are not limited to, Alzheimer's disease, multiple sclerosis, Parkinson's disease, Huntington's disease, and amyotrophic lateral sclerosis. The metabolic disorders include, but are not limited to, obesity and diabetes. The proliferative diseases include, but are not limited to, cancer. Other treated conditions include conditions associated with autophagy and/or aging. The cardiovascular diseases and conditions include, but are not limited to, ischemia-reperfusion injury, stroke, coronary artery disease, and heart attack. In certain embodiments, the condition is a mitochondrial disease, for example, a condition and/or disease associated with the regulation of the mitochondrial permeability transition pore (mPTP) and/or CypD. In certain embodiments, the disease and/or condition is associated with CypD and is ischemia-reperfusion injury (IRI), Alzheimer's disease, Huntington's disease, multiple sclerosis (MS), Parkinson's disease, amyotrophic lateral sclerosis (ALS), X-linked adrenoleukodystrophy (X-ALD), liver cirrhosis, or diabetes. In certain embodiments, the disease and/or condition is ischemia-reperfusion injury (IRI), Alzheimer's disease, Huntington's disease, multiple sclerosis (MS), Parkinson's disease, amyotrophic lateral sclerosis (ALS), X-linked adrenoleukodystrophy (X-ALD), liver cirrhosis, or diabetes. Also provided by the present disclosure are pharmaceutical compositions, kits, methods, and uses of a compound of Formula (I-A), (I-B), or (I-C) as described herein.

### Compounds

Certain aspects of the present disclosure relate to the compounds described herein. The compounds described herein may be useful in treating and/or preventing diseases and/or conditions mitochondrial diseasesin a subject, or inhibiting the activity of a cyclophilin (e.g., CypD, CypE) in a subject, cell, tissue, or biological sample. In certain embodiments, the diseases treated and/or prevented with a compound described herein are associated with CypD. In certain embodiments, the diseases treated and/or prevented with a compound described herein are associated with CypE. In certain embodiments, a compound described herein is a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof. In certain embodiments, a compound described herein is a compound of Formula (I-A), (I-B), or (I-C), or a pharmaceutically acceptable salt thereof.

In certain embodiments, a compound described herein is of Formula (I-A):

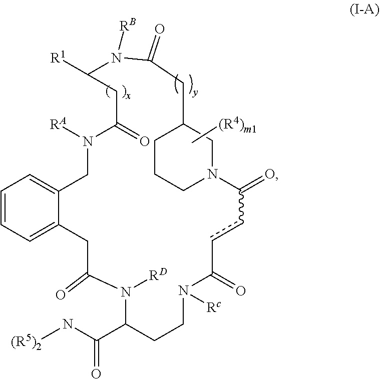

or a pharmaceutically acceptable salt, solvate, hydrate, polymorph, co-crystal, tautomer, stereoisomer, isotopically enriched form, prodrug, or mixture thereof, wherein:
each instance of [symbol] is independently a single or double C—C bond, as valency permits, wherein when [symbol] is a double C—C bond adjacent to [symbol] , then [symbol] indicates that the adjacent C—C double bond may be in a cis or trans configuration; [symbol]
R¹ is

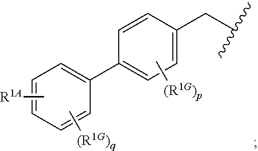

R^{1A} is

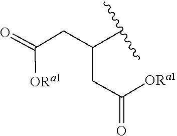

or —(CH₂)ₙN(R^{a1})₂;
each instance of R^{a1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a1} are joined together with the intervening atoms to form a substituted or unsubstituted heterocyclyl ring;
each instance of R^{1G} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{g1}, —NO₂, —N(R^{g2})₂, —SR^{g1}, —SO₂R^{g1}, —CN, or —SCN; R^{g1} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —N(R^{g2})₂, or —O(R^{g3});
each instance of R^{g2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two instances of R^{g2} are taken together to form a ring when attached to nitrogen;
each instance of R^{g3} is independently hydrogen, substituted or unsubstituted alkyl, substituted or unsubstituted acyl, or an oxygen protecting group when attached to an oxygen atom;
R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl;
each instance of R⁵ is independently hydrogen, substituted or unsubstituted alkyl, substituted or unsubstituted alkenyl, substituted or unsubstituted alkynyl, substituted or unsubstituted acyl, or a nitrogen protecting group;
each of R^{A}, R^{B}, R^{C}, and R^{D} is independently hydrogen, substituted or unsubstituted alkyl, substituted or unsubstituted acyl, or a nitrogen protecting group;
x is 0 or 1;
y is 0 or 1;
m1 is 0, 1, 2, 3, 4, 5, or 6;
n is 3, 4, 5, 6, 7, 8, 9, or 10;
p is 0, 1, 2, 3, or 4; and
q is 0, 1, 2, 3, or 4.

In certain embodiments, a compound described herein is of Formula (I-B):

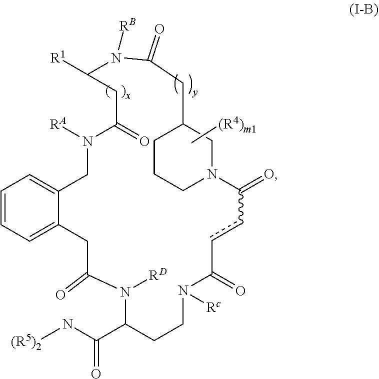

or a pharmaceutically acceptable salt, solvate, hydrate, polymorph, co-crystal, tautomer, stereoisomer, isotopically enriched form, prodrug, or mixture thereof, wherein:
each instance of [symbol] is independently a single or double C—C bond, as valency permits, wherein when [symbol] is a double C—C bond adjacent to [symbol] , then [symbol] indicates that the adjacent C—C double bond may be in a cis or trans configuration;
R¹ is

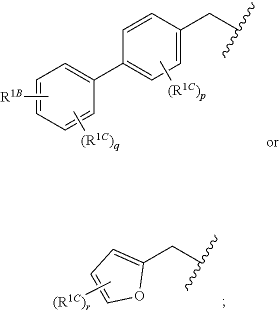

R^{1B} is

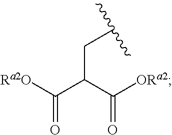

each instance of R^{a2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a2} are joined together with the intervening atoms to form a substituted or unsubstituted heterocyclyl ring;
each instance of R^{1C} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —SO₂R^{c1}, —CN, or —SCN;
R^{c1} is halogen, hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —N(R^{c2})₂, or —O(R^{c3});
each instance of R^{c2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two instances of R^{c2} are taken together to form a ring when attached to nitrogen;
each instance of R^{c3} is independently hydrogen, substituted or unsubstituted alkyl, substituted or unsubstituted acyl, or an oxygen protecting group when attached to an oxygen atom;
R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl;
each instance of R⁵ is independently hydrogen or

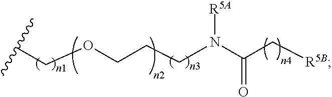

R^{5A} is independently hydrogen, substituted or unsubstituted alkyl, substituted or unsubstituted acyl, or a nitrogen protecting group;
R^{5B} is substituted or unsubstituted carbocyclyl, substituted or unsubstituted aryl, substituted or unsubstituted heterocyclyl, or substituted or unsubstituted heteroaryl;
each of R^{A}, R^{B}, R^{C}, and R^{D} is independently hydrogen, substituted or unsubstituted alkyl, substituted or unsubstituted acyl, or a nitrogen protecting group;
x is 0 or 1;
y is 0 or 1;
m1 is 0, 1, 2, 3, 4, 5, or 6;
p is 0, 1, 2, 3, or 4;
q is 0, 1, 2, 3, or 4;
r is 0, 1, 2, or 3;
n1 is 0, 1, 2, 3, 4, 5, 6, 7, 8, 9, or 10;
n2 is 0, 1, 2, 3, 4, 5, 6, 7, 8, 9, or 10;
n3 is 0, 1, 2, 3, 4, 5, 6, 7, 8, 9, or 10; and
n4 is 0, 1, 2, 3, 4, 5, 6, 7, 8, 9, or 10.

In certain embodiments, a compound described herein is of Formula (I-C):

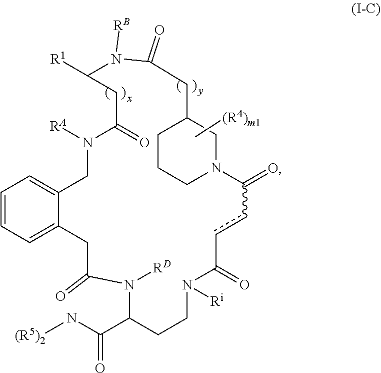

or a pharmaceutically acceptable salt, solvate, hydrate, polymorph, co-crystal, tautomer, stereoisomer, isotopically enriched form, prodrug, or mixture thereof, wherein:
each instance of [symbol] is independently a single or double C—C bond, as valency permits, wherein when [symbol] is a double C—C bond adjacent to [symbol] , then [symbol] indicates that the adjacent C—C double bond may be in a cis or trans configuration;
R¹ is

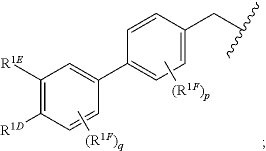

R^{1D} is hydrogen, —B(OR^{a3})₂, or —C(O)R^{a3};
R^{1E} is hydrogen, —B(OR^{a3})₂, or —C(O)R^{a3};
each instance of R^{a3} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a3} are joined together with the intervening atoms to form a substituted or unsubstituted heterocyclyl or heteroaryl ring;
each instance of R^{1F} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{f1}, —NO₂, —N(R^{f2})₂, —SR^{f1}, —SO₂R^{f1}, —CN, or —SCN;
R^{f1} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —N(R^{f2})₂, or —O(R^{f3});
each instance of R^{f2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two instances of R^{f2} are taken together to form a ring when attached to nitrogen;
each instance of R^{f3} is independently hydrogen, substituted or unsubstituted alkyl, substituted or unsubstituted acyl, or an oxygen protecting group when attached to an oxygen atom;
R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl;
each instance of R⁵ is independently hydrogen, substituted or unsubstituted alkyl, substituted or unsubstituted alkenyl, substituted or unsubstituted alkynyl, substituted or unsubstituted acyl, or a nitrogen protecting group;
each of R^{A}, R^{B}, R^{C}, and R^{D} is independently hydrogen, substituted or unsubstituted alkyl, substituted or unsubstituted acyl, or a nitrogen protecting group;
x is 0 or 1;
y is 0 or 1;
m1 is 0, 1, 2, 3, 4, 5, or 6;
p is 0, 1, 2, 3, or 4; and
q is 0, 1,2, or 3;
provided that R¹ is not

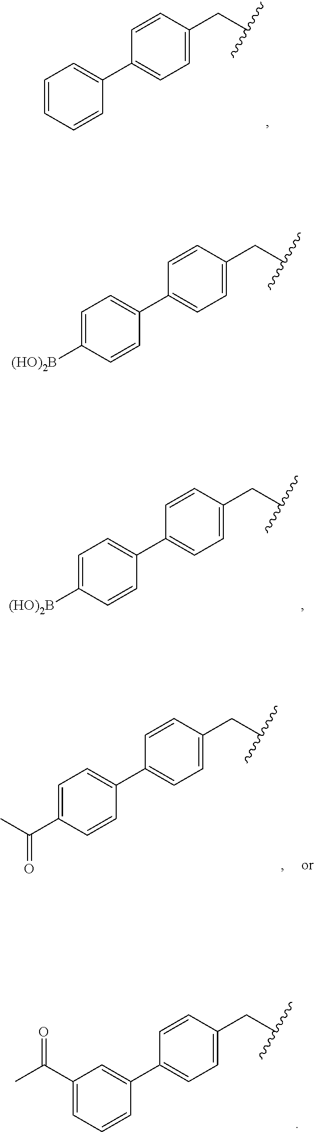 [SMILES: *Cc1ccc(-c2ccccc2)cc1 ; *Cc1ccc(-c2ccc(B(O)O)cc2)cc1 ; *Cc1ccc(-c2ccc(B(O)O)cc2)cc1 ; *Cc1ccc(-c2ccc(C(C)=O)cc2)cc1 ; *Cc1ccc(-c2cccc(C(C)=O)c2)cc1]

In certain embodiments, the compound of Formula (I-A), (I-B), or (I-C) is not of formula:

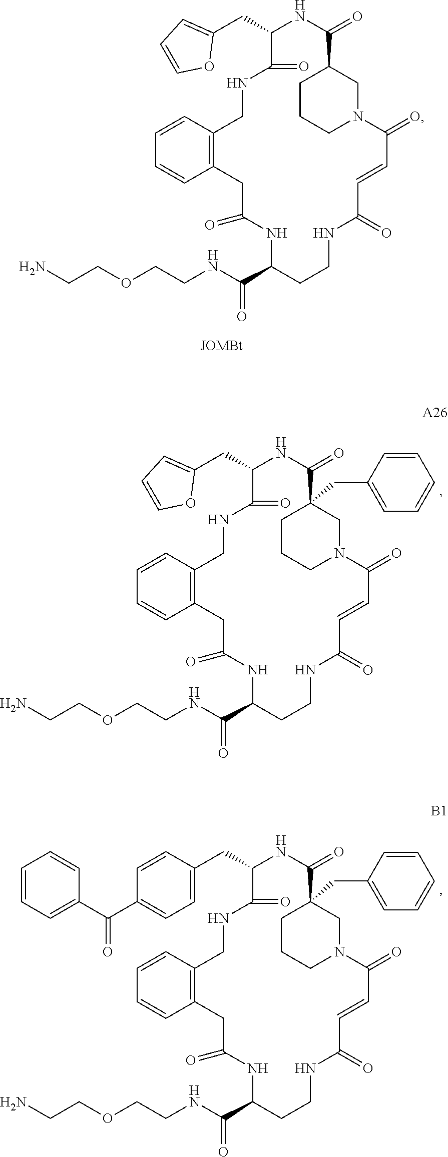 [SMILES: NCCOCCNC(=O)[C@@H]1CCNC(=O)/C=C/C(=O)N2CCC[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(C(=O)c3ccccc3)cc2)C(=O)NCc2ccccc2CC(=O)N1]

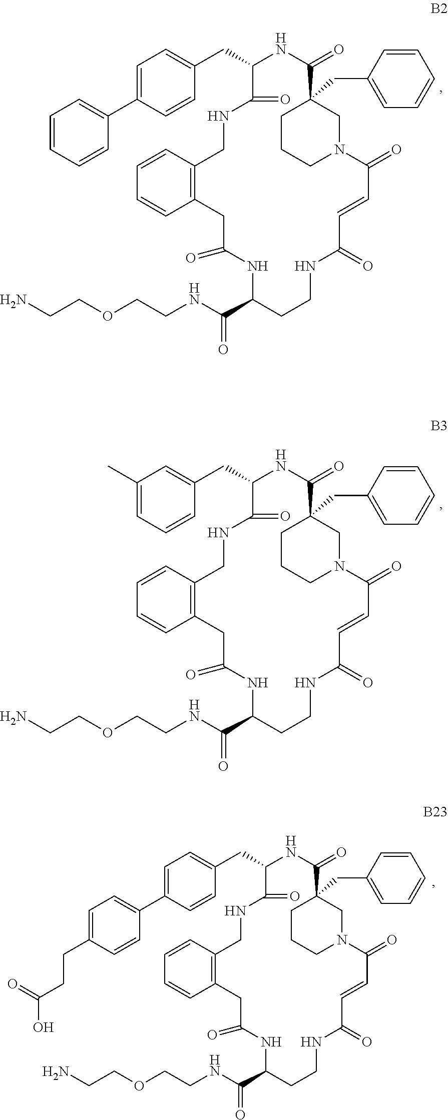 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccccc3)cc2)C(=O)NCc2ccccc2CC(=O)N1 ; Cc1cccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)c1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(CCC(=O)O)cc3)cc2)C(=O)NCc2ccccc2CC(=O)N1]

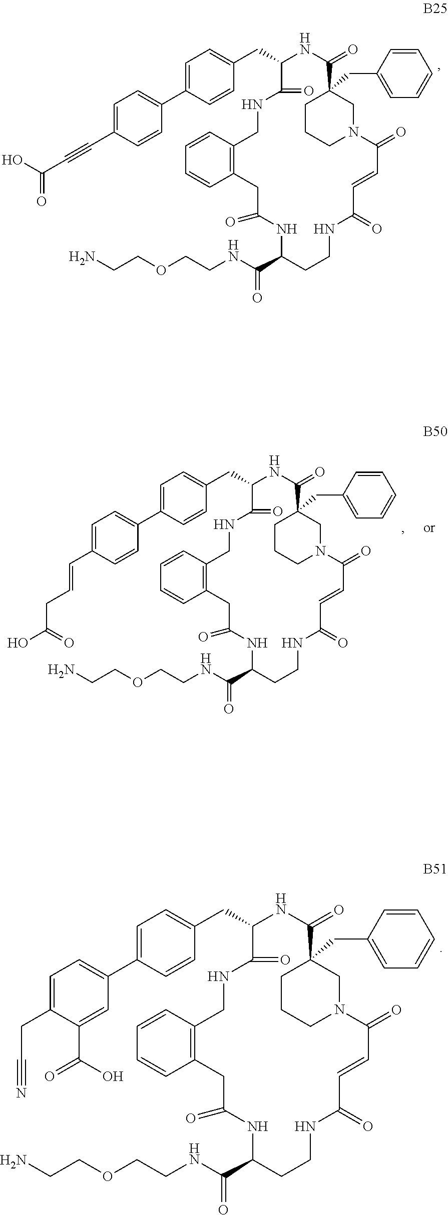 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccc(C#CC(=O)O)cc3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(/C=C/CC(=O)O)cc3)cc2)C(=O)NCc2ccccc2CC(=O)N1 ; N#CCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1C(=O)O]

In certain embodiments, a compound described herein is a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof. In certain embodiments, a compound described herein is a compound of Formula (I-A), (I-B), or (I-C), or a pharmaceutically acceptable salt thereof. In certain embodiments, a compound described herein is a compound of Formula (I-A), or a pharmaceutically acceptable salt, solvate, hydrate, polymorph, co-crystal, tautomer, stereoisomer, isotopically enriched form, prodrug, or mixture thereof. In certain embodiments, a compound described herein is a compound of Formula (I-A), or a pharmaceutically acceptable salt thereof. In certain embodiments, a compound described herein is a compound of Formula (I-B), or a pharmaceutically acceptable salt, solvate, hydrate, polymorph, co-crystal, tautomer, stereoisomer, isotopically enriched form, prodrug, or mixture thereof. In certain embodiments, a compound described herein is a compound of Formula (I-B), or a pharmaceutically acceptable salt thereof. In certain embodiments, a compound described herein is a compound of Formula (I-C), or a pharmaceutically acceptable salt, solvate, hydrate, polymorph, co-crystal, tautomer, stereoisomer, isotopically enriched form, prodrug, or mixture thereof. In certain embodiments, a compound described herein is a compound of Formula (I-C), or a pharmaceutically acceptable salt thereof.

### Formula (I-A);

In certain embodiments, R¹ is of formula:

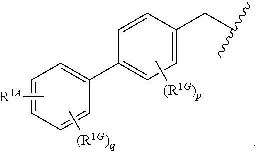

In certain embodiments, R¹ is of formula:

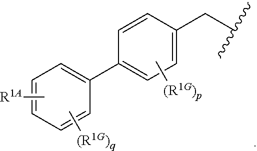

wherein R^{1A} is of formula:

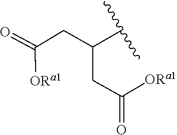

or —(CH₂)ₙN(R^{a1})₂. In certain embodiments, R¹ is of formula:

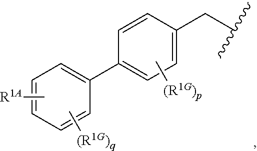

wherein R^{1A} is of formula:

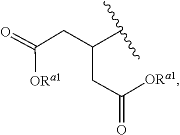

wherein each instance of R^{a1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a1} are joined together with the intervening atoms to form a substituted or unsubstituted heterocyclyl ring; each instance of R^{1G} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{g1}, —NO₂, —N(R^{g2})₂, —SR^{g1}, —SO₂R^{g1}, —CN, or —SCN; R^{g1} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —N(R^{g2})₂, or —O(R^{g3}); each instance of R^{g2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two instances of R^{g2} are taken together to form a ring when attached to nitrogen; each instance of R^{g3} is independently hydrogen, substituted or unsubstituted alkyl, substituted or unsubstituted acyl, or an oxygen protecting group when attached to an oxygen atom; p is 0, 1, 2, 3, or 4; and q is 0, 1, 2, 3, or 4.

In certain embodiments, R¹ is of formula:

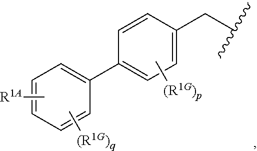

wherein R^{1A} is of formula: —(CH₂)ₙN(R^{a1})₂, wherein each instance of R^{a1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a1} are joined together with the intervening atoms to form a substituted or unsubstituted heterocyclyl ring; each instance of R^{1G} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{g1}, —NO₂, —N(R^{g2})₂, —SR^{g1}, —SO₂R^{g1}, —CN, or —SCN; R^{g1} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —N(R^{g2})₂, or —O(R^{g3}); each instance of R^{g2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two instances of R^{g2} are taken together to form a ring when attached to nitrogen; each instance of R^{g3} is independently hydrogen, substituted or unsubstituted alkyl, substituted or unsubstituted acyl, or an oxygen protecting group when attached to an oxygen atom; n is 3, 4, 5, 6, 7, 8, 9, or 10; p is 0, 1, 2, 3, or 4; and q is 0, 1, 2, 3, or 4.

In certain embodiments, at least one instance of R^{a1} is hydrogen. In certain embodiments, at least one instance of R^{a1} is substituted or unsubstituted acyl (e.g., —C(═O)Me). In certain embodiments, at least one instance of R^{a1} is substituted or unsubstituted alkyl (e.g., substituted or unsubstituted C₁₋₆ alkyl). In certain embodiments, two instances of R^{a1} are substituted or unsubstituted alkyl. In certain embodiments, at least one instance of R^{a1} is substituted or unsubstituted methyl. In certain embodiments, at least one instance of R^{a1} is substituted methyl (e.g., —CF₃). In certain embodiments, at least one instance of R^{a1} is unsubstituted methyl. In certain embodiments, at least one instance of R^{a1} is substituted or unsubstituted ethyl. In certain embodiments, two instances of R^{a1} are substituted or unsubstituted ethyl. In certain embodiments, at least one instance of R^{a1} is substituted ethyl (e.g., —CH₂CH₂OH). In certain embodiments, at least one instance of R^{a1} is —CH₂CH₂OH. In certain embodiments, at least one instance of R^{a1} is —CH₂CH₂OMe. In certain embodiments, at least one instance of R^{a1} is unsubstituted ethyl. In certain embodiments, at least one instance of R^{a1} is substituted or unsubstituted propyl. In certain embodiments, at least one instance of R^{a1} is substituted or unsubstituted butyl (e.g., t-butyl, n-butyl). In certain embodiments, at least one instance of R^{a1} is substituted or unsubstituted t-butyl. In certain embodiments, at least one instance of R^{a1} is unsubstituted t-butyl. In certain embodiments, at least one instance of R^{a1} is substituted or unsubstituted alkenyl (e.g., substituted or unsubstituted C₂₋₆ alkenyl). In certain embodiments, at least one instance of R^{a1} is substituted or unsubstituted alkynyl (e.g., substituted or unsubstituted C₂₋₆ alkynyl). In certain embodiments, at least one instance of R^{a1} is substituted or unsubstituted carbocyclyl (e.g., substituted or unsubstituted, 3- to 10-membered, monocyclic carbocyclyl comprising zero, one, or two double bonds in the carbocyclic ring system). In certain embodiments, at least one instance of R^{a1} is substituted or unsubstituted aryl (e.g., substituted or unsubstituted, 6- to 10-membered aryl). In certain embodiments, at least one instance of R^{a1} is benzyl. In certain embodiments, at least one instance of R^{a1} is substituted or unsubstituted phenyl. In certain embodiments, at least one instance of R^{a1} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{a1} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, two instances of R^{a1} are joined together with the intervening atoms to form a substituted or unsubstituted heterocyclyl group (e.g., substituted or unsubstituted, 3- to 10-membered heterocyclyl).

In certain embodiments, at least one instance of R^{1G} is halogen (e.g., F, Cl, Br, or I). In certain embodiments, at least one instance of R^{1G} is substituted or unsubstituted acyl (e.g., —C(═O)Me). In certain embodiments, at least one instance of R^{1G} is substituted or unsubstituted alkyl (e.g., substituted or unsubstituted C₁₋₆ alkyl). In certain embodiments, at least one instance of R^{1G} is substituted or unsubstituted methyl. In certain embodiments, at least one instance of R^{1G} is substituted methyl (e.g., —CF₃). In certain embodiments, at least one instance of R^{1G} is unsubstituted methyl. In certain embodiments, at least one instance of R^{1G} is substituted or unsubstituted ethyl. In certain embodiments, at least one instance of R^{1G} is unsubstituted ethyl. In certain embodiments, at least one instance of R^{1G} is substituted or unsubstituted propyl. In certain embodiments, at least one instance of R^{1G} is substituted or unsubstituted butyl (e.g., t-butyl, n-butyl). In certain embodiments, at least one instance of R^{1G} is substituted or unsubstituted alkenyl (e.g., substituted or unsubstituted C₂₋₆ alkenyl). In certain embodiments, at least one instance of R^{1G} is substituted or unsubstituted alkynyl (e.g., substituted or unsubstituted C₂₋₆ alkynyl). In certain embodiments, at least one instance of R^{1G} is substituted or unsubstituted carbocyclyl (e.g., substituted or unsubstituted, 3- to 10-membered, monocyclic carbocyclyl comprising zero, one, or two double bonds in the carbocyclic ring system). In certain embodiments, at least one instance of R^{1G} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{1G} is substituted or unsubstituted aryl (e.g., substituted or unsubstituted, 6- to 10-membered aryl). In certain embodiments, at least one instance R^{1G} is benzyl. In certain embodiments, at least one instance of R^{1G} is substituted or unsubstituted phenyl. In certain embodiments, at least one instance of R^{1G} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{1G} is —OR^{g1} (e.g., —OH or —OMe). In certain embodiments, at least one instance of R^{1G} is —O(CH₂)(substituted or unsubstituted aryl). In certain embodiments, at least one instance of R^{1G} is —O(CH₂)(phenyl). In certain embodiments, at least one instance of R^{1G} is —O(substituted or unsubstituted phenyl). In certain embodiments, at least one instance of R^{1G} is —O(substituted or unsubstituted aryl). In certain embodiments, at least one instance of R^{1G} is —NO₂. In certain embodiments, at least one instance of R^{1G} is —N(R^{g2})₂(e.g., —NMe₂). In certain embodiments, at least one instance of R^{1G} is —SR^{g1} (e.g., —SMe). In certain embodiments, at least one instance of R^{1G} is —SO₂, —CN, or —SCN.

In certain embodiments, R^{g1} is halogen, hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —NH₂, —N(R^{g2})₂, —OH, or —O(R^{g3}). In certain embodiments, R^{g1} is halogen (e.g., F, Cl, Br, or I). In certain embodiments, R^{g1} is hydrogen. In certain embodiments, R^{g1} is substituted or unsubstituted acyl (e.g., —C(═O)Me). In certain embodiments, R^{g1} is substituted or unsubstituted alkyl (e.g., substituted or unsubstituted C₁₋₆ alkyl). In certain embodiments, R^{g1} is substituted or unsubstituted methyl. In certain embodiments, R^{g1} is substituted or unsubstituted ethyl. In certain embodiments, R^{g1} is substituted or unsubstituted propyl. In certain embodiments, R^{g1} is substituted or unsubstituted alkenyl (e.g., substituted or unsubstituted C₂₋₆ alkenyl). In certain embodiments, R^{g1} is substituted or unsubstituted alkynyl (e.g., substituted or unsubstituted C₂₋₆ alkynyl). In certain embodiments, R^{g1} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{g1} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{g1} is substituted or unsubstituted aryl (e.g., substituted or unsubstituted, 6- to 10-membered aryl). In certain embodiments, R^{g1} is benzyl. In certain embodiments, R^{g1} is substituted or unsubstituted phenyl. In certain embodiments, R^{g1} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{g1} is an oxygen protecting group when attached to an oxygen atom. In certain embodiments, R^{g1} is a sulfur protecting group when attached to a sulfur atom. In certain embodiments, R^{g1} is —NH₂. In certain embodiments, R^{g1} is —N(R^{g2})₂. In certain embodiments, R^{g1} is —OH. In certain embodiments, R^{g1} is —O(R^{g3}).

In certain embodiments, each instance of R^{g2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group. In certain embodiments, at least one instance of R^{g2} is hydrogen. In certain embodiments, at least one instance of R^{g2} is substituted or unsubstituted acyl (e.g., —C(═O)Me). In certain embodiments, at least one R^{g2} is substituted or unsubstituted alkyl (e.g., substituted or unsubstituted C₁₋₆ alkyl). In certain embodiments, at least one instance of R^{g2} is substituted or unsubstituted methyl. In certain embodiments, at least one instance of R^{g2} is substituted or unsubstituted ethyl. In certain embodiments, at least one instance of R^{g2} is substituted or unsubstituted propyl. In certain embodiments, at least one instance of R^{g2} is substituted or unsubstituted alkenyl (e.g., substituted or unsubstituted C₂₋₆ alkenyl). In certain embodiments, at least one instance of R^{g2} is substituted or unsubstituted alkynyl (e.g., substituted or unsubstituted C₂₋₆ alkynyl). In certain embodiments, at least one instance of R^{g2} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{g2} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{g2} is substituted or unsubstituted aryl (e.g., substituted or unsubstituted, 6- to 10-membered aryl). In certain embodiments, at least one instance of R^{g2} is benzyl. In certain embodiments, at least one instance of R^{g2} is substituted or unsubstituted phenyl. In certain embodiments, at least one instance of R^{g2} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{g2} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, or two instances of R^{g2} are taken together to form a ring when attached to nitrogen.

In certain embodiments, each instance of R^{g3} is independently hydrogen or substituted or unsubstituted alkyl. In certain embodiments, at least one instance of R^{g3} is hydrogen. In certain embodiments, at least one instance of R^{g3} is substituted or unsubstituted acyl (e.g., —C(═O)Me). In certain embodiments, at least one R^{g3} is substituted or unsubstituted alkyl (e.g., substituted or unsubstituted C₁₋₆ alkyl). In certain embodiments, at least one instance of R^{g3} is substituted or unsubstituted methyl. In certain embodiments, at least one instance of R^{g3} is substituted or unsubstituted ethyl. In certain embodiments, at least one instance of R^{g3} is substituted or unsubstituted propyl. In certain embodiments, R^{g3} is substituted or unsubstituted acyl (e.g., —C(═O)Me). In certain embodiments, R^{g3} is an oxygen protecting group when attached to an oxygen atom.

In certain embodiments, R¹ is of formula:

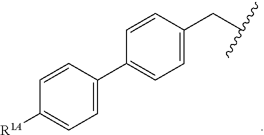

In certain embodiments, R¹ is of formula:

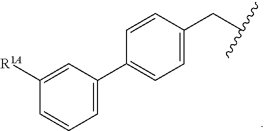

In certain embodiments, R¹ is of formula:

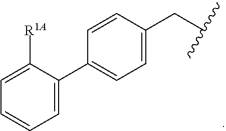

In certain embodiments, R¹ is of formula:

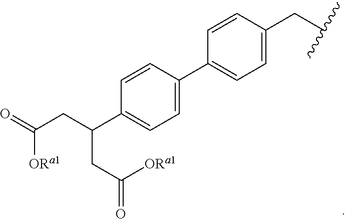

In certain embodiments, R¹ is

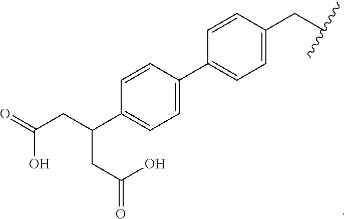 [SMILES: *Cc1ccc(-c2ccc(C(CC(=O)O)CC(=O)O)cc2)cc1]

### Formula (I-B)

In certain embodiments, R¹ is of formula:

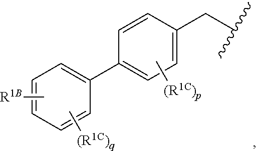

In certain embodiments, R¹ is of formula:

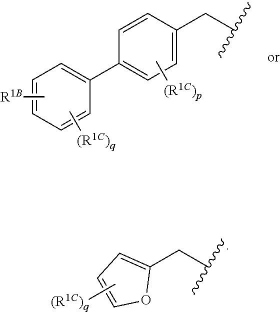

wherein R^{1B} is of formula:

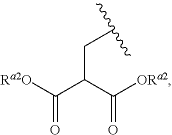

wherein each instance of R^{a2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a2} are joined together with the intervening atoms to form a substituted or unsubstituted heterocyclyl ring. In certain embodiments, at least one instance of R^{a2} is hydrogen. In certain embodiments, at least one instance of R^{a2} is substituted or unsubstituted acyl (e.g., —C(═O)Me). In certain embodiments, at least one instance of R^{a2} is substituted or unsubstituted alkyl (e.g., substituted or unsubstituted C₁₋₆ alkyl). In certain embodiments, two instances of R^{a2} are substituted or unsubstituted alkyl. In certain embodiments, at least one instance of R^{a2} is substituted or unsubstituted methyl. In certain embodiments, at least one instance of R^{a2} is substituted methyl (e.g., —CF₃). In certain embodiments, at least one instance of R^{a2} is unsubstituted methyl. In certain embodiments, at least one instance of R^{a2} is substituted or unsubstituted ethyl. In certain embodiments, two instances of R^{a2} are substituted or unsubstituted ethyl. In certain embodiments, at least one instance of R^{a2} is substituted ethyl (e.g., —CH₂CH₂OH). In certain embodiments, at least one instance of R^{a2} is —CH₂CH₂OH. In certain embodiments, at least one instance of R^{a2} is —CH₂CH₂OMe. In certain embodiments, at least one instance of R^{a2} is unsubstituted ethyl. In certain embodiments, at least one instance of R^{a2} is substituted or unsubstituted propyl. In certain embodiments, at least one instance of R^{a2} is substituted or unsubstituted butyl (e.g., t-butyl, n-butyl). In certain embodiments, at least one instance of R^{a2} is substituted or unsubstituted t-butyl. In certain embodiments, at least one instance of R^{a2} is unsubstituted t-butyl. In certain embodiments, at least one instance of R^{a2} is substituted or unsubstituted alkenyl (e.g., substituted or unsubstituted C₂₋₆ alkenyl). In certain embodiments, at least one instance of R^{a2} is substituted or unsubstituted alkynyl (e.g., substituted or unsubstituted C₂₋₆ alkynyl). In certain embodiments, at least one instance of R^{a2} is substituted or unsubstituted carbocyclyl (e.g., substituted or unsubstituted, 3- to 10-membered, monocyclic carbocyclyl comprising zero, one, or two double bonds in the carbocyclic ring system). In certain embodiments, at least one instance of R^{a2} is substituted or unsubstituted aryl (e.g., substituted or unsubstituted, 6- to 10-membered aryl). In certain embodiments, at least one instance of R^{a2} is benzyl. In certain embodiments, at least one instance of R^{a2} is substituted or unsubstituted phenyl. In certain embodiments, at least one instance of R^{a2} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{a2} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, two instances of R^{a2} are joined together with the intervening atoms to form a substituted or unsubstituted heterocyclyl group (e.g., substituted or unsubstituted, 3- to 10-membered heterocyclyl). In certain embodiments, R¹ is of formula:

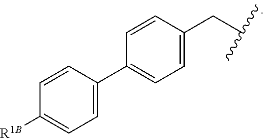

In certain embodiments, R¹ is of formula:

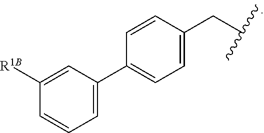

In certain embodiments, R¹ is of formula:

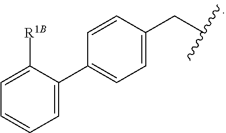

In certain embodiments, R¹ is of formula:

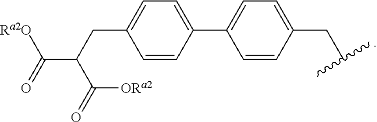

In certain embodiments, R¹ is

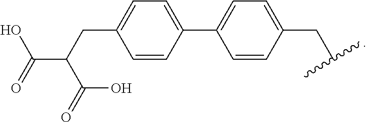 [SMILES: *Cc1ccc(-c2ccc(CC(C(=O)O)C(=O)O)cc2)cc1]

In certain embodiments, R¹ is of formula:

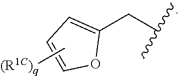

In certain embodiments, R¹ is of formula:

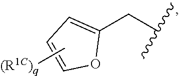

wherein each instance of R^{1C} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —SO₂R^{c1}, —CN, or —SCN. In certain embodiments, at least one instance of R^{1C} is halogen (e.g., F, Cl, Br, or I). In certain embodiments, at least one instance of R^{1C} is substituted or unsubstituted acyl (e.g., —C(═O)Me). In certain embodiments, at least one instance of R^{1C} is substituted or unsubstituted alkyl (e.g., substituted or unsubstituted C₁₋₆ alkyl). In certain embodiments, at least one instance of R^{1C} is substituted or unsubstituted methyl. In certain embodiments, at least one instance of R^{1C} is substituted methyl (e.g., —CF₃). In certain embodiments, at least one instance of R^{1C} is unsubstituted methyl. In certain embodiments, at least one instance of R^{1C} is substituted or unsubstituted ethyl. In certain embodiments, at least one instance of R^{1C} is unsubstituted ethyl. In certain embodiments, at least one instance of R^{1C} is substituted or unsubstituted propyl. In certain embodiments, at least one instance of R^{1C} is substituted or unsubstituted butyl (e.g., t-butyl, n-butyl). In certain embodiments, at least one instance of R^{1C} is substituted or unsubstituted alkenyl (e.g., substituted or unsubstituted C₂₋₆ alkenyl). In certain embodiments, at least one instance of R^{1C} is substituted or unsubstituted alkynyl (e.g., substituted or unsubstituted C₂₋₆ alkynyl). In certain embodiments, at least one instance of R^{1C} is substituted or unsubstituted carbocyclyl (e.g., substituted or unsubstituted, 3- to 10-membered, monocyclic carbocyclyl comprising zero, one, or two double bonds in the carbocyclic ring system). In certain embodiments, at least one instance of R^{1C} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{1C} is substituted or unsubstituted aryl (e.g., substituted or unsubstituted, 6- to 10-membered aryl). In certain embodiments, at least one instance R^{1C} is benzyl. In certain embodiments, at least one instance of R^{1C} is substituted or unsubstituted phenyl. In certain embodiments, at least one instance of R^{1C} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{1C} is —OR^{c1} (e.g., —OH or —OMe). In certain embodiments, at least one instance of R^{1C} is —O(CH₂)(substituted or unsubstituted aryl). In certain embodiments, at least one instance of R^{1C} is —O(CH₂)(phenyl). In certain embodiments, at least one instance of R^{1C} is —O(substituted or unsubstituted phenyl). In certain embodiments, at least one instance of R^{1C} is —O(substituted or unsubstituted aryl). In certain embodiments, at least one instance of R^{1C} is —NO₂. In certain embodiments, at least one instance of R^{1C} is —N(R^{c2})₂ (e.g., —NMe₂). In certain embodiments, at least one instance of R^{1C} is —SR^{c1} (e.g., —SMe). In certain embodiments, at least one instance of R^{1C} is —SO₂, —CN, or —SCN. In certain embodiments, R¹ is of formula:

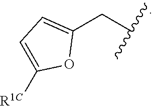

In certain embodiments, R¹ is of formula:

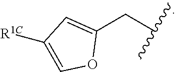

In certain embodiments, R¹ is of formula:

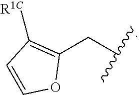

In certain embodiments, R¹ is

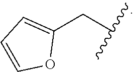 [SMILES: *Cc1ccco1]

In certain embodiments, R¹ is halogen, hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —NH₂, —N(R^{c2})₂, —OH, or —O(R^{c3}). In certain embodiments, R^{c1} is halogen (e.g., F, Cl, Br, or I). In certain embodiments, R^{c1} is hydrogen. In certain embodiments, R^{c1} is substituted or unsubstituted acyl (e.g., —C(═O)Me). In certain embodiments, R^{c1} is substituted or unsubstituted alkyl (e.g., substituted or unsubstituted C₁₋₆ alkyl). In certain embodiments, R^{c1} is substituted or unsubstituted methyl. In certain embodiments, R^{c1} is substituted or unsubstituted ethyl. In certain embodiments, R^{c1} is substituted or unsubstituted propyl. In certain embodiments, R^{c1} is substituted or unsubstituted alkenyl (e.g., substituted or unsubstituted C₂₋₆ alkenyl). In certain embodiments, R^{c1} is substituted or unsubstituted alkynyl (e.g., substituted or unsubstituted C₂₋₆ alkynyl). In certain embodiments, R^{c1} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{c1} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{c1} is substituted or unsubstituted aryl (e.g., substituted or unsubstituted, 6- to 10-membered aryl). In certain embodiments, R^{c1} is benzyl. In certain embodiments, R^{c1} is substituted or unsubstituted phenyl. In certain embodiments, R^{c1} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{c1} is an oxygen protecting group when attached to an oxygen atom. In certain embodiments, R^{c1} is a sulfur protecting group when attached to a sulfur atom. In certain embodiments, R^{c1} is —NH₂. In certain embodiments, R^{c1} is —N(R^{c2})₂. In certain embodiments, R^{c1} is —OH. In certain embodiments, R^{c1} is —O(R^{c3}).

In certain embodiments, each instance of R^{c2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group. In certain embodiments, at least one instance of R^{c2} is hydrogen. In certain embodiments, at least one instance of R^{c2} is substituted or unsubstituted acyl (e.g., —C(═O)Me). In certain embodiments, at least one R^{c2} is substituted or unsubstituted alkyl (e.g., substituted or unsubstituted C₁₋₆ alkyl). In certain embodiments, at least one instance of R^{c2} is substituted or unsubstituted methyl. In certain embodiments, at least one instance of R^{c2} is substituted or unsubstituted ethyl. In certain embodiments, at least one instance of R^{c2} is substituted or unsubstituted propyl. In certain embodiments, at least one instance of R^{c2} is substituted or unsubstituted alkenyl (e.g., substituted or unsubstituted C₂₋₆ alkenyl). In certain embodiments, at least one instance of R^{c2} is substituted or unsubstituted alkynyl (e.g., substituted or unsubstituted C₂₋₆ alkynyl). In certain embodiments, at least one instance of R^{c2} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{c2} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{c2} is substituted or unsubstituted aryl (e.g., substituted or unsubstituted, 6- to 10-membered aryl). In certain embodiments, at least one instance of R^{c2} is benzyl. In certain embodiments, at least one instance of R^{c2} is substituted or unsubstituted phenyl. In certain embodiments, at least one instance of R^{c2} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R² is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, or two instances of R^{c2} are taken together to form a ring when attached to nitrogen.

In certain embodiments, each instance of R^{c3} is independently hydrogen or substituted or unsubstituted alkyl. In certain embodiments, at least one instance of R^{c3} is hydrogen. In certain embodiments, at least one instance of R^{c3} is substituted or unsubstituted acyl (e.g., —C(═O)Me). In certain embodiments, at least one R^{c3} is substituted or unsubstituted alkyl (e.g., substituted or unsubstituted C₁₋₆ alkyl). In certain embodiments, at least one instance of R^{c3} is substituted or unsubstituted methyl. In certain embodiments, at least one instance of R^{c3} is substituted or unsubstituted ethyl. In certain embodiments, at least one instance of R^{c3} is substituted or unsubstituted propyl. In certain embodiments, R^{c3} is substituted or unsubstituted acyl (e.g., —C(═O)Me). In certain embodiments, R^{c3} is an oxygen protecting group when attached to an oxygen atom.

In certain embodiments, each instance of R⁵ is independently hydrogen or

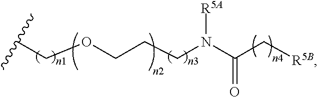

wherein R^{5A} is independently hydrogen, substituted or unsubstituted alkyl, substituted or unsubstituted acyl, or a nitrogen protecting group; and R^{5B} is substituted or unsubstituted carbocyclyl, substituted or unsubstituted aryl, substituted or unsubstituted heterocyclyl, or substituted or unsubstituted heteroaryl. In certain embodiments, at least one instance of R⁵ is hydrogen. In certain embodiments, at least one instance of R⁵ is

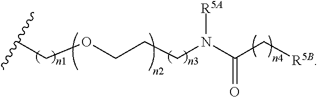

In certain embodiments, R^{5A} is hydrogen. In certain embodiments, R^{5A} is substituted or unsubstituted alkyl (e.g., substituted or unsubstituted C₁₋₆ alkyl). In certain embodiments, R^{5A} is substituted or unsubstituted C₁₋₆ alkyl. In certain embodiments, R^{5A} is substituted or unsubstituted methyl. In certain embodiments, R^{5A} is unsubstituted methyl. In certain embodiments, R^{5A} is substituted or unsubstituted acyl (e.g., —C(═O)Me). In certain embodiments, R^{5A} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, R^{5B} is substituted or unsubstituted carbocyclyl (e.g., substituted or unsubstituted, 3- to 10-membered, monocyclic carbocyclyl comprising zero, one, or two double bonds in the carbocyclic ring system). In certain embodiments, R^{5B} is substituted or unsubstituted aryl (e.g., substituted or unsubstituted, 6- to 10-membered aryl). In certain embodiments, R^{5B} is substituted or unsubstituted phenyl. In certain embodiments, R^{5B} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{5B} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, each instance of R⁵ is hydrogen. In certain embodiments, at least one instance of R⁵ is

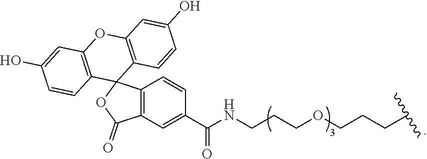

In certain embodiments, one instance of R⁵ is hydrogen, and one instance of R⁵ is

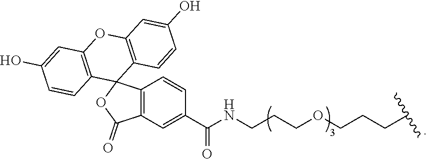

In certain embodiments, one instance of R⁵ is

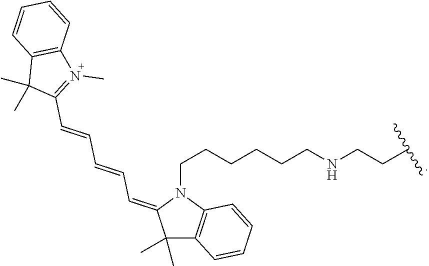

In certain embodiments, one instance of R⁵ is hydrogen, and one instance of R⁵ is

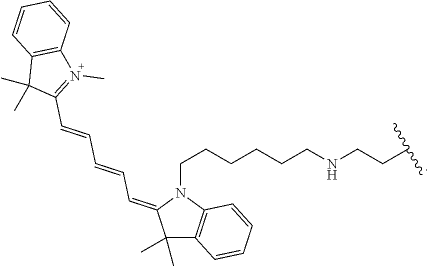

### Formula (I-C)

In certain embodiments, R¹ is of formula:

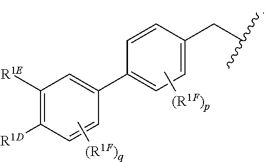

In certain embodiments, R¹ is of formula:

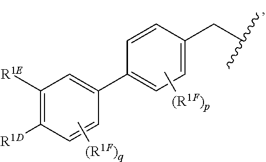

wherein R^{1D} is hydrogen, —B(OR^{a3})₂, or —C(O)R^{a3}. In certain embodiments, R^{1D} is hydrogen. In certain embodiments, R^{1D} is —B(OR^{a3})₂. In certain embodiments, R^{1D} is —C(O)R^{a3}. In certain embodiments, R¹ is of formula:

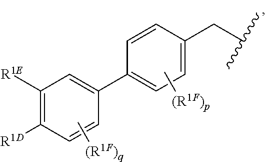

wherein R^{1E} is hydrogen, —B(OR^{a3})₂, or —C(O)R^{a3}. In certain embodiments, R^{1E} is hydrogen. In certain embodiments, R^{1E} is —B(OR^{a3})₂. In certain embodiments, R^{1E} is —C(O)R^{a3}. In certain embodiments, if R^{1D} is hydrogen, then R^{1E} is —B(OR^{a3})₂. In certain embodiments, if R^{1D} is hydrogen, then R^{1E} is —C(O)R^{a3}. In certain embodiments, if R^{1D} is —B(OH)₂, then R^{1E} is —B(OR^{a3})₂. In certain embodiments, if R^{1D} is —B(OH)₂, then R^{1E} is —C(O)R^{a3}. In certain embodiments, if R^{1D} is —C(O)CH₃, then R^{1E} is —B(OR^{a3})₂. In certain embodiments, if R^{1D} is —C(O)CH₃, then R^{1E} is —C(O)R^{a3}. In certain embodiments, if R^{1D} is hydrogen, —B(OH)₂, or —C(O)CH₃, then R^{1E} is —B(OR^{a3})₂, or —C(O)R^{a3}. In certain embodiments, each instance of R^{a3} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a3} are joined together with the intervening atoms to form a substituted or unsubstituted heterocyclyl or heteroaryl ring. In certain embodiments, R¹ is of formula:

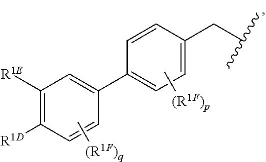

wherein each instance of R^{1F} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{f1}, —NO₂, —N(R^{f2})₂, —SR^{f1}, —SO₂R^{f1}, —CN, or —SCN. In certain embodiments, R¹ is of formula:

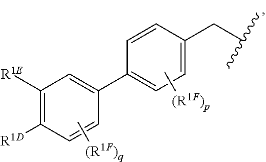

wherein R^{1D} is hydrogen, —B(OR^{a3})₂, or —C(O)R^{a3}; R^{1E} is hydrogen, —B(OR^{a3})₂, or —C(O)R^{a3}; and each instance of R^{1F} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{f1}, —NO₂, —N(R^{f2})₂, —SR^{f1}, —SO₂R^{f1}, —CN, or —SCN. In certain embodiments, at least one instance of R^{a3} is hydrogen. In certain embodiments, at least one instance of R^{a3} is substituted or unsubstituted acyl (e.g., —C(═O)Me). In certain embodiments, at least one instance of R^{a3} is substituted or unsubstituted alkyl (e.g., substituted or unsubstituted C₁₋₆ alkyl). In certain embodiments, at least one instance of R^{a3} is substituted or unsubstituted methyl. In certain embodiments, at least one instance of R^{a} is substituted methyl (e.g., —CF₃). In certain embodiments, at least one instance of R^{a3} is unsubstituted methyl. In certain embodiments, at least one instance of R^{a3} is substituted or unsubstituted ethyl. In certain embodiments, at least one instance of R^{a3} is substituted ethyl (e.g., —CH₂CH₂OH). In certain embodiments, at least one instance of R^{a3} is —CH₂CH₂OH. In certain embodiments, at least one instance of R^{a3} is —CH₂CH₂OMe. In certain embodiments, at least one instance of R^{a3} is unsubstituted ethyl. In certain embodiments, at least one instance of R^{a3} is substituted or unsubstituted propyl. In certain embodiments, at least one instance of R^{a3} is substituted or unsubstituted butyl (e.g., t-butyl, n-butyl). In certain embodiments, at least one instance of R^{a3} is substituted or unsubstituted t-butyl. In certain embodiments, at least one instance of R^{a3} is unsubstituted t-butyl. In certain embodiments, at least one instance of R^{a3} is substituted or unsubstituted alkenyl (e.g., substituted or unsubstituted C₂₋₆ alkenyl). In certain embodiments, at least one instance of R^{a3} is substituted or unsubstituted alkynyl (e.g., substituted or unsubstituted C₂₋₆ alkynyl). In certain embodiments, at least one instance of R^{a3} is substituted or unsubstituted carbocyclyl (e.g., substituted or unsubstituted, 3- to 10-membered, monocyclic carbocyclyl comprising zero, one, or two double bonds in the carbocyclic ring system). In certain embodiments, at least one instance of R^{a3} is substituted or unsubstituted aryl (e.g., substituted or unsubstituted, 6- to 10-membered aryl). In certain embodiments, at least one instance of R^{a3} is benzyl. In certain embodiments, at least one instance of R^{a3} is substituted or unsubstituted phenyl. In certain embodiments, at least one instance of R^{a3} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{a3} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, two instances of R^{a3} are joined together with the intervening atoms to form a substituted or unsubstituted heterocyclyl group (e.g., substituted or unsubstituted, 3- to 10-membered heterocyclyl).

In certain embodiments, each instance of R^{1F} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{f1}, —NO₂, —N(R^{f2})₂, —SR^{f1}, —SO₂R^{f1}, —CN, or —SCN. In certain embodiments, at least one instance of R^{1F} is halogen (e.g., F, Cl, Br, or I). In certain embodiments, at least one instance of R^{1F} is substituted or unsubstituted acyl (e.g., —C(═O)Me). In certain embodiments, at least one instance of R^{1F} is substituted or unsubstituted alkyl (e.g., substituted or unsubstituted C₁₋₆ alkyl). In certain embodiments, at least one instance of R^{1F} is substituted or unsubstituted methyl. In certain embodiments, at least one instance of R^{1F} is substituted methyl (e.g., —CF₃). In certain embodiments, at least one instance of R^{1F} is unsubstituted methyl. In certain embodiments, at least one instance of R^{1F} is substituted or unsubstituted ethyl. In certain embodiments, at least one instance of R^{1F} is unsubstituted ethyl. In certain embodiments, at least one instance of R^{1F} is substituted or unsubstituted propyl. In certain embodiments, at least one instance of R^{1F} is substituted or unsubstituted butyl (e.g., t-butyl, n-butyl). In certain embodiments, at least one instance of R^{1F} is substituted or unsubstituted alkenyl (e.g., substituted or unsubstituted C₂₋₆ alkenyl). In certain embodiments, at least one instance of R^{1F} is substituted or unsubstituted alkynyl (e.g., substituted or unsubstituted C₂₋₆ alkynyl). In certain embodiments, at least one instance of R^{1F} is substituted or unsubstituted carbocyclyl (e.g., substituted or unsubstituted, 3- to 10-membered, monocyclic carbocyclyl comprising zero, one, or two double bonds in the carbocyclic ring system). In certain embodiments, at least one instance of R^{1F} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{1F} is substituted or unsubstituted aryl (e.g., substituted or unsubstituted, 6- to 10-membered aryl). In certain embodiments, at least one instance R^{1F} is benzyl. In certain embodiments, at least one instance of R^{1F} is substituted or unsubstituted phenyl. In certain embodiments, at least one instance of R^{1F} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{1F} is —OR^{f1} (e.g., —OH or —OMe). In certain embodiments, at least one instance of R^{1F} is —O(CH₂)(substituted or unsubstituted aryl). In certain embodiments, at least one instance of R^{1F} is —O(CH₂)(phenyl). In certain embodiments, at least one instance of R^{1F} is —O(substituted or unsubstituted phenyl). In certain embodiments, at least one instance of R^{1F} is —O(substituted or unsubstituted aryl). In certain embodiments, at least one instance of R^{1F} is —NO₂. In certain embodiments, at least one instance of R^{1F} is —N(R^{f2})₂ (e.g., —NMe₂). In certain embodiments, at least one instance of R^{1F} is —SR^{f1} (e.g., —SMe). In certain embodiments, at least one instance of R^{1F} is —SO₂, —CN, or —SCN.

In certain embodiments, R^{f1} is halogen, hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —NH₂, —N(R^{f2})₂, —OH, or —O(R^{f3}). In certain embodiments, R^{f1} is halogen (e.g., F, Cl, Br, or I). In certain embodiments, R^{f1} is hydrogen. In certain embodiments, R^{f1} is substituted or unsubstituted acyl (e.g., —C(═O)Me). In certain embodiments, R^{f1} is substituted or unsubstituted alkyl (e.g., substituted or unsubstituted C₁₋₆ alkyl). In certain embodiments, R^{f1} is substituted or unsubstituted methyl. In certain embodiments, R^{f1} is substituted or unsubstituted ethyl. In certain embodiments, R^{f1} is substituted or unsubstituted propyl. In certain embodiments, R^{f1} is substituted or unsubstituted alkenyl (e.g., substituted or unsubstituted C₂₋₆ alkenyl). In certain embodiments, R^{f1} is substituted or unsubstituted alkynyl (e.g., substituted or unsubstituted C₂₋₆ alkynyl). In certain embodiments, R^{f1} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{f1} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{f1} is substituted or unsubstituted aryl (e.g., substituted or unsubstituted, 6- to 10-membered aryl). In certain embodiments, R^{f1} is benzyl. In certain embodiments, R^{f1} is substituted or unsubstituted phenyl. In certain embodiments, R^{f1} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{f1} is an oxygen protecting group when attached to an oxygen atom. In certain embodiments, R^{f1} is a sulfur protecting group when attached to a sulfur atom. In certain embodiments, R^{f1} is —NH₂. In certain embodiments, R^{f1} is —N(R^{f2})₂. In certain embodiments, R^{f1} is —OH. In certain embodiments, R^{f1} is —O(R^{f3}).

In certain embodiments, each instance of R^{f2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group. In certain embodiments, at least one instance of R^{f2} is hydrogen. In certain embodiments, at least one instance of R^{f2} is substituted or unsubstituted acyl (e.g., —C(═O)Me). In certain embodiments, at least one R^{f2} is substituted or unsubstituted alkyl (e.g., substituted or unsubstituted C₁₋₆ alkyl). In certain embodiments, at least one instance of R^{f2} is substituted or unsubstituted methyl. In certain embodiments, at least one instance of RV is substituted or unsubstituted ethyl. In certain embodiments, at least one instance of RV is substituted or unsubstituted propyl. In certain embodiments, at least one instance of R^{f2} is substituted or unsubstituted alkenyl (e.g., substituted or unsubstituted C₂₋₆ alkenyl). In certain embodiments, at least one instance of R^{f2} is substituted or unsubstituted alkynyl (e.g., substituted or unsubstituted C₂₋₆ alkynyl). In certain embodiments, at least one instance of R^{f2} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{f2} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R² is substituted or unsubstituted aryl (e.g., substituted or unsubstituted, 6- to 10-membered aryl). In certain embodiments, at least one instance of R^{f2} is benzyl. In certain embodiments, at least one instance of R^{f2} is substituted or unsubstituted phenyl. In certain embodiments, at least one instance of R^{f2} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{f2} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, or two instances of R^{f2} are taken together to form a ring when attached to nitrogen.

In certain embodiments, each instance of R^{f3} is independently hydrogen or substituted or unsubstituted alkyl. In certain embodiments, at least one instance of R^{f3} is hydrogen. In certain embodiments, at least one instance of R^{f3} is substituted or unsubstituted acyl (e.g., —C(═O)Me). In certain embodiments, at least one R^{f3} is substituted or unsubstituted alkyl (e.g., substituted or unsubstituted C₁₋₆ alkyl). In certain embodiments, at least one instance of R^{f3} is substituted or unsubstituted methyl. In certain embodiments, at least one instance of R^{f3} is substituted or unsubstituted ethyl. In certain embodiments, at least one instance of R^{f3} is substituted or unsubstituted propyl. In certain embodiments, R^{f3} is substituted or unsubstituted acyl (e.g., —C(═O)Me). In certain embodiments, R^{f3} is an oxygen protecting group when attached to an oxygen atom.

In certain embodiments, R¹ is

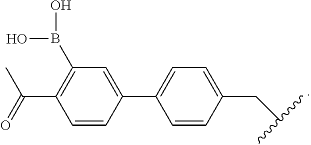 [SMILES: *Cc1ccc(-c2ccc(C(C)=O)c(B(O)O)c2)cc1]

In certain embodiments, R¹ is

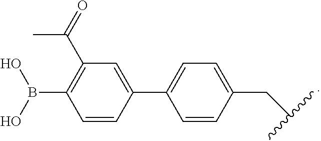 [SMILES: *Cc1ccc(-c2ccc(B(O)O)c(C(C)=O)c2)cc1]

In certain embodiments, R¹ is

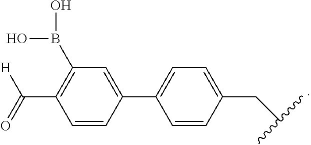 [SMILES: *Cc1ccc(-c2ccc(C=O)c(B(O)O)c2)cc1]

In certain embodiments, R¹ is

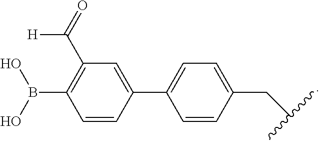 [SMILES: *Cc1ccc(-c2ccc(B(O)O)c(C=O)c2)cc1]

In certain embodiments, R¹ is

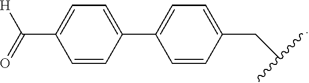 [SMILES: *Cc1ccc(-c2ccc(C=O)cc2)cc1]

In certain embodiments, R¹ is

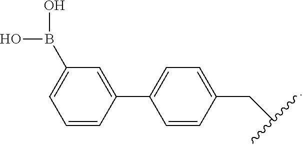 [SMILES: *Cc1ccc(-c2cccc(B(O)O)c2)cc1]

In certain embodiments, R¹ is not

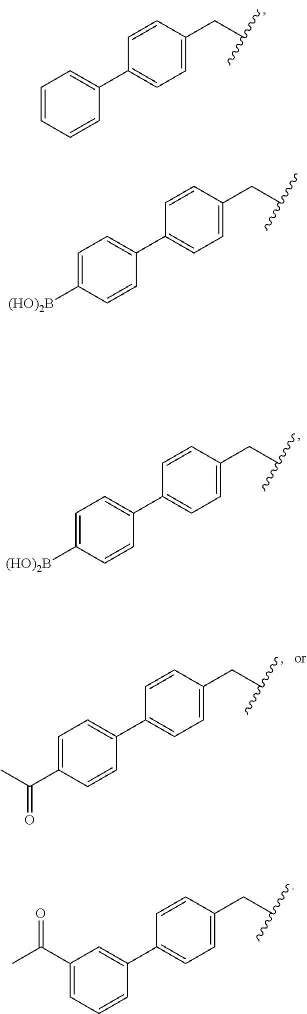 [SMILES: *Cc1ccc(-c2ccccc2)cc1 ; *Cc1ccc(-c2ccc(B(O)O)cc2)cc1 ; *Cc1ccc(-c2ccc(B(O)O)cc2)cc1 ; *Cc1ccc(-c2ccc(C(C)=O)cc2)cc1 ; *Cc1ccc(-c2cccc(C(C)=O)c2)cc1]

In certain embodiments, if R^{1D} is hydrogen, —B(OH)₂, or —C(O)CH₃, then R^{1E} is —B(OR^{a3})₂, or —C(O)R^{a3}.

### Formulae (I-A) and (I-C)

In certain embodiments, each instance of R⁵ is independently hydrogen, or substituted or unsubstituted alkyl, substituted or unsubstituted alkenyl, substituted or unsubstituted alkynyl, substituted or unsubstituted acyl, or a nitrogen protecting group. In certain embodiments, at least one instance of R⁵ is hydrogen. In certain embodiments, at least one instance of R⁵ is substituted or unsubstituted alkyl (e.g., substituted or unsubstituted C₁₋₆ alkyl). In certain embodiments, at least one instance of R⁵ is substituted or unsubstituted methyl. In certain embodiments, at least one instance of R⁵ is substituted methyl (e.g., —CF₃). In certain embodiments, at least one instance of R⁵ is unsubstituted methyl. In certain embodiments, at least one instance of R⁵ is substituted or unsubstituted ethyl. In certain embodiments, at least one instance of R⁵ is substituted ethyl (e.g., —CH₂CH₂OH). In certain embodiments, at least one instance of R⁵ is —CH₂CH₂OH. In certain embodiments, at least one instance of R⁵ is —CH₂CH₂OMe. In certain embodiments, at least one instance of R⁵ is unsubstituted ethyl. In certain embodiments, at least one instance of R⁵ is substituted or unsubstituted propyl. In certain embodiments, at least one instance of R⁵ is substituted or unsubstituted butyl (e.g., t-butyl, n-butyl). In certain embodiments, at least one instance of R⁵ is substituted or unsubstituted t-butyl. In certain embodiments, at least one instance of R⁵ is unsubstituted t-butyl. In certain embodiments, at least one instance of R⁴ is substituted or unsubstituted alkenyl (e.g., substituted or unsubstituted C₂₋₆ alkenyl). In certain embodiments, at least one instance of R⁴ is substituted or unsubstituted alkynyl (e.g., substituted or unsubstituted C₂₋₆ alkynyl). In certain embodiments, at least one instance of R⁴ is substituted or unsubstituted acyl (e.g., —C(═O)Me). In certain embodiments, at least one instance of R^{f2} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, each instance of R⁵ is hydrogen. In certain embodiments, at least one instance of R⁵ is

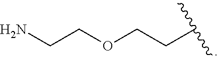 [SMILES: *CCOCCN]

In certain embodiments, one instance of R⁵ is hydrogen, and one instance of R⁵ is

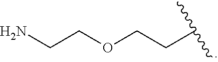 [SMILES: *CCOCCN]

In certain embodiments, at least one instance of R⁵ is

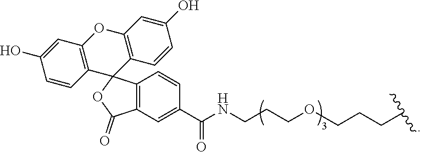

In certain embodiments, one instance of R⁵ is hydrogen, and one instance of R⁵ is

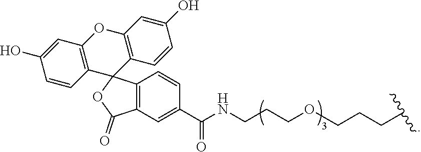

### Formulae (I-A), (I-B), and (I-C)

In certain embodiments, each instance of R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl. In certain embodiments, at least one instance of R⁴ is halogen (e.g., F, Cl, Br, or I). In certain embodiments, at least one instance of R⁴ is substituted or unsubstituted acyl (e.g., —C(═O)Me). In certain embodiments, at least one instance of R⁴ is substituted or unsubstituted alkyl (e.g., substituted or unsubstituted C₁₋₆ alkyl). In certain embodiments, at least one instance of R⁴ is substituted or unsubstituted methyl. In certain embodiments, at least one instance of R⁴ is substituted methyl (e.g., —CF₃). In certain embodiments, at least one instance of R⁴ is unsubstituted methyl. In certain embodiments, at least one instance of R⁴ is substituted or unsubstituted ethyl. In certain embodiments, at least one instance of R⁴ is substituted ethyl (e.g., —CH₂CH₂OH). In certain embodiments, at least one instance of R⁴ is —CH₂CH₂OH. In certain embodiments, at least one instance of R⁴ is —CH₂CH₂OMe. In certain embodiments, at least one instance of R⁴ is unsubstituted ethyl. In certain embodiments, at least one instance of R⁴ is substituted or unsubstituted propyl. In certain embodiments, at least one instance of R⁴ is substituted or unsubstituted butyl (e.g., t-butyl, n-butyl). In certain embodiments, at least one instance of R⁴ is substituted or unsubstituted t-butyl. In certain embodiments, at least one instance of R⁴ is unsubstituted t-butyl. In certain embodiments, at least one instance of R⁴ is substituted or unsubstituted alkenyl (e.g., substituted or unsubstituted C₂₋₆ alkenyl). In certain embodiments, at least one instance of R⁴ is substituted or unsubstituted alkynyl (e.g., substituted or unsubstituted C₂₋₆ alkynyl). In certain embodiments, at least one instance of R⁴ is substituted or unsubstituted carbocyclyl (e.g., substituted or unsubstituted, 3- to 10-membered, monocyclic carbocyclyl comprising zero, one, or two double bonds in the carbocyclic ring system). In certain embodiments, at least one instance of R⁴ is substituted or unsubstituted aryl (e.g., substituted or unsubstituted, 6- to 10-membered aryl). In certain embodiments, at least one instance of R⁴ is benzyl. In certain embodiments, at least one instance of R⁴ is substituted or unsubstituted phenyl. In certain embodiments, at least one instance of R⁴ is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R⁴ is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur).

In certain embodiments, the moiety

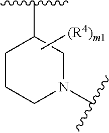

is

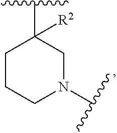

wherein R² is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl. In certain embodiments, R² is hydrogen. In certain embodiments, R² is substituted or unsubstituted acyl (e.g., —C(═O)Me). In certain embodiments, R² is substituted or unsubstituted alkyl (e.g., substituted or unsubstituted C₁₋₆ alkyl). In certain embodiments, R² is substituted or unsubstituted C₁₋₆ alkyl. In certain embodiments, R² is substituted or unsubstituted methyl. In certain embodiments, R² is methyl substituted or unsubstituted with —OR¹, wherein R^{c1} is hydrogen, substituted or unsubstituted acyl, substituted or unsubstituted alkyl, substituted or unsubstituted alkenyl, substituted or unsubstituted alkynyl, or oxygen protecting group. In certain embodiments, R² is methyl substituted or unsubstituted with —OH, —O(substituted or unsubstituted C₁₋₆ alkyl), or —O(substituted or unsubstituted C₂₋₆ alkenyl). In certain embodiments, R² is substituted or unsubstituted ethyl. In certain embodiments, R² is substituted or unsubstituted propyl. In certain embodiments, R² is substituted or unsubstituted alkenyl (e.g., substituted or unsubstituted C₂₋₆ alkenyl). In certain embodiments, R² is

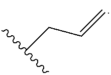 [SMILES: *CC=C]

In certain embodiments, R² is substituted or unsubstituted alkynyl (e.g., substituted or unsubstituted C₂₋₆ alkynyl). In certain embodiments, R² is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R² is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R² is substituted or unsubstituted aryl (e.g., substituted or unsubstituted, 6- to 10-membered aryl). In certain embodiments, R² is benzyl. In certain embodiments, R² is substituted or unsubstituted benzyl. In certain embodiments, R² is substituted or unsubstituted phenyl. In certain embodiments, R² is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur).

In certain embodiments, the moiety

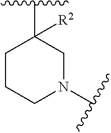

is

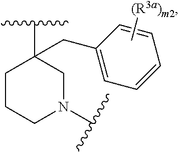

wherein each instance of R^{3a} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN; and m2 is 0, 1, 2, 3, 4, or 5. In certain embodiments, in the moiety

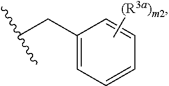

there are zero instances of R^{3a}. In certain embodiments, there are zero instances of R^{3a}. In certain embodiments, m2 is 0. In certain embodiments, there are one or more instances of R^{3a}. In certain embodiments, m2 is 1. In certain embodiments, at least one instance of m2 is 2. In certain embodiments, at least one instance of m2 is 3. In certain embodiments, at least one instance of m2 is 4. In certain embodiments, at least one instance of m2 is 5. In certain embodiments, at least one instance of R^{3a} is halogen (e.g., F, Cl, Br, or I). In certain embodiments, m2 is 2 and both instances of R^{3a} are halogen (e.g., F, Cl, Br, or I). In certain embodiments, at least one instance of R^{3a} is substituted or unsubstituted acyl (e.g., —C(═O)Me). In certain embodiments, at least one instance of R^{3a} is substituted or unsubstituted alkyl (e.g., substituted or unsubstituted C₁₋₆ alkyl). In certain embodiments, at least one instance of R^{3a} is substituted or unsubstituted methyl. In certain embodiments, at least one instance of R^{3a} is methyl substituted or unsubstituted with halogen. In certain embodiments, at least one instance of R^{3a} is —CF₃. In certain embodiments, at least one instance of R^{3a} is substituted or unsubstituted ethyl. In certain embodiments, at least one instance of R^{3a} is substituted or unsubstituted propyl. In certain embodiments, at least one instance of R^{3a} is substituted or unsubstituted butyl (e.g., substituted or unsubstituted n-butyl or substituted or unsubstituted t-butyl). In certain embodiments, at least one instance of R^{3a} is substituted or unsubstituted t-butyl. In certain embodiments, at least one instance of R^{3a} is unsubstituted t-butyl. In certain embodiments, at least one instance of R^{3a} is substituted or unsubstituted alkenyl (e.g., substituted or unsubstituted C₂₋₆ alkenyl). In certain embodiments, at least one instance of R^{3a} is substituted or unsubstituted alkynyl (e.g., substituted or unsubstituted C₂₋₆ alkynyl). In certain embodiments, at least one instance of R^{3a} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{3a} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{3a} is substituted or unsubstituted aryl (e.g., substituted or unsubstituted, 6- to 10-membered aryl). In certain embodiments, at least one instance of R^{3a} is benzyl. In certain embodiments, at least one instance of R^{3a} is substituted or unsubstituted phenyl. In certain embodiments, at least one instance of R^{3a} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{3a} is —OR^{c1} (e.g., —OH or —OMe). In certain embodiments, at least one instance of R^{3a} is —O(substituted or unsubstituted C₁₋₆ alkyl). In certain embodiments, at least one instance of R^{3a} is —OMe. In certain embodiments, at least one instance of R^{3a} is -OEt. In certain embodiments, at least one instance of R^{3a} is —O(substituted or unsubstituted C₂₋₆ alkenyl). In certain embodiments, at least one instance of R^{3a} is

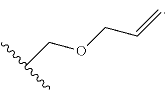 [SMILES: *COCC=C]

In certain embodiments, at least one instance of R^{3a} is —NO₂. In certain embodiments, at least one instance of R^{3a} is —N(R^{c2})₂(e.g., —NMe₂). In certain embodiments, at least one instance of R^{3a} is —SR^{c1} (e.g., —SMe). In certain embodiments, at least one instance of R^{3a} is —CN. In certain embodiments, at least one instance of R^{3a} is —SCN.

In certain embodiments, each of R^{A}, R^{B}, R^{C}, and R^{D} is independently hydrogen, substituted or unsubstituted alkyl, substituted or unsubstituted acyl, or a nitrogen protecting group. In certain embodiments, R^{A} is hydrogen. In certain embodiments, R^{A} is substituted or unsubstituted acyl (e.g., —C(═O)Me). In certain embodiments, R^{A} is substituted or unsubstituted alkyl (e.g., substituted or unsubstituted C₁₋₆ alkyl). In certain embodiments, R^{A} is substituted or unsubstituted C₁₋₆ alkyl. In certain embodiments, R^{A} is substituted or unsubstituted methyl. In certain embodiments, R^{A} is unsubstituted methyl. In certain embodiments, R^{A} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, R^{B} is hydrogen. In certain embodiments, R^{B} is substituted or unsubstituted acyl (e.g., —C(═O)Me). In certain embodiments, R^{B} is substituted or unsubstituted alkyl (e.g., substituted or unsubstituted C₁₋₆ alkyl). In certain embodiments, R^{B} is substituted or unsubstituted C₁₋₆ alkyl. In certain embodiments, R^{B} is substituted or unsubstituted methyl. In certain embodiments, R^{B} is unsubstituted methyl. In certain embodiments, R^{B} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, R^{C} is hydrogen. In certain embodiments, R^{C} is substituted or unsubstituted acyl (e.g., —C(═O)Me). In certain embodiments, R^{C} is substituted or unsubstituted alkyl (e.g., substituted or unsubstituted C₁₋₆ alkyl). In certain embodiments, R^{C} is substituted or unsubstituted C₁₋₆ alkyl. In certain embodiments, R^{C} is substituted or unsubstituted methyl. In certain embodiments, R^{C} is substituted or unsubstituted ethyl. In certain embodiments, R^{C} is unsubstituted methyl. In certain embodiments, R^{C} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, R^{D} is hydrogen. In certain embodiments, R^{D} is substituted or unsubstituted acyl (e.g., —C(═O)Me). In certain embodiments, R^{D} is substituted or unsubstituted alkyl (e.g., substituted or unsubstituted C₁₋₆ alkyl). In certain embodiments, R^{D} is substituted or unsubstituted C₁₋₆ alkyl. In certain embodiments, R^{D} is substituted or unsubstituted methyl. In certain embodiments, R^{D} is unsubstituted methyl. In certain embodiments, R^{D} is substituted or unsubstituted ethyl. In certain embodiments, R^{D} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, each of R^{A}, R^{B}, R^{C}, and R^{D} is hydrogen. In certain embodiments, one of R^{A}, R^{B}, R^{C}, and R^{D} is substituted or unsubstituted C₁₋₆ alkyl, and the rest of R^{A}, R^{B}, R^{C}, and R^{D} are each hydrogen. In certain embodiments, R^{B} is substituted or unsubstituted C₁₋₆ alkyl (e.g., methyl), and the rest of R^{A}, R^{C}, and R^{D} are each hydrogen. In certain embodiments, R^{B} is methyl, and the rest of R^{A}, R^{C}, and R^{D} are each hydrogen. In certain embodiments, one of R^{A}, R^{B}, R^{C}, and R^{D} is substituted or unsubstituted acyl (e.g., —C(═O)Me), and the rest of R^{A}, R^{B}, R^{C}, and R^{D} are each hydrogen. In certain embodiments, one of R^{A}, R^{B}, R^{C}, and R^{D} is substituted or unsubstituted acyl (e.g., —C(═O)Me), and the rest of R^{A}, R^{B}, R^{C}, and R^{D} are each hydrogen. In certain embodiments, one of R^{A}, R^{B}, R^{C}, and R^{D} is a nitrogen protecting group, and the rest of R^{A}, R^{B}, R^{C}, and R^{D} are each hydrogen. In certain embodiments, R^{A} is hydrogen, and the rest of of R^{B}, R^{C}, and R^{D} are each independently hydrogen, substituted or unsubstituted alkyl, substituted or unsubstituted acyl, or a nitrogen protecting group. In certain embodiments, R^{B} is hydrogen, and the rest of of R^{A}, R^{C}, and R^{D} are each independently hydrogen, substituted or unsubstituted alkyl, substituted or unsubstituted acyl, or a nitrogen protecting group. In certain embodiments, R^{C} is hydrogen, and the rest of of R^{A}, R^{B}, and R^{D} are each independently hydrogen, substituted or unsubstituted alkyl, substituted or unsubstituted acyl, or a nitrogen protecting group. In certain embodiments, R^{D} is hydrogen, and the rest of of R^{A}, R^{B}, and R^{C} are each independently hydrogen, substituted or unsubstituted alkyl, substituted or unsubstituted acyl, or a nitrogen protecting group.

In certain embodiments, x is 0. In certain embodiments, x is 1.

In certain embodiments, y is 0. In certain embodiments, y is 1.

In certain embodiments, x is 0 and y is 0. In certain embodiments, x is 0 and y is 1. In certain embodiments, x is 1 and y is 0. In certain embodiments, x is 1 and y is 1.

In certain embodiments, m1 is 0. In certain embodiments, m1 is 1. In certain embodiments, m1 is 2. In certain embodiments, m1 is 3. In certain embodiments, m1 is 4. In certain embodiments, m1 is 5. In certain embodiments, m1 is 6.

In certain embodiments, q is 0. In certain embodiments, q is 1. In certain embodiments, q is 2. In certain embodiments, q is 3. In certain embodiments, q is 4.

In certain embodiments, p is 0. In certain embodiments, p is 1. In certain embodiments, p is 2. In certain embodiments, p is 3. In certain embodiments, p is 4.

In certain embodiments, r is 0. In certain embodiments, r is 1. In certain embodiments, r is 2. In certain embodiments, r is 3.

In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, n1 is 0. In certain embodiments, n1 is 1. In certain embodiments, n1 is 2. In certain embodiments, n1 is 3. In certain embodiments, n1 is 4. In certain embodiments, n1 is 5. In certain embodiments, n1 is 6. In certain embodiments, n1 is 7. In certain embodiments, n1 is 8. In certain embodiments, n1 is 9. In certain embodiments, n1 is 10.

In certain embodiments, n2 is 0. In certain embodiments, n2 is 1. In certain embodiments, n2 is 2. In certain embodiments, n2 is 3. In certain embodiments, n2 is 4. In certain embodiments, n2 is 5. In certain embodiments, n2 is 6. In certain embodiments, n2 is 7. In certain embodiments, n2 is 8. In certain embodiments, n2 is 9. In certain embodiments, n2 is 10.

In certain embodiments, n3 is 0. In certain embodiments, n3 is 1. In certain embodiments, n3 is 2. In certain embodiments, n3 is 3. In certain embodiments, n3 is 4. In certain embodiments, n3 is 5. In certain embodiments, n3 is 6. In certain embodiments, n3 is 7. In certain embodiments, n3 is 8. In certain embodiments, n3 is 9. In certain embodiments, n3 is 10.

In certain embodiments, n4 is 0. In certain embodiments, n4 is 1. In certain embodiments, n4 is 2. In certain embodiments, n4 is 3. In certain embodiments, n4 is 4. In certain embodiments, n4 is 5. In certain embodiments, n4 is 6. In certain embodiments, n4 is 7. In certain embodiments, n4 is 8. In certain embodiments, n4 is 9. In certain embodiments, n4 is 10.

In certain embodiments, m1 is 0, p is 0, and q is 0. In certain embodiments, m1 is 1, p is 0, and q is 0. In certain embodiments, m1 is 1, p is 0, and q is 1. In certain embodiments, m1 is 1, p is 0, and q is 2. In certain embodiments, m1 is 1, p is 0, and q is 3. In certain embodiments, m1 is 1, p is 0, and q is 4. In certain embodiments, m1 is 1, p is 1, and q is 0. In certain embodiments, m1 is 1, p is 1, and q is 1. In certain embodiments, m1 is 1, p is 1, and q is 2. In certain embodiments, m1 is 1, p is 1, and q is 3. In certain embodiments, m1 is 1, p is 1, and q is 4. In certain embodiments, m1 is 1, p is 2, and q is 0. In certain embodiments, m1 is 1, p is 2, and q is 1. In certain embodiments, m1 is 1, p is 2, and q is 2. In certain embodiments, m1 is 1, p is 2, and q is 3. In certain embodiments, m1 is 1, p is 2, and q is 4. In certain embodiments, m1 is 1, p is 3, and q is 0. In certain embodiments, m1 is 1, p is 3, and q is 1. In certain embodiments, m1 is 1, p is 3, and q is 2. In certain embodiments, m1 is 1, p is 3, and q is 3. In certain embodiments, m1 is 1, p is 3, and q is 4. In certain embodiments, m1 is 1, p is 4, and q is 0. In certain embodiments, m1 is 1, p is 4, and q is 1. In certain embodiments, m1 is 1, p is 4, and q is 2. In certain embodiments, m1 is 1, p is 4, and q is 3. In certain embodiments, m1 is 1, p is 4, and q is 4. In certain embodiments, x is 0, y is 0, m1 is 1, p is 0, and q is 0.

In certain embodiments, m1 is 0, n is 3, p is 0, and q is 0. In certain embodiments, m1 is 1, n is 3, p is 0, and q is 0. In certain embodiments, m1 is 1, n is 3, p is 0, and q is 1. In certain embodiments, m1 is 1, n is 3, p is 0, and q is 2. In certain embodiments, m1 is 1, n is 3, p is 0, and q is 3. In certain embodiments, m1 is 1, n is 3, p is 0, and q is 4. In certain embodiments, m1 is 1, n is 3, p is 1, and q is 0. In certain embodiments, m1 is 1, n is 3, p is 1, and q is 1. In certain embodiments, m1 is 1, n is 3, p is 1, and q is 2. In certain embodiments, m1 is 1, n is 3, p is 1, and q is 3. In certain embodiments, m1 is 1, n is 3, p is 1, and q is 4. In certain embodiments, m1 is 1, n is 3, p is 2, and q is 0. In certain embodiments, m1 is 1, n is 3, p is 2, and q is 1. In certain embodiments, m1 is 1, n is 3, p is 2, and q is 2. In certain embodiments, m1 is 1, n is 3, p is 2, and q is 3. In certain embodiments, m1 is 1, n is 3, p is 2, and q is 4. In certain embodiments, m1 is 1, n is 3, p is 3, and q is 0. In certain embodiments, m1 is 1, n is 3, p is 3, and q is 1. In certain embodiments, m1 is 1, n is 3, p is 3, and q is 2. In certain embodiments, m1 is 1, n is 3, p is 3, and q is 3. In certain embodiments, m1 is 1, n is 3, p is 3, and q is 4. In certain embodiments, m1 is 1, n is 3, p is 4, and q is 0. In certain embodiments, m1 is 1, n is 3, p is 4, and q is 1. In certain embodiments, m1 is 1, n is 3, p is 4, and q is 2. In certain embodiments, m1 is 1, n is 3, p is 4, and q is 3. In certain embodiments, m1 is 1, n is 3, p is 4, and q is 4. In certain embodiments, x is 0, y is 0, m1 is 1, n is 3, p is 0, and q is 0.

In certain embodiments, m1 is 0 and r is 0. In certain embodiments, m1 is 1 and r is 0. In certain embodiments, m1 is 1 and r is 1. In certain embodiments, m1 is 1 and r is 2. In certain embodiments, m1 is 1 and r is 3.

In certain embodiments, n1 is 3, n2 is 0, n3 is 0, and n4 is 0. In certain embodiments, n1 is 3, n2 is 1, n3 is 0, and n4 is 0. In certain embodiments, n1 is 3, n2 is 2, n3 is 0, and n4 is 0. In certain embodiments, n1 is 3, n2 is 3, n3 is 0, and n4 is 0. In certain embodiments, n1 is 3, n2 is 3, n3 is 1, and n4 is 0. In certain embodiments, n1 is 3, n2 is 3, n3 is 2, and n4 is 0. In certain embodiments, n1 is 3, n2 is 3, n3 is 1, and n4 is 1. In certain embodiments, n1 is 3, n2 is 3, n3 is 2, and n4 is 1. In certain embodiments, m1 is 1, r is 0, n1 is 3, n2 is 3, n3 is 1, and n4 is 0. In certain embodiments, m1 is 1, p is 0, q is 0, n1 is 3, n2 is 3, n3 is 1, and n4 is 0.

In certain embodiments, n1 is 1, n2 is 0, n3 is 1, and n4 is 0. In certain embodiments, n1 is 1, n2 is 0, n3 is 1, and n4 is 1. In certain embodiments, n1 is 1, n2 is 0, n3 is 1, and n4 is 2. In certain embodiments, n1 is 1, n2 is 0, n3 is 1, and n4 is 3. In certain embodiments, n1 is 1, n2 is 0, n3 is 1, and n4 is 4. In certain embodiments, n1 is 1, n2 is 0, n3 is 1, and n4 is 5. In certain embodiments, n1 is 2, n2 is 0, n3 is 0, and n4 is 5. In certain embodiments, n1 is 0, n2 is 0, n3 is 2, and n4 is 5.

In certain embodiments, the compound of Formula (I-A), (I-B), or (I-C) is of formula:

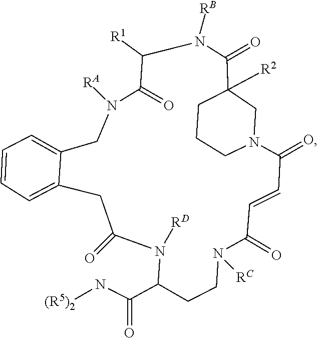

or a pharmaceutically acceptable salt, solvate, hydrate, polymorph, co-crystal, tautomer, stereoisomer, isotopically enriched form, prodrug, or mixture thereof, wherein substituents R¹, R², R⁵, R^{A}, R^{B}, R^{C}, and R^{D} are defined as described herein.

In certain embodiments, the compound of Formula (I-A), (I-B), or (I-C) is of formula:

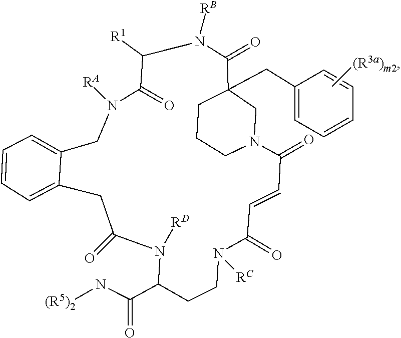

or a pharmaceutically acceptable salt, solvate, hydrate, polymorph, co-crystal, tautomer, stereoisomer, isotopically enriched form, prodrug, or mixture thereof, wherein: substituents R¹, R⁵, R^{3a}, R^{A}, R^{B}, R^{C}, and R^{D} are defined as described herein.

In certain embodiments, the compound of Formula (I-A) is of formula:

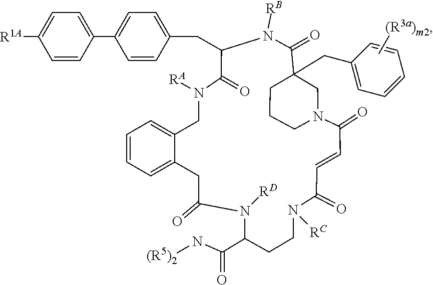

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-A) is of formula:

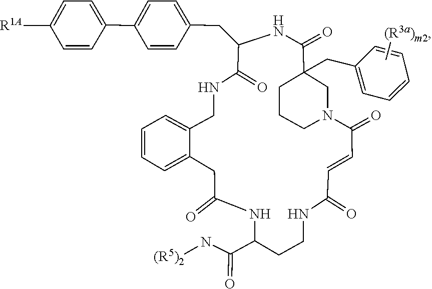

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-A) is of formula:

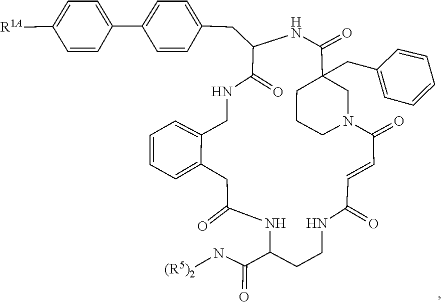

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-A) is of formula:

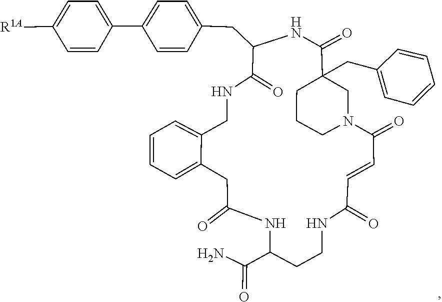

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-A) is of formula:

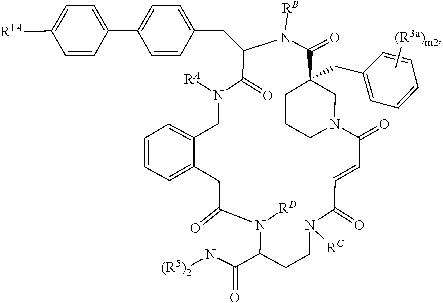

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-A) is of formula:

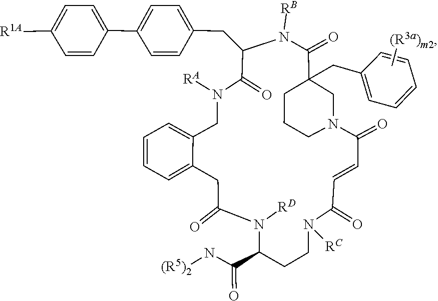

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-A) is of formula:

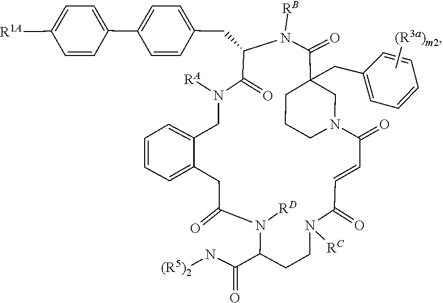

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-A) is of formula:

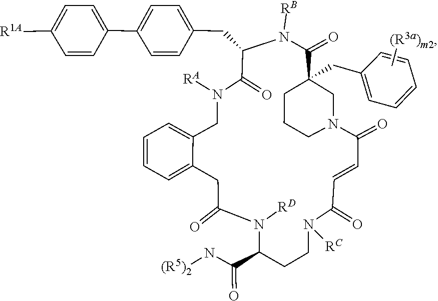

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-A) is of formula:

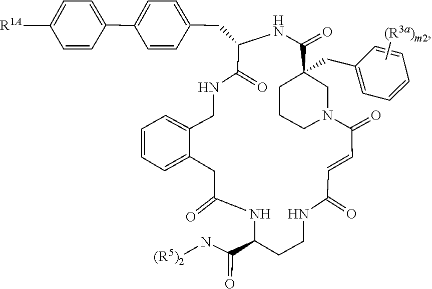

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-A) is of formula:

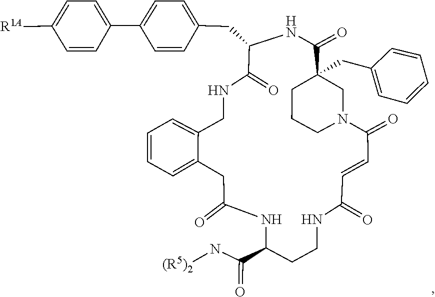

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-A) is of formula:

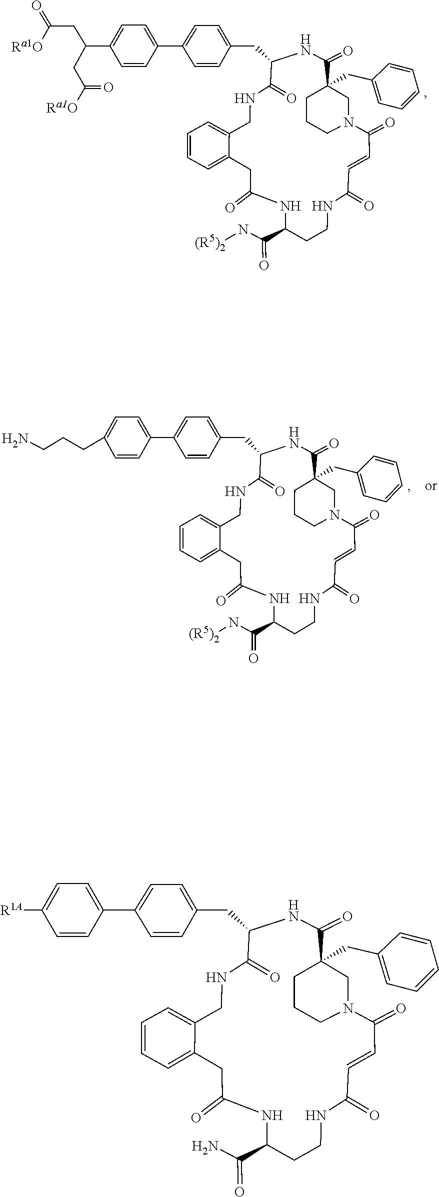

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

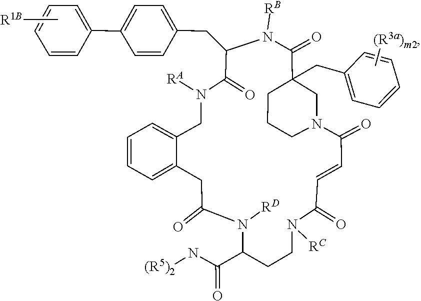

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

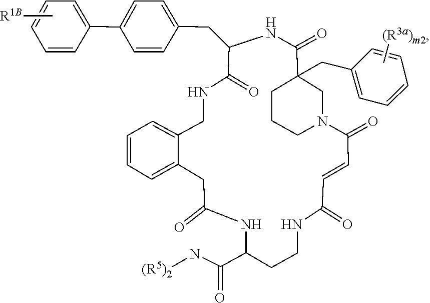

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

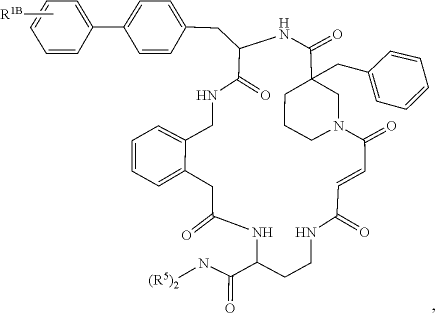

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

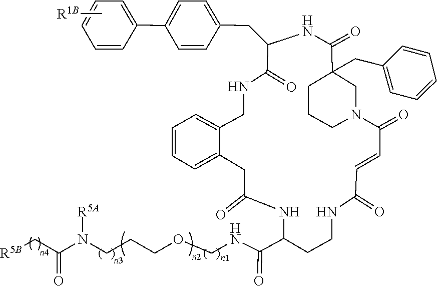

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

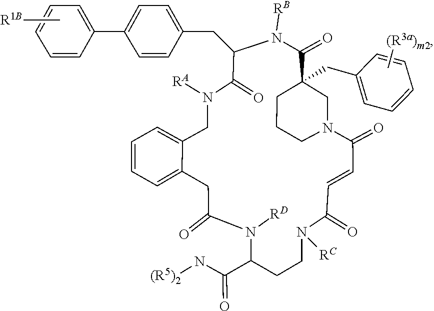

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

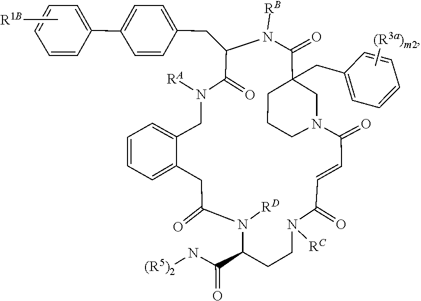

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

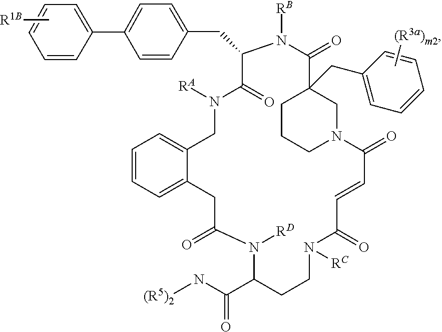

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

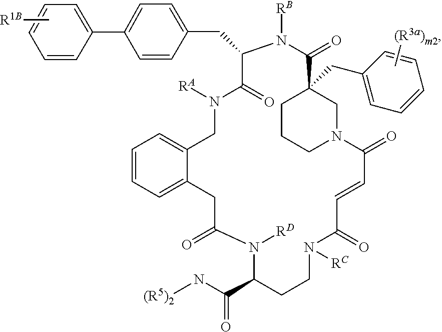

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

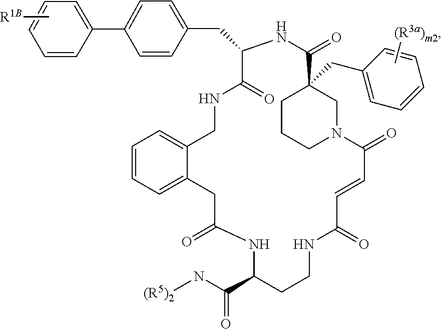

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

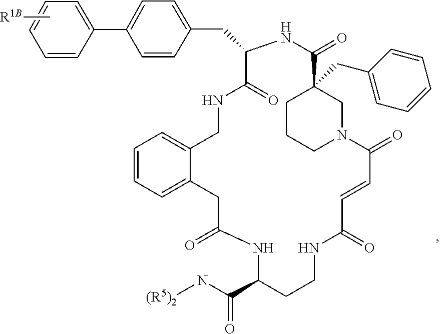

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

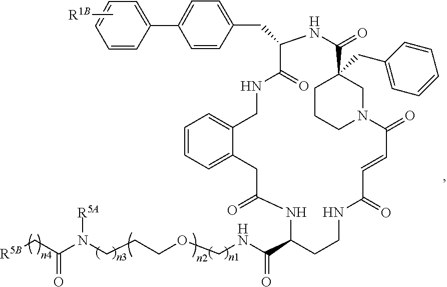

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

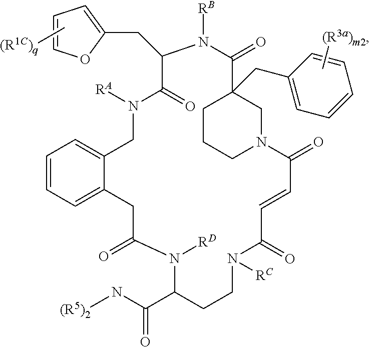

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

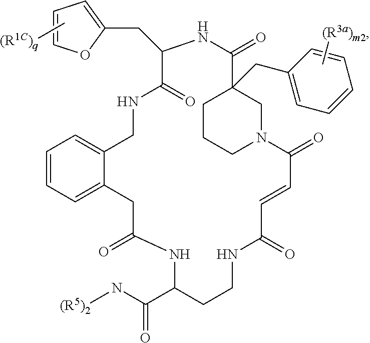

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

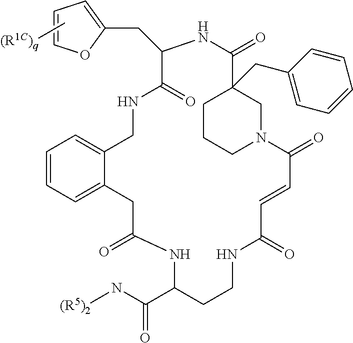

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

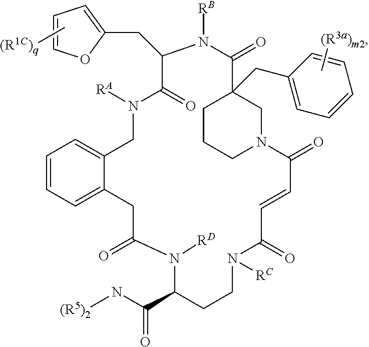

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

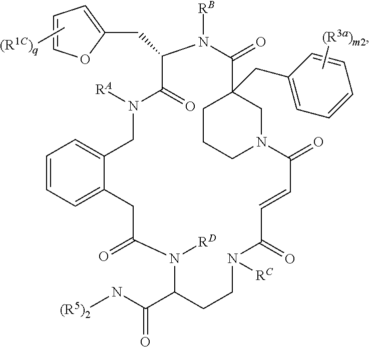

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

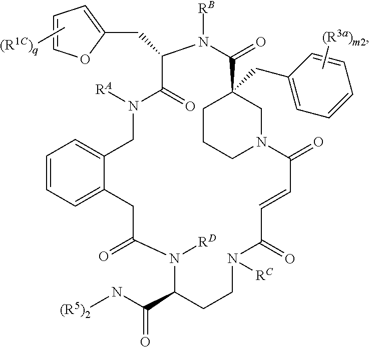

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

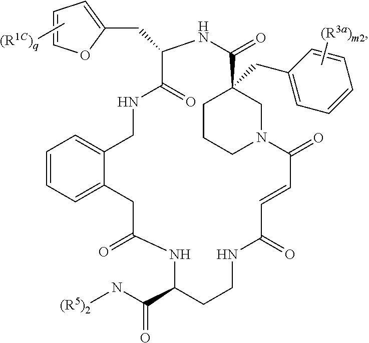

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

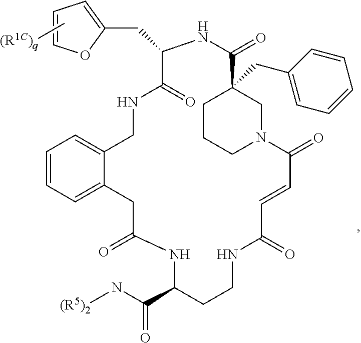

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

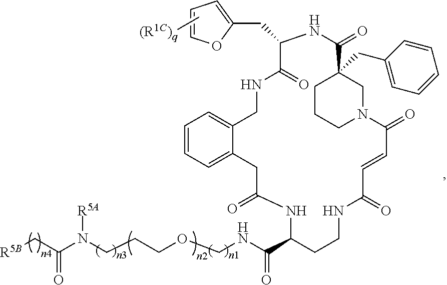

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-C) is of formula:

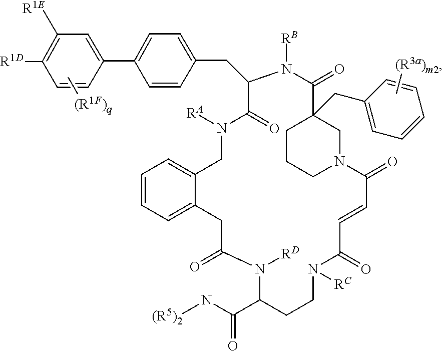

or a pharmaceutically acceptable salt, solvate, hydrate, polymorph, co-crystal, tautomer, stereoisomer, isotopically enriched form, prodrug, or mixture thereof.

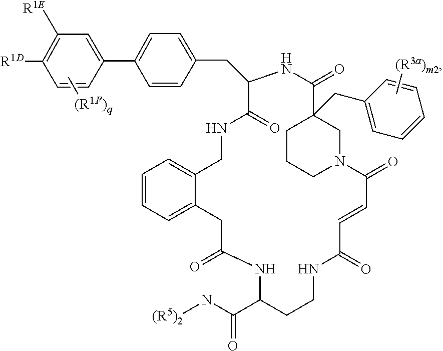

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-C) is of formula:

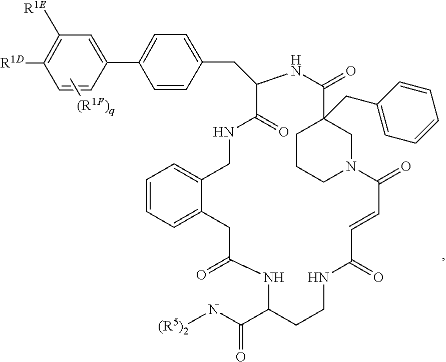

or a pharmaceutically acceptable salt, solvate, hydrate, polymorph, co-crystal, tautomer, stereoisomer, isotopically enriched form, prodrug, or mixture thereof.

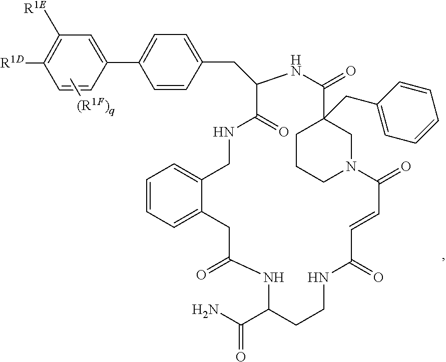

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-C) is of formula:

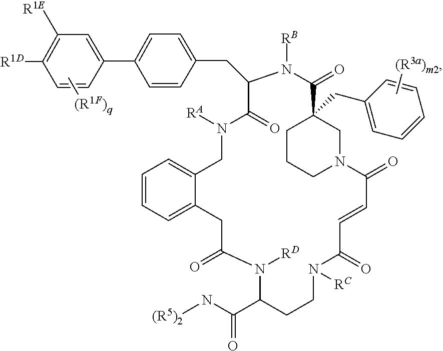

or a pharmaceutically acceptable salt, solvate, hydrate, polymorph, co-crystal, tautomer, stereoisomer, isotopically enriched form, prodrug, or mixture thereof.

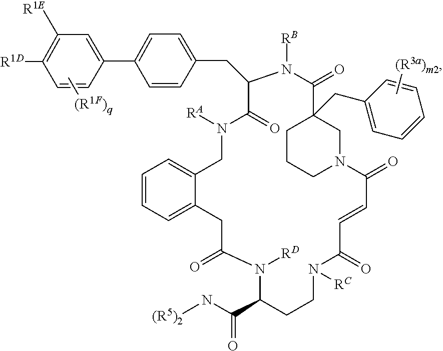

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-C) is of formula:

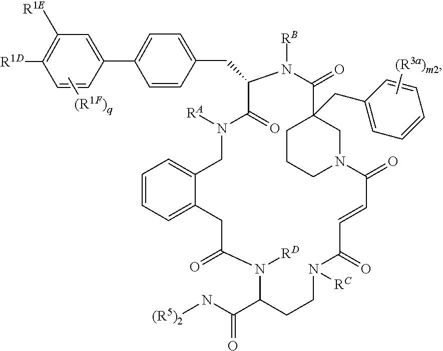

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-C) is of formula:

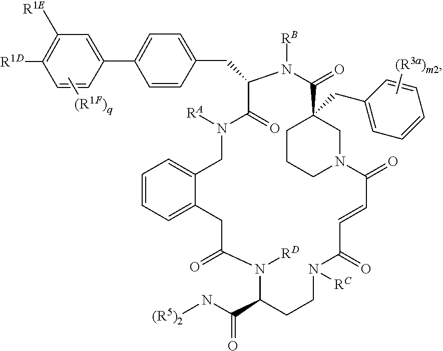

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-C) is of formula:

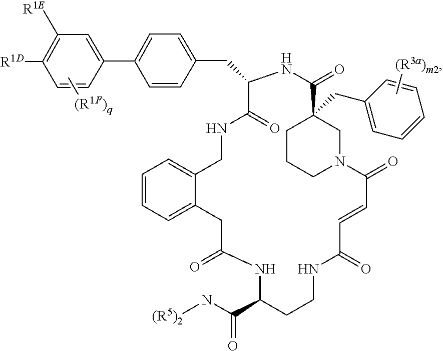

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-C) is of formula:

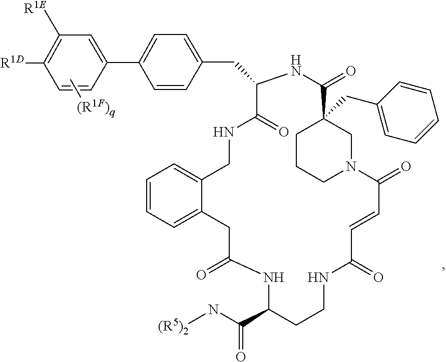

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-C) is of formula:

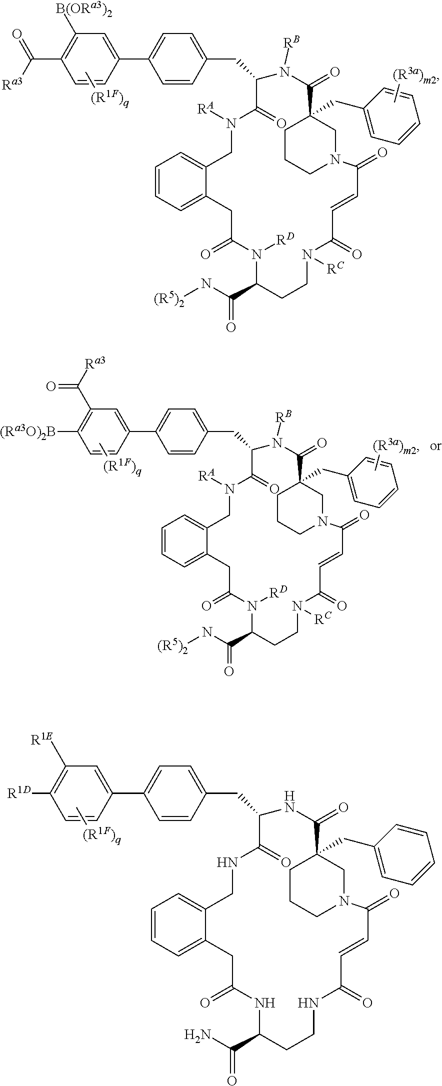

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-A) is of formula:

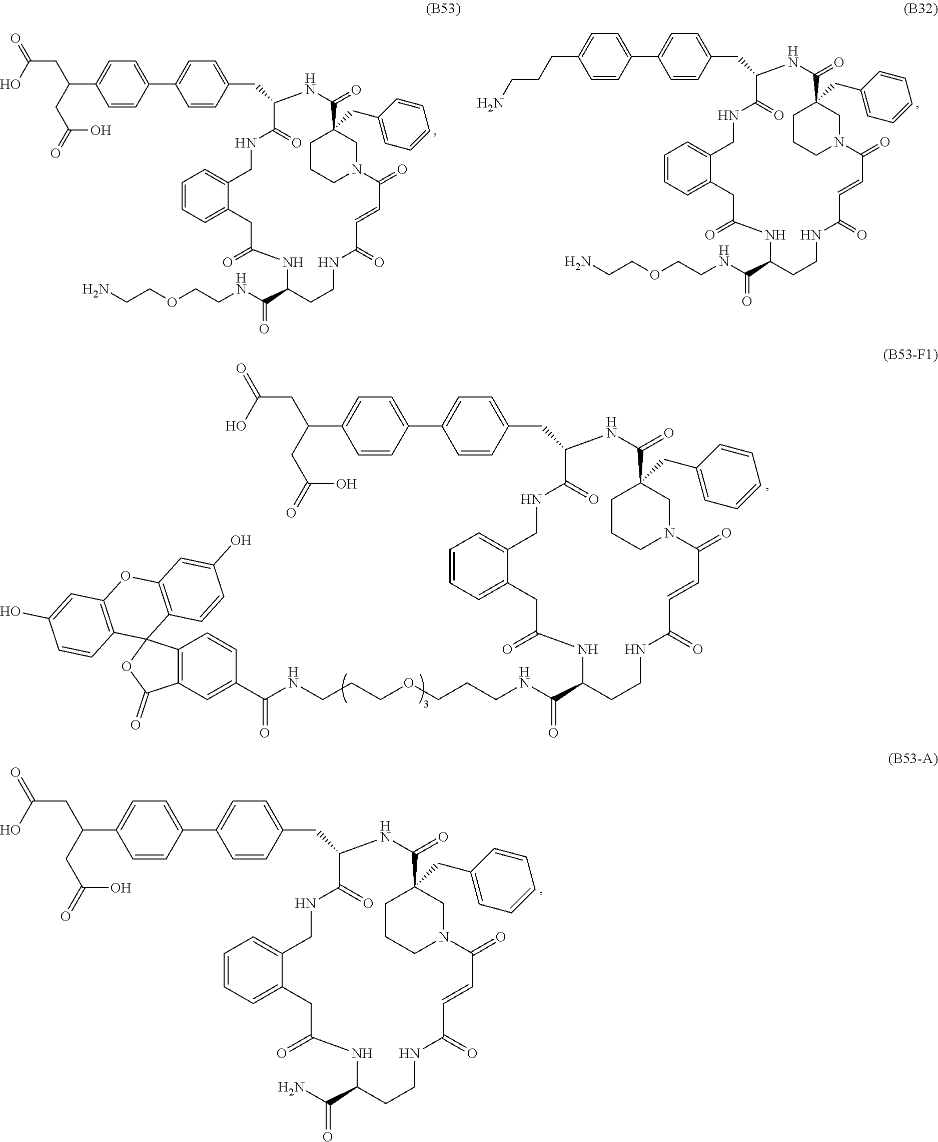

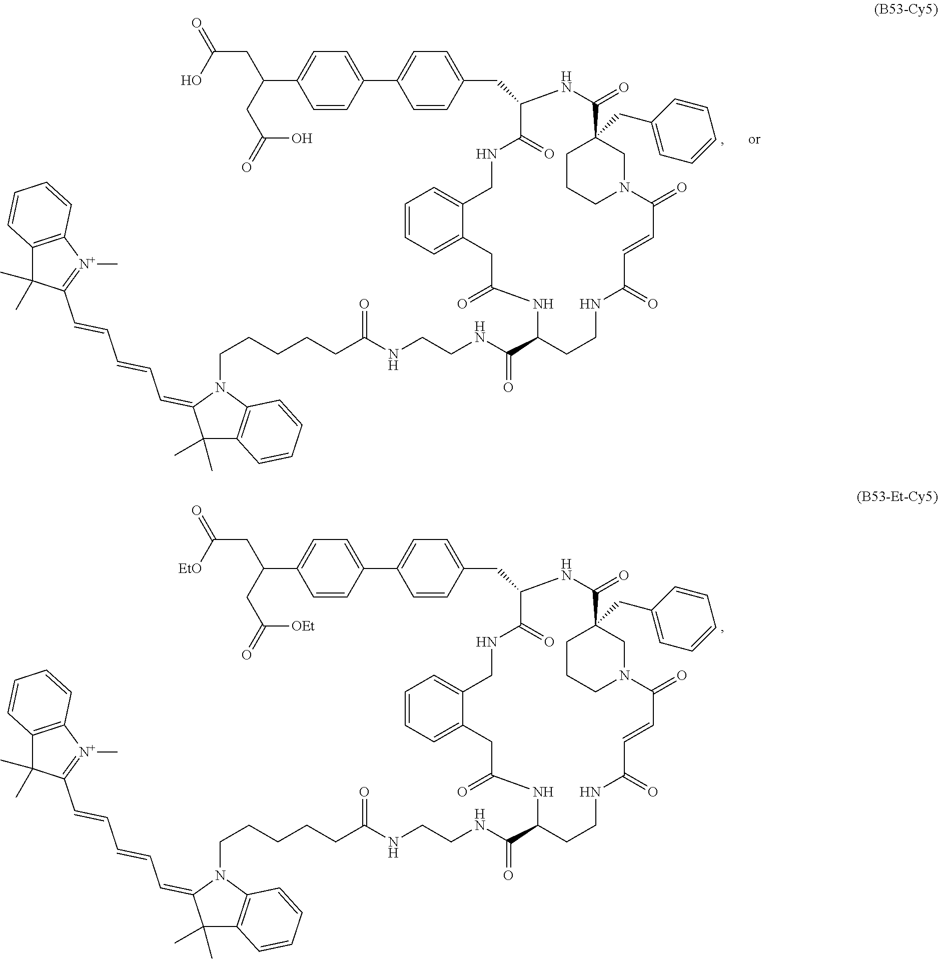 [SMILES: C[N+]1=C(/C=C/C=C/C=C2\N(CCCCCC(=O)NCCNC(=O)[C@@H]3CCNC(=O)/C=C/C(=O)N4CCC[C@](Cc5ccccc5)(C4)C(=O)N[C@@H](Cc4ccc(-c5ccc(C(CC(=O)O)CC(=O)O)cc5)cc4)C(=O)NCc4ccccc4CC(=O)N3)c3ccccc3C2(C)C)C(C)(C)c2ccccc21 ; CCOC(=O)CC(CC(=O)OCC)c1ccc(-c2ccc(C[C@@H]3NC(=O)[C@]4(Cc5ccccc5)CCCN(C4)C(=O)/C=C/C(=O)NCC[C@@H](C(=O)NCCNC(=O)CCCCCN4\C(=C/C=C/C=C/C5=[N+](C)c6ccccc6C5(C)C)C(C)(C)c5ccccc54)NC(=O)Cc4ccccc4CNC3=O)cc2)cc1]

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-B) is of formula:

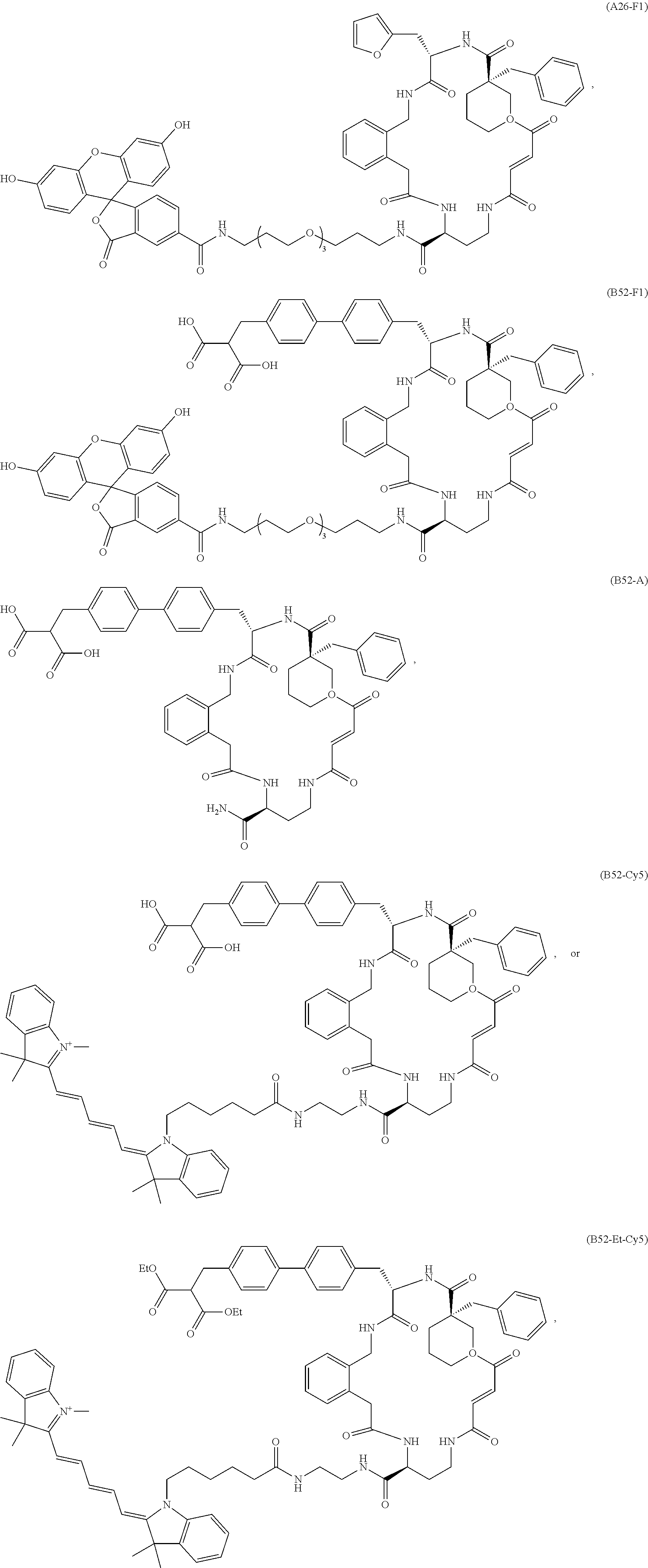

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-C) is of formula:

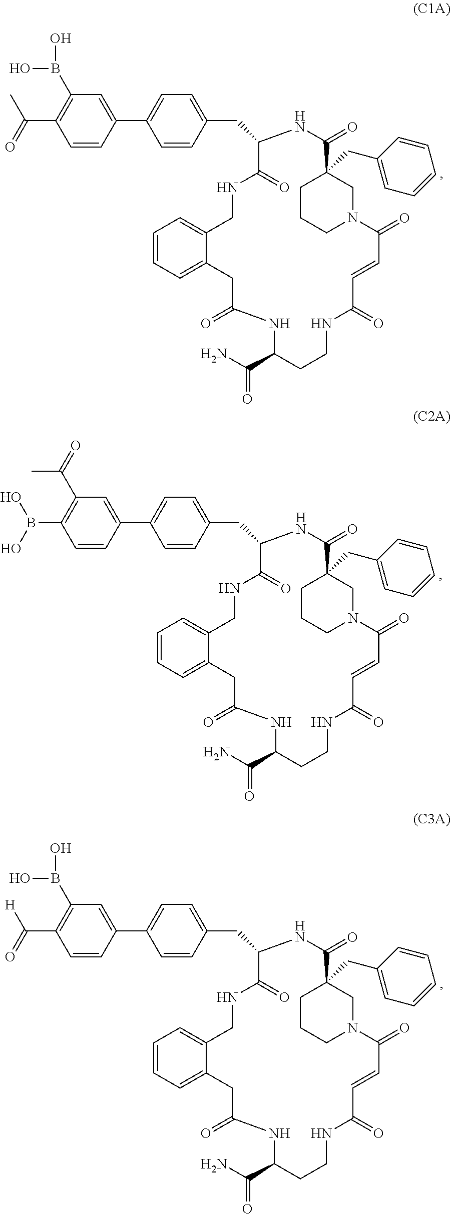 [SMILES: CC(=O)c1ccc(-c2ccc(C[C@@H]3NC(=O)[C@]4(Cc5ccccc5)CCCN(C4)C(=O)/C=C/C(=O)NCC[C@@H](C(N)=O)NC(=O)Cc4ccccc4CNC3=O)cc2)cc1B(O)O ; CC(=O)c1cc(-c2ccc(C[C@@H]3NC(=O)[C@]4(Cc5ccccc5)CCCN(C4)C(=O)/C=C/C(=O)NCC[C@@H](C(N)=O)NC(=O)Cc4ccccc4CNC3=O)cc2)ccc1B(O)O ; NC(=O)[C@@H]1CCNC(=O)/C=C/C(=O)N2CCC[C@](Cc3ccccc3)(C2)C(=O)N[C@@H](Cc2ccc(-c3ccc(C=O)c(B(O)O)c3)cc2)C(=O)NCc2ccccc2CC(=O)N1]

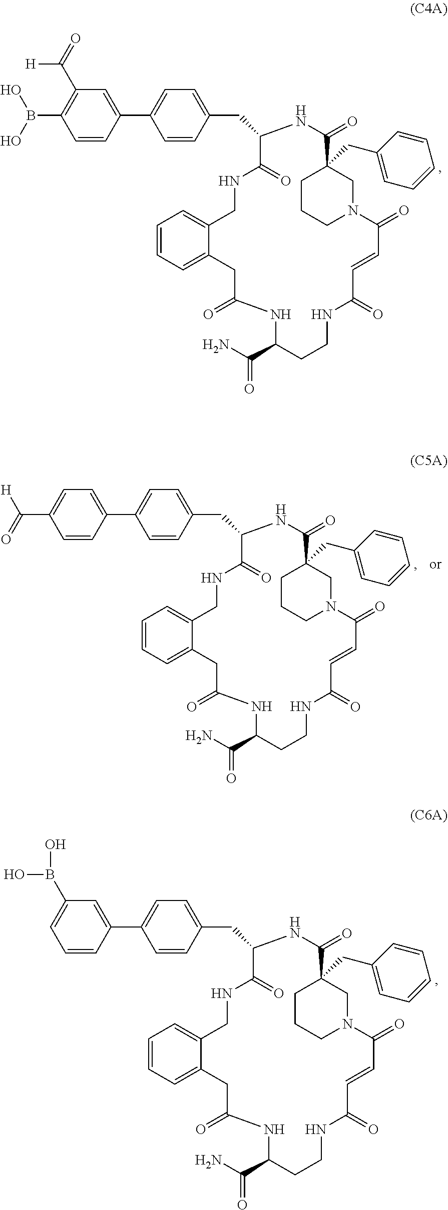 [SMILES: NC(=O)[C@@H]1CCNC(=O)/C=C/C(=O)N2CCC[C@](Cc3ccccc3)(C2)C(=O)N[C@@H](Cc2ccc(-c3ccc(B(O)O)c(C=O)c3)cc2)C(=O)NCc2ccccc2CC(=O)N1 ; NC(=O)[C@@H]1CCNC(=O)/C=C/C(=O)N2CCC[C@](Cc3ccccc3)(C2)C(=O)N[C@@H](Cc2ccc(-c3ccc(C=O)cc3)cc2)C(=O)NCc2ccccc2CC(=O)N1 ; NC(=O)[C@@H]1CCNC(=O)/C=C/C(=O)N2CCC[C@](Cc3ccccc3)(C2)C(=O)N[C@@H](Cc2ccc(-c3cccc(B(O)O)c3)cc2)C(=O)NCc2ccccc2CC(=O)N1]

or a pharmaceutically acceptable salt, solvate, hydrate, polymorph, co-crystal, tautomer, stereoisomer, isotopically enriched form, prodrug, or mixture thereof.

In certain embodiments, the compound of Formula (I-A) is of formula B53, B32, B53-Fl, B53-A, B53-Cy5, B53-Et-Cy5, or a pharmaceutically acceptable salt thereof.

In certain embodiments, the compound of Formula (I-B) is of formula A26-Fl, B52-Fl, B52-A, B52-Cy5, B52-Et-Cy5, or a pharmaceutically acceptable salt thereof.

In certain embodiments, the compound of Formula (I-C) is of formula C1A, C2A, C3A, C4A, C5A, or C6A, or a pharmaceutically acceptable salt thereof.

In certain embodiments, the compound of Formula (I-A), (I-B), or (I-C) is not of formula:

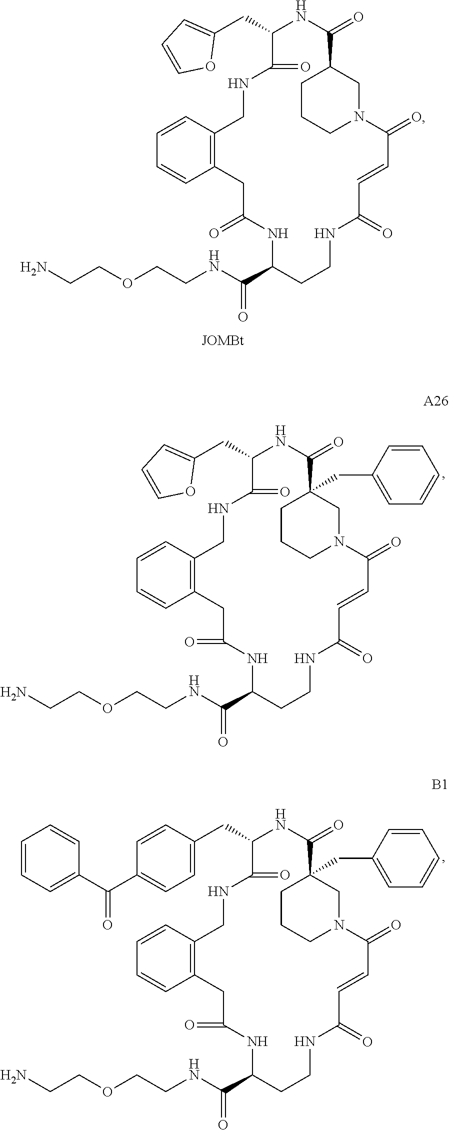 [SMILES: NCCOCCNC(=O)[C@@H]1CCNC(=O)/C=C/C(=O)N2CCC[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(C(=O)c3ccccc3)cc2)C(=O)NCc2ccccc2CC(=O)N1]

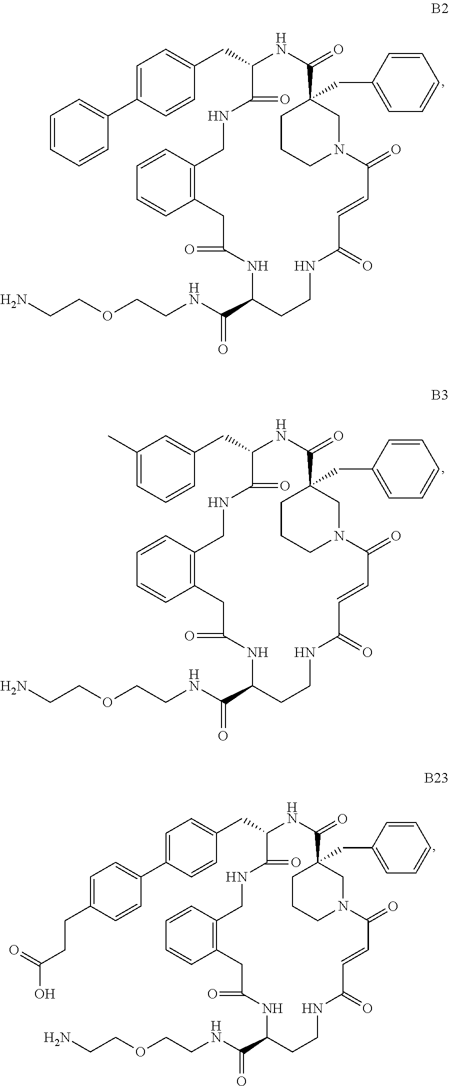 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccccc3)cc2)C(=O)NCc2ccccc2CC(=O)N1 ; Cc1cccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)c1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(CCC(=O)O)cc3)cc2)C(=O)NCc2ccccc2CC(=O)N1]

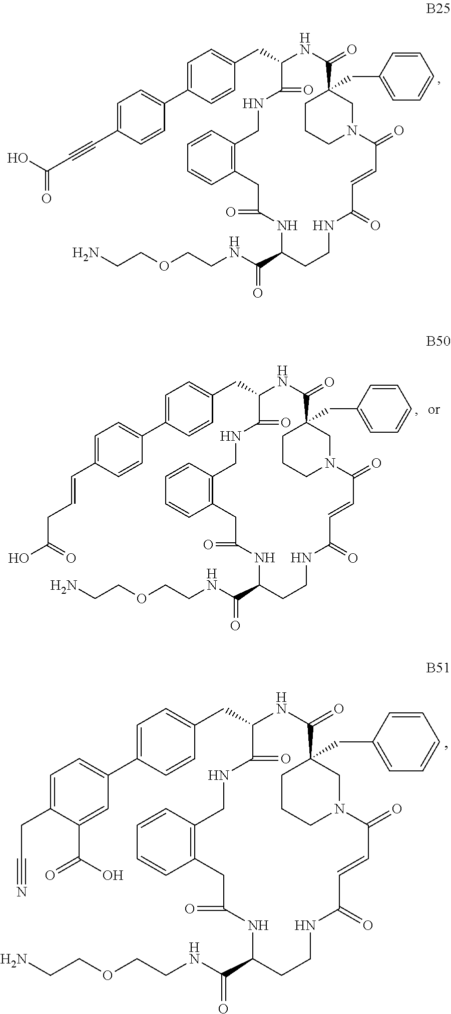 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccc(C#CC(=O)O)cc3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(/C=C/CC(=O)O)cc3)cc2)C(=O)NCc2ccccc2CC(=O)N1 ; N#CCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1C(=O)O]

or a pharmaceutically acceptable salt, solvate, hydrate, polymorph, co-crystal, tautomer, stereoisomer, isotopically enriched form, prodrug, or mixture thereof.

In certain embodiments, the compound Formula (I-A), (I-B), or (I-C) is a compound provided in any one of the Examples below. In certain embodiments, a compound described herein is a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof. In certain embodiments, a compound described herein is a compound of Formula (I-A), (I-B), or (I-C), or a pharmaceutically acceptable salt thereof.

Certain compounds described herein bind, covalently modify, and/or inhibit a cyclophilin. In certain embodiments, the compounds described herein irreversibly inhibit a cyclophilin. In certain embodiments, the compounds described herein reversibly inhibit a cyclophilin. In certain embodiments, the cyclophilin is a cyclophilin A. In certain embodiments, the cyclophilin is cyclophilin B. In certain embodiments, the cyclophilin is cyclophilin C. In certain embodiments, the cyclophilin is cyclophilin D (CypD). In certain embodiments, the cyclophilin is cyclophilin E (CypE). In certain embodiments, the cyclophilin is cyclophilin G. In certain embodiments, the cyclophilin is cyclophilin H. In certain embodiments, the cyclophilin is cyclophilin 40. In certain embodiments, the cyclophilin is PPWD1. In certain embodiments, the cyclophilin is PPIL1. In certain embodiments, the cyclophilin is NKTR. In certain embodiments, the compounds described herein covalently modify the cyclophilin (e.g., CypB, CypC, CypD, CypE, CypG, CypH, Cyp40, PPWD1, PPIL1, NKTR). In certain embodiments, the compounds described herein covalently modify the cyclophilin (e.g., CypD, CypE). In certain embodiments, the compounds described herein covalently modify CypD. In certain embodiments, the compounds described herein covalently modify CypE. In certain embodiments, the compounds described herein reversibly bind to the cyclophilin (e.g., CypB, CypC, CypD, CypE, CypG, CypH, Cyp40, PPWD1, PPIL1, NKTR). In certain embodiments, the compounds described herein reversibly bind to the cyclophilin (e.g., CypD, CypE). In certain embodiments, the compounds described herein reversibly bind to CypD. In certain embodiments, the compounds described herein reversibly bind to CypE. In certain embodiments, the compounds described herein non-reversibly bind to the cyclophilin (e.g., CypB, CypC, CypD, CypE, CypG, CypH, Cyp40, PPWD1, PPIL1, NKTR). In certain embodiments, the compounds described herein non-reversibly bind to the cyclophilin (e.g., CypD, CypE). In certain embodiments, the compounds described herein non-reversibly bind to CypD. In certain embodiments, the compounds described herein non-reversibly bind to CypE. In certain embodiments, the compounds described herein modulate the activity of a cyclophilin (e.g., CypB, CypC, CypD, CypE, CypG, CypH, Cyp40, PPWD1, PPIL1, NKTR). In certain embodiments, the compounds described herein modulate the activity of the cyclophilin (e.g., CypD, CypE). In certain embodiments, the compounds described herein modulate the activity of CypD. In certain embodiments, the compounds described herein modulate the activity of CypE. In certain embodiments, the compounds described herein inhibit the activity of the cyclophilin (e.g., CypB, CypC, CypD, CypE, CypG, CypH, Cyp40, PPWD1, PPIL1, NKTR). In certain embodiments, the compounds described herein inhibit the activity of the cyclophilin (e.g., CypD, CypE). In certain embodiments, the compounds described herein inhibit the activity of CypD. In certain embodiments, the compounds described herein inhibit the activity of CypE. In certain embodiments, the compounds described herein reversibly inhibit the activity of a cyclophilin (e.g., CypB, CypC, CypD, CypE, CypG, CypH, Cyp40, PPWD1, PPIL1, NKTR). In certain embodiments, the compounds described herein reversibly inhibit the activity of the cyclophilin (e.g., CypD, CypE). In certain embodiments, the compounds described herein reversibly inhibit the activity of CypD. In certain embodiments, the compounds described herein reversibly inhibit the activity of CypE.

The binding affinity of a compound described herein to a cyclophilin (e.g., CypB, CypC, CypD, CypE, CypG, CypH, Cyp40, PPWD1, PPIL1, NKTR) may be measured by the dissociation constant (K_{d}) value of an adduct of the compound and the cyclophilin (e.g., CypB, CypC, CypD, CypE, CypG, CypH, Cyp40, PPWD1, PPIL1, NKTR) using methods known in the art (e.g., isothermal titration calorimetry (ITC)). In certain embodiments, the K_{d} value of the adduct is not more than about 100 μM, not more than about 10 μM, not more than about 1 μM, not more than about 100 nM, not more than about 10 nM, or not more than about 1 nM.

In certain embodiments, the activity of a cyclophilin (e.g., CypB, CypC, CypD, CypE, CypG, CypH, Cyp40, PPWD1, PPIL1, NKTR) is inhibited by a compound described herein. The inhibition of the activity of a cyclophilin (e.g., CypB, CypC, CypD, CypE, CypG, CypH, Cyp40, PPWD1, PPIL1, NKTR) by a compound described herein may be measured by determining the half maximal inhibitory concentration (IC₅₀) of the compound when the compound, or a pharmaceutical composition thereof, is contacted with the cyclophilin (e.g., CypB, CypC, CypD, CypE, CypG, CypH, Cyp40, PPWD1, PPIL1, NKTR). The IC₅₀ values may be obtained using methods known in the art (e.g., by a competition binding assay). In certain embodiments, the IC₅₀ value of a compound described herein is not more than about 1 mM, not more than about 100 μM, not more than about 10 μM, not more than about 1 μM, not more than about 100 nM, not more than about 10 nM, or not more than about 1 nM.

The compounds described herein may selectively modulate the activity of a cyclophilin (e.g., CypB, CypC, CypD, CypE, CypG, CypH, Cyp40, PPWD1, PPIL1, NKTR). In certain embodiments, the compounds selectively increase the activity of a cyclophilin (e.g., CypB, CypC, CypD, CypE, CypG, CypH, Cyp40, PPWD1, PPIL1, NKTR). In certain embodiments, the compounds selectively inhibit the activity of a cyclophilin (e.g., CypB, CypC, CypD, CypE, CypG, CypH, Cyp40, PPWD1, PPIL1, NKTR) over other cyclophilins. In certain embodiments, the compounds inhibit the activity of two or more cyclophilins (e.g., CypB, CypC, CypD, CypE, CypG, CypH, Cyp40, PPWD1, PPIL1, NKTR) to the same extent.

The selectivity of a compound described herein in inhibiting the activity of a first cyclophilin (e.g., CypD, CypE) over a second cyclophilin may be measured by the quotient of the IC₅₀ value of the compound in inhibiting the activity of the second cyclophilin over the IC₅₀ value of the compound in inhibiting the activity of the first cyclophilin. The selectivity of a compound described herein in modulating the activity of a first cyclophilin over a second cyclophilin may also be measured by the quotient of the K_{d} value of an adduct of the compound and the second cyclophilin over the K_{d} value of an adduct of the compound and the first cyclophilin (e.g., CypD, CypE). In certain embodiments, the selectivity is at least 2-fold, at least 3-fold, at least 5-fold, at least 10-fold, at least 30-fold, at least 100-fold, at least 300-fold, at least 750-fold, at least 1,000-fold, at least 3,000-fold, at least 10,000-fold, at least 30,000-fold, or at least 100,000-fold. In certain embodiments, the selectivity is not more than 100,000-fold, not more than 10,000-fold, not more than 1,000-fold, not more than 100-fold, not more than 10-fold, or not more than 2-fold. Combinations of the above-referenced ranges (e.g., at least 2-fold and not more than 10,000-fold) are also within the scope of the disclosure. In certain embodiments, the selectivity is at least about 1 at least 2-fold, 5-fold, 10-fold, or more. In certain embodiments, the selectivity is at least 20-fold. In certain embodiments, the selectivity is at least 30-fold. In certain embodiments, the selectivity is at least 100-fold.

In certain embodiments, the compounds of Formula (I-A), (I-B), or (I-C) are selective for cyclophilin D compared to other cyclophilins (e.g., at least 2-fold, 5-fold, 10-fold, or more selective for cyclophilin D). In certain embodiments, the compounds of Formula (I-A), (I-B), or (I-C) are selective for cyclophilin D compared to cyclophilin E (e.g., at least 2-fold, 5-fold, 10-fold, or more selective for cyclophilin D). In certain embodiments, the compounds of Formula (I-A), (I-B), or (I-C) are selective for cyclophilin D compared to cyclophilin A (e.g., at least 2-fold, 5-fold, 10-fold, or more selective for cyclophilin D). In certain embodiments, the compounds of Formula (I-A), (I-B), or (I-C) are selective for cyclophilin D compared to cyclophilin B (e.g., at least 2-fold, 5-fold, 10-fold, or more selective for cyclophilin D). In certain embodiments, the compounds of Formula (I-A), (I-B), or (I-C) are selective for cyclophilin D compared to cyclophilins A, B, and/or E (e.g., at least 2-fold, 5-fold, 10-fold, or more selective for cyclophilin D). In certain embodiments, the compounds of Formula (I-A) or (I-B) are selective for cyclophilin D compared to other cyclophilins (e.g., at least 2-fold, 5-fold, 10-fold, or more selective for cyclophilin D). In certain embodiments, the compounds of Formula (I-A) or (I-B) are selective for cyclophilin D compared to cyclophilin E (e.g., at least 2-fold, 5-fold, 10-fold, or more selective for cyclophilin D). In certain embodiments, the compounds of Formula (I-A) or (I-B) are selective for cyclophilin D compared to cyclophilin A (e.g., at least 2-fold, 5-fold, 10-fold, or more selective for cyclophilin D). In certain embodiments, the compounds of Formula (I-A) or (I-B) are selective for cyclophilin D compared to cyclophilin B (e.g., at least 2-fold, 5-fold, 10-fold, or more selective for cyclophilin D). In certain embodiments, the compounds of Formula (I-A) or (I-B) are selective for cyclophilin D compared to cyclophilins A, B, and/or E (e.g., at least 2-fold, 5-fold, 10-fold, or more selective for cyclophilin D). In certain embodiments, the compounds of Formula (I-A) are selective for cyclophilin D compared to other cyclophilins (e.g., at least 2-fold, 5-fold, 10-fold, or more selective for cyclophilin D). In certain embodiments, the compounds of Formula (I-A) are selective for cyclophilin D compared to cyclophilin E (e.g., at least 2-fold, 5-fold, 10-fold, or more selective for cyclophilin D). In certain embodiments, the compounds of Formula (I-A) are selective for cyclophilin D compared to cyclophilin A (e.g., at least 2-fold, 5-fold, 10-fold, or more selective for cyclophilin D). In certain embodiments, the compounds of Formula (I-A) are selective for cyclophilin D compared to cyclophilin B (e.g., at least 2-fold, 5-fold, 10-fold, or more selective for cyclophilin D). In certain embodiments, the compounds of Formula (I-A) are selective for cyclophilin D compared to cyclophilins A, B, and/or E (e.g., at least 2-fold, 5-fold, 10-fold, or more selective for cyclophilin D). In certain embodiments, the compounds of Formula (I-B) are selective for cyclophilin D compared to other cyclophilins (e.g., at least 2-fold, 5-fold, 10-fold, or more selective for cyclophilin D). In certain embodiments, the compounds of Formula (I-B) are selective for cyclophilin D compared to cyclophilin E (e.g., at least 2-fold, 5-fold, 10-fold, or more selective for cyclophilin D). In certain embodiments, the compounds of Formula (I-B) are selective for cyclophilin D compared to cyclophilin A (e.g., at least 2-fold, 5-fold, 10-fold, or more selective for cyclophilin D). In certain embodiments, the compounds of Formula (I-B) are selective for cyclophilin D compared to cyclophilin B (e.g., at least 2-fold, 5-fold, 10-fold, or more selective for cyclophilin D). In certain embodiments, the compounds of Formula (I-B) are selective for cyclophilin D compared to cyclophilins A, B, and/or E (e.g., at least 2-fold, 5-fold, 10-fold, or more selective for cyclophilin D).

In certain embodiments, the compounds of Formula (I-A), (I-B), or (I-C) are selective for cyclophilin E compared to other cyclophilins (e.g., at least 2-fold, 5-fold, 10-fold, or more selective for cyclophilin E). In certain embodiments, the compounds of Formula (I-A), (I-B), or (I-C) are selective for cyclophilin E compared to cyclophilin D (e.g., at least 2-fold, 5-fold, 10-fold, or more selective for cyclophilin E). In certain embodiments, the compounds of Formula (I-A), (I-B), or (I-C) are selective for cyclophilin E compared to cyclophilin A (e.g., at least 2-fold, 5-fold, 10-fold, or more selective for cyclophilin E). In certain embodiments, the compounds of Formula (I-A), (I-B), or (I-C) are selective for cyclophilin E compared to cyclophilin B (e.g., at least 2-fold, 5-fold, 10-fold, or more selective for cyclophilin E). In certain embodiments, the compounds of Formula (I-A), (I-B), or (I-C) are selective for cyclophilin E compared to cyclophilins A, B, and/or D (e.g., at least 2-fold, 5-fold, 10-fold, or more selective for cyclophilin D). In certain embodiments, the compounds of Formula (I-C) are selective for cyclophilin E compared to other cyclophilins (e.g., at least 2-fold, 5-fold, 10-fold, or more selective for cyclophilin E). In certain embodiments, the compounds of Formula (I-C) are selective for cyclophilin E compared to cyclophilin D (e.g., at least 2-fold, 5-fold, 10-fold, or more selective for cyclophilin E). In certain embodiments, the compounds of Formula (I-C) are selective for cyclophilin E compared to cyclophilin A (e.g., at least 2-fold, 5-fold, 10-fold, or more selective for cyclophilin E). In certain embodiments, the compounds of Formula (I-C) are selective for cyclophilin E compared to cyclophilin B (e.g., at least 2-fold, 5-fold, 10-fold, or more selective for cyclophilin E). In certain embodiments, the compounds of Formula (I-C) are selective for cyclophilin E compared to cyclophilins A, B, and/or D (e.g., at least 2-fold, 5-fold, 10-fold, or more selective for cyclophilin D). In certain embodiments, selectivity for inhibiting a first cyclophilin over other cyclophilins is measured by in vitro inhibition (IC₅₀) assays using a chymotrypsin coupled PPIase assay with Suc-AAPF-AMC as the peptide substrate was used, whereby isomerization of a peptide substrate Suc-AAPF-AMC from the cis to trans conformation allowed for proteolysis via excess α-chymotrypsin, releasing the C-terminal coumarin fluorophoreas. In certain embodiments, selectivity for inhibiting a first cyclophilin over other cyclophilins is measured by Surface Plasmon Resonance (SPR) assays.

It is expected that the compounds described herein may be useful in treating and/or preventing diseases associated with the activity (e.g., increased activity, decreased activity, undesired activity, aberrant activity, peptidyl-prolyl-isomerase (PPIase) activity (e.g., increased PPIase activity, decreased PPIase activity, undesired PPIase activity, aberrant PPIase activity)) of a cyclophilin (e.g., CypB, CypC, CypD, CypE, CypG, CypH, Cyp40, PPWD1, PPIL1, NKTR). It is known in the art that cyclophilins are implicated in a wide range of diseases and conditions, such as neurological (e.g., neurodegenerative) diseases, metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases, and conditions associated with regulation of the mitochondrial permeability transition pore (mPTP), autophagy, aging; and oxidative stress. Therefore, the compounds described herein are expected to be useful in treating and/or preventing diseases (e.g., neurological (e.g., neurodegenerative) diseases, metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases, and conditions associated with regulation of the mitochondrial permeability transition pore (mPTP), autophagy, aging; and oxidative stress). The compounds described herein that bind, covalently modify, and/or inhibit CypD are expected to be use useful in treating and/or preventing diseases associated with CypD (e.g., ischemia-reperfusion injury (IRI), Alzheimer's disease, Huntington's disease, multiple sclerosis, Parkinson's disease, amyotrophic lateral sclerosis, X-linked adrenoleukodystrophy, liver cirrhosis, or diabetes).

### Pharmaceutical Compositions, Kits, and Administration

The present disclosure also provides pharmaceutical compositions comprising a compound described herein and a pharmaceutically acceptable excipient. In certain embodiments, a pharmaceutical composition described herein comprises a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof, and a pharmaceutically acceptable excipient. In certain embodiments, a pharmaceutical composition described herein comprises a compound of Formula (I-A), (I-B), or (I-C), or a pharmaceutically acceptable salt thereof, and a pharmaceutically acceptable excipient. In certain embodiments, a pharmaceutical composition described herein for treating the diseases and/or conditions described herein comprises a compound of Formula (I-A), (I-B), or (I-C), or a pharmaceutically acceptable salt thereof, and a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, a therapeutically effective amount is an amount effective for inhibiting the activity (e.g., increased activity, decreased activity, undesired activity, aberrant activity, peptidyl-prolyl-isomerase (PPIase) activity (e.g., increased PPIase activity, decreased PPIase activity, undesired PPIase activity, aberrant PPIase activity)) of a cyclophilin (e.g., CypB, CypC, CypD, CypE, CypG, CypH, Cyp40, PPWD1, PPIL1, NKTR). In certain embodiments, a therapeutically effective amount is an amount effective for inhibiting the peptidyl-prolyl-isomerase (PPIase) activity (e.g., increased PPIase activity, decreased PPIase activity, undesired PPIase activity, PPIase aberrant activity) of a cyclophilin (e.g., CypB, CypC, CypD, CypE, CypG, CypH, Cyp40, PPWD1, PPIL1, NKTR). In certain embodiments, a therapeutically effective amount is an amount effective for treating a disease. In certain embodiments, a therapeutically effective amount is an amount effective for inhibiting the aberrant activity of a cyclophilin (e.g., CypB, CypC, CypD, CypE, CypG, CypH, Cyp40, PPWD1, PPIL1, NKTR) and treating a disease (e.g., a disease associated with aberrant activity of a cyclophilin (e.g., CypB, CypC, CypD, CypE, CypG, CypH, Cyp40, PPWD1, PPIL1, NKTR)). In certain embodiments, a therapeutically effective amount is an amount effective for inducing apoptosis of a cell (e.g., cell in vivo or in vitro). In certain embodiments, a prophylactically effective amount is an amount effective for inhibiting the aberrant activity of a cyclophilin (e.g., CypB, CypC, CypD, CypE, CypG, CypH, Cyp40, PPWD1, PPIL1, NKTR). In certain embodiments, a prophylactically effective amount is an amount effective for preventing or keeping a subject in need thereof in remission of a disease. In certain embodiments, a prophylactically effective amount is an amount effective for inhibiting the aberrant activity of a cyclophilin, and preventing or keeping a subject in need thereof in remission of a disease (e.g., a disease associated with aberrant activity of a cyclophilin). In certain embodiments, the pharmaceutical composition comprises a therapeutically effective amount of the compound for use in treating a disease (e.g., a disease associated with aberrant activity of a cyclophilin), in a subject in need thereof. In certain embodiments, the pharmaceutical composition comprises a therapeutically effective amount of the compound for use in treating a disease and/or condition associated with CypD (e.g., ischemia-reperfusion injury (IRI), Alzheimer's disease, Huntington's disease, multiple sclerosis, Parkinson's disease, amyotrophic lateral sclerosis, X-linked adrenoleukodystrophy, liver cirrhosis, or diabetes) in a subject in need thereof.

In certain embodiments, the effective amount is an amount effective for inhibiting the activity of a cyclophilin (e.g., CypB, CypC, CypD, CypE, CypG, CypH, Cyp40, PPWD1, PPIL1, NKTR) by at least 10%, at least 20%, at least 30%, at least 40%, at least 50%, at least 60%, at least 70%, at least 80%, at least 90%, at least 95%, or at least 98%. In certain embodiments, the effective amount is an amount effective for inhibiting the activity of a cyclophilin (e.g., CypD, CypE) by at least 10%, at least 20%, at least 30%, at least 40%, at least 50%, at least 60%, at least 70%, at least 80%, at least 90%, at least 95%, or at least 98%. In certain embodiments, the effective amount is an amount effective for inhibiting the activity of CypD by at least 10%, at least 20%, at least 30%, at least 40%, at least 50%, at least 60%, at least 70%, at least 80%, at least 90%, at least 95%, or at least 98%. In certain embodiments, the effective amount is an amount effective for inhibiting the activity of CypE by at least 10%, at least 20%, at least 30%, at least 40%, at least 50%, at least 60%, at least 70%, at least 80%, at least 90%, at least 95%, or at least 98%. In certain embodiments, the effective amount is an amount effective for inhibiting the activity of a cyclophilin (e.g., CypB, CypC, CypD, CypE, CypG, CypH, Cyp40, PPWD1, PPIL1, NKTR) by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for inhibiting the activity of a cyclophilin (e.g., CypD, CypE) by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for inhibiting the activity of CypD by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for inhibiting the activity of CypE by not more than 10%, not more than 20%, not more than 30%, not more than 40%, not more than 50%, not more than 60%, not more than 70%, not more than 80%, not more than 90%, not more than 95%, or not more than 98%.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell being contacted with a compound or composition described herein is in vitro. In certain embodiments, the cell being contacted with a compound or composition described herein is in vivo.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyfrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms foral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a cell, tissue, or biological sample, any two doses of the multiple doses include different or substantially the same amounts of a compound described herein. In certain embodiments, when multiple doses are administered to a subject or applied to a cell, tissue, or biological sample, the frequency of administering the multiple doses to the subject or applying the multiple doses to the cell, tissue, or biological sample is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the cell, tissue, or biological sample is one dose per day. In certain embodiments, the frequency of administering the multiple doses to the subject or applying the multiple doses to the cell, tissue, or biological sample is two doses per day. In certain embodiments, the frequency of administering the multiple doses to the subject or applying the multiple doses to the cell, tissue, or biological sample is three doses per day. In certain embodiments, when multiple doses are administered to a subject or applied to a cell, tissue, or biological sample, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in inhibiting the activity of a cyclophilin (e.g., CypB, CypC, CypD, CypE, CypG, CypH, Cyp40, PPWD1, PPIL1, NKTR) in a subject, cell, tissue, or biological sample), improve bioavailability, improve safety, reduce drug resistance, reduce and/or modify metabolism, inhibit excretion, and/or modify distribution in a subject, cell, tissue, or biological sample. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., neurological (e.g., neurodegenerative) disease (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases, or other diseases associated with cyclophilins (e.g., CypD, CypE)). In certain embodiments, the additional pharmaceutical agent is a pharmaceutical agent useful for treating diseases associated with cyclophilins (e.g., CypD, CypE). In certain embodiments, the additional pharmaceutical agent is a pharmaceutical agent useful for treating diseases associated with CypD. In certain embodiments, the additional pharmaceutical agent is a pharmaceutical agent useful for treating diseases associated with CypE. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In certain embodiments, the additional pharmaceutical agent is an anti-proliferative agent (e.g., anti-cancer agent). In certain embodiments, the additional pharmaceutical agent is an anti-leukemia agent. In certain embodiments, the additional pharmaceutical agent is ABITREXATE (methotrexate), ADE, Adriamycin RDF (doxorubicin hydrochloride), Ambochlorin (chlorambucil), ARRANON (nelarabine), ARZERRA (ofatumumab), BOSULIF (bosutinib), BUSULFEX (busulfan), CAMPATH (alemtuzumab), CERUBIDINE (daunorubicin hydrochloride), CLAFEN (cyclophosphamide), CLOFAREX (clofarabine), CLOLAR (clofarabine), CVP, CYTOSAR-U (cytarabine), CYTOXAN (cyclophosphamide), ERWINAZE (Asparaginase Erwinia Chrysanthemi), FLUDARA (fludarabine phosphate), FOLEX (methotrexate), FOLEX PFS (methotrexate), GAZYVA (obinutuzumab), GLEEVEC (imatinib mesylate), Hyper-CVAD, ICLUSIG (ponatinib hydrochloride), IMBRUVICA (ibrutinib), LEUKERAN (chlorambucil), LINFOLIZIN (chlorambucil), MARQIBO (vincristine sulfate liposome), METHOTREXATE LPF (methorexate), MEXATE (methotrexate), MEXATE-AQ (methotrexate), mitoxantrone hydrochloride, MUSTARGEN (mechlorethamine hydrochloride), MYLERAN (busulfan), NEOSAR (cyclophosphamide), ONCASPAR (Pegaspargase), PURINETHOL (mercaptopurine), PURIXAN (mercaptopurine), Rubidomycin (daunorubicin hydrochloride), SPRYCEL (dasatinib), SYNRIBO (omacetaxine mepesuccinate), TARABINE PFS (cytarabine), TASIGNA (nilotinib), TREANDA (bendamustine hydrochloride), TRISENOX (arsenic trioxide), VINCASAR PFS (vincristine sulfate), ZYDELIG (idelalisib), or a combination thereof. In certain embodiments, the additional pharmaceutical agent is an anti-lymphoma agent. In certain embodiments, the additional pharmaceutical agent is ABITREXATE (methotrexate), ABVD, ABVE, ABVE-PC, ADCETRIS (brentuximab vedotin), ADRIAMYCIN PFS (doxorubicin hydrochloride), ADRIAMYCIN RDF (doxorubicin hydrochloride), AMBOCHLORIN (chlorambucil), AMBOCLORIN (chlorambucil), ARRANON (nelarabine), BEACOPP, BECENUM (carmustine), BELEODAQ (belinostat), BEXXAR (tositumomab and iodine 1131 tositumomab), BICNU (carmustine), BLENOXANE (bleomycin), CARMUBRIS (carmustine), CHOP, CLAFEN (cyclophosphamide), COPP, COPP-ABV, CVP, CYTOXAN (cyclophosphamide), DEPOCYT (liposomal cytarabine), DTIC-DOME (dacarbazine), EPOCH, FOLEX (methotrexate), FOLEX PFS (methotrexate), FOLOTYN (pralatrexate), HYPER-CVAD, ICE, IMBRUVICA (ibrutinib), INTRON A (recombinant interferon alfa-2b), ISTODAX (romidepsin), LEUKERAN (chlorambucil), LINFOLIZIN (chlorambucil), Lomustine, MATULANE (procarbazine hydrochloride), METHOTREXATE LPF (methotrexate), MEXATE (methotrexate), MEXATE-AQ (methotrexate), MOPP, MOZOBIL (plerixafor), MUSTARGEN (mechlorethamine hydrochloride), NEOSAR (cyclophosphamide), OEPA, ONTAK (denileukin diftitox), OPPA, R—CHOP, REVLIMID (lenalidomide), RITUXAN (rituximab), STANFORD V, TREANDA (bendamustine hydrochloride), VAMP, VELBAN (vinblastine sulfate), VELCADE (bortezomib), VELSAR (vinblastine sulfate), VINCASAR PFS (vincristine sulfate), ZEVALIN (ibritumomab tiuxetan), ZOLINZA (vorinostat), ZYDELIG (idelalisib), or a combination thereof. In certain embodiments, the additional pharmaceutical agent is REVLIMID (lenalidomide), DACOGEN (decitabine), VIDAZA (azacitidine), CYTOSAR-U (cytarabine), IDAMYCIN (idarubicin), CERUBIDINE (daunorubicin), LEUKERAN (chlorambucil), NEOSAR (cyclophosphamide), FLUDARA (fludarabine), LEUSTATIN (cladribine), or a combination thereof. In certain embodiments, the additional pharmaceutical agent is ABITREXATE (methotrexate), ABRAXANE (paclitaxel albumin-stabilized nanoparticle formulation), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), AVASTIN (bevacizumab), BECENUM (carmustine), BEP, BICNU (carmustine), BLENOXANE (bleomycin), CAF, CAMPTOSAR (irinotecan hydrochloride), CAPOX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CLAFEN (cyclophosphamide), CMF, COMETRIQ (cabozantinib-s-malate), COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), ERBITUX (cetuximab), ERIVEDGE (vismodegib), ETOPOPHOS (etoposide phosphate), EVACET (doxorubicin hydrochloride liposome), FARESTON (toremifene), FASLODEX (fulvestrant), FEC, FEMARA (letrozole), FLUOROPLEX (fluorouracil), FOLEX (methotrexate), FOLEX PFS (methotrexate), FOLFIRI, FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOVALDEX (tamoxifen citrate), OFF, PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), REVLIMID (lenalidomide), RUBIDOMYCIN (daunorubicin hydrochloride), SPRYCEL (dasatinib), STIVARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TRISENOX (arsenic trioxide), TYKERB (lapatinib ditosylate), VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZOLADEX (goserelin acetate), ZOMETA (zoledronic acid), ZYKADIA (ceritinib), ZYTIGA (abiraterone acetate), ENMD-2076, PCI-32765, AC220, dovitinib lactate (TK1258, CHIR-258), BIBW 2992 (TOVOK™) SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, or a combination thereof. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. In certain embodiments, the compounds described herein or pharmaceutical compositions can be administered in combination with a pharmaceutical agent useful for treating and/or preventing a neurological (e.g., neurodegenerative) disease (e.g., Alzheimer's disease, multiple sclerosis, Parkinson's disease, Huntington's disease, amyotrophic lateral sclerosis). In certain embodiments, the compounds described herein or pharmaceutical compositions can be administered in combination with a pharmaceutical agent for treating and/or preventing Parkinson's disease that is levodopa, carbidopa, or a dopamine agonist. In certain embodiments, the additional pharmaceutical agent is an agent for treating Alzheimer's disease (e.g., cholinesterase inhibitors, memantine). In certain embodiments, the additional pharmaceutical agent is an agent for treating Huntington's disease (e.g., tetrabenazine). In certain embodiments, the additional pharmaceutical agent is an agent for treating amyotrophic lateral sclerosis (ALS) (e.g., glutamate blockers, edaravone). In certain embodiments, the additional pharmaceutical agent is an agent for treating multiple sclerosis (e.g., interferon beta, glatiramer acetate, CD52 antibody, sphingosine-1-phospate receptor modulators, dihydroorotate dehydrogenase (DHODH) inhibitors). In certain embodiments, the compounds described herein or pharmaceutical compositions can be administered in combination with a pharmaceutical agent useful for treating and/or preventing oxidative stress. In certain embodiments, the compounds described herein or pharmaceutical compositions can be administered in combination with a pharmaceutical agent useful for treating and/or preventing a mitochondrial disease (e.g., condition associated with modulating (e.g., regulating) the mPTP, condition related to autophagy autophagy (e.g., neurodegenerative disease, infection, cancer, aging, heart disease)). In certain embodiments, the compounds described herein or pharmaceutical compositions can be administered in combination with a pharmaceutical agent useful for treating and/or preventing a cardiovascular condition (e.g., ischemia-reperfusion injury), stroke, heart attack. In certain embodiments, the additional pharmaceutical agent is an agent for treating ischemia-reperfusion injury (e.g., blood thinners, arterial dilators).

Also encompassed by the disclosure are kits (e.g., pharmaceutical packs). The kits provided may comprise a pharmaceutical composition or compound, or a pharmaceutically acceptable salt, solvate, hydrate, polymorph, co-crystal, tautomer, stereoisomer, isotopically labeled derivative, prodrug, or mixture thereof, described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease in a subject in need thereof. In certain embodiments, the kits are useful for preventing a disease in a subject in need thereof. In certain embodiments, the kits are useful for preventing a disease and/or condition associated with CypD. In certain embodiments, the kits are useful for inhibiting the activity (e.g., increased activity, decreased activity, undesired activity, aberrant activity, peptidyl-prolyl-isomerase (PPIase) activity (e.g., increased PPIase activity, decreased PPIase activity, undesired PPIase activity, aberrant PPIase activity)) of a cyclophilin (e.g., CypB, CypC, CypD, CypE, CypG, CypH, Cyp40, PPWD1, PPIL1, NKTR) in a subject, cell, tissue, or biological sample.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease in a subject in need thereof. In certain embodiments, the kits and instructions provide for preventing a disease in a subject in need thereof. In certain embodiments, the kits and instructions provide for modulating (e.g., inhibiting) the activity (e.g., increased activity, decreased activity, undesired activity, aberrant activity, peptidyl-prolyl-isomerase (PPIase) activity (e.g., increased PPIase activity, decreased PPIase activity, undesired PPIase activity, aberrant PPIase activity)) of a cyclophilin (e.g., CypB, CypC, CypD, CypE, CypG, CypH, Cyp40, PPWD1, PPIL1, NKTR) in a subject, cell, tissue, or biological sample. In certain embodiments, the kits and instructions provide for reducing oxidative stress in a subject in need thereof or in a cell, tissue, or biological sample. In certain embodiments, the kits and instructions provide for administering to a subject or contacting a cell, tissue, or biological sample with the compound, or a pharmaceutically acceptable salt, solvate, hydrate, polymorph, co-crystal, tautomer, stereoisomer, isotopically labeled derivative, prodrug, or mixture thereof, or a pharmaceutical composition thereof. A kit described herein may include one or more additional pharmaceutical agents described herein as a separate composition.

### Methods of Treatment and Uses

The present disclosure provides methods of modulating (e.g., inhibiting or increasing) the activity (e.g., increased activity, decreased activity, undesired activity, aberrant activity, peptidyl-prolyl-isomerase (PPIase) activity (e.g., increased PPIase activity, decreased PPIase activity, undesired PPIase activity, aberrant PPIase activity)) of a cyclophilin (e.g., CypB, CypC, CypD, CypE, CypG, CypH, Cyp40, PPWD1, PPIL1, NKTR). The present disclosure provides methods of modulating (e.g., inhibiting or increasing) the peptidyl-prolyl-isomerase (PPIase) activity (e.g., increased PPIase activity, decreased PPIase activity, undesired PPIase activity, PPIase aberrant activity) of a cyclophilin (e.g., CypB, CypC, CypD, CypE, CypG, CypH, Cyp40, PPWD1, PPIL1, NKTR). The present disclosure provides methods of modulating (e.g., inhibiting or increasing) the activity (e.g., increased activity, decreased activity, undesired activity, aberrant activity, peptidyl-prolyl-isomerase (PPIase) activity (e.g., increased PPIase activity, decreased PPIase activity, undesired PPIase activity, aberrant PPIase activity)) of a cyclophilin (e.g., CypB, CypC, CypD, CypE, CypG, CypH, Cyp40, PPWD1, PPIL1, NKTR) in a subject, cell, tissue, or biological sample. The present disclosure provides methods of reducing oxidative stress in a subject, cell, tissue, or biological sample. The present disclosure also provides methods for the treatment of a wide range of diseases, such as diseases associated with the activity (e.g., increased activity, decreased activity, undesired activity, aberrant activity, peptidyl-prolyl-isomerase (PPIase) activity (e.g., increased PPIase activity, decreased PPIase activity, undesired PPIase activity, aberrant PPIase activity)) of a cyclophilin (e.g., CypB, CypC, CypD, CypE, CypG, CypH, Cyp40, PPWD1, PPIL1, NKTR), e.g., neurological (e.g., neurodegenerative) disease (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases, or other diseases associated with cyclophilins (e.g., CypD, CypE) in a subject in need thereof. The present disclosure also provides methods for the treatment of diseases and/or conditions associated with CypD (e.g., ischemia-reperfusion injury (IRI), Alzheimer's disease, Huntington's disease, multiple sclerosis, Parkinson's disease, amyotrophic lateral sclerosis, X-linked adrenoleukodystrophy, liver cirrhosis, or diabetes). The present disclosure provides methods for the treatment and/or prevention of a neurological disease (e.g., neurodegenerative (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases, or other diseases associated with cyclophilins (e.g., CypD, CypE) in a subject in need thereof. In certain embodiments, the diseases treated and/or prevented with a compound described herein are associated with CypD. In certain embodiments, the diseases treated and/or prevented with a compound described herein are associated with CypE.

The present disclosure also provides a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof, or a pharmaceutical composition thereof, for use in the treatment of a disease, such as a neurological disease (e.g., neurodegenerative) (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, ischemia-reperfusion injury, conditions associated with oxidative stress, mitochondrial diseases, or other diseases associated with cyclophilins (e.g., CypD, CypE), in a subject in need thereof. The present disclosure also provides a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof, or a pharmaceutical composition thereof, for use in the treatment of a disease, such as neurological disease (e.g., neurodegenerative) (e.g., Alzheimer's disease, multiple sclerosis, Parkinson's disease, Huntington's disease, amyotrophic lateral sclerosis), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, ischemia-reperfusion injury, conditions associated with oxidative stress, mitochondrial diseases, or other diseases associated with cyclophilins (e.g., CypD, CypE), in a subject in need thereof. The present disclosure also provides a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof, or a pharmaceutical composition thereof, for use in the treatment of a disease and/or condition associated with CypD (e.g., ischemia-reperfusion injury (IRI), Alzheimer's disease, Huntington's disease, multiple sclerosis, Parkinson's disease, amyotrophic lateral sclerosis, X-linked adrenoleukodystrophy, liver cirrhosis, or diabetes), in a subject in need thereof.

The present disclosure also provides uses of a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof, or a pharmaceutical composition thereof, in the manufacture of a medicament for the treatment of a disease, such as neurological disease (e.g., neurodegenerative) (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases, or other diseases associated with cyclophilins (e.g., CypD, CypE), in a subject in need thereof. The present disclosure also provides a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof, or a pharmaceutical composition thereof, for use in the manufacture of a medicament for the treatment of a disease and/or condition (e.g. ischemia-reperfusion injury (IRI), Alzheimer's disease, Huntington's disease, multiple sclerosis, Parkinson's disease, amyotrophic lateral sclerosis, X-linked adrenoleukodystrophy, liver cirrhosis, or diabetes), in a subject in need thereof.

The present disclosure also provides uses of a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof, or a pharmaceutical composition thereof, to treat a disease and/or condition, such as neurological disease (e.g., neurodegenerative) (e.g., Alzheimer's disease, multiple sclerosis, Parkinson's disease, Huntington's disease, amyotrophic lateral sclerosis), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, ischemia-reperfusion injury, conditions associated with oxidative stress, mitochondrial diseases, or other diseases associated with cyclophilins (e.g., CypD, CypE), in a subject in need thereof. The present disclosure also provides uses of a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof, or a pharmaceutical composition thereof, to treat a disease and/or condition associated with CypD, such as ischemia-reperfusion injury (IRI), Alzheimer's disease, Huntington's disease, multiple sclerosis, Parkinson's disease, amyotrophic lateral sclerosis, X-linked adrenoleukodystrophy, liver cirrhosis, or diabetes, in a subject in need thereof.

In another aspect, the present disclosure provides methods of treating a disease, such as neurological disease (e.g., neurodegenerative) (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases, or other diseases associated with cyclophilins (e.g., CypD, CypE), the methods comprising administering to the subject an effective amount of a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof, or a pharmaceutical composition thereof.

In another aspect, the present disclosure provides methods of modulating the activity of a cyclophilin (e.g., CypB, CypC, CypD, CypE, CypG, CypH, Cyp40, PPWD1, PPIL1, NKTR) in a subject, cell, tissue, or biological sample. In certain embodiments, provided are methods of inhibiting a cyclophilin in a subject. In certain embodiments, provided are methods of inhibiting a cyclophilin in a cell. In certain embodiments, provided are methods of inhibiting the activity of a cyclophilin in a subject. In certain embodiments, provided are methods of inhibiting the activity of a cyclophilin in a cell. The compounds described herein may exhibit cyclophilin inhibitory activity; the ability to inhibit a cyclophilin; the ability to inhibit CypB, without inhibiting another cyclophilin; the ability to inhibit CypC, without inhibiting another cyclophilin; the ability to inhibit CypD, without inhibiting another cyclophilin; the ability to inhibit CypE, without inhibiting another cyclophilin; the ability to inhibit CypG, without inhibiting another cyclophilin; the ability to inhibit CypH, without inhibiting another cyclophilin; the ability to inhibit Cyp40, without inhibiting another cyclophilin; the ability to inhibit PPWD1, without inhibiting another cyclophilin; the ability to inhibit PPIL1, without inhibiting another cyclophilin; the ability to inhibit NKTR, without inhibiting another cyclophilin; a therapeutic effect and/or preventative effect in the treatment of neurological (e.g., neurodegenerative) disease (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases, or other diseases associated with cyclophilins (e.g., CypD, CypE)) in a subject in need thereof. In certain embodiments, the compound being administered or used inhibits a cyclophilin (e.g., CypB, CypC, CypD, CypE, CypG, CypH, Cyp40, PPWD1, PPIL1, NKTR) in a subject, cell, tissue, or biological sample, treats and/or prevents a disease; and/or has a therapeutic profile (e.g., optimum safety and curative effect) that is superior to existing chemotherapeutic agents, or agents for treating diseases in a subject in need thereof. In certain embodiments, the compound being administered or used inhibits a cyclophilin (e.g., CypB, CypC, CypD, CypE, CypG, CypH, Cyp40, PPWD1, PPIL1, NKTR) in a subject, cell, tissue, or biological sample, treats and/or prevents a disease.

In certain embodiments, provided are methods of decreasing the activity of a cyclophilin (e.g., CypB, CypC, CypD, CypE, CypG, CypH, Cyp40, PPWD1, PPIL1, NKTR) in a subject, cell, tissue, or biological sample by a method described herein by at least about 1%, at least about 3%, at least about 10%, at least about 20%, at least about 30%, at least about 40%, at least about 50%, at least about 60%, at least about 70%, at least about 80%, or at least about 90%. In certain embodiments, the activity of a cyclophilin (e.g., CypB, CypC, CypD, CypE, CypG, CypH, Cyp40, PPWD1, PPIL1, NKTR) in a subject, cell, tissue, or biological sample is decreased by a method described herein by at least about 1%, at least about 3%, at least about 10%, at least about 20%, at least about 30%, at least about 40%, at least about 50%, at least about 60%, at least about 70%, at least about 80%, or at least about 90%. In some embodiments, the activity of a cyclophilin (e.g., CypB, CypC, CypD, CypE, CypG, CypH, Cyp40, PPWD1, PPIL1, NKTR) in a subject, cell, tissue, or biological sample is selectively inhibited by the method. In some embodiments, the activity of a cyclophilin (e.g., CypB, CypC, CypD, CypE, CypG, CypH, Cyp40, PPWD1, PPIL1, NKTR) in a subject, cell, tissue, or biological sample is selectively decreased by the method.

Without wishing to be bound by any particular theory, the compounds described herein are able to bind to the cyclophilin being inhibited. In certain embodiments, a compound described herein is able to bind to the cyclophilin. In certain embodiments, the compound described herein is able to bind to a central pocket of the cyclophilin. In certain embodiments, the compound is capable of binding to the S2 pocket of CypD. In certain embodiments, the compound is capable of binding to the gatekeeper residues of CypD (serine and/or arginine). In certain embodiments, the compound is capable of binding to the gatekeeper residues of CypD (Ser81, Arg82). In certain embodiments, the compound is capable of binding to the gatekeeper residues of CypD (Ser81, Arg82, Serine 123, and/or Arginine 124). In certain embodiments, the compound is capable of binding to the gatekeeper residues of CypD (Serine 123 and/or Arginine 124). In some embodiments, the compound is capable of binding to the gatekeeper residues of CypD (Serine 123 and Arginine 124). In certain embodiments, the compound is capable of binding to the gatekeeper region of CypD (e.g., the gatekeeper residues of CypD including Serine 123 and Arginine 124). In certain embodiments, the compound is capable of binding to the active site, the S2 pocket, and/or the gatekeeper region of CypD (e.g., the gatekeeper residues of CypD including Serine 123 and Arginine 124). In certain embodiments, the compound is capable of binding to the active site, the S2 pocket, and the gatekeeper region of CypD (e.g., the gatekeeper residues of CypD including Serine 123 and Arginine 124). In certain embodiments, the compound described herein is able to selectively bind CypD over other cyclophilins. In certain embodiments, the compound described herein is able to selectively inhibit CypD over other cyclophilins. In certain embodiments, the compound is capable of covalently binding CypD. In certain embodiments, the compound is capable of covalently modifying CypD (e.g., S2 pocket of CypD). In certain embodiments, the compound is capable of covalently modifying the S2 pocket of CyPD. In certain embodiments, the compound is capable of preventing mPTP opening.

In certain embodiments, the compound is capable of binding and/or covalently modifying the S2 pocket of CypE. In certain embodiments, the compound is capable of binding and/or covalently modifying the gatekeeper residues of CypE (lysine). In certain embodiments, the compound is capable of binding and/or covalently modifying the gatekeeper residues of CypE (Lys217 and/or Lys218). In certain embodiments, the compound is capable of binding and/or covalently modifying the gatekeeper residues of CypE (Lysine 217 and/or Lysine 218). In certain embodiments, the compound is capable of binding and/or covalently modifying the gatekeeper residues of CypE (Lysine 217 and/or Lysine 218). In certain embodiments, the compound is capable of binding and/or covalently modifying the gatekeeper residues of CypE (Lysine 217 and Lysine 218). In certain embodiments, the compound is capable of binding and/or covalently modifying the gatekeeper region of CypE (e.g., the gatekeeper residues of CypE including Lysine 217 and/or Lysine 218). In certain embodiments, the compound is capable of binding and/or covalently modifying the active site, the S2 pocket, and/or the gatekeeper region of CypE (e.g., the gatekeeper residues of CypE including Lysine 217 and/or Lysine 218). In certain embodiments, the compound is capable of binding and/or covalently modifying the active site, the S2 pocket, and the gatekeeper region of CypE (e.g., the gatekeeper residues of CypE including Lysine 217 and/or Lysine 218). In certain embodiments, the compound described herein is able to selectively bind CypE over other cyclophilins. In certain embodiments, the compound described herein is able to selectively inhibit CypE over other cyclophilins. In certain embodiments, the compound is capable of covalently binding CypE. In certain embodiments, the compound is capable of covalently modifying CypE (e.g., S2 pocket of CypE). In certain embodiments, the compound is capable of covalently modifying the S2 pocket of CypE.

In certain embodiments, the compound is capable of binding CypB. In certain embodiments, the compound is capable of binding CypC. In certain embodiments, the compound is capable of binding CypD. In certain embodiments, the compound is capable of binding CypE. In certain embodiments, the compound is capable of binding CypG. In certain embodiments, the compound is capable of binding CypH. In certain embodiments, the compound is capable of binding Cyp40. In certain embodiments, the compound is capable of binding PPWD1. In certain embodiments, the compound is capable of binding PPIL1. In certain embodiments, the compound is capable of binding NKTR. In certain embodiments, the compound is capable of binding CypB, CypC, CypD, CypE, CypG, CypH, Cyp40, PPWD1, PPIL1, or NKTR.

In certain embodiments, the compound is capable of covalently modifying CypD. In certain embodiments, the compound is capable of covalently modifying CypE. In certain embodiments, the compound is capable of covalently modifying CypE. In certain embodiments, the compound is capable of covalently modifying CypB, CypC, CypD, CypE, CypG, CypH, Cyp40, PPWD1, PPIL1, or NKTR. In certain embodiments, the compound is capable of covalently modifying CypB, CypC, CypD, CypE, CypG, CypH, Cyp40, PPWD1, PPIL1, or NKTR.

In certain embodiments, the compound is capable of inhibiting CypD. In certain embodiments, the compound is capable of inhibiting CypE. In certain embodiments, the compound is capable of inhibiting CypE. In certain embodiments, the compound is capable of inhibiting CypB, CypC, CypD, CypE, CypG, CypH, Cyp40, PPWD1, PPIL1, or NKTR. In certain embodiments, the compound is capable of inhibiting CypB, CypC, CypD, CypE, CypG, CypH, Cyp40, PPWD1, PPIL1, or NKTR.

In another aspect, the present disclosure provides methods of inhibiting the activity of a cyclophilin in a subject, the methods comprising administering to the subject an effective amount (e.g., therapeutically effective amount) of a compound, or pharmaceutical composition thereof, as described herein. In another aspect, the present disclosure provides methods of inhibiting the activity of a cyclophilin (e.g., CypB, CypC, CypD, CypE, CypG, CypH, Cyp40, PPWD1, PPIL1, NKTR) in a cell, tissue, or biological sample, the methods comprising contacting the cell, tissue, or biological sample with an effective amount of a compound, or a pharmaceutically acceptable salt, solvate, hydrate, polymorph, co-crystal, tautomer, stereoisomer, isotopically enriched form, prodrug, or mixture thereof, or a pharmaceutical composition thereof, as described herein. In another aspect, the present disclosure provides methods of inhibiting the activity of a cyclophilin in a cell, tissue, or biological sample, the methods comprising contacting the cell, tissue, or biological sample with an effective amount of a compound, or a pharmaceutically acceptable salt, solvate, hydrate, polymorph, co-crystal, tautomer, stereoisomer, isotopically enriched form, prodrug, or mixture thereof, or a pharmaceutical composition thereof, as described herein.

In another aspect, the present disclosure provides methods of inhibiting the activity of a cyclophilin (e.g., CypB, CypC, CypD, CypE, CypG, CypH, Cyp40, PPWD1, PPIL1, NKTR) in a cell, tissue, or biological sample, the methods comprising contacting the cell, tissue, or biological sample with an effective amount of a compound, or a pharmaceutically acceptable salt, solvate, hydrate, polymorph, co-crystal, tautomer, stereoisomer, isotopically enriched form, prodrug, or mixture thereof, or a pharmaceutical composition thereof, as described herein. In another aspect, the present disclosure provides methods of inhibiting (e.g., inhibiting the activity of) a cyclophilin (e.g., CypB, CypC, CypD, CypE, CypG, CypH, Cyp40, PPWD1, PPIL1, NKTR) in a subject, cell, tissue, or biological sample, the methods comprising administering to the subject or contacting the cell, tissue, or biological sample with an effective amount of a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof, or a pharmaceutical composition thereof.

In another aspect, the present disclosure provides methods of reducing oxidative stress in a subject, cell, tissue, or biological sample, the methods comprising administering to the subject or contacting the cell, tissue, or biological sample with an effective amount of a compound, or a pharmaceutically acceptable salt, solvate, hydrate, polymorph, co-crystal, tautomer, stereoisomer, isotopically enriched form, prodrug, or mixture thereof, or a pharmaceutical composition thereof, as described herein. In another aspect, the present disclosure provides methods of reducing oxidative stress in a subject, cell, tissue, or biological sample, the methods comprising administering to the subject or contacting the cell, tissue, or biological sample with an effective amount of a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof, or a pharmaceutical composition thereof, as described herein. In another aspect, the present disclosure provides methods of reducing oxidative stress in a cell, tissue, or biological sample, the methods comprising contacting the cell, tissue, or biological sample with an effective amount of a compound, or a pharmaceutically acceptable salt, solvate, hydrate, polymorph, co-crystal, tautomer, stereoisomer, isotopically enriched form, prodrug, or mixture thereof, or pharmaceutical composition thereof, as described herein. In another aspect, the present disclosure provides methods of reducing oxidative stress in a subject, the methods comprising administering to the subject an effective amount of a compound, or a pharmaceutically acceptable salt, solvate, hydrate, polymorph, co-crystal, tautomer, stereoisomer, isotopically enriched form, prodrug, or mixture thereof, or pharmaceutical composition thereof, as described herein.

In another aspect, the present disclosure provides methods of binding a cyclophilin in a subject, cell, tissue, or biological sample, the methods comprising administering to the subject or contacting the cell, tissue, or biological sample with an effective amount of a compound, or a pharmaceutically acceptable salt, solvate, hydrate, polymorph, co-crystal, tautomer, stereoisomer, isotopically enriched form, prodrug, or mixture thereof, or a pharmaceutical composition thereof, as described herein. In another aspect, the present disclosure provides methods of binding a cyclophilin in subject, cell, tissue, or biological sample, the methods comprising administering to the subject or contacting the cell, tissue, or biological sample) with an effective amount of a compound of Formula (I-A), (I-B), or (I-C), or a pharmaceutically acceptable salt, solvate, hydrate, polymorph, co-crystal, tautomer, stereoisomer, isotopically enriched form, prodrug, or mixture thereof, or a pharmaceutical composition thereof, as described herein. In another aspect, the present disclosure provides methods of binding a cyclophilin in a cell, tissue, or biological sample, the methods comprising contacting the cell, tissue, or biological sample with an effective amount of a compound, or a pharmaceutically acceptable salt, solvate, hydrate, polymorph, co-crystal, tautomer, stereoisomer, isotopically enriched form, prodrug, or mixture thereof, or pharmaceutical composition thereof, as described herein. In another aspect, the present disclosure provides methods of binding a cyclophilin in a subject, the methods comprising administering to the subject an effective amount of a compound, or a pharmaceutically acceptable salt, solvate, hydrate, polymorph, co-crystal, tautomer, stereoisomer, isotopically enriched form, prodrug, or mixture thereof, or pharmaceutical composition thereof, as described herein.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

In certain embodiments, the biological sample being contacted with the compound or composition is breast tissue, bone marrow, lymph node, lymph tissue, spleen, or blood. In certain embodiments, the biological sample being contacted with the compound or composition is a tumor cancerous tissue. In certain embodiments, the biological sample being contacted with the compound or composition is serum, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

In certain embodiments, the cell or tissue being contacted with the compound or composition is present in vitro. In certain embodiments, the cell or tissue being contacted with the compound or composition is present in vivo. In certain embodiments, the cell or tissue being contacted with the compound or composition is present ex vivo. In certain embodiments, the cell or tissue being contacted with the compound or composition is a malignant cell (e.g., malignant blood cell). In certain embodiments, the cell being contacted with the compound or composition is a malignant hematopoietic stem cell (e.g., malignant myeloid cell or malignant lymphoid cell). In certain embodiments, the cell being contacted with the compound or composition is a malignant lymphocyte (e.g., malignant T-cell or malignant B-cell). In certain embodiments, the cell being contacted with the compound or composition is a malignant white blood cell. In certain embodiments, the cell being contacted with the compound or composition is a malignant neutrophil, malignant macrophage, or malignant plasma cell. In certain embodiments, the cell being contacted with the compound or composition is a carcinoma cell. In certain embodiments, the cell being contacted with the compound or composition is a breast carcinoma cell. In certain embodiments, the cell being contacted with the compound or composition is a sarcoma cell. In certain embodiments, the cell being contacted with the compound or composition is a sarcoma cell from breast tissue.

The disease (e.g., neurological (e.g., neurodegenerative) disease (e.g., Alzheimer's disease, multiple sclerosis, Parkinson's disease, Huntington's disease, amyotrophic lateral sclerosis), metabolic disorder (e.g., obesity, diabetes, X-linked adrenoleukodystrophy (X-ALD)), proliferative disease (e.g., cancers), hepatic disease (e.g., liver cirrhosis), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases) to be treated or prevented using the compounds described herein may be associated with activity of a cyclophilin (e.g., CypB, CypC, CypD, CypE, CypG, CypH, Cyp40, PPWD1, PPIL1, NKTR). The disease to be treated or prevented using the compounds described herein may be associated with the peptidyl-prolyl-isomerase (PPIase) activity (e.g., increased PPIase activity, decreased PPIase activity, undesired PPIase activity, PPIase aberrant activity) of a cyclophilin (e.g., CypB, CypC, CypD, CypE, CypG, CypH, Cyp40, PPWD1, PPIL1, NKTR). The disease to be treated or prevented using the compounds described herein may be associated with the overexpression of a cyclophilin (e.g., CypB, CypC, CypD, CypE, CypG, CypH, Cyp40, PPWD1, PPIL1, NKTR).

In certain embodiments, the disease to be treated or prevented using the compounds described herein may be associated with the overexpression of a cyclophilin (e.g., CypB, CypC, CypD, CypE, CypG, CypH, Cyp40, PPWD1, PPIL1, NKTR). A disease may be associated with aberrant activity of a cyclophilin (e.g., CypB, CypC, CypD, CypE, CypG, CypH, Cyp40, PPWD1, PPIL1, NKTR). Aberrant activity of a cyclophilin (e.g., CypB, CypC, CypD, CypE, CypG, CypH, Cyp40, PPWD1, PPIL1, NKTR) may be elevated and/or inappropriate or undesired activity of the cyclophilin. The compounds described herein, and pharmaceutically acceptable salts, solvates, hydrates, polymorphs, co-crystals, tautomers, stereoisomers, isotopically enriched forms, prodrugs, or mixtures thereof, may inhibit the activity of a cyclophilin (e.g., CypB, CypC, CypD, CypE, CypG, CypH, Cyp40, PPWD1, PPIL1, NKTR) and be useful in treating and/or preventing diseases

All types of biological samples described herein or known in the art are contemplated as being within the scope of the invention. In certain embodiments, the neurological disease to be treated or prevented using the compounds described herein is a neurodegenerative disease. In certain embodiments, the neurodegenerative disease is Alzheimer's disease. In certain embodiments, the neurodegenerative disease is multiple sclerosis. In certain embodiments, the neurological disease is Parkinson's disease. In certain embodiments, the neurological disease is Huntington's disease. In certain embodiments, the neurological disease is amyotrophic lateral sclerosis. In certain embodiments, the metabolic disorder to be treated or prevented using the compounds described herein is diabetes (e.g., Type I diabetes, Type II diabetes, gestational diabetes). In some embodiments, the metabolic disorder is hyperglycemia. In some embodiments, the metabolic disorder is hyperinsulinemia. In some embodiments, the metabolic disorder is insulin resistance. In some embodiments, the metabolic disorder is obesity.

In certain embodiments, the proliferative disease to be treated or prevented using the compounds described herein is cancer. All types of cancers disclosed herein or known in the art are contemplated as being within the scope of the invention. In some embodiments, the proliferative disease is a benign neoplasm. All types of benign neoplasms disclosed herein or known in the art are contemplated as being within the scope of the invention. In some embodiments, the proliferative disease is associated with angiogenesis. All types of angiogenesis disclosed herein or known in the art are contemplated as being within the scope of the invention. In certain embodiments, the cancer is colorectal cancer.

In certain embodiments, the disease and/or condition to be treated or prevented using the compounds described herein is a condition associated with the mitochondria (e.g., a mitochondrial disease). In certain embodiments, the mitochondrial disease and/or condition to be treated or prevented is associated with regulation of the mitochondrial permeability transition pore (mPTP). In certain embodiments, the mitochondrial disease and/or condition to be treated or prevented is associated with regulation of the opening and/or closing of the mPTP. In certain embodiments, the condition to be treated or prevented using the compounds described herein is a condition associated with autophagy and/or aging. In certain embodiments, the condition to be treated or prevented using the compounds described herein is a cardiovascular condition (e.g., ischemia-reperfusion injury), stroke, heart attack, conditions associated with oxidative stress, mitochondrial diseases. In certain embodiments, the cardiovascular condition is ischemia-reperfusion injury. In certain embodiments, the cardiovascular condition is stroke or heart attack.

In certain embodiments, the condition associated with autophagy to be treated or prevented using the compounds described herein is neurodegenerative disease (e.g., Alzheimer's disease, multiple sclerosis, Parkinson's disease, Huntington's disease, amyotrophic lateral sclerosis), infection (e.g., infection by a bacteria, virus, or other microbes), cancer, condition associated with aging, or heart disease.

One aspect of the disclosure relates to methods of reducing oxidative stress in a subject, cell, tissue, or biological sample, the method comprising administering to the subject or contacting the cell, tissue, or biological sample with a therapeutically effective amount of compounds described herein.

Another aspect of the disclosure relates to methods of inhibiting the activity of a cyclophilin in a subject, cell, tissue, or biological sample. In certain embodiments, the cyclophilin is a CypB, CypC, CypD, CypE, CypG, CypH, Cyp40, PPWD1, PPIL1, NKTR. In certain embodiments, the activity of the cyclophilin is aberrant activity of the cyclophilin. In certain embodiments, the activity of the cyclophilin is increased activity of the cyclophilin. In certain embodiments, the activity of the cyclophilin is undesired activity of the cyclophilin. In certain embodiments, the inhibition of the activity of the cyclophilin is irreversible. In other embodiments, the inhibition of the activity of the cyclophilin is reversible. In certain embodiments, the methods of inhibiting the activity of the cyclophilin include attaching a compound described herein to the cyclophilin (e.g., CypB, CypC, CypD, CypE, CypG, CypH, Cyp40, PPWD1, PPIL1, NKTR). In certain embodiments, the methods comprise covalently inhibiting a cyclophilin. In certain embodiments, the methods comprise covalently inhibiting a cyclophilin (e.g., CypD, CypC). In certain embodiments, the methods comprise reversibly inhibiting a cyclophilin (e.g., CypB, CypC, CypD, CypE, CypG, CypH, Cyp40, PPWD1, PPIL1, NKTR).

In certain embodiments, the methods described herein include administering to a subject or contacting a cell, tissue, or biological sample with an effective amount of a compound described herein, or a pharmaceutically acceptable salt, solvate, hydrate, polymorph, co-crystal, tautomer, stereoisomer, isotopically enriched form, prodrug, or mixture thereof, or a pharmaceutical composition thereof. In certain embodiments, the methods described herein include administering to a subject or contacting a cell, tissue, or biological sample with an effective amount of a compound described herein, or a pharmaceutically acceptable salt thereof, or a pharmaceutical composition thereof. In certain embodiments, the compound is contacted with a cell, tissue, or biological sample. In certain embodiments, the compound is administered to a subject. In certain embodiments, the compound is administered in combination with one or more additional pharmaceutical agents described herein. The additional pharmaceutical agent may be an agent for treating a neurological (e.g., neurodegenerative) disease. The additional pharmaceutical agent may be an agent for treating a metabolic disorder. The additional pharmaceutical agent may be an anti-aging agent. The additional pharmaceutical agent may be an agent for treating a cardiovascular condition (e.g., ischemia-reperfusion injury), stroke, heart attack, conditions associated with oxidative stress, mitochondrial diseases. The additional pharmaceutical agent may be an anti-proliferative agent. In certain embodiments, the additional pharmaceutical agent is an anti-cancer agent. The additional pharmaceutical agent may also be a cyclophilin inhibitor. In certain embodiments, the additional pharmaceutical agent is an inhibitor of CypD. In certain embodiments, the additional pharmaceutical agent is an inhibitor of CypE. In certain embodiments, the additional pharmaceutical agent is an inhibitor of a cyclophilin (e.g., CypB, CypC, CypD, CypE, CypG, CypH, Cyp40, PPWD1, PPIL1, NKTR). In certain embodiments, the additional pharmaceutical agent is a selective inhibitor of cyclophilin (e.g., CypB, CypC, CypD, CypE, CypG, CypH, Cyp40, PPWD1, PPIL1, NKTR). In certain embodiments, the additional pharmaceutical agent is a non-selective inhibitor of cyclophilin (e.g., CypB, CypC, CypD, CypE, CypG, CypH, Cyp40, PPWD1, PPIL1, NKTR)

The inventive compounds or compositions may synergistically augment inhibition of cyclophilins induced by the additional pharmaceutical agent(s) in the subject, cell, tissue, or biological sample. Thus, the combination of the inventive compounds or compositions and the additional pharmaceutical agent(s) may be useful in treating proliferative diseases resistant to a treatment using the additional pharmaceutical agent(s) without the inventive compounds or compositions.

In some embodiments, the activity of a cyclophilin is non-selectively inhibited by the compounds or pharmaceutical compositions described herein. In some embodiments, the activity of the cyclophilin being inhibited is selectively inhibited by the compounds or pharmaceutical compositions described herein, compared to the activity of a cyclophilin (e.g., a different cyclophilin). In certain embodiments, the activity of a cyclophilin (e.g., CypB, CypC, CypD, CypE, CypG, CypH, Cyp40, PPWD1, PPIL1, NKTR) is selectively inhibited by a compound or pharmaceutical composition described herein, compared to the activity of a different protein. In certain embodiments, the activity of CypD is selectively inhibited by a compound or pharmaceutical composition described herein, compared to the activity of another cyclophilin (e.g., CypB, CypC, CypE, CypG, CypH, Cyp40, PPWD1, PPIL1, NKTR). In certain embodiments, the activity of CypE is selectively inhibited by a compound or pharmaceutical composition described herein, compared to the activity of another cyclophilin (e.g., CypB, CypC, CypE, CypG, CypH, Cyp40, PPWD1, PPIL1, NKTR). In certain embodiments, the activity of a cyclophilin (e.g., CypB, CypC, CypD, CypE, CypG, CypH, Cyp40, PPWD1, PPIL1, NKTR) is selectively inhibited by a compound or pharmaceutical composition described herein, compared to the activity of another cyclophilin.

In certain embodiments, a kit described herein includes a first container comprising a compound or pharmaceutical composition described herein. In certain embodiments, a kit described herein is useful in treating and/or preventing a diseasein a subject in need thereof, inhibiting the activity of a cyclophilin (e.g., CypB, CypC, CypD, CypE, CypG, CypH, Cyp40, PPWD1, PPIL1, NKTR) in a subject, cell, tissue, or biological sample, and/or reducing oxidative stress in a subject, cell, tissue, or biological sample.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a proliferative disease in a subject in need thereof, preventing a disease in a subject in need thereof, inhibiting the activity of a cyclophilin (e.g., CypB, CypC, CypD, CypE, CypG, CypH, Cyp40, PPWD1, PPIL1, NKTR) in a subject, cell, tissue, or biological sample, and/or reducing oxidative stress in a subject, cell, tissue, or biological sample. A kit described herein may include one or more additional pharmaceutical agents described herein as a separate composition.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope. The compounds provided herein can be prepared from readily available starting materials using the following general methods and procedures or methods known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

### Example 1. Development of CypD-Selective Inhibitors

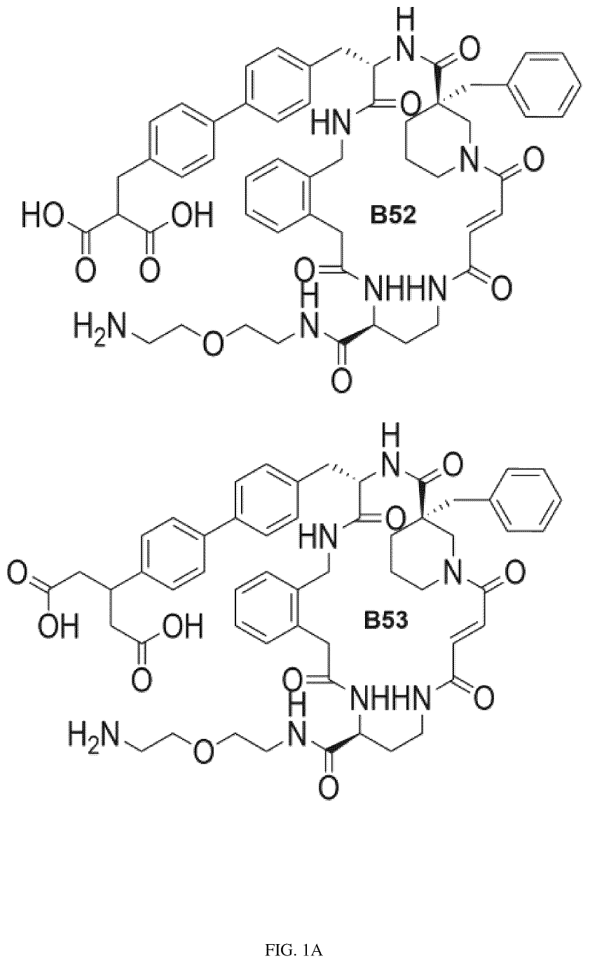

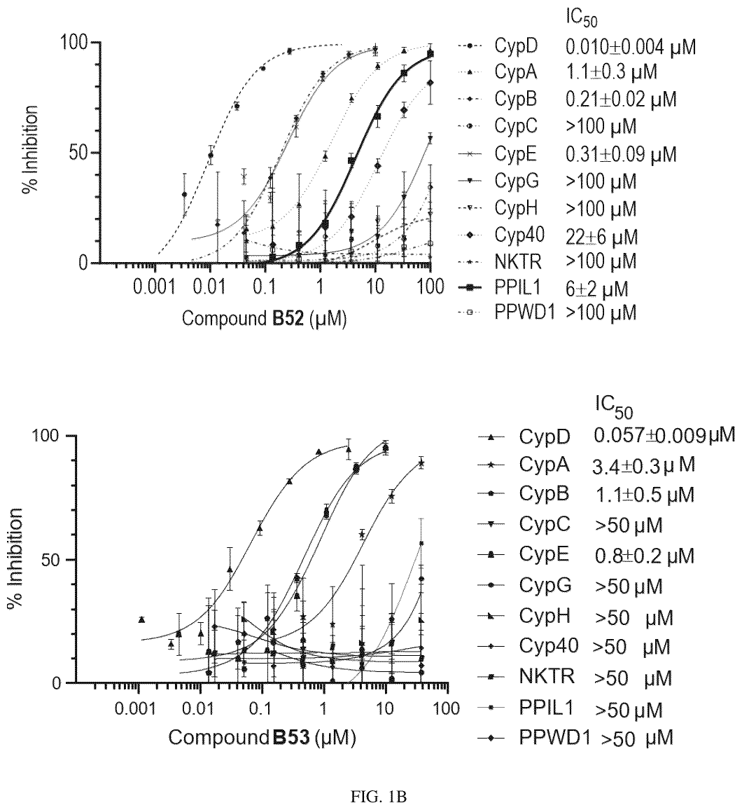

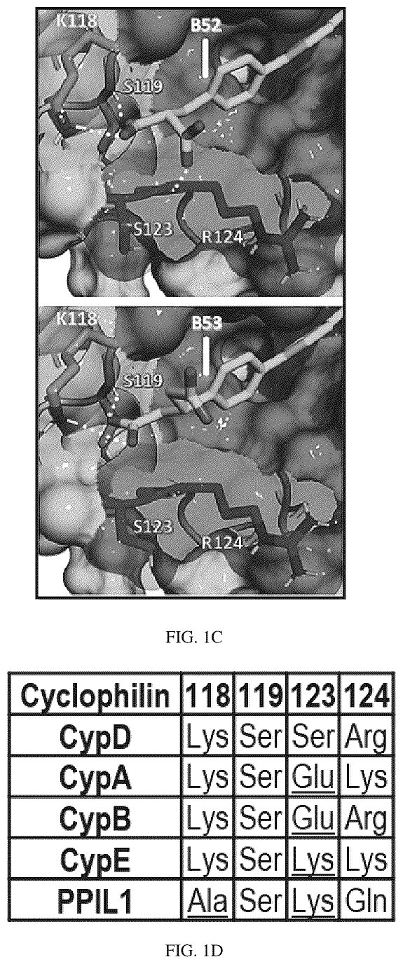

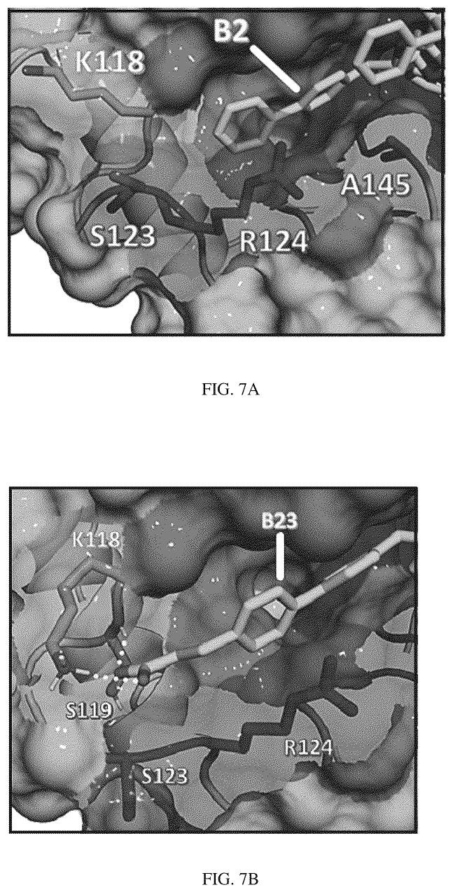

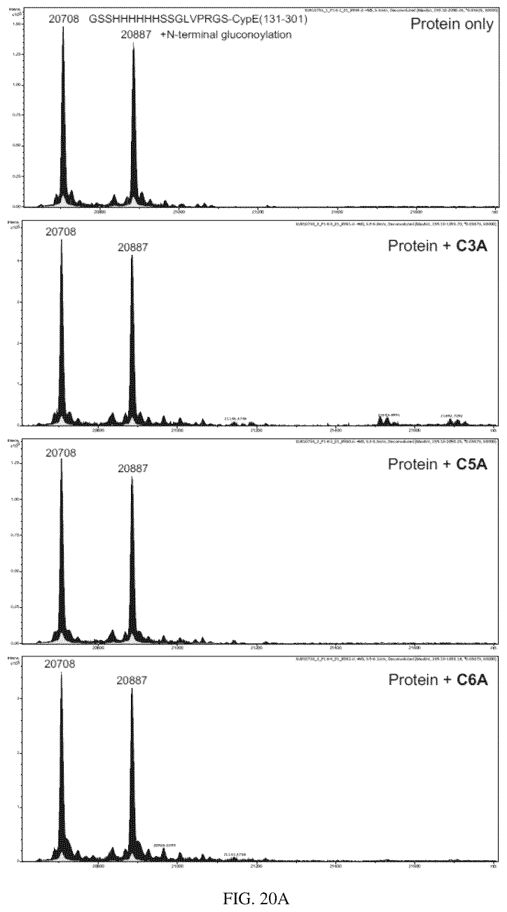

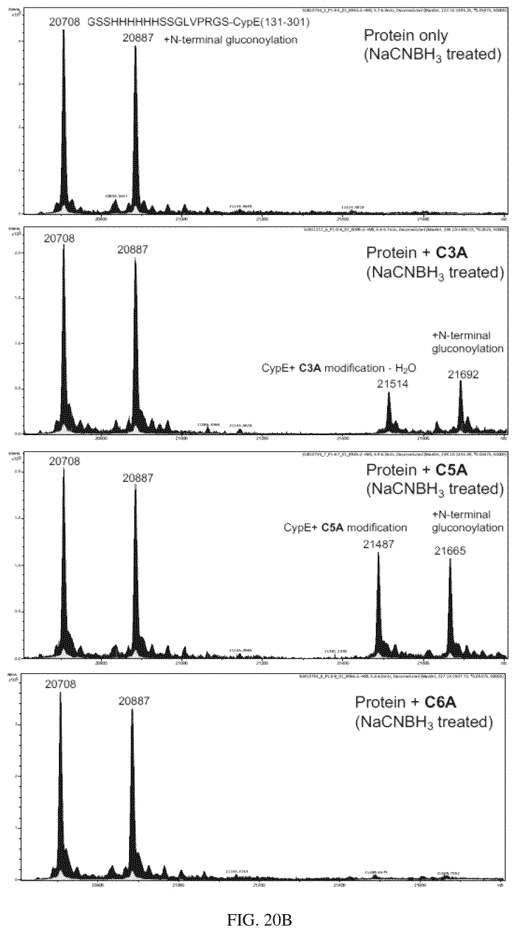

Starting from a slightly promiscuous inhibitor B2, selectivity for CypD was improved by placing carboxylates that could hydrogen bond with the fully conserved S119 residue and a salt bridge with the semi-conserved K118 residue, with carboxylate containing inhibitors B23 and B25 achieving selectivity over non-lysine-containing PPIL1, Cyp40, CypC, and PPWD1. In particular, while CypD's K118 side-chain is normally oriented away from the S2 pocket, this residue can be ligand directed inside the S2 pocket by B23 and B25 to form a salt bridge (FIGS. 7A-7B) and grant CypD tolerance of a negative charge within its S2 pocket. To impart selectivity over K118-containing CypA, CypB, and CypE, a second carboxylate was installed to interact to the analogous 123 gatekeeper residue on each cyclophilin, building on the observation that mono-carboxylates B23 and B25 exert small conformational and selectivity effects on CypD's flexible S123 loop region (FIG. 7A, FIGS. 20A-20B). This approach resulted in compounds B52 and B53, which show high potency and selectivity for CypD (FIGS. 1A-1D). CypD's S123 can sterically and electronically tolerate dicarboxylates, while Glu at the analogous CypD position 123 in CypA and CypB presumably repel the dicarboxylate, and CypE's more bulky lysine may sterically clash with the dicarboxylate (FIGS. 1D, 29, 30, Table 4). Complementing these trends was the overall migration of S2 pocket residues of CypD to accommodate ligand binding (FIG. 7B).

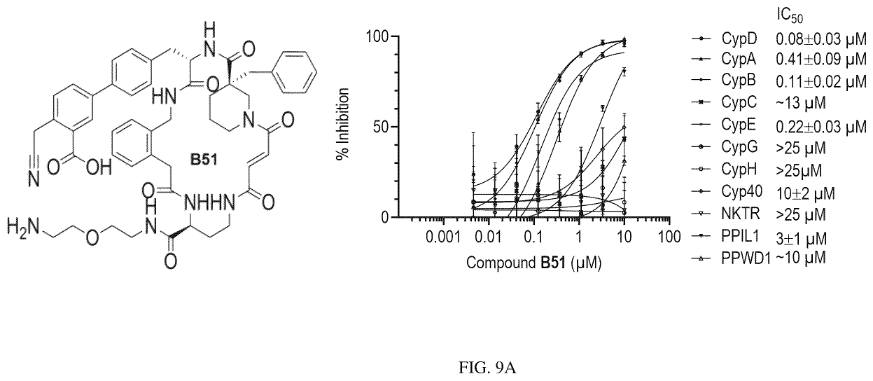

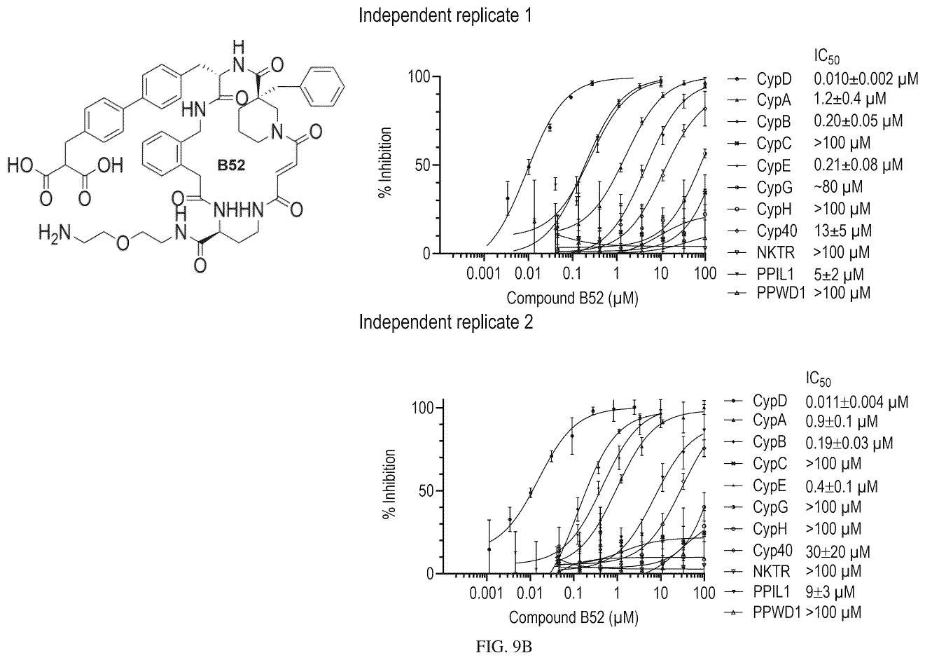

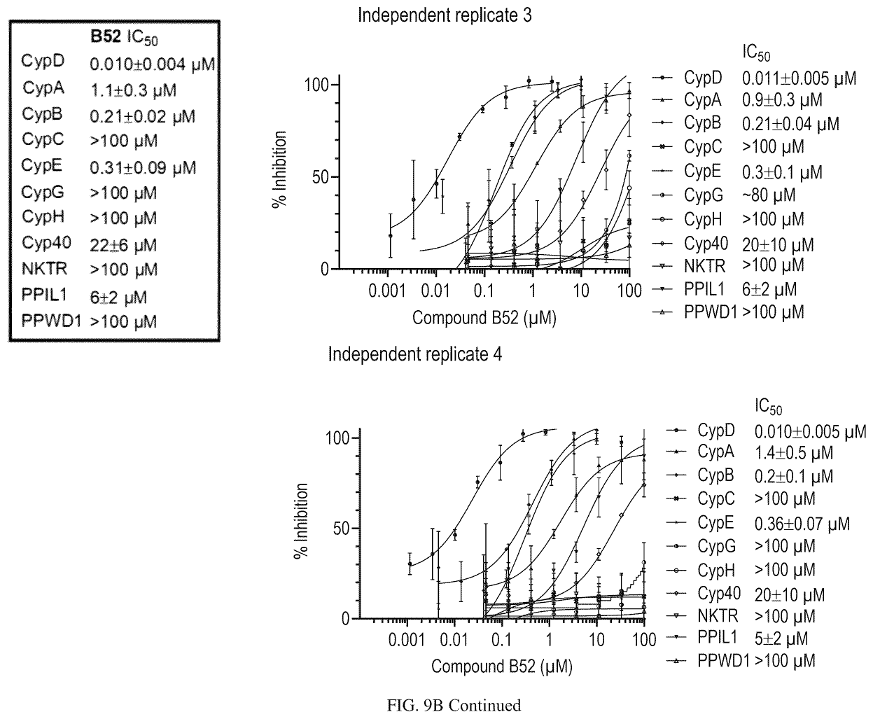

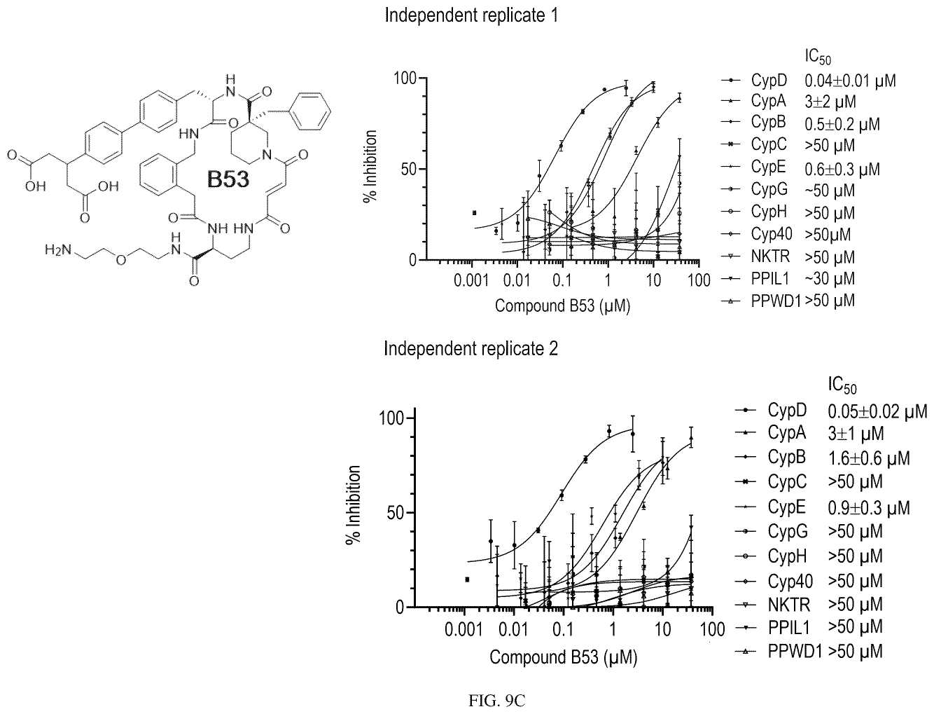

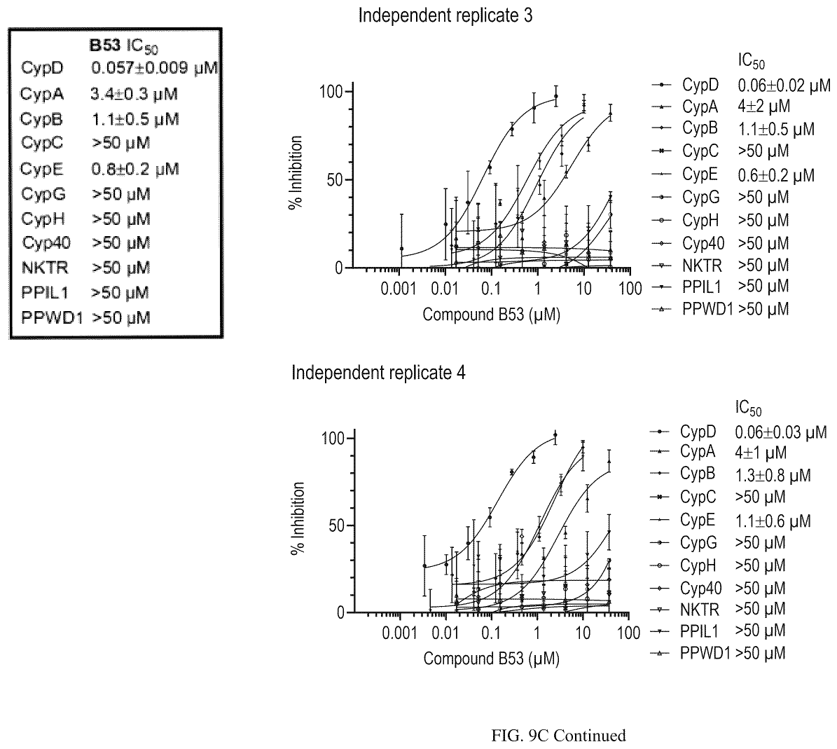

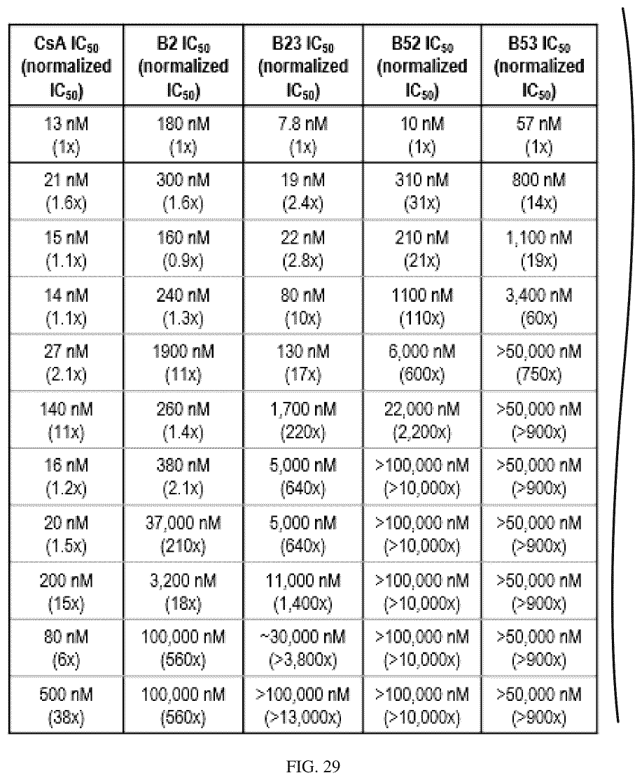

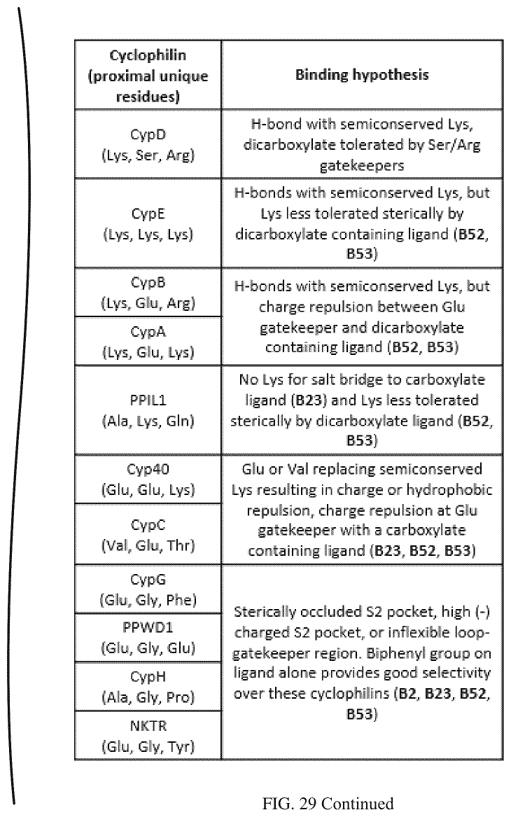

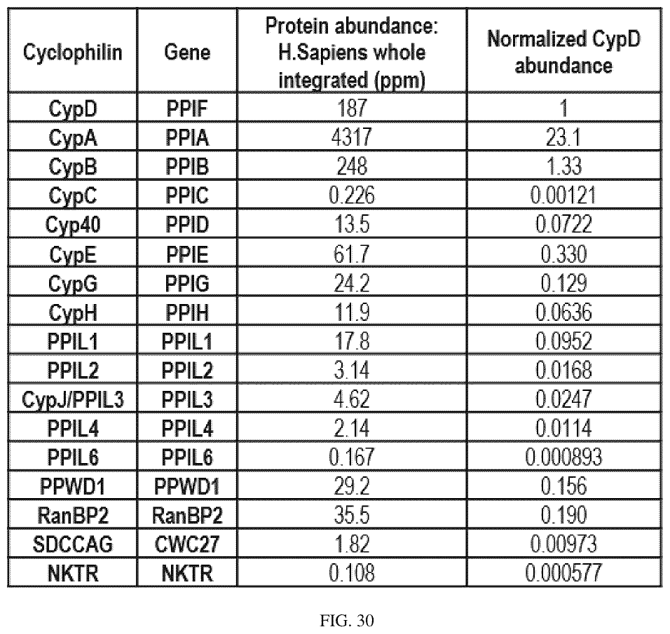

While the nitrile of B51 hampers the selectivity compared to the B23 carboxylate (FIG. 9A), B52 and B53, which contain malonate or glutarate moieties, respectively, retain CypD potency (B52 IC₅₀^{CypD}=0.010 μM; B53 IC₅₀^{CypD}=0.057 μM) compared to B23, while further improving CypD selectivity (FIGS. 1A-1B and 9B-9C). B52 and B53 show ˜15- to 20-fold selectivity for inhibiting CypD over the most closely related cyclophilins, CypE and CypB, 100- to 500-fold selectivity over CypA and PPIL1, and >750-fold selectivity against the remaining six cyclophilins (FIGS. 29, 30). The 100-fold selectivity of B52 and B53 for CypD over CypA is especially noteworthy since CypA is abundantly expressed cyclophilin in human cells, and one of the most abundantly expressed intracellular proteins (FIG. 30)⁴⁷. Gaining selectivity over this cyclophilin therefore facilitates towards intracellular subtype-selective CypD inhibition, as the excess of CypA present could sequester more promiscuous inhibitors. Co-crystal structures of B52 and B53 bound to CypD were solved (FIG. 1C, PDB IDs 7THD, 7THF). A similar binding mode as B23 was observed, maintaining a salt bridge with K118, but now presenting a second carboxylate in the proximity of the S123 and R124 residues, as designed. For B52, a novel hydrogen bond with the backbone atoms between S123 and R124 was also observed. Therefore, the selectivity of B52 and B53 for CypD over CypA, CypB, and CypE was attributed to the presentation of the second carboxylate near S123 (FIG. 1D).

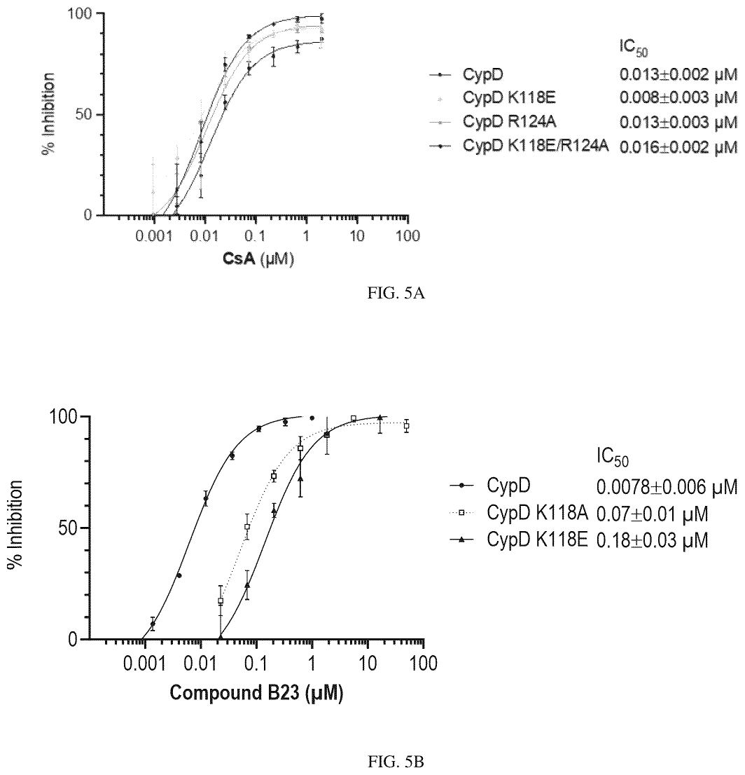

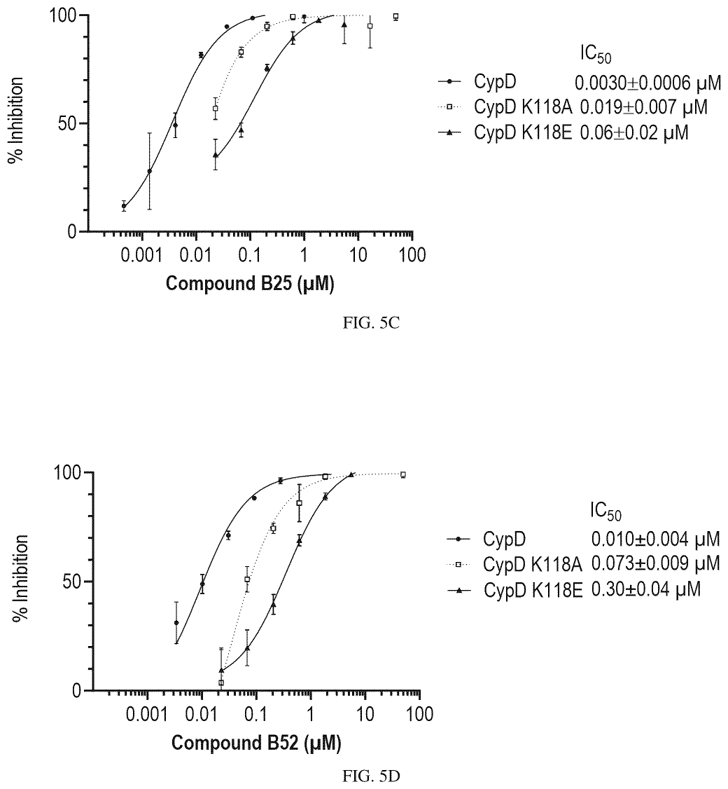

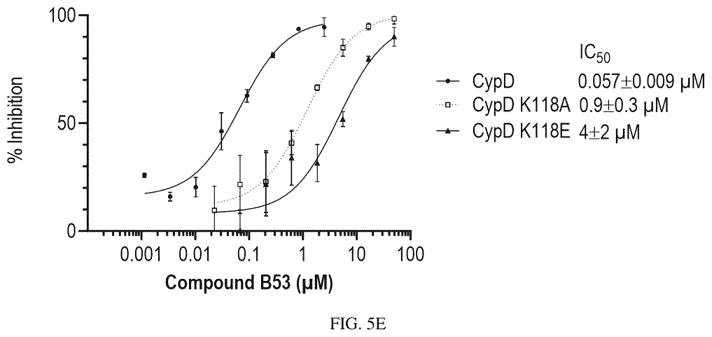

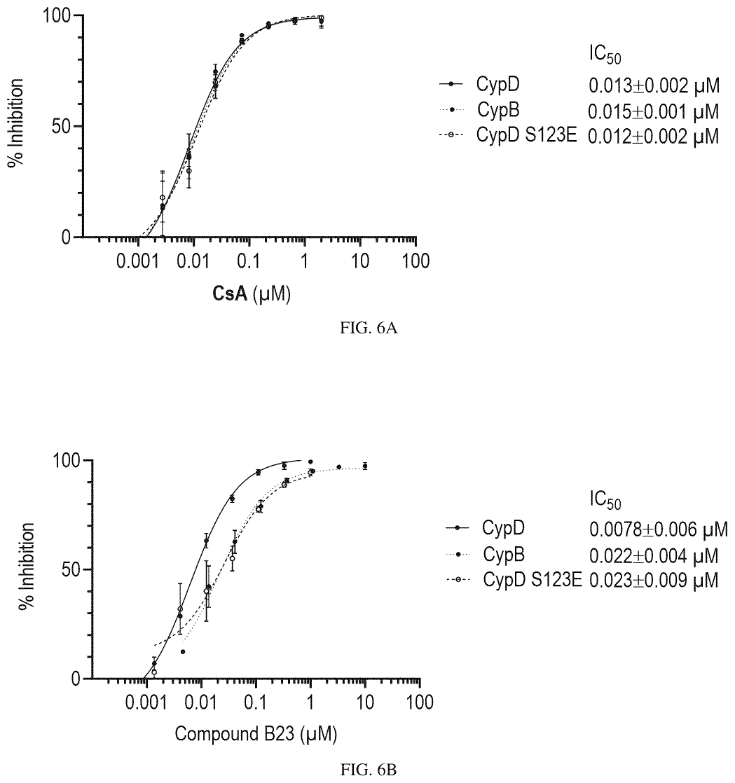

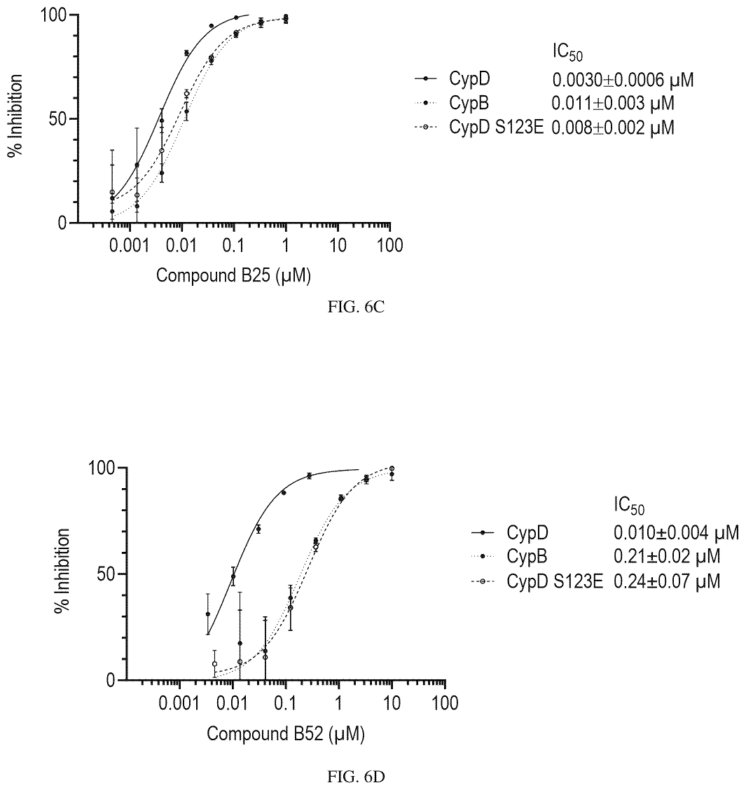

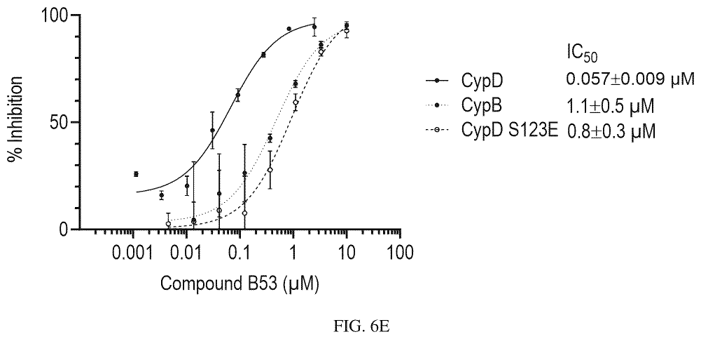

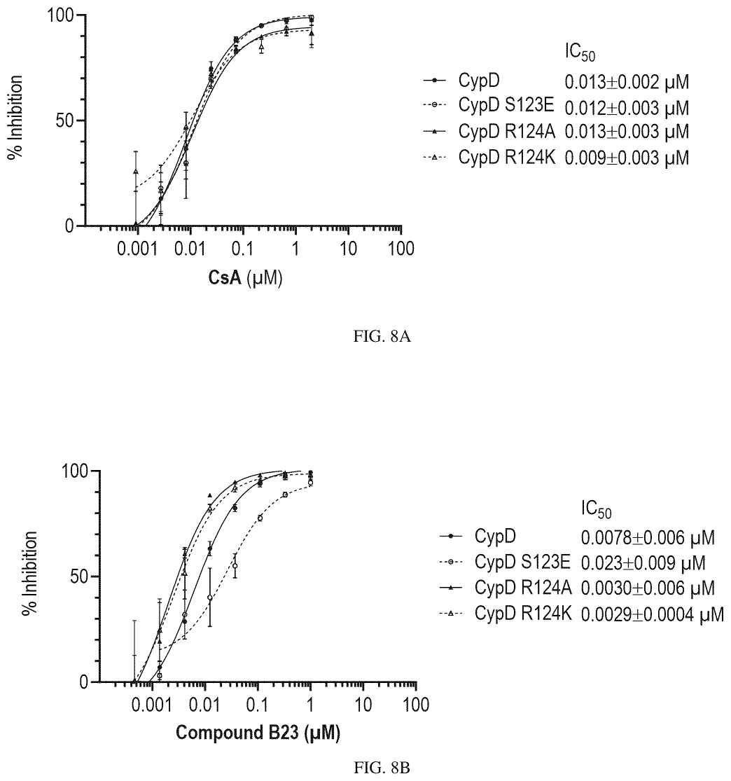

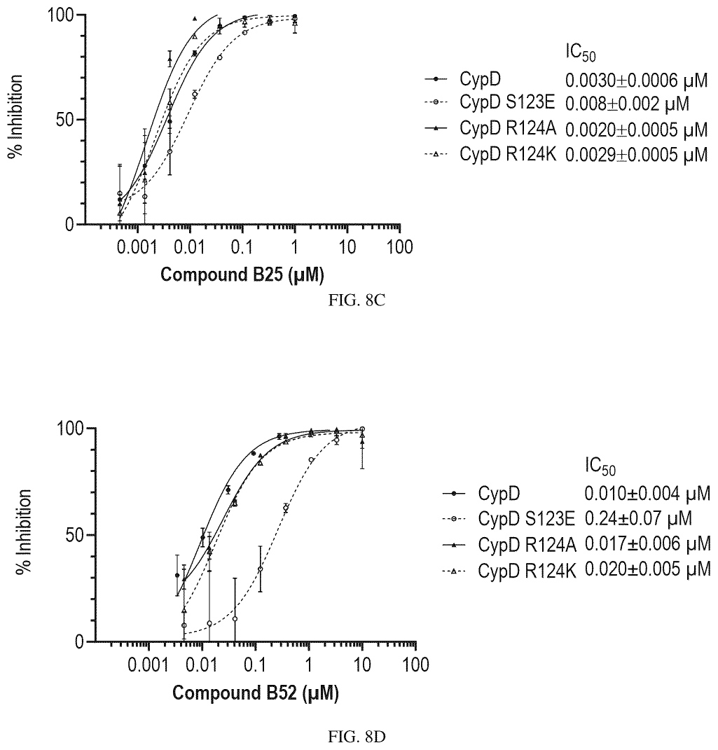

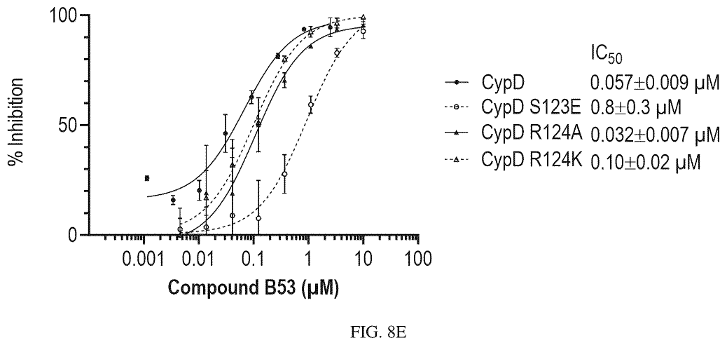

To test this basis of selectivity, B52 and B53 were assayed against CypD gatekeeper mutants S123E, R124A, and R124K (FIGS. 6D-6E and 8D-8E), followed by CypD mutants K118A and K118E (FIGS. 5D-5E). Similar but improved trends as seen with mono-carboxylates B23 and B25 compared to wild-type CypD were observed-decreased potency for CypD S123E (B52 IC₅₀^{CypDS123E}=0.24 μM; B53 IC₅₀^{CypDS123E}=0.8 μM) roughly equal to CypB (B52 IC₅₀^{CypB}=0.21 μM; B53 IC₅₀^{CypB}=1.1 μM), little to no ablation of potency for the CypD R124A and R124K mutants compared to wild-type CypD, and a decrease in potency for CypD K118A (B52 IC₅₀^{CypDK118A}=0.073 μM; B53 IC₅₀^{CypDK118A}=0.9 μM) and K118E (B52 IC₅₀^{CypDK118E}=0.3 μM; B53 IC₅₀^{CypDK118A}=4 μM) mutants compared to wild-type CypD upon removing the possibility of an inhibitor:protein salt bridge. These results suggest that B52 and B53 achieved substantial CypD selectivity based on favorable contacts with its K118 and S123 residues, and the absence of such interactions with homologous positions on other cyclophilin isoforms. Dicarboxylate (malonic or glutaric acid) groups on the inhibitor scaffolds developed in this study thus provided a chemical environment that CypD, but not other cyclophilins, could accommodate.

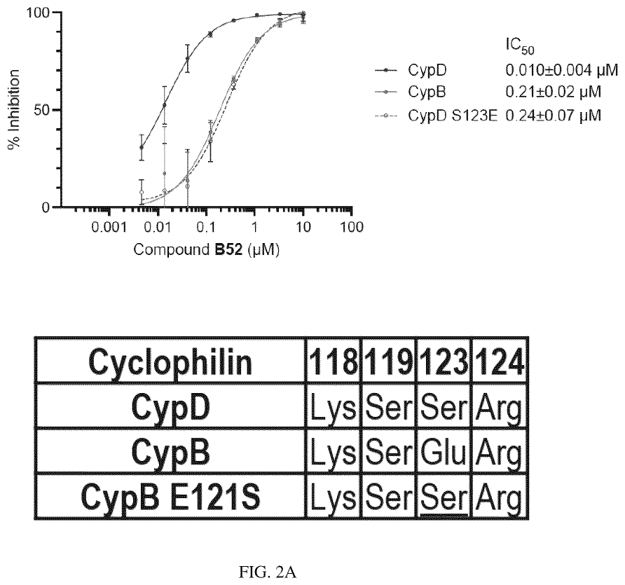

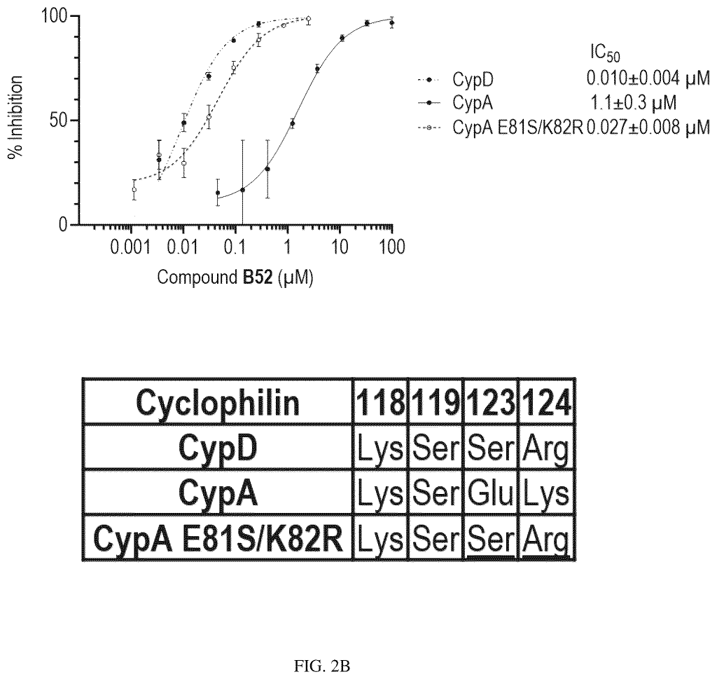

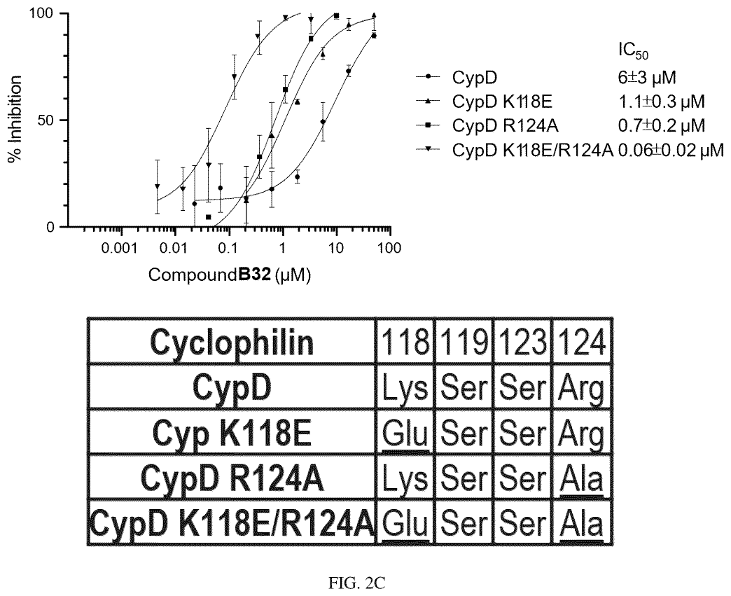

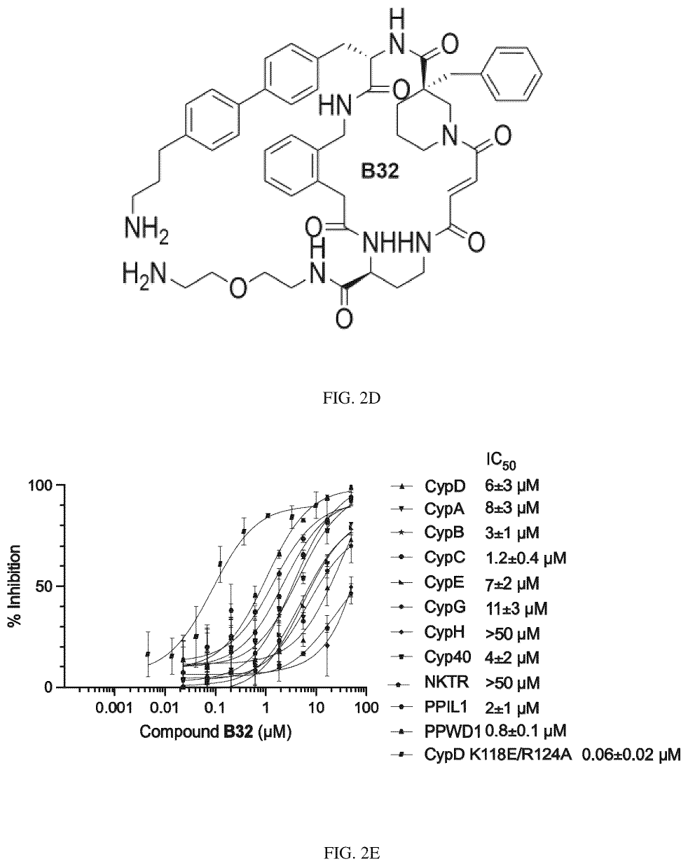

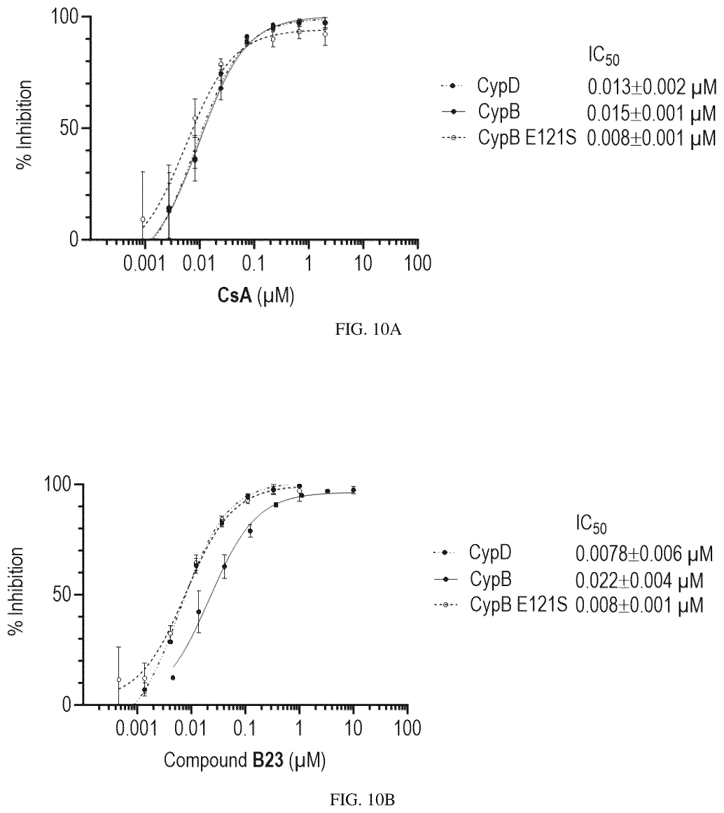

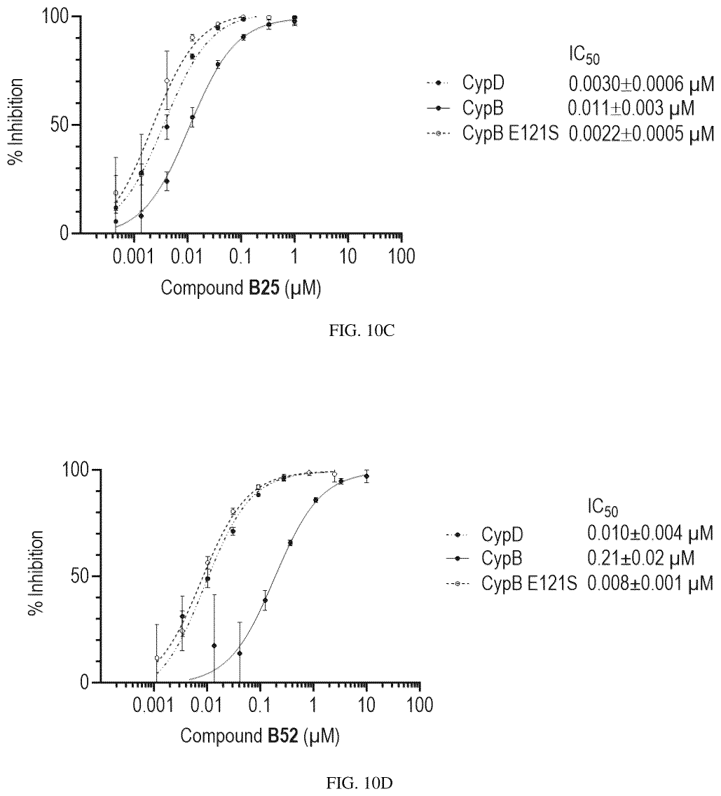

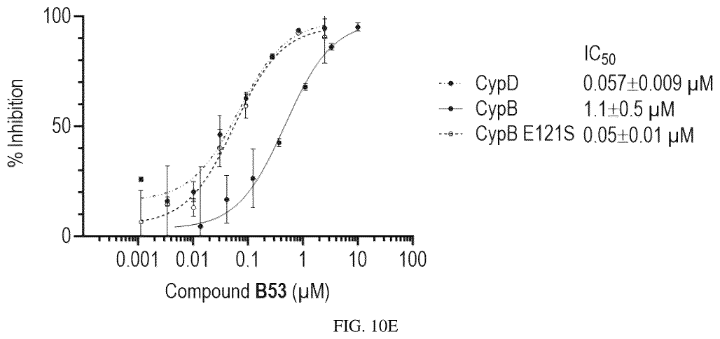

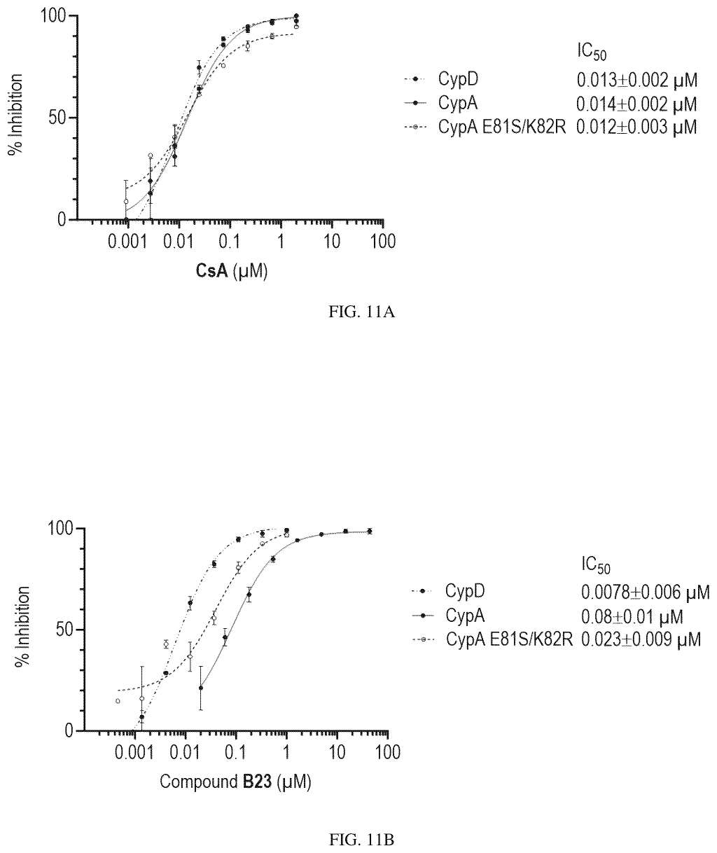

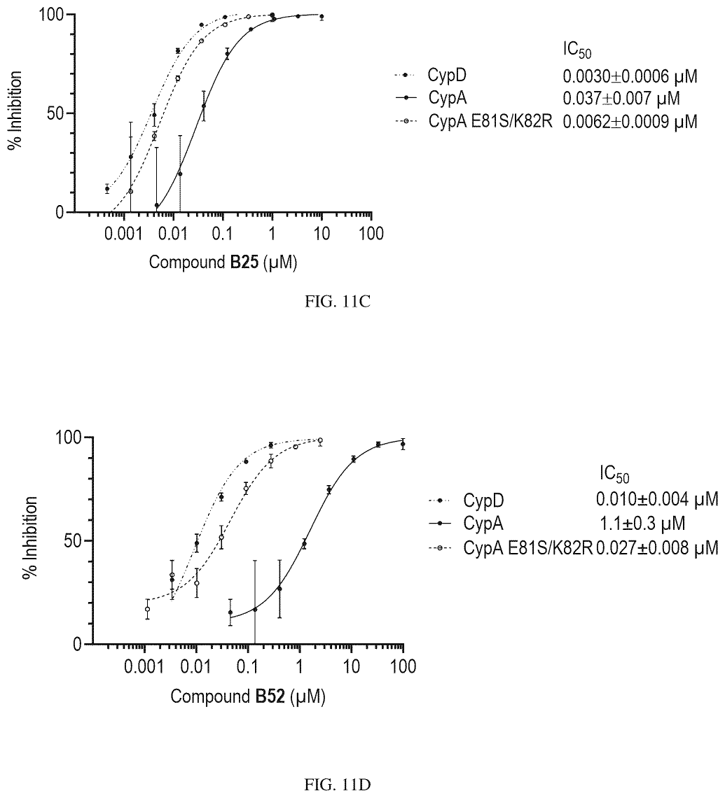

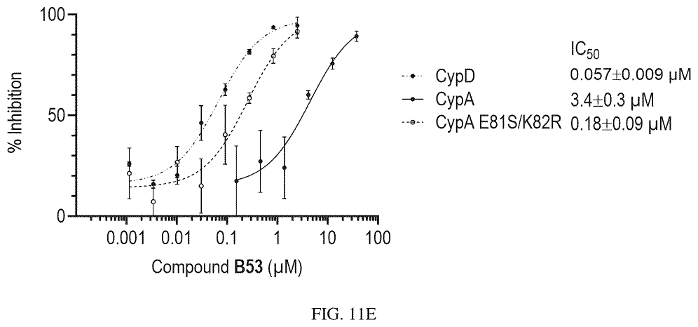

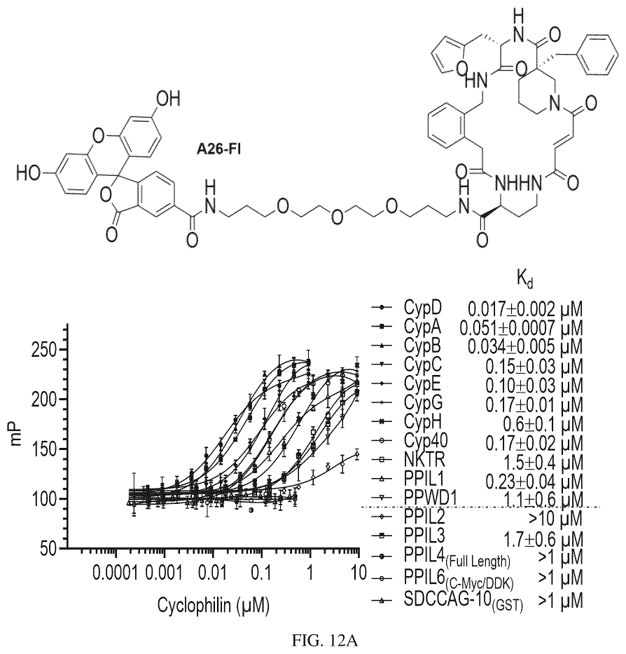

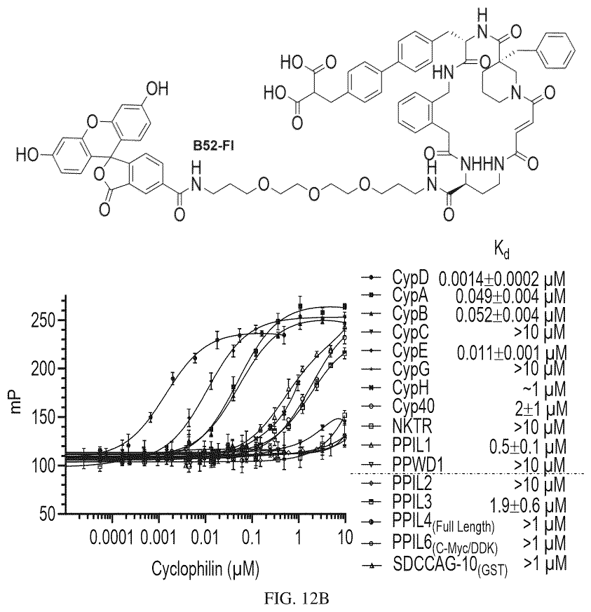

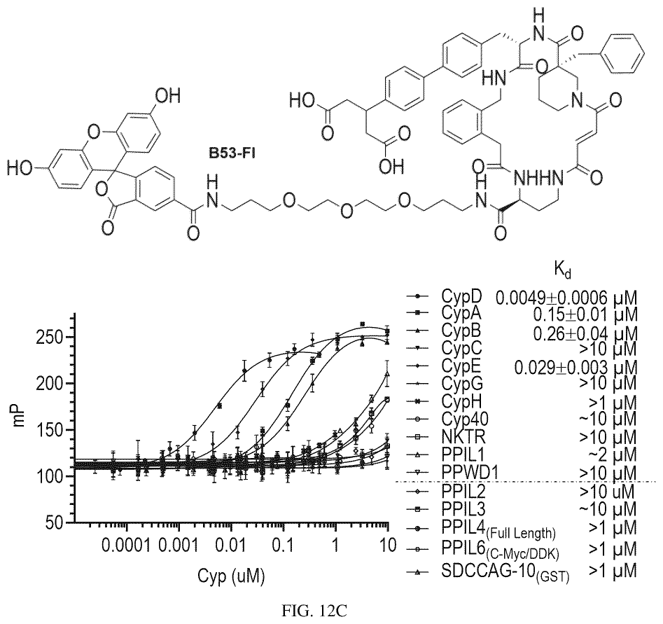

To further establish the dependence of CypD inhibitor potency on S2 pocket residues, B52 and B53's attenuated potency for CypB and CypA was rescued by installing gatekeeper mutations to match CypD's residues in this pocket (CypB E121S, CypA E81S/K82R). As expected, both B52 and B53 had similar potencies for CypD as CypB E121S (B52, IC₅₀^{CypD}=0.010 μM, IC₅₀^{CypBE121S}=0.008 μM; B53, IC₅₀^{CypD}=0.057 μM, IC₅₀^{CypBE121S}=0.05 μM), rescuing the 15-20-fold inhibition potency difference of B52 and B53 for CypD versus wild-type CypB (B52, IC₅₀^{CypB}=0.21 μM; B53, IC₅₀^{CypB}=1.1 μM) (FIGS. 2A and 10D-10E). Similarly, B52 and B53 were shown to have similar potencies for CypD and CypA E81S/K82R (B52, IC₅₀^{CypD}=0.010 μM, IC₅₀^{CypAE81S/K82R}=0.027 μM; B53, IC₅₀^{CypD}=0.057 μM, IC₅₀^{CypAE81S/K82R}=0.18 μM), rescuing the ˜100-fold inhibition potency difference of B52 and B53 for CypD versus wild-type CypA (B52, IC₅₀^{CypA}=1.1 μM; B53, IC₅₀^{CypA}=3.4 μM) (FIGS. 2B and 11D-11E). These trends were also conserved for monocarboxylates B23 and B25, albeit to a lesser degree (FIGS. 10B-10C AND 11B-11C). Both CypB E121S and CypA E81S/K82R mutants also retained their WT prolyl-isomerase activity. These prolyl isomerase inhibition trends were maintained with fluorescein-labeled A26 (A26-Fl), B52 (B52-Fl), and B53 (B53-Fl), using a previously reported fluorescence polarization assay to measure cyclophilin binding (FIGS. 12A-12C). While A26-Fl promiscuously binds the 11 prolyl-isomerase-active cyclophilins, B52-Fl and B53-Fl exhibited selective binding for CypD, corroborating results from the prolyl-isomerase screens for B52 and B53 (FIG. 1B). The binding activity of five non-prolyl isomerase active cyclophilins (PPIL2, PPIL3, PPIL4, PPIL6, SDCCAG-10) was also assayed, and very weak or no binding with promiscuous A26-Fl or CypD-selective B52-Fl or B53-Fl was observed, suggesting that their macrocycle scaffold could not target these non-prolyl isomerase cyclophilins. Collectively, these results indicate that interactions between inhibitors and S2 pocket residues are strong determinants of CypD potency and selectivity.

Since mutating CypA's and CypB's S2 pocket improved binding to dicarboxylate-containing inhibitors, it was determined that CypD's S2 pocket could also be modified to accommodate an alternate ligand from the series of inhibitors. The amine containing compound B32 was used, as it shows an alternate selectivity profile compared to the majority of other studied inhibitors, but has overall poor potency for each cyclophilin (FIGS. 2D-2E). Given that CypD has a relatively sterically unconcluded, positively charged S2 pocket, it was determined that replacing some of the residues' neutral or negatively charged amino acids would improve potency with B32 (FIG. 2C). Starting with CypD mutants K118E and R124A, it was observed that B32 displayed a 5- to 6-fold inhibition potency improvement compared to wild-type CypD (IC₅₀^{CypD}=6 μM, IC₅₀^{CypDK118E}=1.1 μM, IC₅₀^{CypDR124A}=0.7 μM). Combining these two mutants resulted in an additive increase in potency (IC₅₀^{CypDK11E/R124A}=0.06 μM), a 100-fold improvement compared to wild-type CypD (FIG. 2C). Compared to the rest of the prolyl-isomerase active cyclophilin family, B32 exhibited good selectivity for CypD K118E/R124A, with at least 13-fold preference over the nearest cyclophilin, PPWD1 (FIG. 2E). B32 showed over 100-fold selectivity over CypA, the most abundantly expressed cyclophilin in human cells (FIG. 30). Of particular note, B32 had increased potency for CypD R124A containing mutants, suggesting a novel binding mode contingent on this residue, in contrast to wild-type CypD and B52/B53. In tandem with CypD and B52/B53, CypD K118E/R124A and B32 provides another pair of cyclophilin-ligand pairs with selective inhibition dependent on S2 pocket identity.

### Example 2. CypD Inhibitors are Active in Mitochondria and can Enter Mammalian Cells as Prodrugs

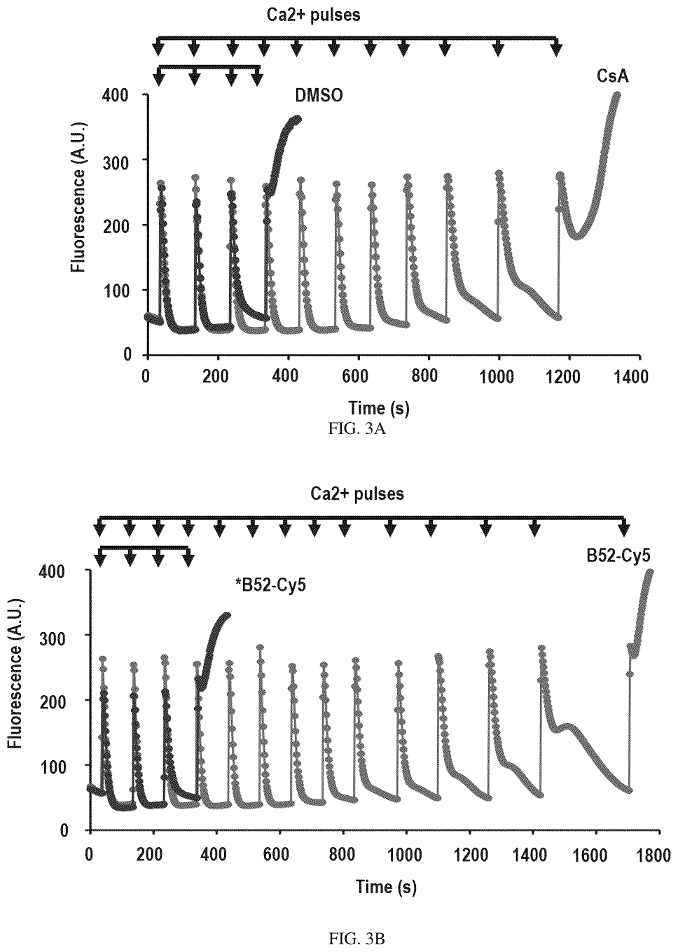

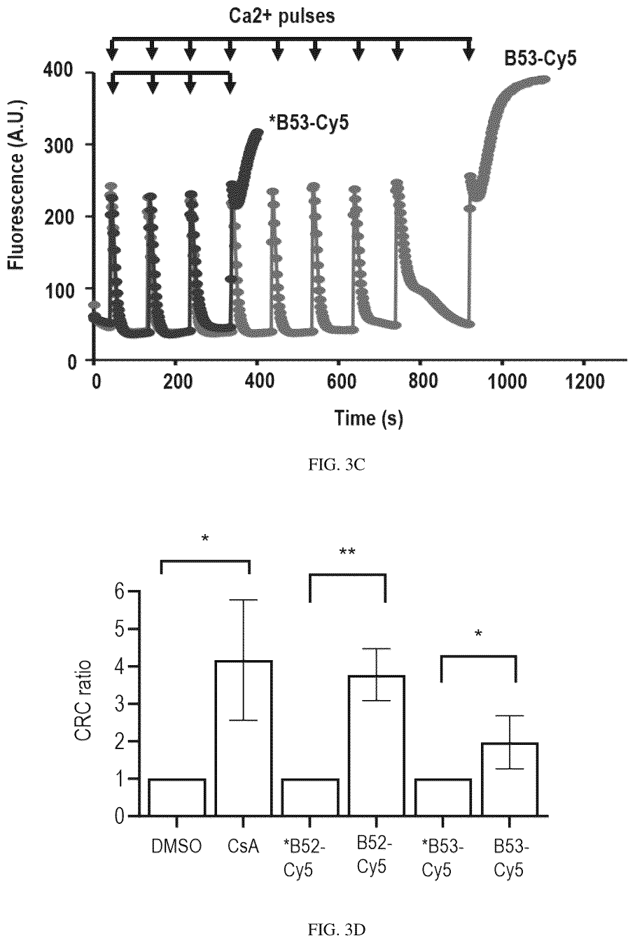

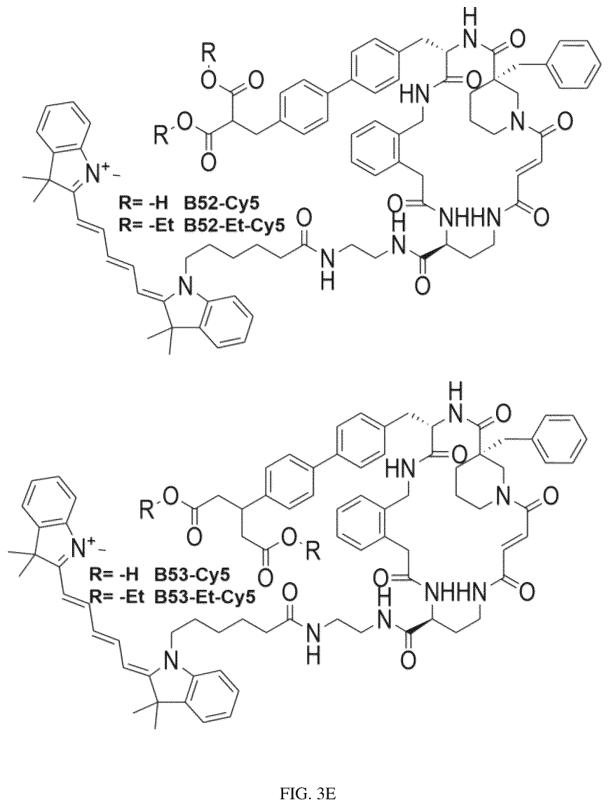

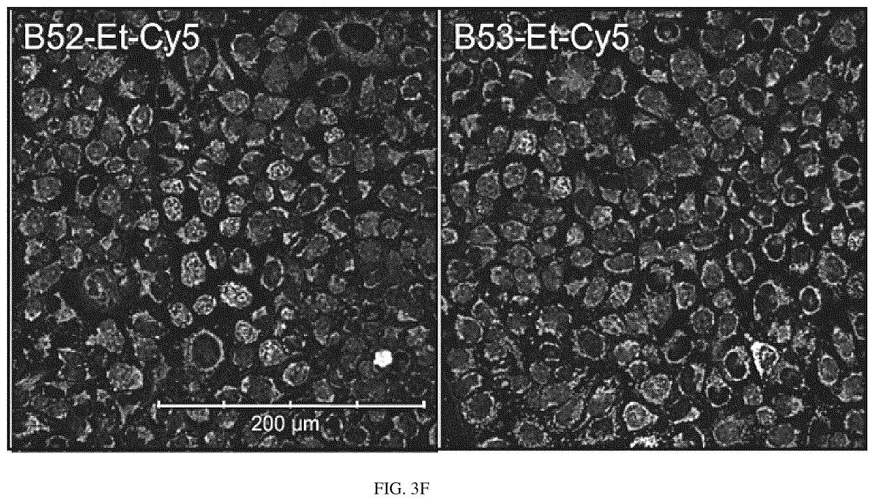

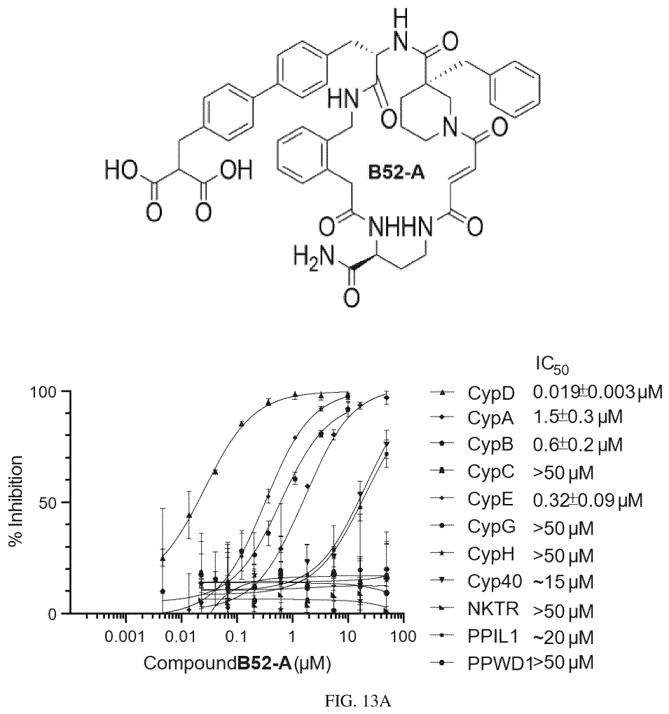

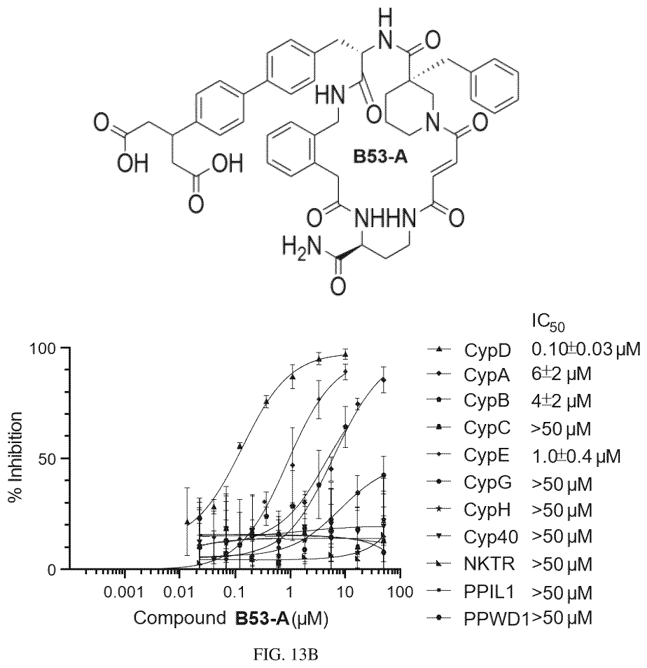

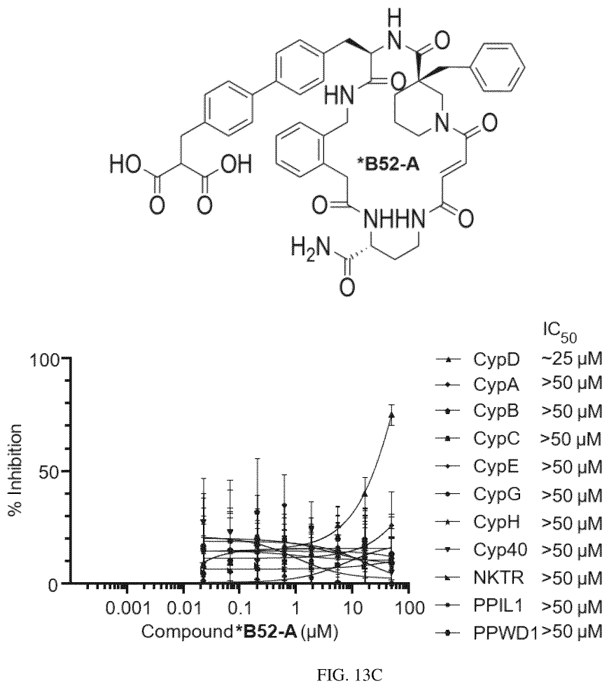

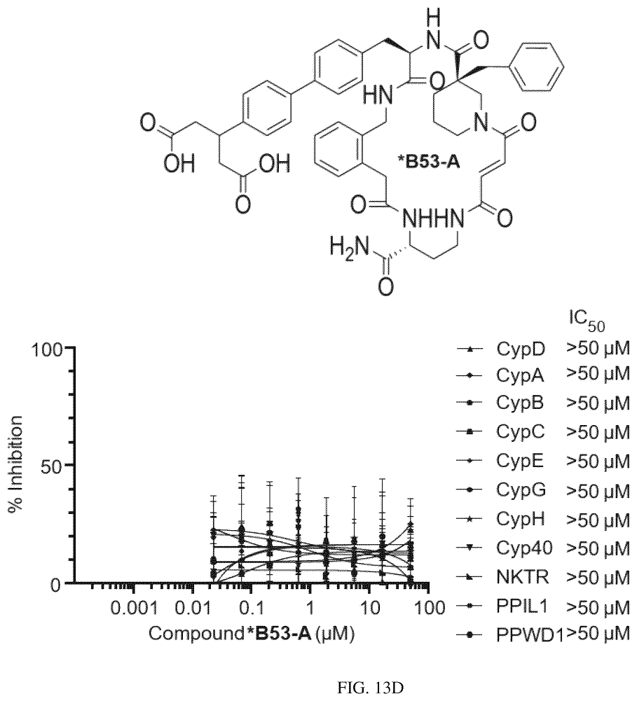

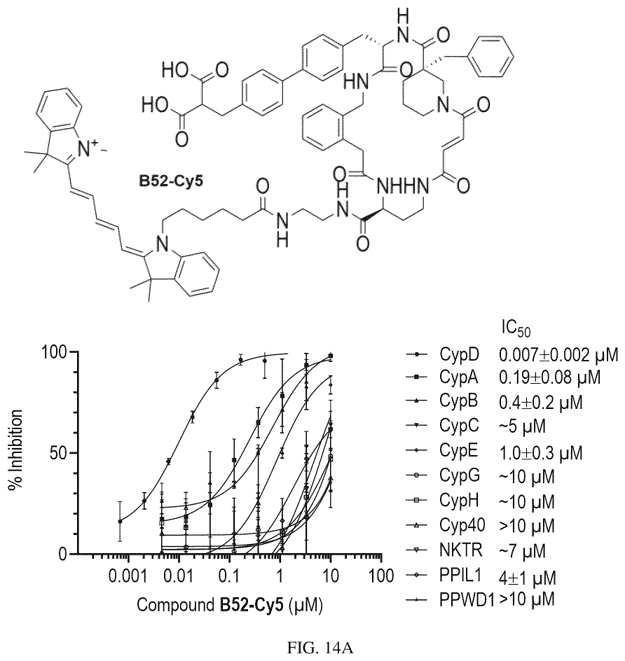

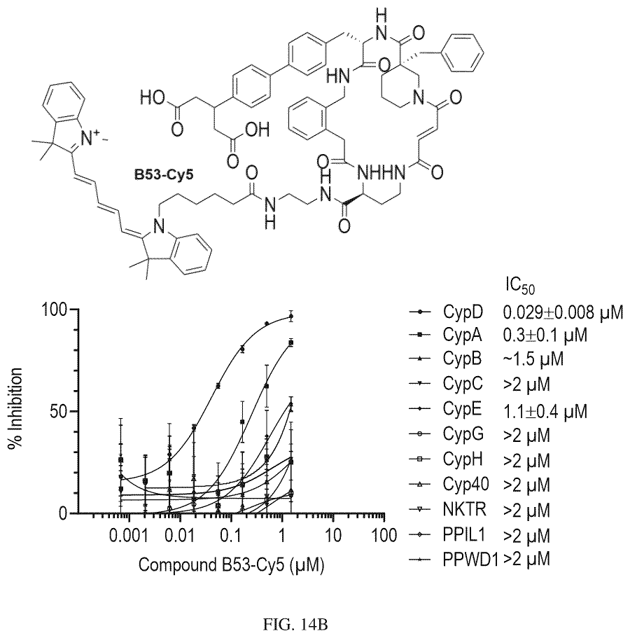

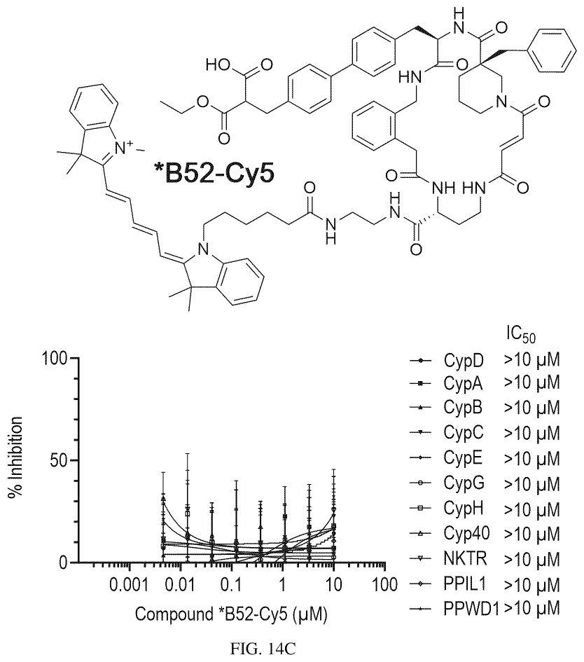

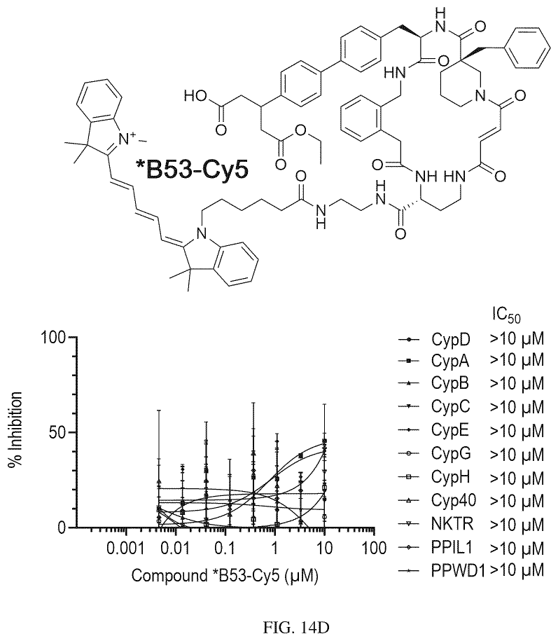

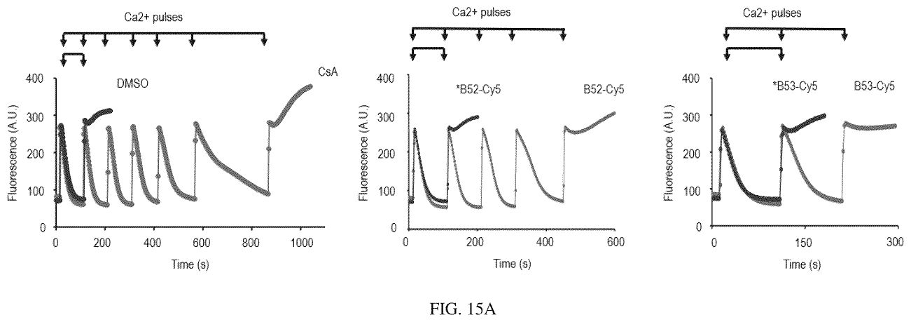

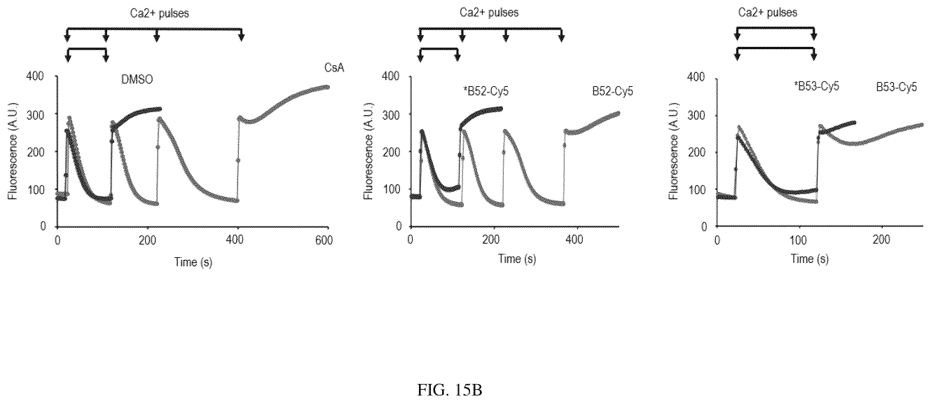

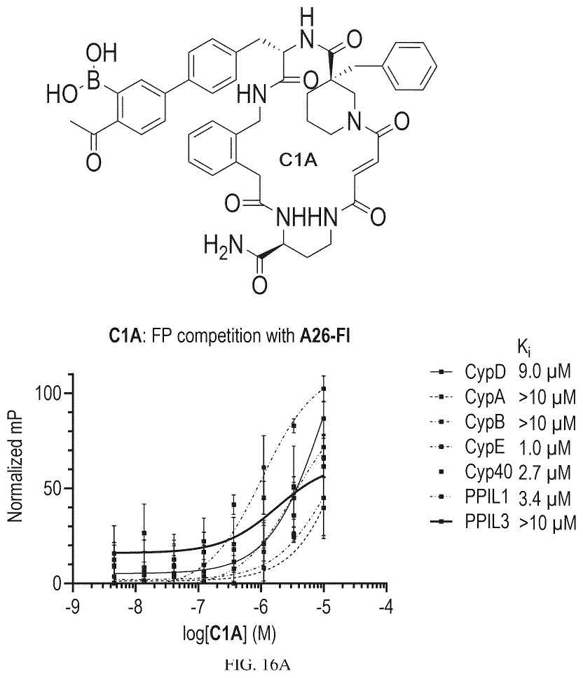

The ability of the CypD-selective macrocycles to inhibit CypD in active mitochondria isolated from mouse liver was tested. First, it was verified that varying groups on the ‘tail’ position of B52 and B53 (for example, B52-A, B53-A) did not affect potency or specificity, and that their enantiomers did not inhibit any cyclophilin (*B52-A, *B53-A) (FIGS. 13A-13D). Since CypD is exclusively found in mitochondria, Cy5, an established mitochondrial localization group⁴⁸, was appended to all four macrocycles at the ‘tail’ position (B52-Cy5, B53-Cy5, *B52-Cy5, *B53-Cy5). Minimal change in potency or selectivity for CypD in vitro upon addition of the Cy5 group (FIGS. 14A-14D) was observed. Compounds B52-Cy5 and B53-Cy5 were tested in isolated mouse liver mitochondria, measuring their calcium retention before a mPTP opening event. Compared to DMSO control, increased calcium retention capacity was observed prior to mPTP opening when pretreated with CsA, B52-Cy5, or B53-Cy5(FIGS. 3A-3D and 15A-15B). *B52-Cy5 and *B53-Cy5, the enantiomers of B52-Cy5 and B53-Cy5, did not inhibit CypD in vitro (FIGS. 3A-3D and 15A-15B) and did not influence calcium retention capacity prior to mPTP opening, strongly supporting CypD-dependent inhibition of mPTP opening by B52-Cy5 and B53-Cy5.

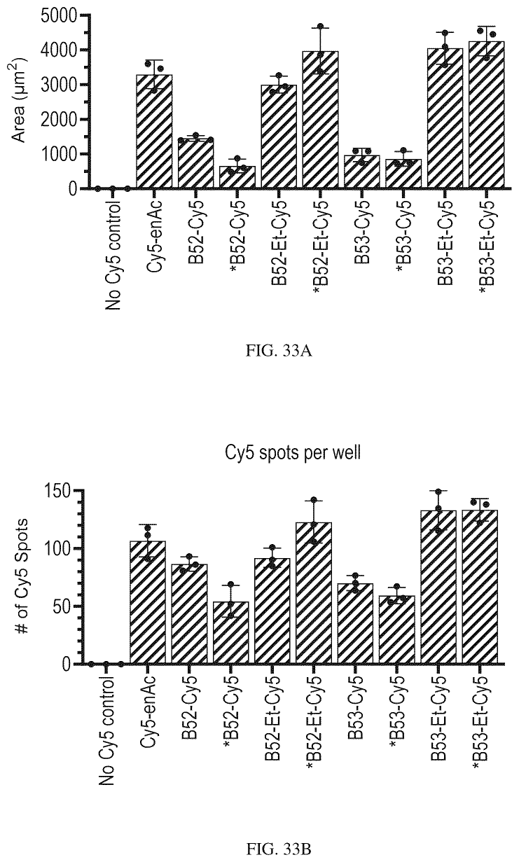

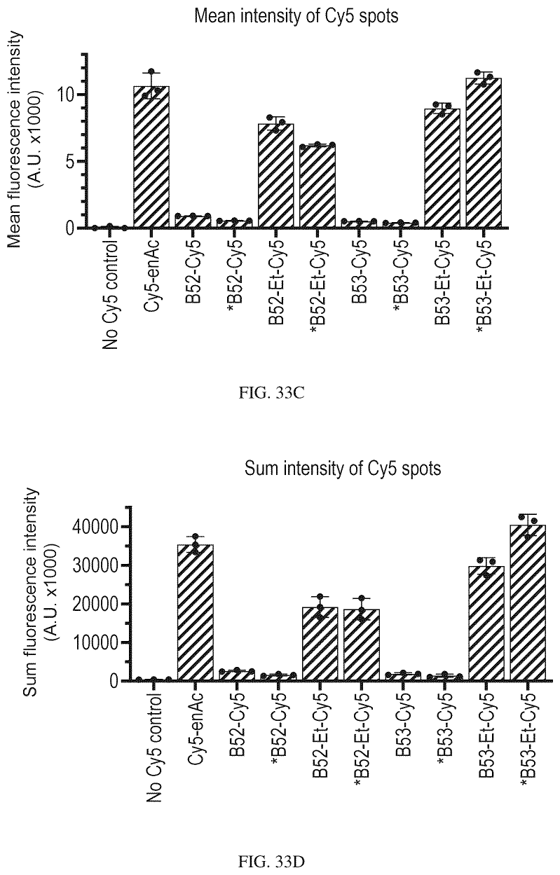

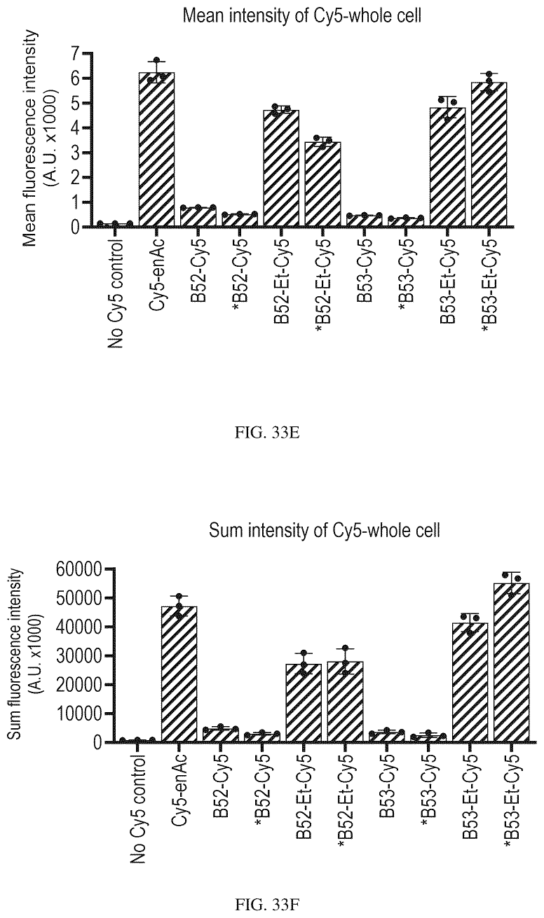

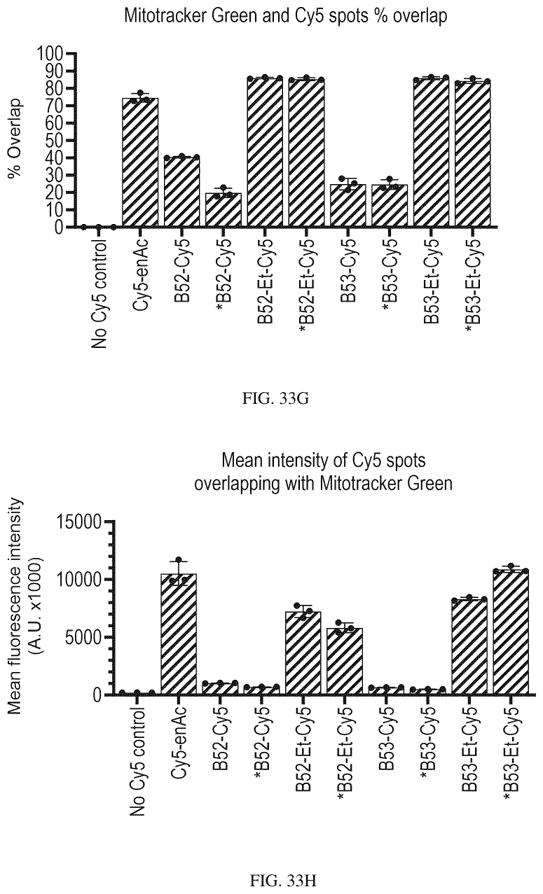

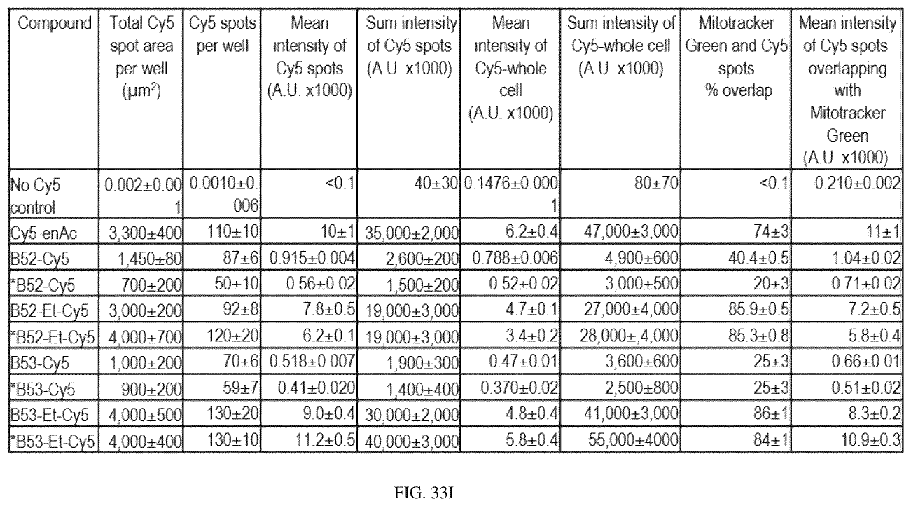

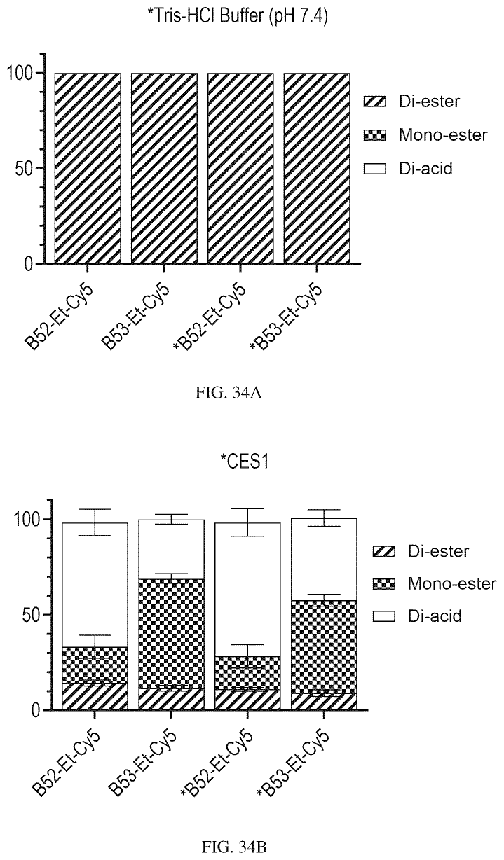

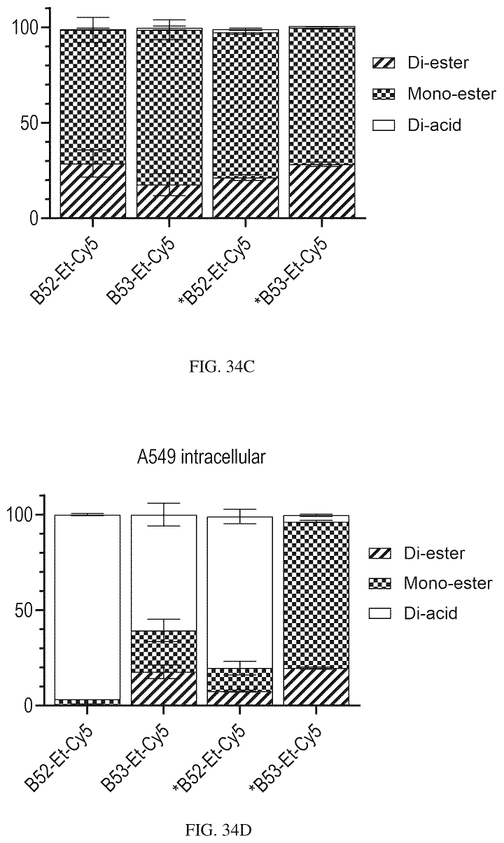

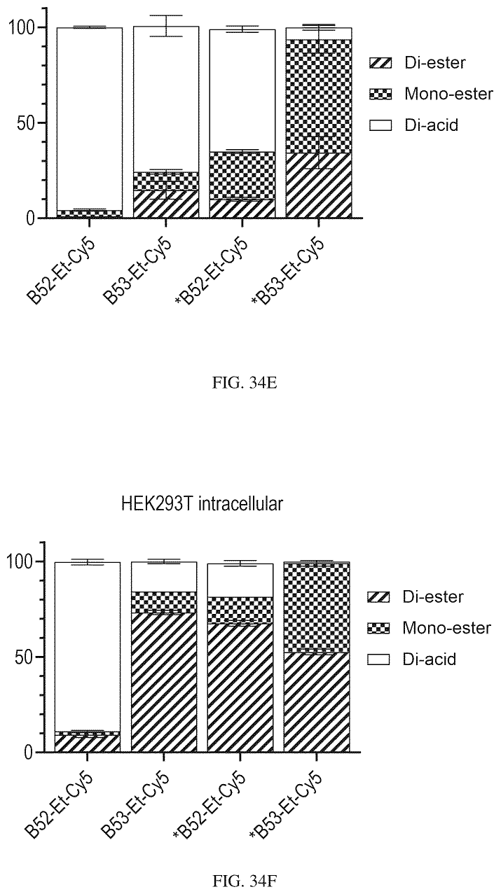

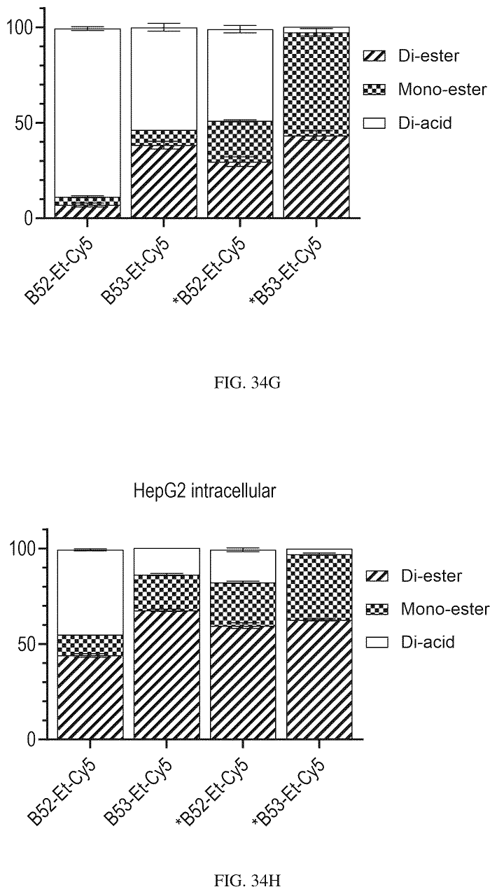

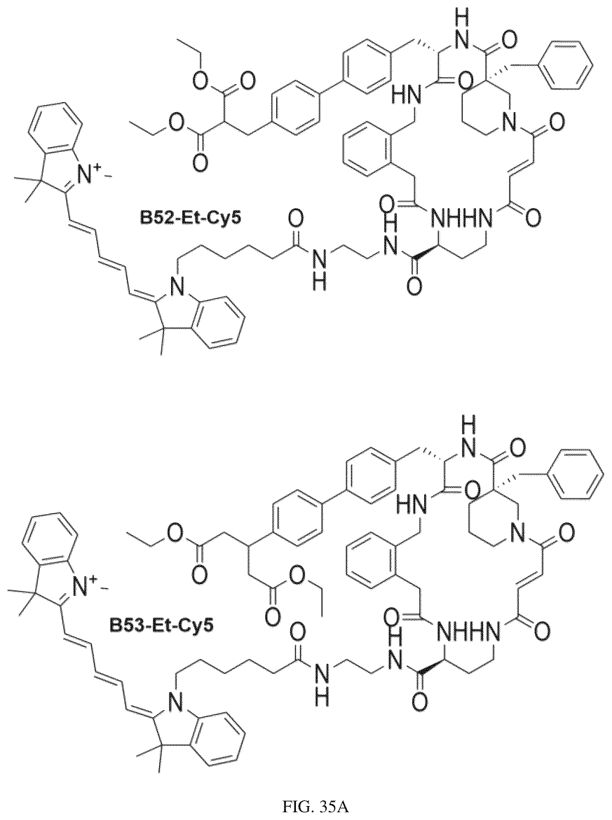

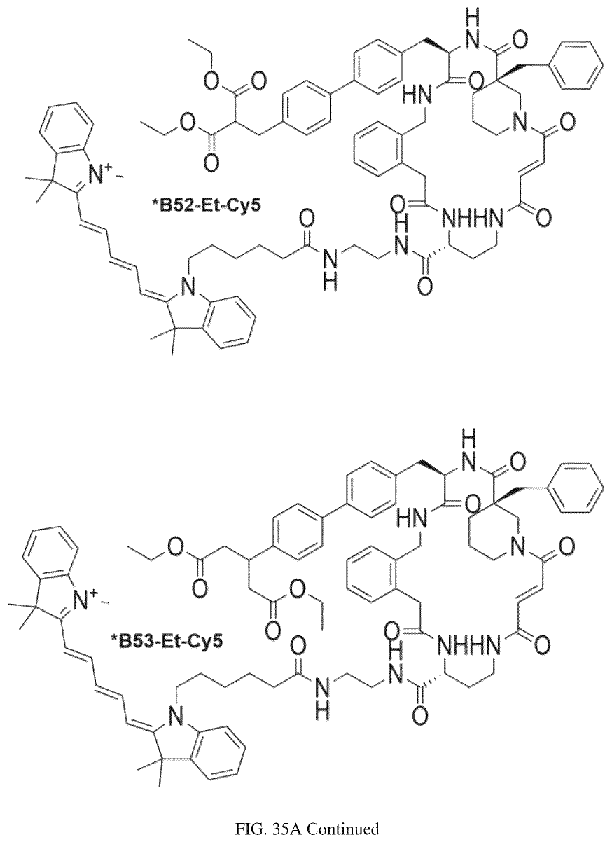

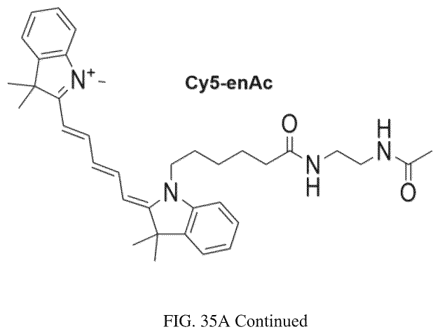

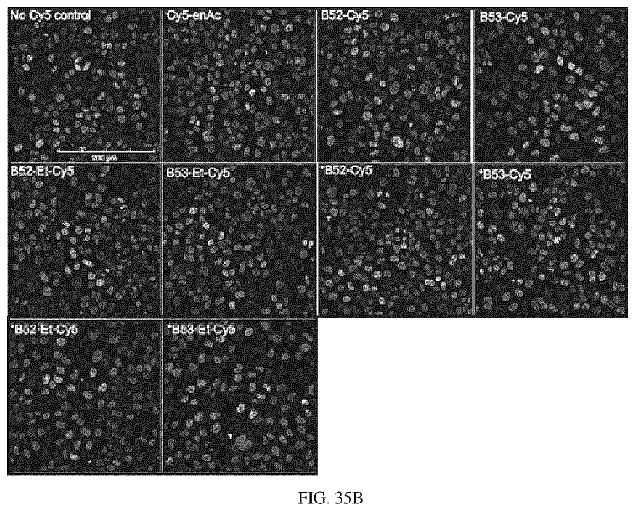

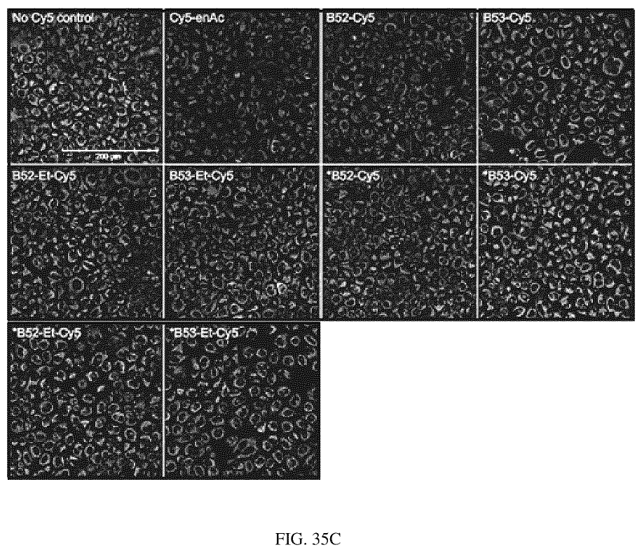

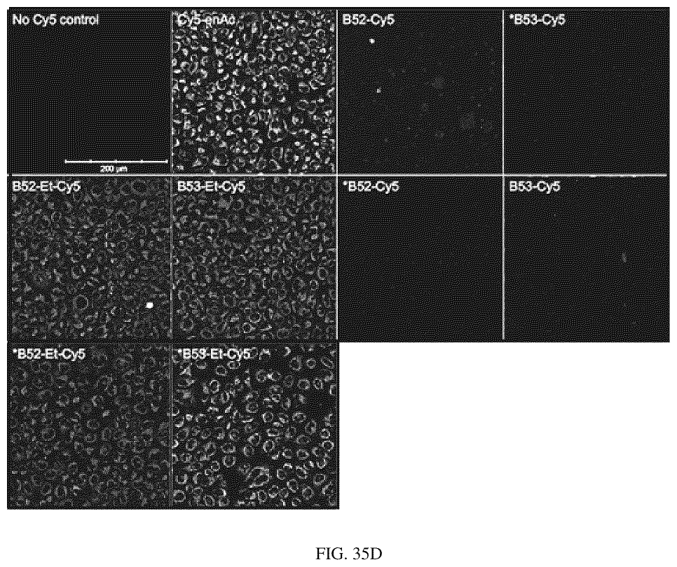

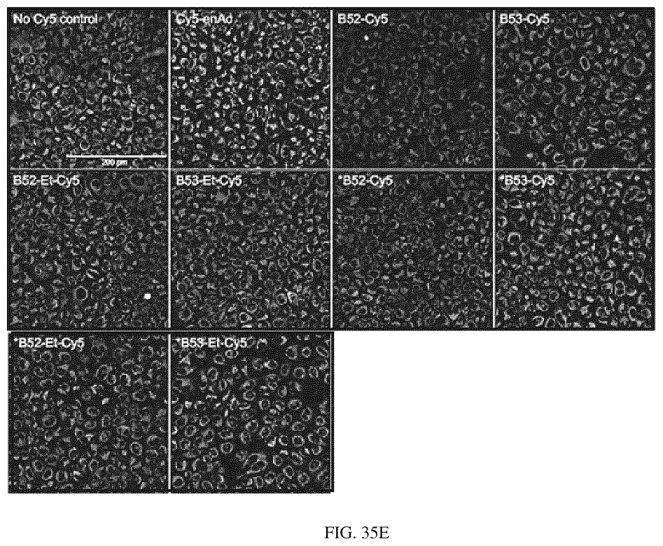

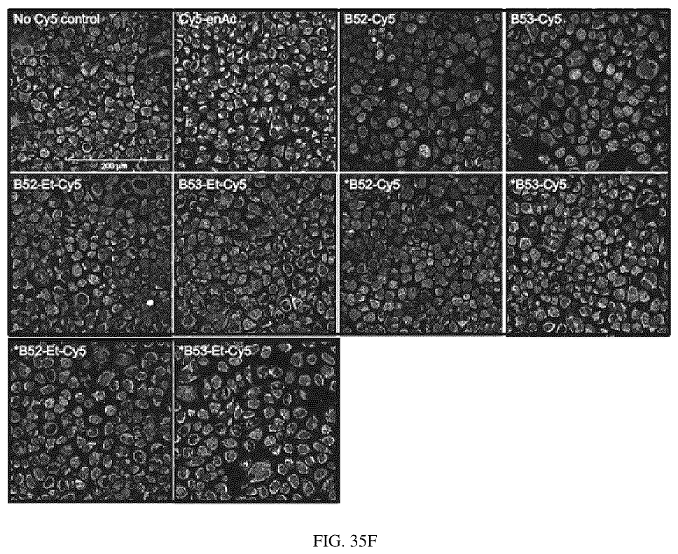

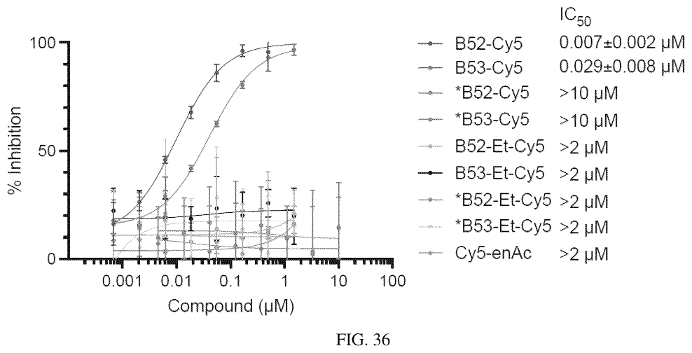

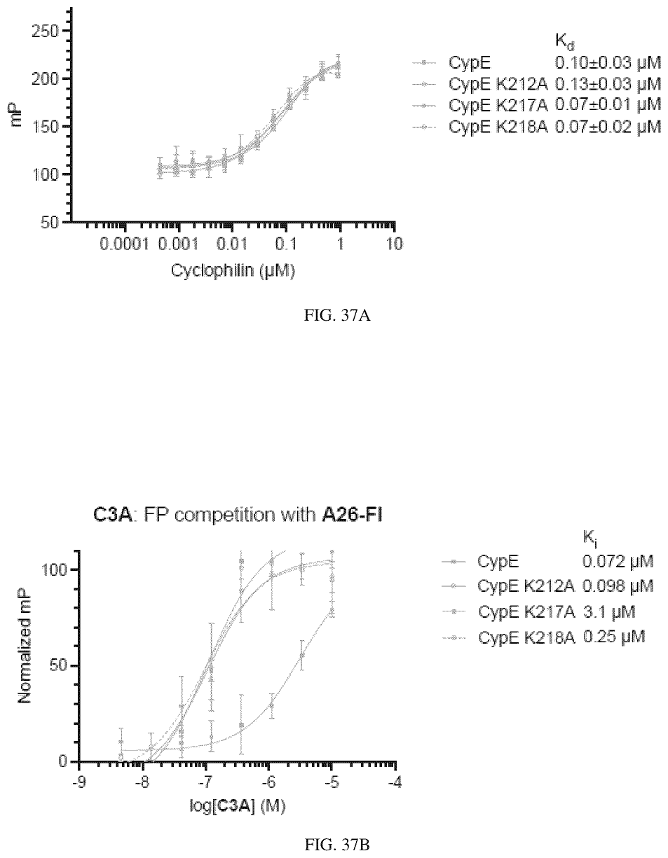

While B52-Cy5 and B53-Cy5 engage CypD in isolated mitochondria, efficient plasma membrane permeability of B52-Cy5, B53-Cy5, or their inactive enantiomers was not observed by fluorescence microscopy (FIGS. 33A-34I, FIGS. 35B-36F), presumably due to the presence of their dicarboxylate groups. To improve cell permeability, a prodrug strategy was used to and prepare both sets of active and inactive enantiomers as ethyl esters (B52-Et-Cy5 (117), B53-Et-Cy5(118), *B52-Et-Cy5(119), *B53-Et-Cy5(120)) (FIG. 3E, FIG. 35A). Strong mammalian cell permeability and mitochondrial localization were observed for all four ester containing compounds (FIG. 3F, FIGS. 33A-34I, FIGS. 35B-36F). These ester derivatives did not potently inhibit CypD's prolyl-isomerase activity, consistent with the importance of the dicarboxylate groups (FIG. 36). All four prodrug compounds were hydrolyzed to their active dicarboxylate forms in vitro with human carboxylesterase (CES1), and B52-Et-Cy5, *B52-Et-Cy5, and B53-Et-Cy5 were also readily converted to the corresponding di-acid intracellularly by endogenous esterases in a variety of human and mouse cell lines (A549, HeLa, HEK293T, HepG2, and MEFs) (FIGS. 34A-35H).

These findings, coupled with target engagement and robust CypD-dependent inhibition of mPTP opening in isolated mitochondria, together suggest that prodrug versions of Cy5-conjugated B52 and B53 can access mitochondria and release active CypD-selective inhibitors in mammalian cells. These observations make esterified B52 and B53 attractive candidates for potent and selective probes of CypD activity in biological systems. These compounds therefore may serve as tools to explore the mechanism of action of the mPTP or as probes of the role of CypD in disease models with mPTP-oxidative stress as a phenotype, such as neurodegenerative disorders, IRI, liver diseases, and mitochondrial disorders.

### Example 3. Development of CypE-Selective Inhibitors

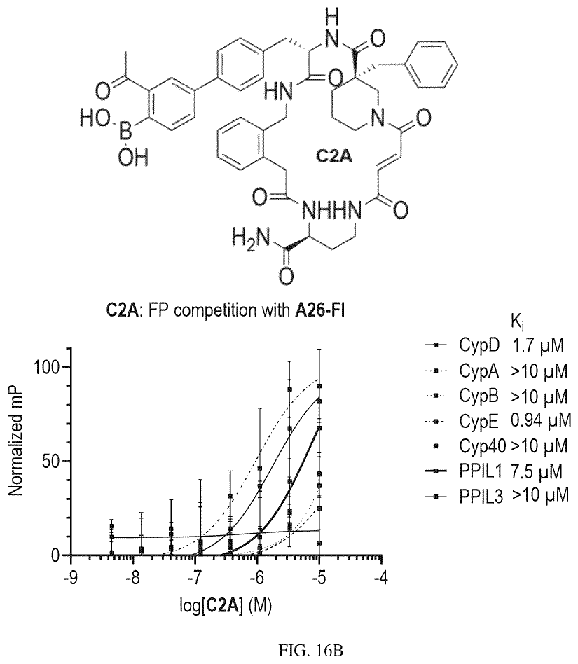

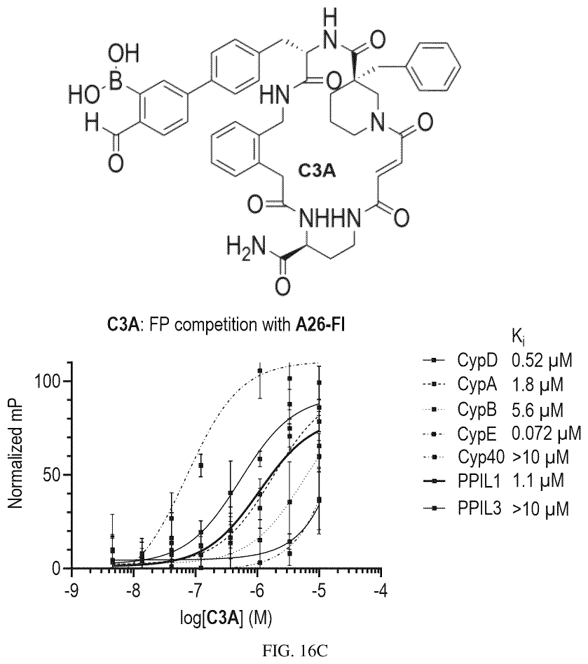

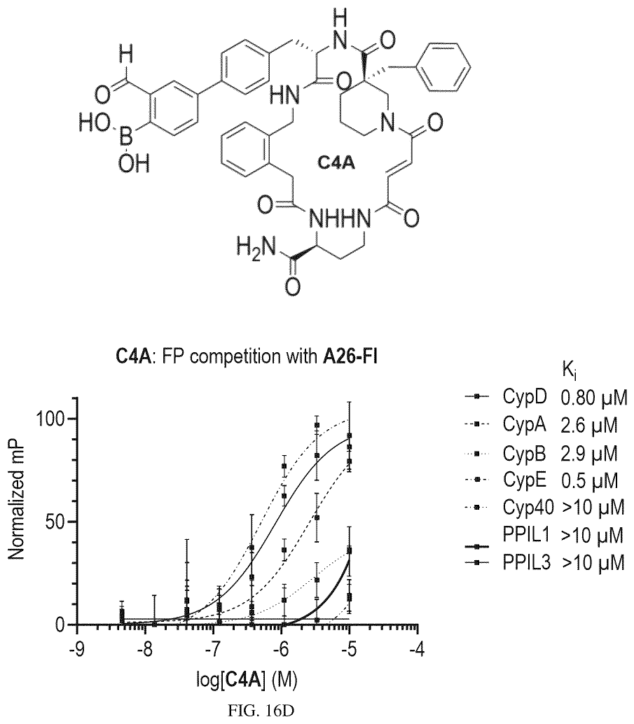

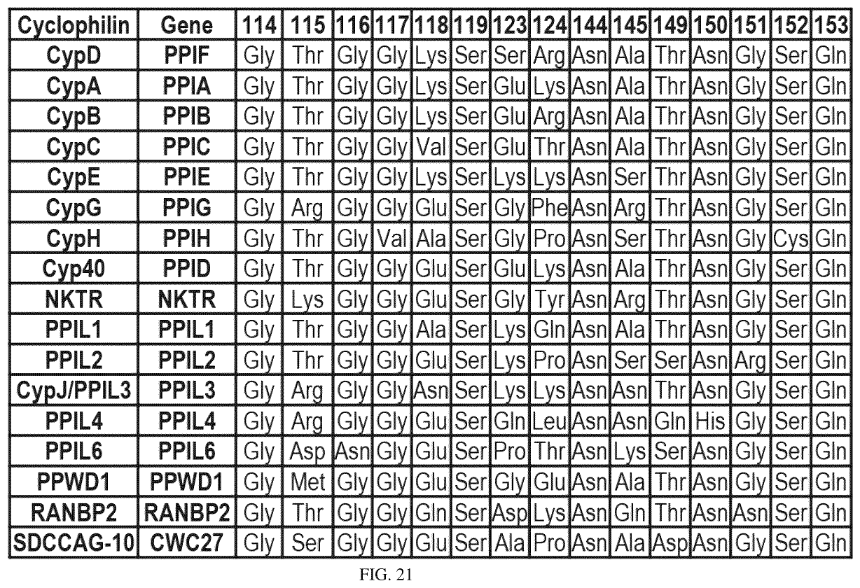

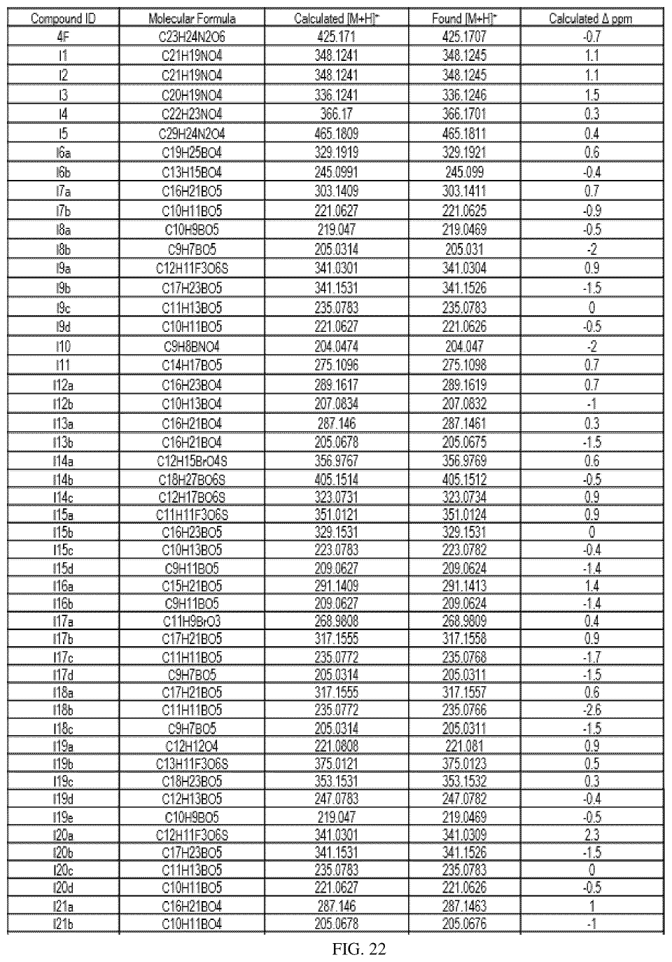

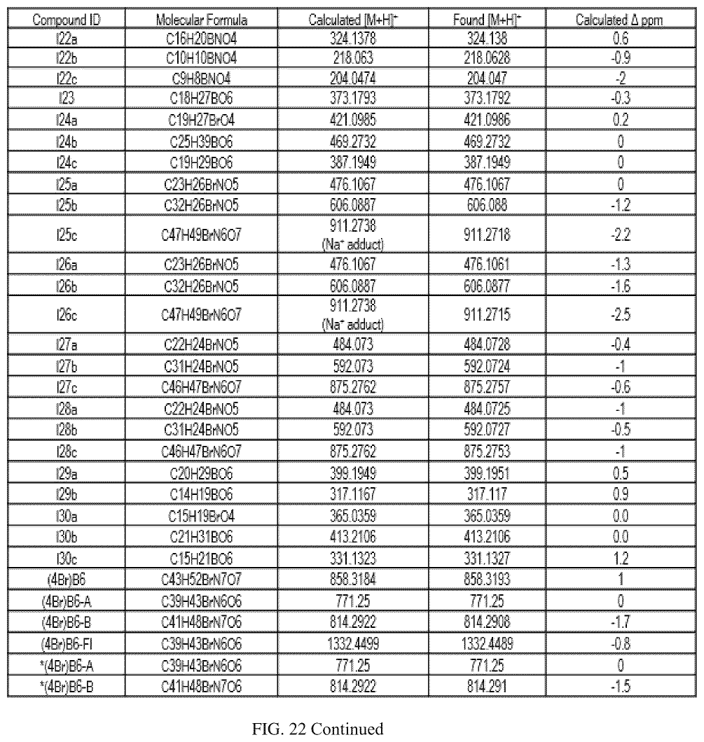

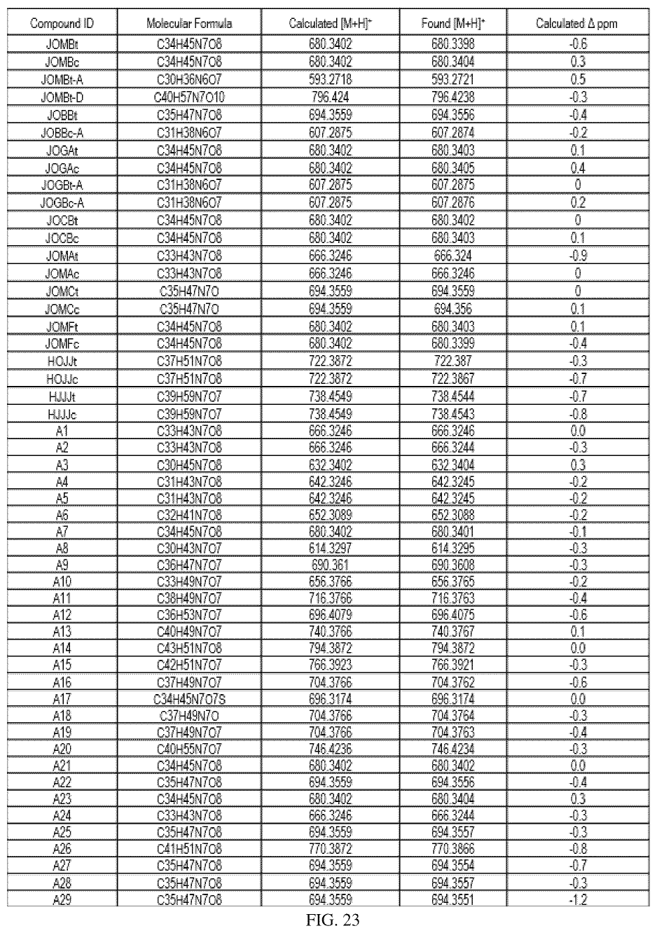

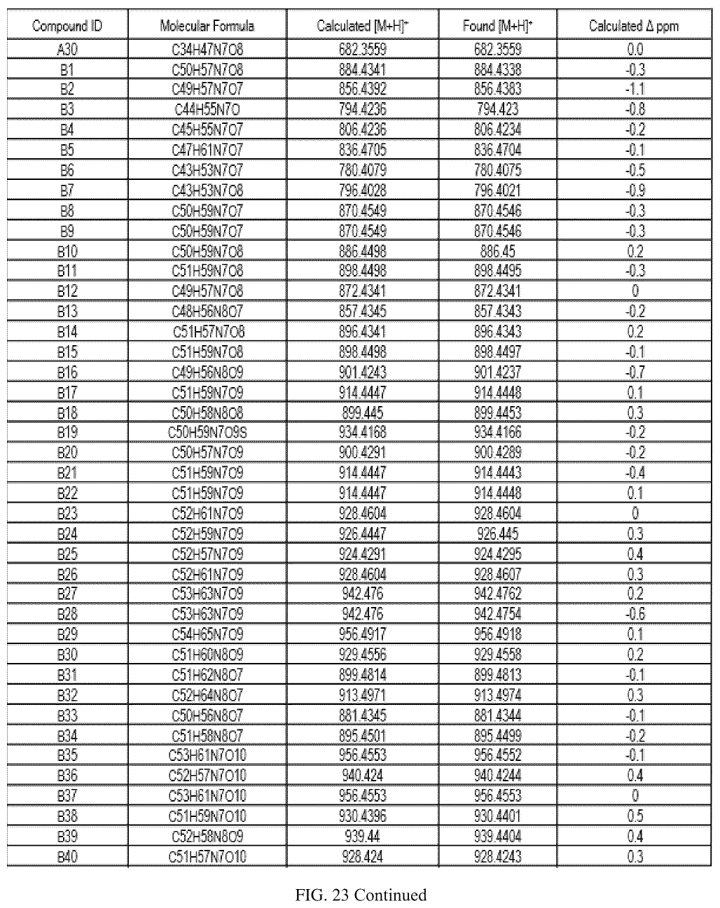

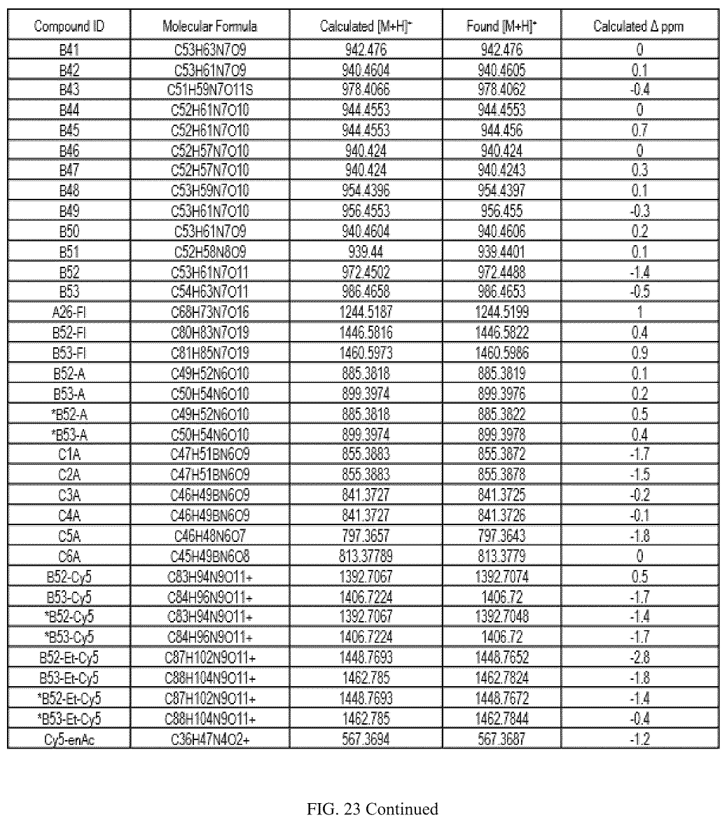

Since CypD selective inhibitors B52 and B53 were designed from a slightly promiscuous inhibitor B2, the possibility of accessing other inhibitors selective for an endogenous cyclophilin was determined. To access a novel chemical space outside of the established inhibitor series, the current repertoire of reported covalent binding moieties was used. While most covalent inhibitors to date target catalytic residues, the non-conserved residues in cyclophilin S2 pockets are both solvent-exposed and non-catalytic. Although non-catalytic lysines are typically protonated at physiological pH and are non-nucleophilic, aryl boronic acid carbonyls can modify lysine and N-terminal groups covalently and reversibly through the formation of iminoboronates. Previous studies demonstrated that incorporating aryl boronic acid carbonyl warhead on inhibitors can allow them to covalently modify non-catalytic lysines^{49,50}. Notably, many cyclophilins contain non-catalytic lysines in their S2 pocket, including CypA, CypB, Cyp40, CypE, PPIL1, and PPIL3 (FIG. 21). Therefore, it is possible that installing this reactive group on the biphenyl of inhibitor B2 might allow a reversible covalent interaction with one of these cyclophilins, potentially providing a new subtype-selective inhibitor. Ketone boronic acids C1A, C2A and aldehyde boronic acids C3A, C4A that could place this warhead within these cyclophilin's S2 pockets close to their lysine residues were synthesized (FIGS. 16A-16D).

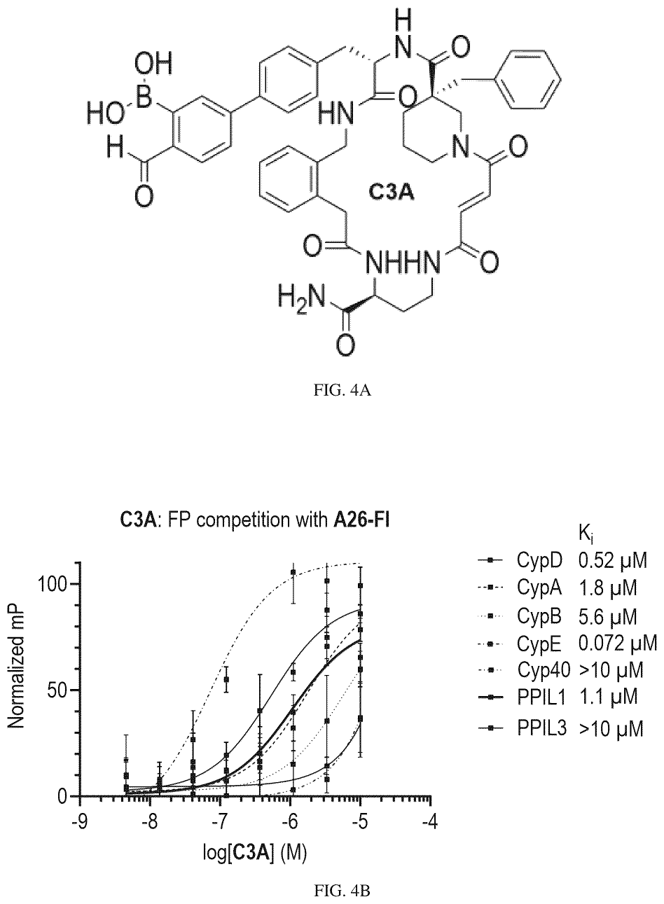

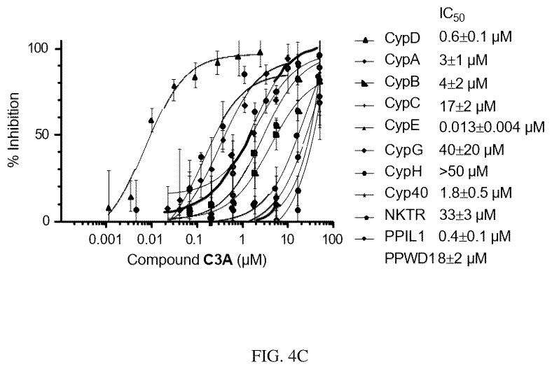

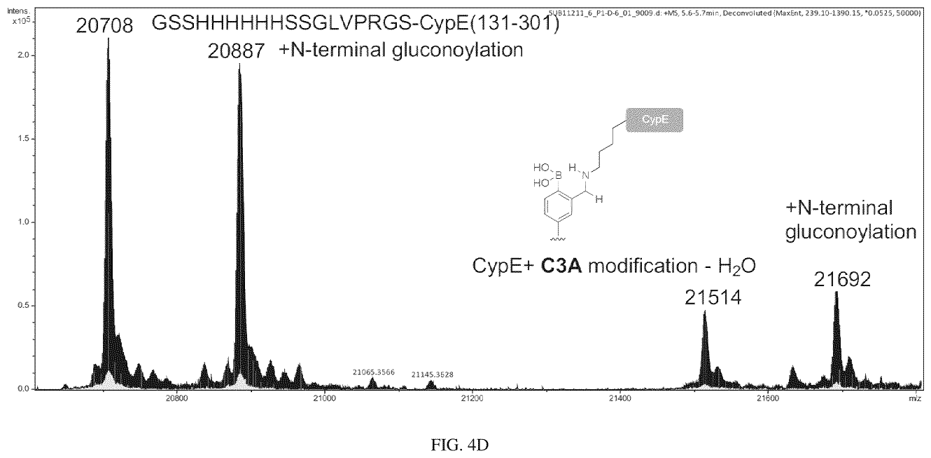

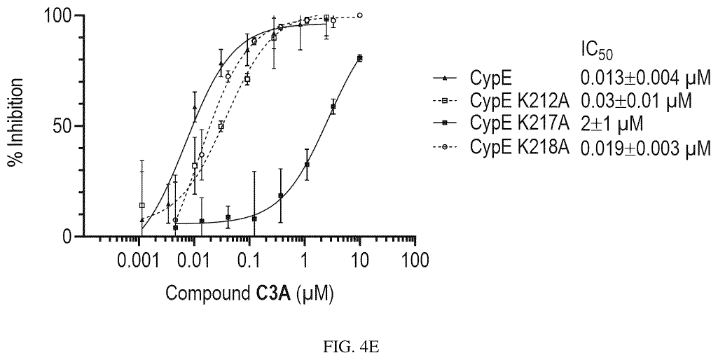

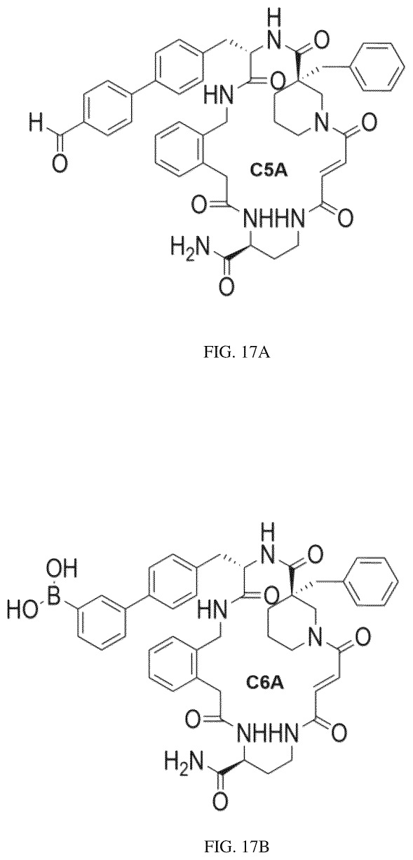

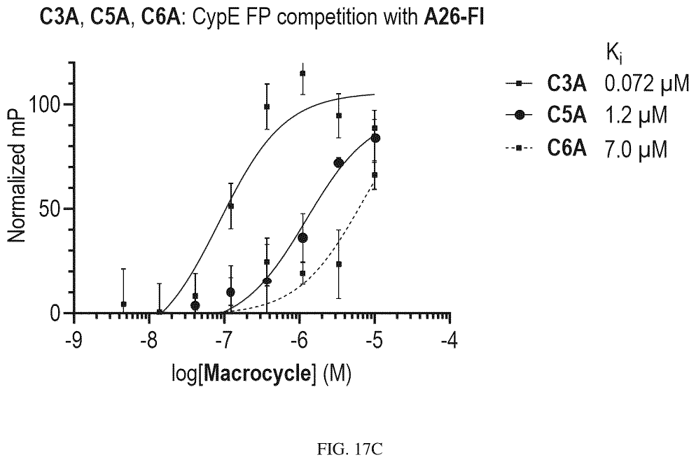

Compounds C1A, C2A, C3A, and C4A were secreened in a fluorescence polarization competition assay with A26-Fl against the above-mentioned lysine-containing cyclophilins (FIGS. 16A-16D). C3A was identified as a potent inhibitor of A26-Fl binding to CypE, with a Kᵢ of 0.072 μM (FIGS. 4A-4B and 16C). Additionally, C3A showed >10-fold more potency for CypE than for other tested cyclophilins. Shifting the aldehyde group to the meta position of the ring (C4A) attenuated CypE potency and selectivity, suggesting that the aryl boronic acid carbonyl of C4A was not properly placed to react with the lysines in CypE (FIG. 16D). Replacing the aldehyde with a ketone (C1A, C2A) also decreased potency for CypE compared to C3A (FIGS. 16A-16B). Compounds C5A and C6A containing either only the aldehyde or the boronic acid, respectively, were synthesized, and significant decreases in potency by 16-fold and 100-fold, respectively, relative to C3A was observed (FIGS. 17A-17C) Since C3A possesses both the boronic acid and the aldehyde, these results suggested C3A was acting covalently through iminoboronate formation with CypE, consistent with CypE's lysine-rich S2 pocket.

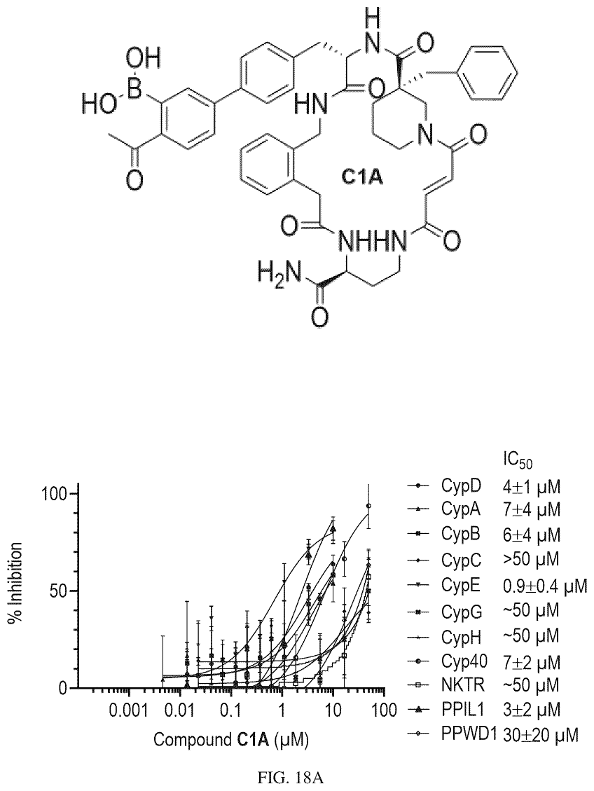

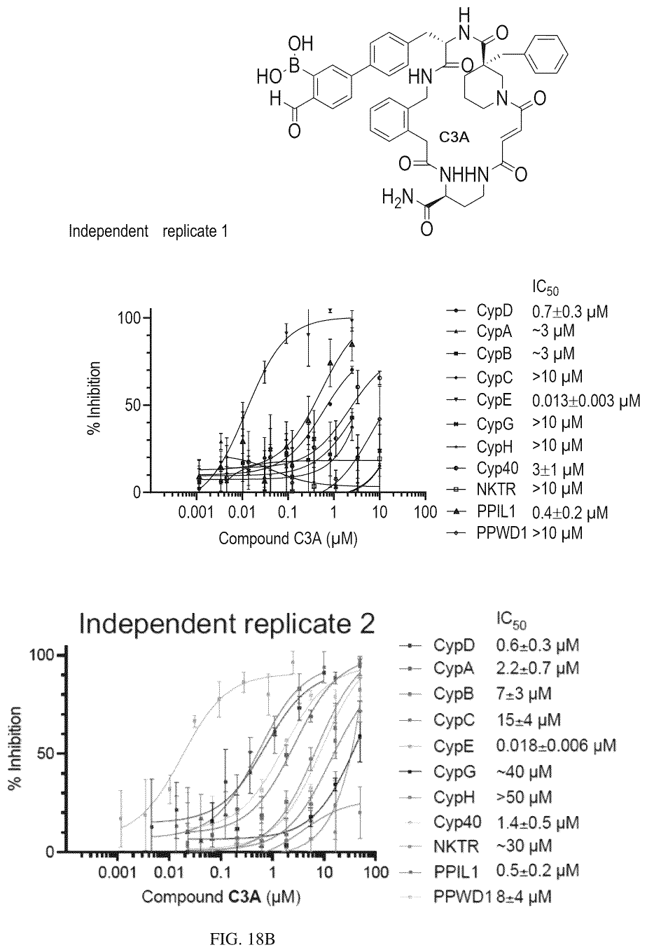

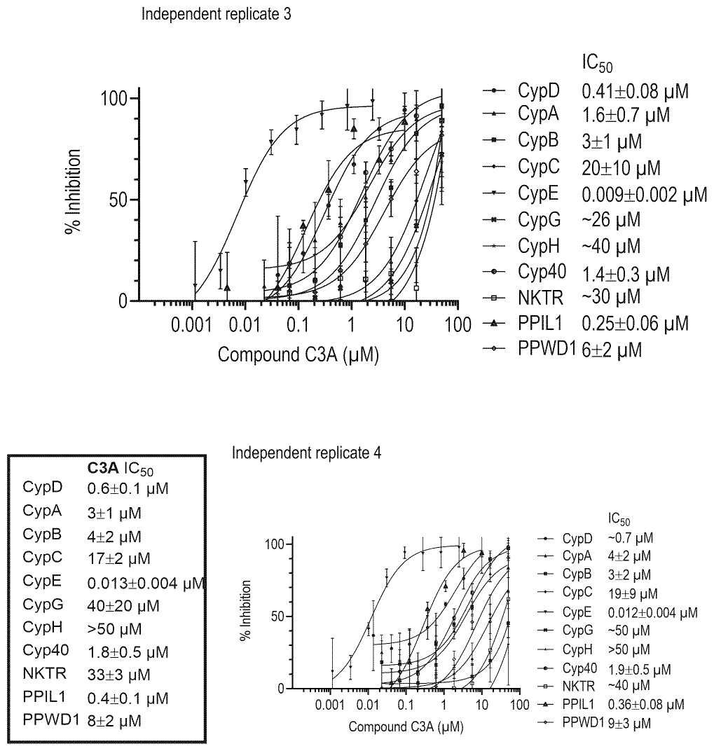

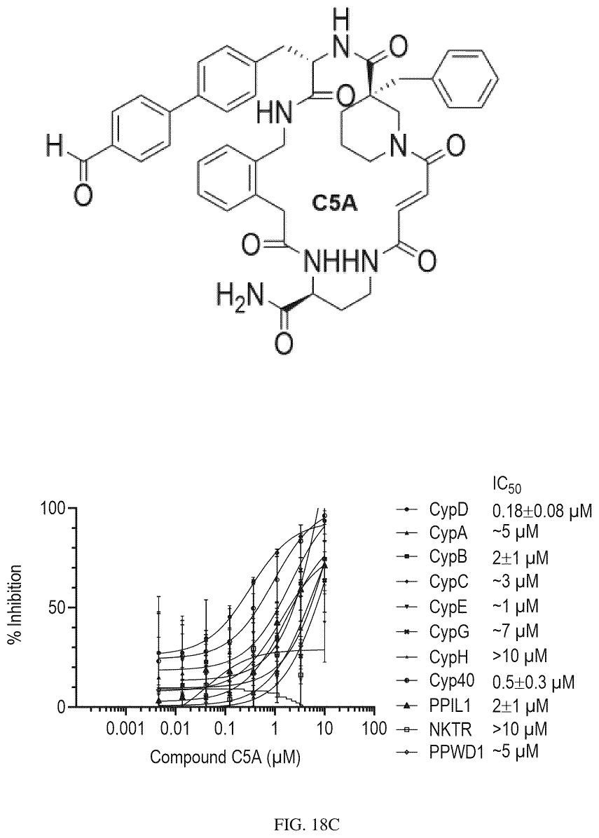

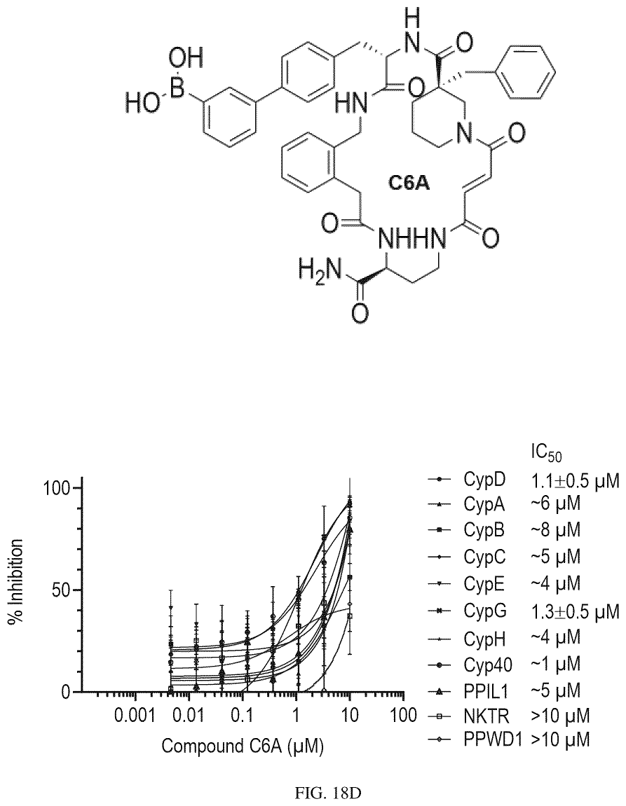

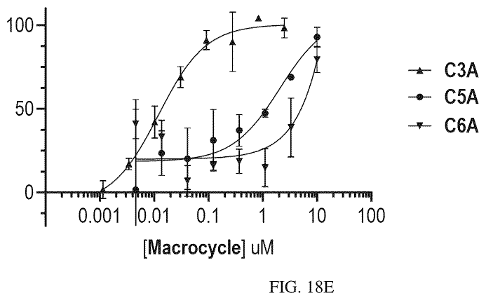

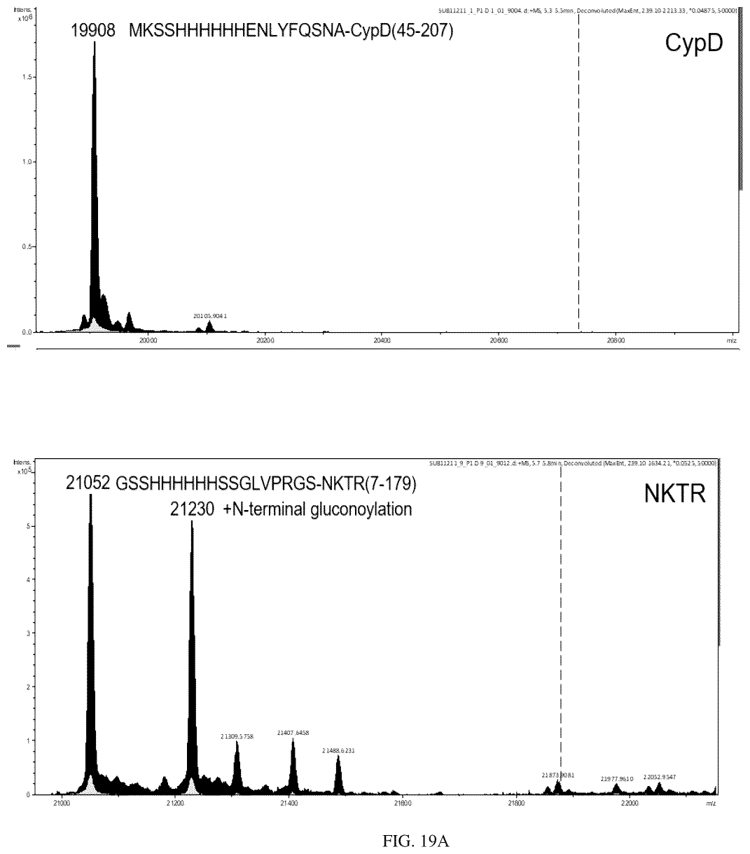

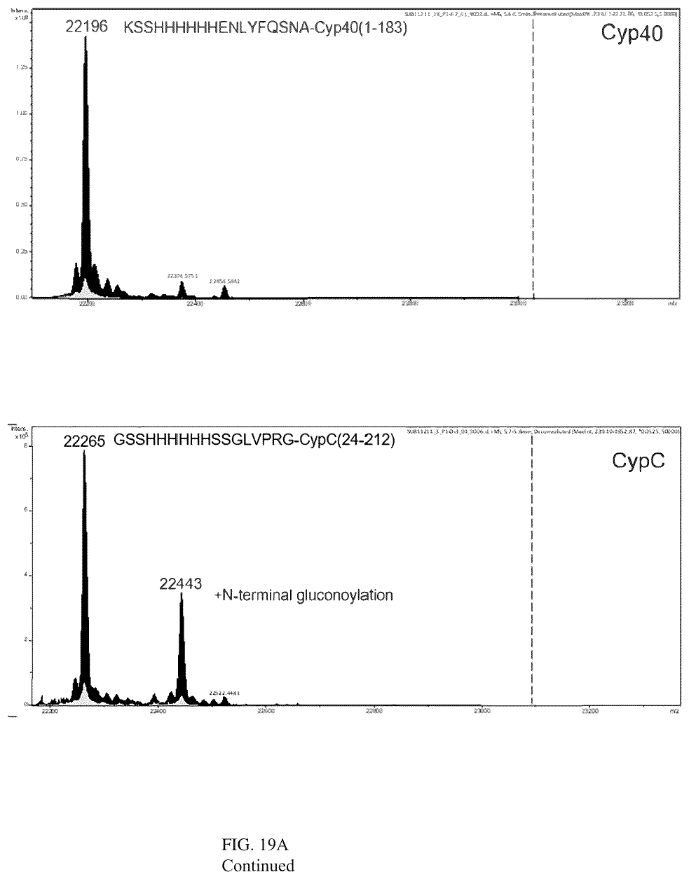

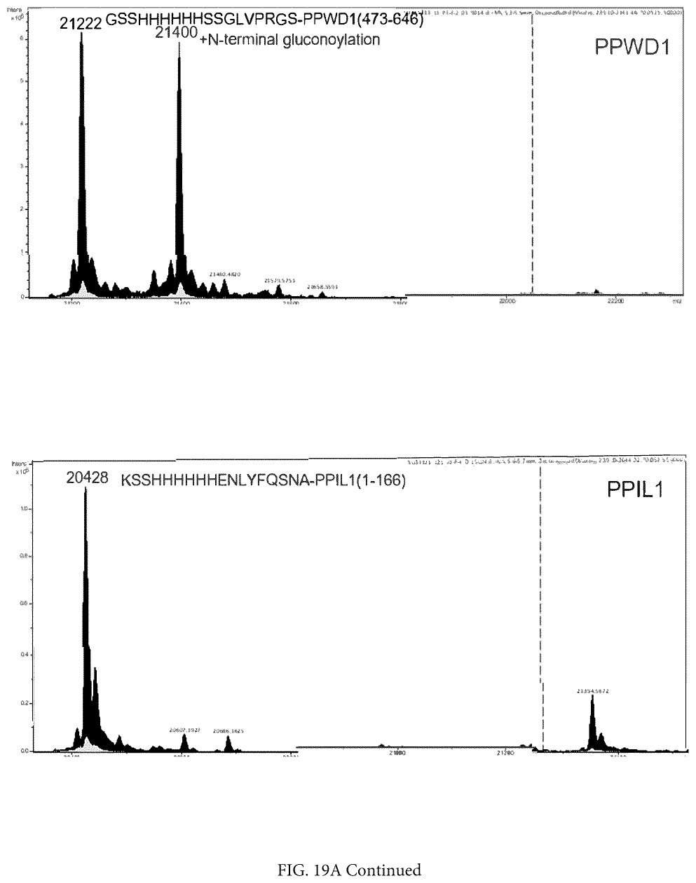

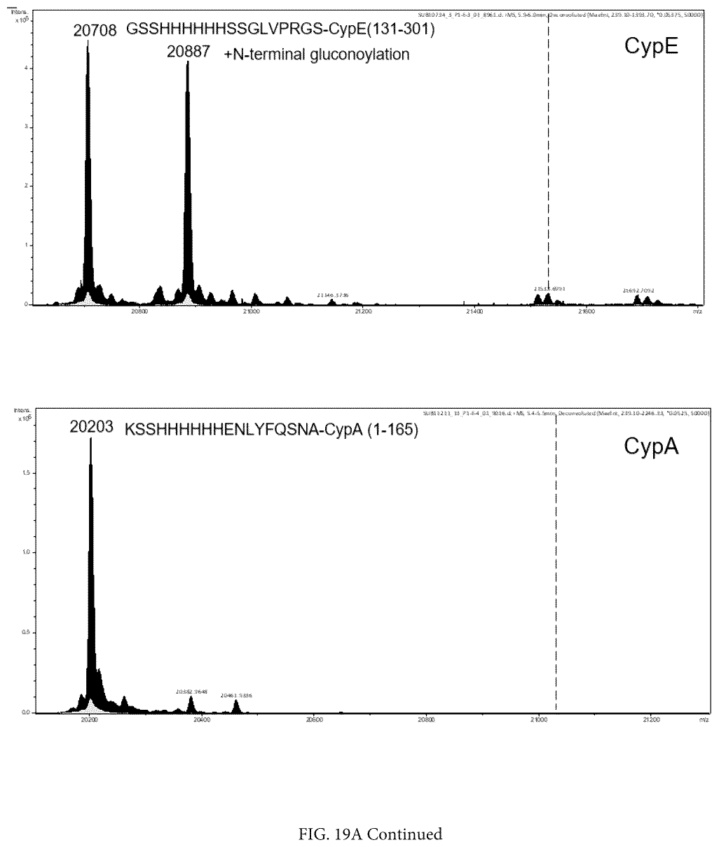

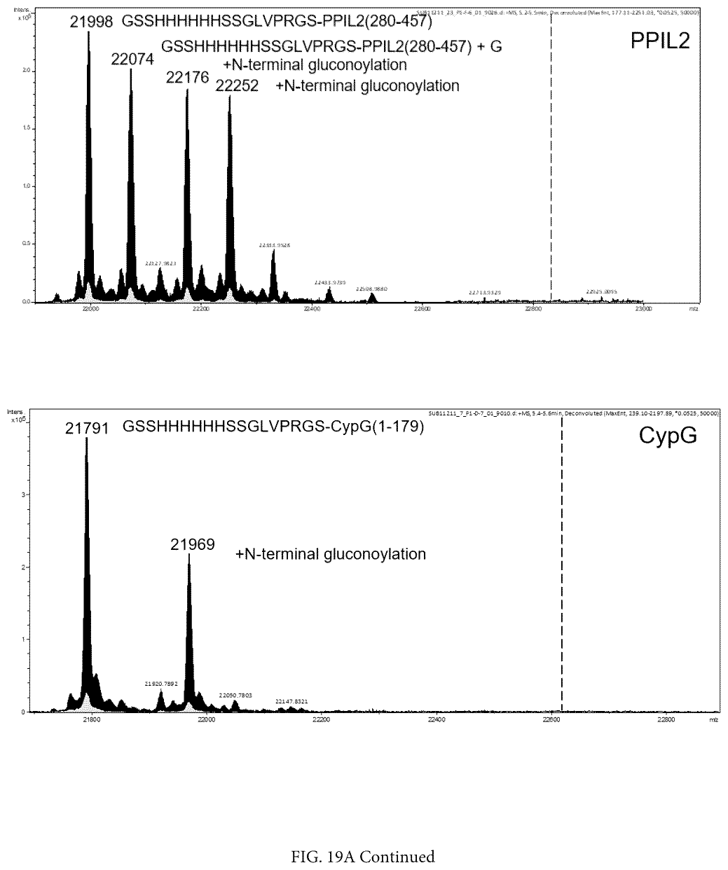

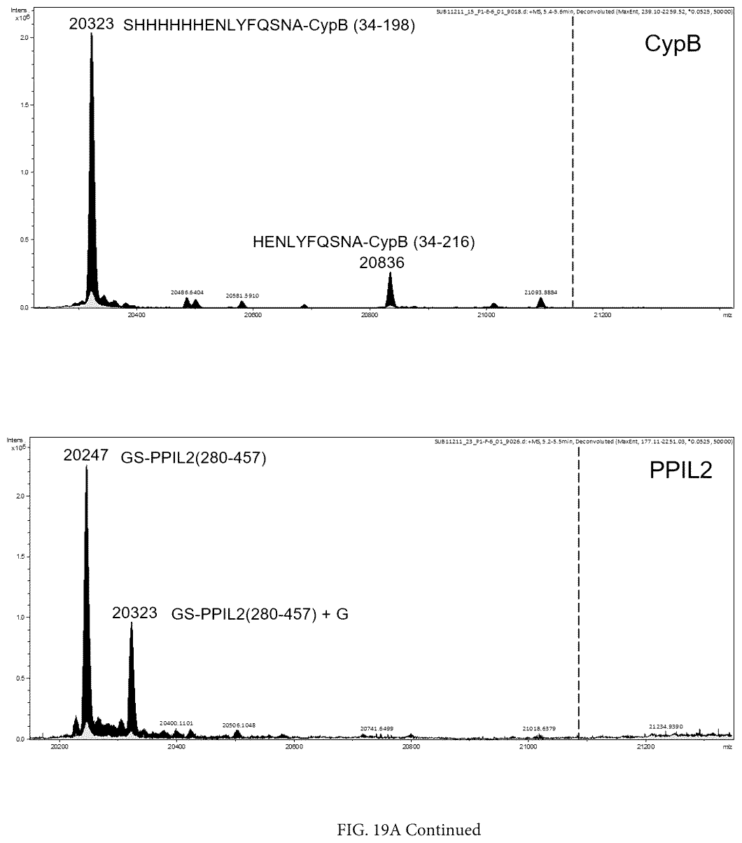

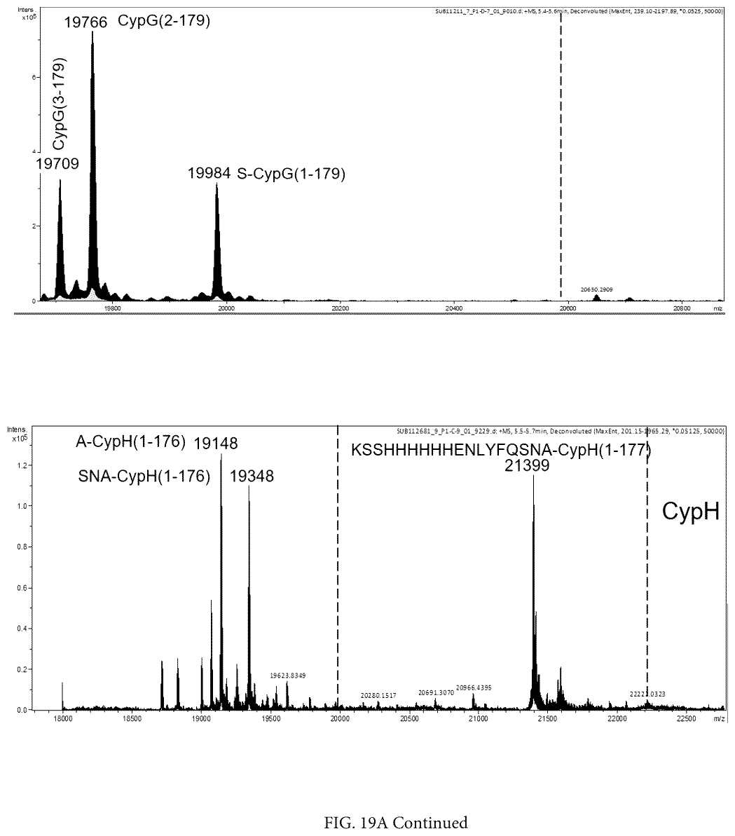

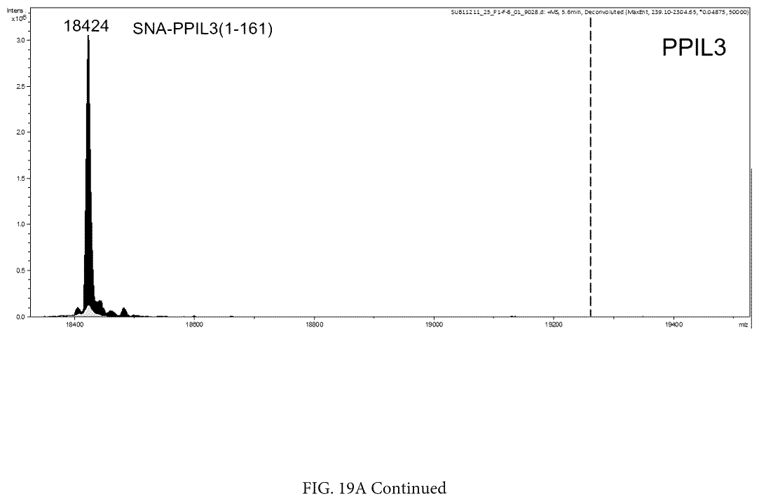

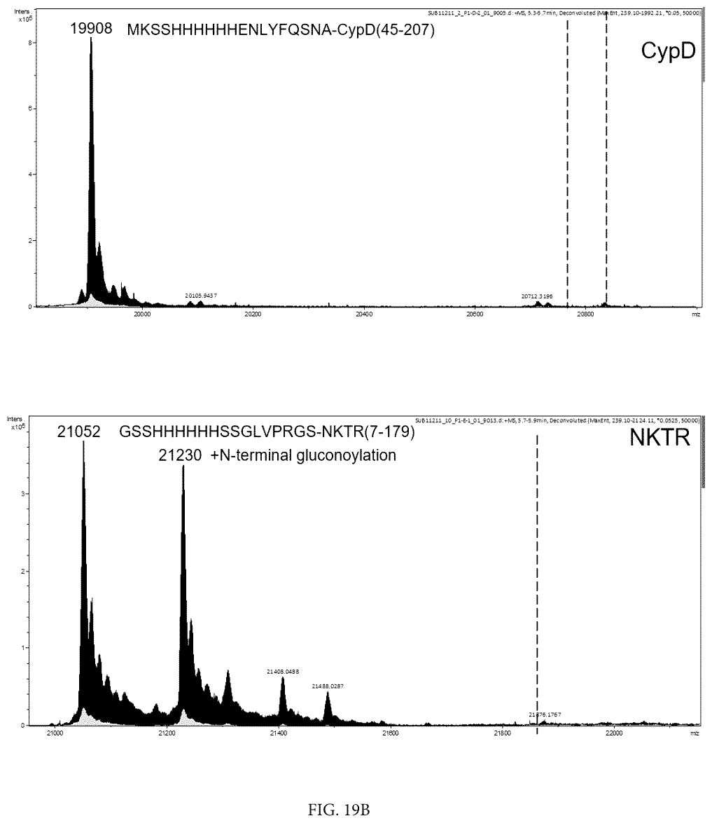

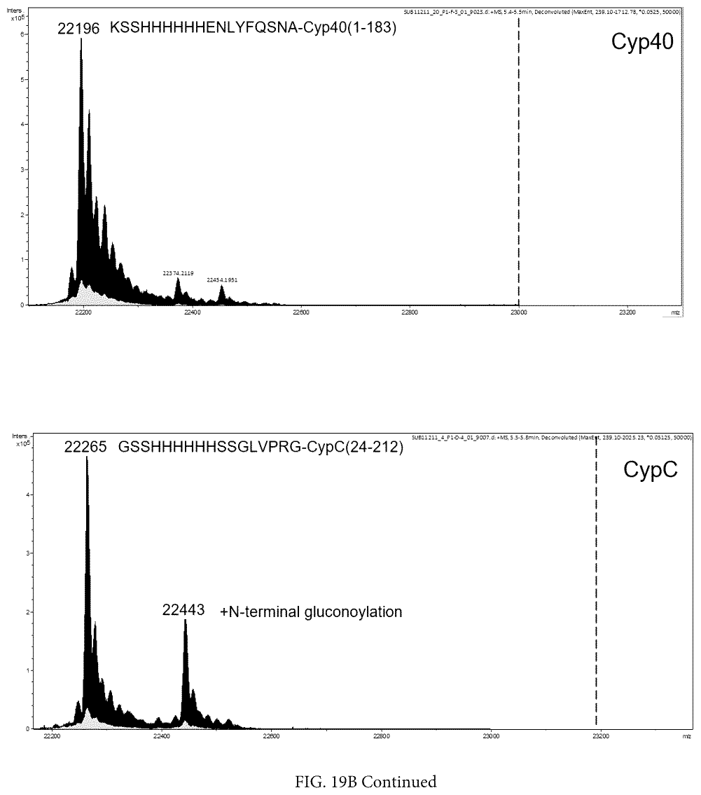

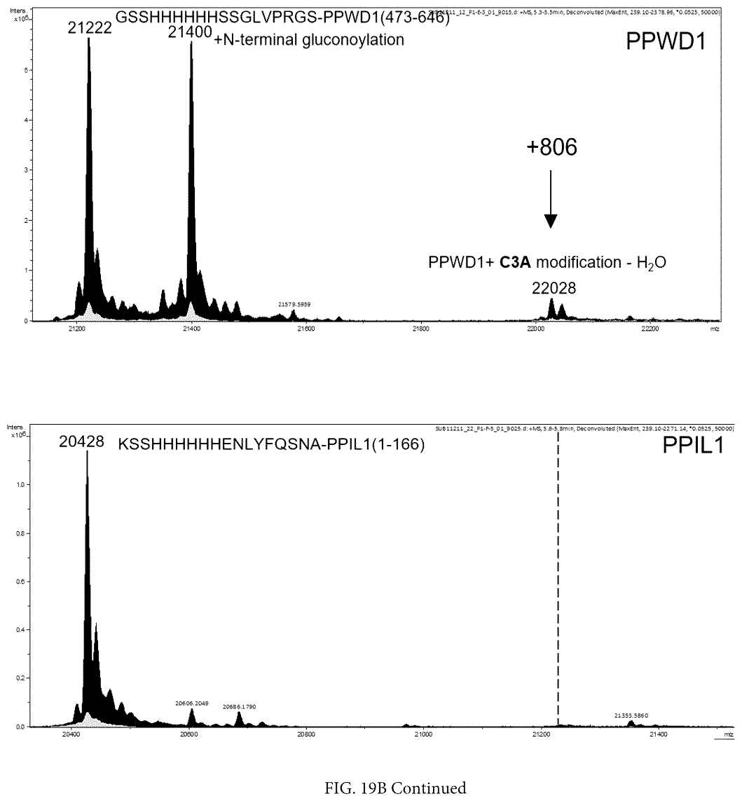

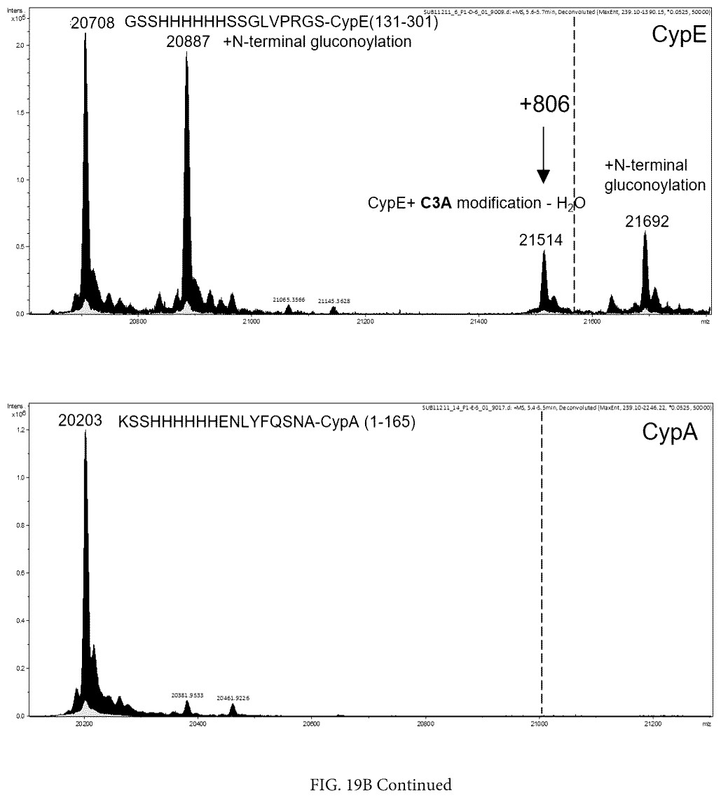

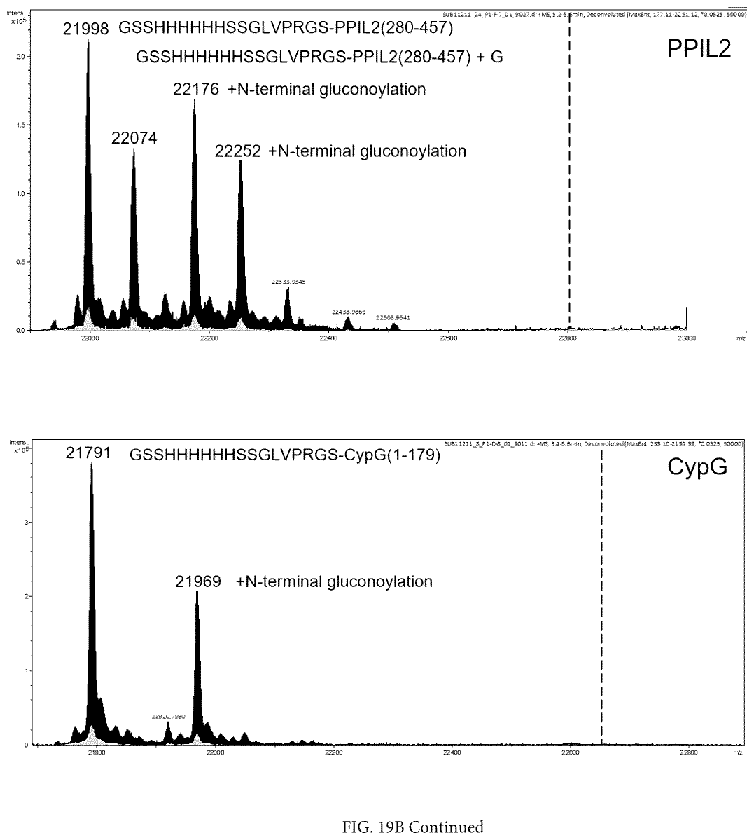

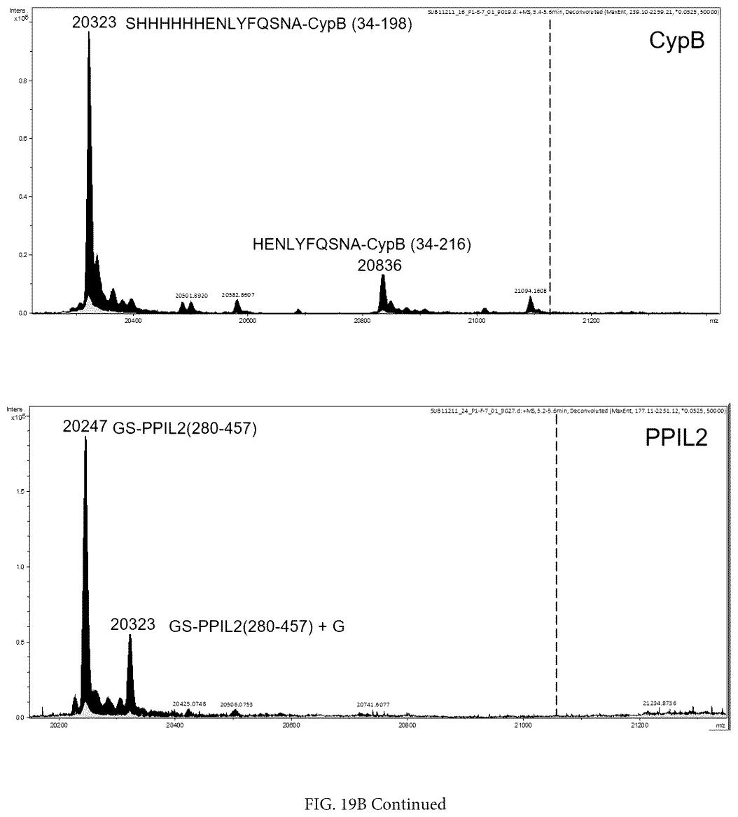

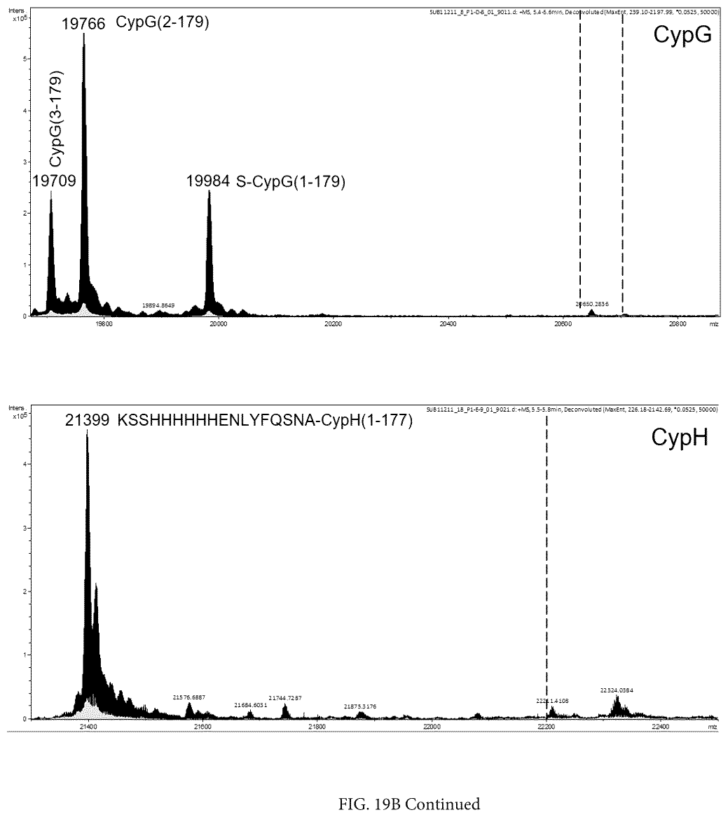

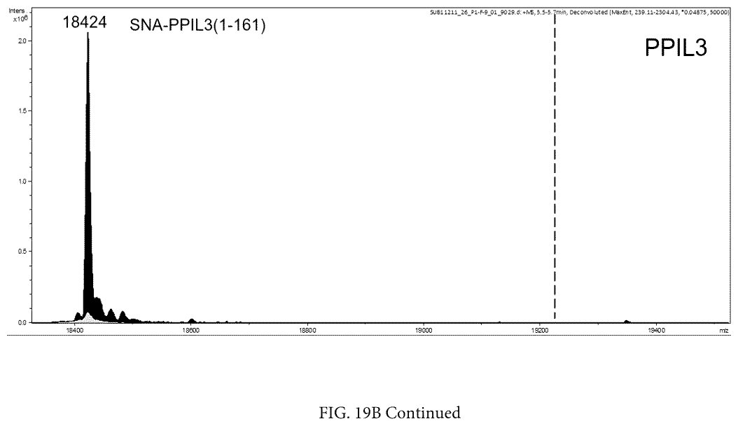

After demonstrating that C3A was a potent and selective binder for CypE, it was assessed whether C3A was also a potent and selective inhibitor of CypE enzymatic activity. This was determined by screening C3A for prolyl isomerase inhibition as previously described. It was determined that C3A was a potent inhibitor against CypE prolyl isomerase activity, with an IC₅₀ of 13 nM (FIGS. 4C and 18B). C3A was also selective for CypE with an IC₅₀ that is least 30-750-fold more potent for CypE than for the other ten cyclophilins screened. Once again, replacement of the aldehyde with a ketone (C1A, IC₅₀^{CypE}=0.9 μM), or removal of either the boronic acid (C5A, IC₅₀^{CypE}=1 μM) or the aldehyde (C6A, IC₅₀^{CypE}=4 μM) reduced both the potency and selectivity compared to C3A, again supporting that C3A inhibits CypE covalently (FIGS. 18A-18E). However, mass spectrometric analysis after incubation of C3A with CypE did not reveal a lysine-modified covalent adduct; the m/z of C3A and CypE were observed (FIG. 19A).

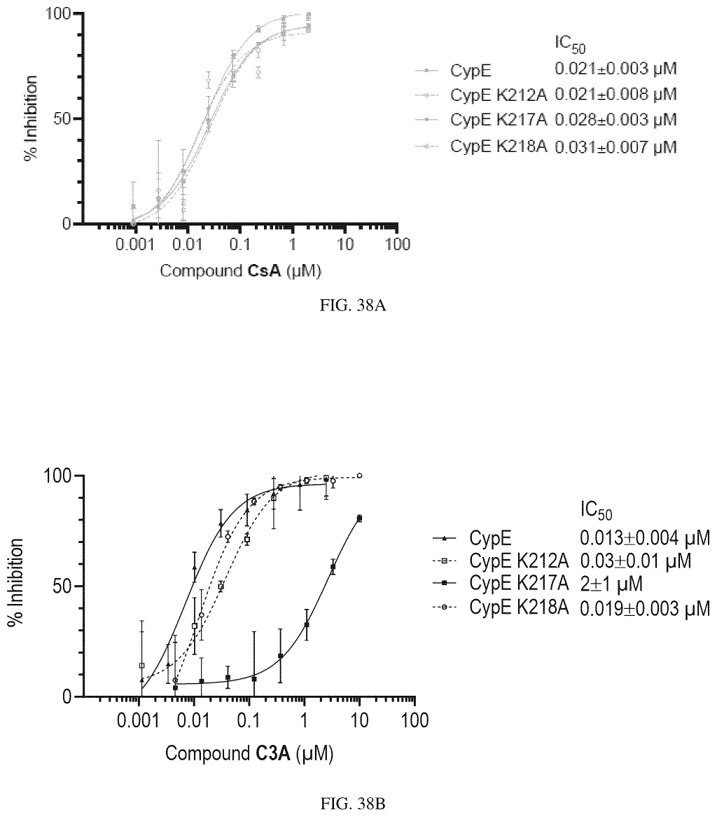

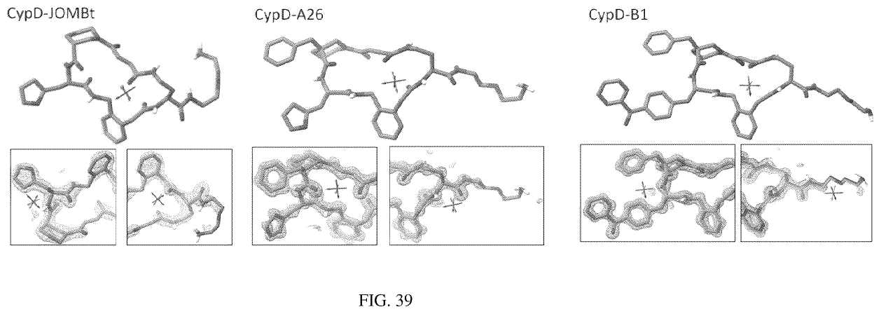

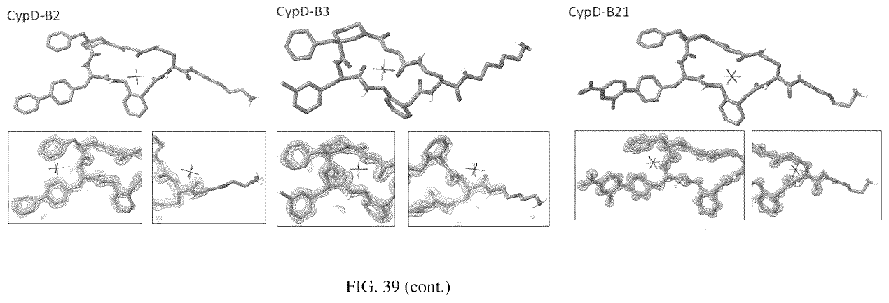

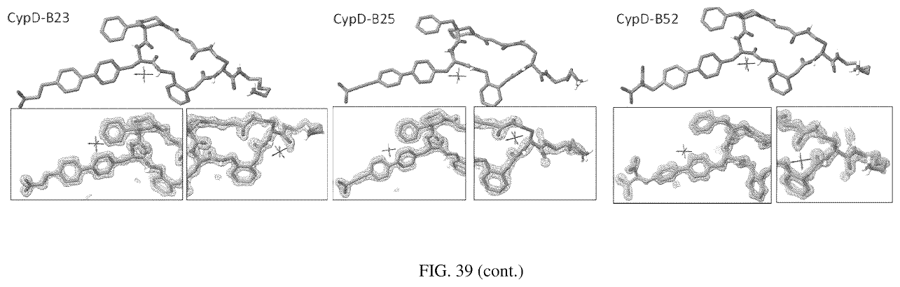

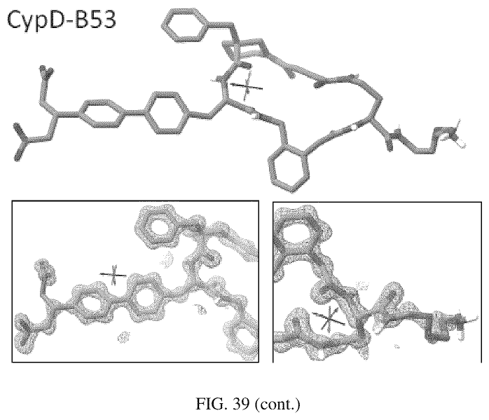

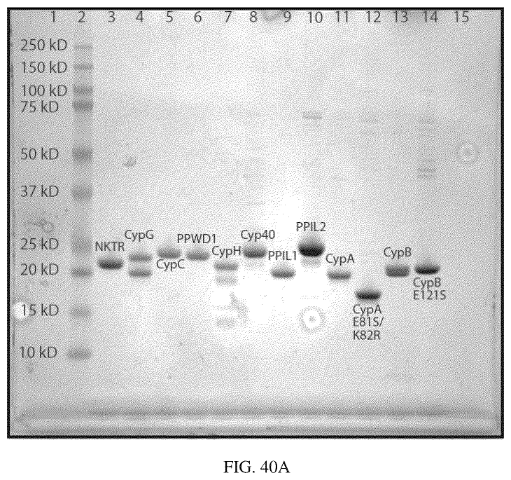

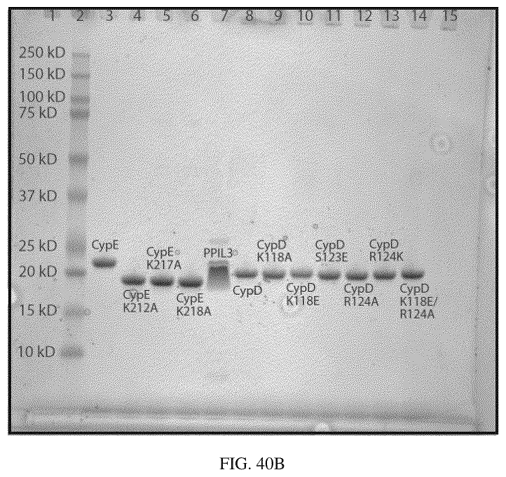

It was possible that reducing the iminoboronate intermediate with sodium cyanoborohydride (NaCNBH₃) might enable trapping of the covalent adduct. Indeed, co-incubating C3A in 5-fold excess for 1 hour with CypE followed by treatment with 25 mM NaCNBH₃ resulted in observation of the mass shift of a +807 peak, corresponding to loss of water for aryl boronic acid aldehyde C3A (FIG. 4D), but not for boronic acid C6A (FIG. 19A). The loss of water peak was also reported for other aryl boronic acid carbonyl inhibitors treated with NaCNBH₃^{49,50}. These findings suggested that C3A functions in a reversible covalent manner. A +780 Da covalent adduct formation was also observed under these conditions with C5A, which only contained an aldehyde, indicating that carbonyl groups at this position can form imines with CypE's S2 pocket lysines (FIG. 19B). Nonetheless, the 75-fold higher CypE inhibition potency of C3A over C5A suggested that this reversible interaction is substantially stronger for C3A. Little or no covalent modification was observed for 12 other cyclophilins when treated with C3A and NaCNBH₃, establishing that this covalent interaction is important to CypE (FIGS. 19A-19B). To determine which CypE lysine residue is covalently modified by C3A, alanine point mutants were generated at each of the three CypE S2 pocket lysines (K212A, K217A, K218A). CypE K212A and K218A mutants showed similar binding and inhibition potency by C3A as wild-type CypE (FIGS. 4E, 38B, 39B). However, C3A was 40-fold less potent at competing A26-Fl off of CypE K217A (K^{CypEK217A}i=3.1 μM) compared to wild-type CypE (K^{CypE}i=0.072 μM) and was 150-fold less potent at inhibiting K217A prolyl isomerase activity (IC^{CypEK217A}₅₀=2 μM) compared to wild-type CypE (IC^{CypE}₅₀=0.013 μM).

Collectively, these results indicate that our generalized model for achieving cyclophilin selective inhibition can be translated to other cyclophilin proteins, resulting in potent and selective CypE inhibition through reversible covalent bonding with CypE's S2 pocket K217 residue.

### Example 4. General Experimental Methods

Fmoc-protected amino acids and peptide coupling reagents were purchased from Chem-Impex International Inc. Boronic acids or aryl halides were purchased from Combi-Blocks Inc. and Enamine Ltd. All other chemical reagents, PPIL4 (Full length), and PPIL6 (C-Myc/DDK), were purchased from Millipore-Sigma. SDCCAG-10 (GST) was purchased form Abnova. NMR spectra were gathered using a Bruker Ascend™ 400 MHz NMR. Quantification of DNA was completed using a NanoDrop™ One Microvolume UV-Vis Spectrophotometers (ThermoFisher Scientific). Preparative HPLC reverse phase purification was conducted with an Agilent 6100 Quadrupole LC/MS system using a Kinetex® 5 μm C18 100 Å, AXIA Packed LC Column 150×30.0 mm (Phenomenex®). Silica gel column chromatography was conducted with a Biotage® SP1 Flash Chromatography system. Recombinantly expressed proteins were purified using a ÄKTA™ pure FPLC. qPCR analysis was conducted using a CFX96 Touch Deep Well Real-Time PCR System (Bio-Rad). ¹H-NMR spectra for all reported compounds are provided in a separate section of the SI. HRMS data is provided in FIGS. 9-10. All tested compounds were quantified with CH₂Cl₂ internal standard by ¹H-NMR.

General mammalian cell culture conditions. HEK293T (American Type Culture Collection (ATCC) CRL-3216), HeLa (ATCC CCL-2), mouse embryonic fibroblasts (MEFs) (ATCC CRL-2991), HepG2 (ATCC HB-8065), and A549 (ATCC CCL-185) cells were purchased from ATCC and cultured and passaged in DMEM plus GlutaMAX (Thermo Fisher Scientific) for HEK293T/HeLa/MEFs, MEM (Corning) for HepG2, and F-12K (ATCC) for A549, each supplemented with 10% (v/v) FBS (Gibco, qualified). All cell types were incubated, maintained, and cultured at 37° C. with 5% CO2. Cell lines were authenticated by their respective suppliers and tested negative for mycoplasma.

HPLC purity analyses of key compounds. Analytical analyses for compound purity was performed using an Agilent 6100 Quadrupole LC/MS system with a KinetexR 5 μm C18 100 Å LC Column 150×2.1 mm (PhenomenexR). 5 μL of 0.5-1 mM compound in d6-DMSO was injected and run for 3 minutes at 10% water/acetonitrile with 0.1% trifluoroacetic acid to elute off d6-DMSO. From 3 minutes to 15 minutes, gradient was increased to 100% acetonitrile, and held for 2 more minutes. Compound peaks were identified using the MS trace. Purity analyses was quantified by % area of the compound peak at 214 nM absorbance relative to all identified peaks within the 3-17 minute analysis window (to exclude the DMSO peak at 1.5 minutes).

Generation of His₆-Cyp expression constructs: Geneblock sequences for PPIF (UniProtKB Entry P30405: res 45-207), PPIA (UniProtKB Entry P62937: res 1-165), PPIB (UniProtKB Entry P23284: res 34-216), PPID (UniProtKB Entry Q08752: res 1-183), PPIH (UniProtKB Entry 043447: res 1-177), PPIL1 (UniProtKB Entry Q9Y3C6: res 1-166), and PPIL3 (UniProtKB Entry Q9H2H8: res 1-161) were purchased from IDT and cloned into 2BT (UC Berkeley QB3 MacroLab) using Ligation Independent Cloning. Gatekeeper mutations for PPIA and PPIB were introduced using site-directed mutagenesis with Q5 DNA polymerase (NEB) and primers from IDT. All constructs were verified using Sanger sequencing. E. coli containing the constructs were cultured overnight at 37° C. in 2xYT media (31 g in 1 L) containing 100 μg mL⁻¹ ampicillin. PPIL2 (UniProtKB Entry Q13356: res 280-457) cloned into pET28a LIC (Addgene Plasmid #25601), PPIG (UniProtKB Entry Q13427: res 1-179) cloned into pET28a LIC (Addgene Plasmid #25137), PPIE (UniProtKB Entry Q9UNP9: res 131-301) cloned into pET28a LIC (Addgene Plasmid #25605), PPWD1 (UniProtKB Entry Q96BP3: res 473-646) cloned into pET28a LIC (Addgene Plasmid #25600), PPIC (UniProtKB Entry P45877: res 24-212) cloned into pET28a LIC (Addgene Plasmid #25606), and NKTR (UniProtKB Entry P30414: res 7-179) cloned into pET28a LIC (Addgene Plasmid #25597) were purchased as bacterial agar stabs and expressed in BL21DE3 cells. E. coli containing the constructs were cultured overnight at 37° C. in 2xYT media (31 g in 1 L) containing 50 μg mL⁻¹ kanamycin.

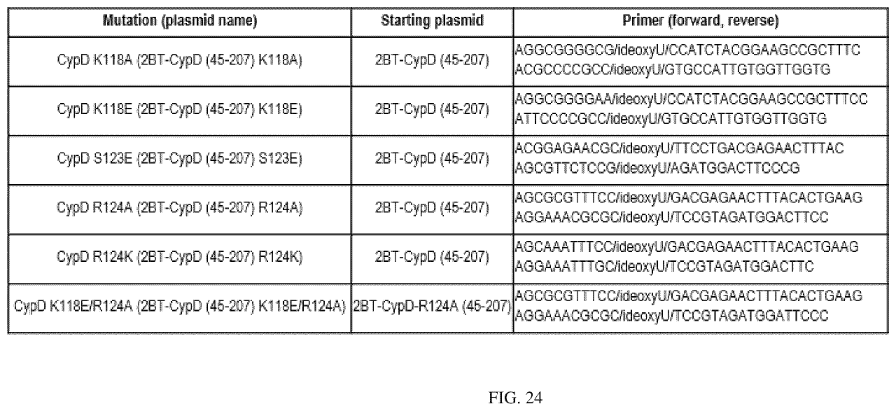

Site-Directed Mutagenesis of CypD: Mutant CypD constructs were generated with primers for 1-piece uracil-specific excision reactions (USER) containing a mutant overhang by PCR amplifying starting plasmid (FIG. 24). PCR product was purified on microcentrifuge membrane columns (MinElute®, Qiagen) and quantified by Nanodrop. Fragment (0.2 μmol, 7.5 μL) was combined in a 10 μL reaction mixture containing 0.75 μL (15 Units) DpnI (NEB), 0.75 μL of USER® mix (Endonuclease VIII and Uracil-DNA Glycosylase, NEB), 1 μL 10× CutSmart® Buffer (NEB). Reactions were incubated at 37° C. for 30 minutes followed by heating to 80° C. for 3 minutes and slow cooling to 12° C. at 0.1° C./sec. Constructs were directly transformed into One Shot® Mach1™ T1 Phage-Resistant Chemically Competent E. coli (Invitrogen™) by heat-shock for 30 seconds and streaked on 100 μg/mL carbenicillin containing LB agar. Selected colonies with correct construct, verified by Sanger sequencing, were cultured overnight in 2xYT supplemented with 100 μg/mL carbenicillin. Plasmid was extracted by microcentrifuge membrane columns (QIAprep Spin Miniprep Kit, Qiagen) as per manufacturer's instructions and quantified by Nanodrop.

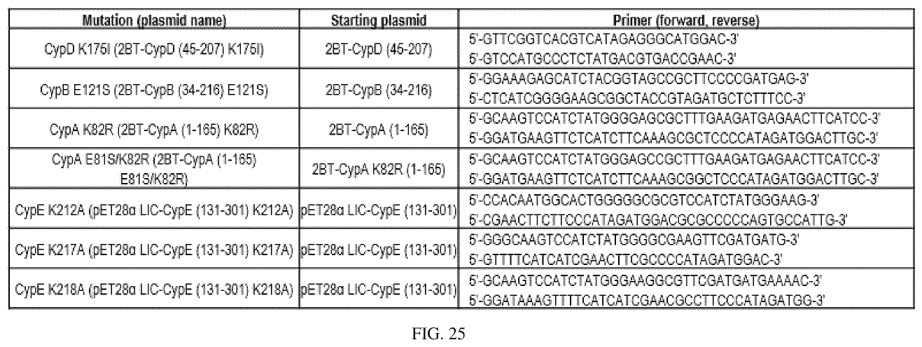

Site-Directed Mutagenesis of CypA, CypB, CypD (for K175I mutant), and CypE: The K175I mutation for reduction of surface entropy⁷⁵ was introduced into PPIF (CypD) construct and the gatekeeper residue mutations were introduced into the PPIA (CypA), PPIB (CypB), and PPIE (CypE) constructs via Quikchange Site-Directed Mutagenesis (Agilent Technologies). Primers for the respective mutations are included in FIG. 25. The template plasmid (˜100 ng, 1 μL) was combined in a 25 μL mixture with forward and reverse primers (125 ng, 1.25 μL each), Q5 High-Fidelity DNA Polymerase (NEB) (1 μL, 2000 U/mL), dNTP mix (10 mM, 1 μL), Q5 Reaction Buffer (NEB) (5×, 5 μL), and deionized H₂O (14 μL). Reactions were incubated at 98° C. for 120 seconds, followed by 30 cycles of 98° C. (10 seconds—melting), 55-60° C. (30 seconds—annealing), and 72° C. (5 minutes—elongation), followed by a final elongation cycle of 7 minutes. PCR products were transformed into E. coli DH5a Competent cells with a heat shock at 42° C. for 45 seconds and streaked onto Luria Broth agar plates containing 100 μg/mL ampicillin. Single colonies were isolated for inoculation and plasmid extraction via microcentrifuge membrane columns (QIAprep Spin Miniprep Kit, Qiagen), and mutations were verified by Sanger sequencing.

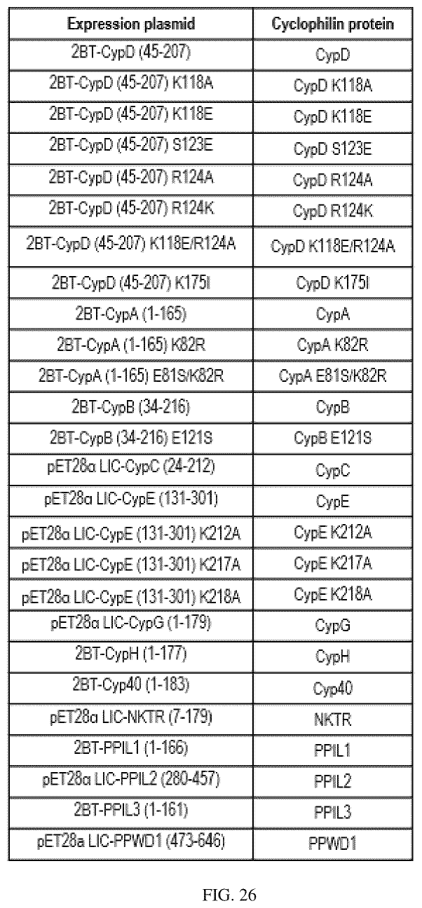

Recombinant expression and isolation of CypD and CypD Mutants: CypD proteins were obtained from expression plasmids (FIG. 26) by transforming into One Shot® BL21(DE3) Chemically Competent E. coli (Invitrogen™) by heat shock at 42° C. for 30 seconds. Cells were streaked onto agar plates containing 100 μg/mL carbenicillin and incubated at 37° C. for 16 hours. Individual colonies were grown up in a 2 L culture of LB media supplemented with 100 μg/mL carbenicillin at 37° C. until optical density reached 0.8. The culture was then cooled to 16° C. for 1 hour and protein production was induced by adding 2 mL of 1 M Isopropyl β-d-1-thiogalactopyranoside and left to incubate for 16 hours. Cells were pelleted at 4000 g for 5 minutes at 4° C. and resuspended in 50 mL of cold Tris-HCl pH 8.0, 50 mM NaCl, 5% glycerol (NiA Low Salt). Two Pierce Protease Inhibitor Tablets (ThermoFisher Scientific) was added to the suspension and cells were subsequently lysed using an Avestin Emulsiflex C3 homogenizer at 17,000-20,000 PSI. Lysed cells were pelleted, and supernatant was purified by FPLC affinity chromatography using a Histrap™ HP 5 mL (GE) and a gradient of 0-100% NiA Low Salt/NiB Low Salt (NiB Low Salt=NiA Low Salt+500 mM imidazole). Protein eluted off around 60-70%, confirmed by SDS-PAGE analysis of fractions. Isolated fractions were additionally purified by FPLC cation exchange, using a HiTrap™ SP 5 mL column (GE) and a gradient of 0-100% SA Buffer/SB Buffer (SA Buffer=Tris-HCl pH 7.0, 1 mM DTT, 5% glycerol, SB Buffer=SA Buffer+1M NaCl). Fractions corresponding to >90% pure CypD came off around 40%, confirmed by SDS-PAGE. Combined fractions were dialyzed in 20 mM Tris-HCl pH 8.0, 50 mM NaCl, 1 mM DTT, 5% glycerol, using a Slid-A-Lyzer™ MWCO=3,000 dialysis cassette following manufacturer's instructions, overnight at 4° C. Protein purity was >90% based on SDS-PAGE gel electrophoresis and Coomassie staining. Pooled fractions were concentrated, flash-frozen in liquid nitrogen, and stored at −80° C. Protein was quantified using Pierce™ BCA Protein Assay Kit.

Recombinant expression and purification of CypD, CypA, CypB, CypC, CypE, CypG, CypH, Cyp40, NKTR, PPIL1, PPIL2, PPIL3, PPWD1: The expression plasmids for wild-type and mutant protein constructs (FIG. 26) were transformed into E. coli BL21(DE3) competent cells with heat shock at 42° C. for 45 seconds. Cells were streaked onto Luria Broth agar plates containing 100 μg/mL ampicillin and incubated overnight at 37° C. Single colonies were inoculated into 2xYT media supplemented with 1% glucose, 1 mM Mg^{2+ 51}, and 100 μg/mL ampicillin and shaken at 37° C. until reaching an OD₆₀₀=˜0.6-0.8. The cultures were then cooled to 16° C. for one hour, and protein production was induced with 1 mM Isopropyl β-d-1-thiogalactopyranoside overnight for 16-18 hours. Cells were pelleted at 4000 g for 10 minutes at 4° C. before being homogenized in an Avestin Emulsiflex-C3 High Pressure Homogenizer at 17,000-20,000 PSI three times and resuspended in buffer containing 20 mM Tris pH 8.0, 50 mM NaCl, and 5% glycerol. Lysates were centrifuged for one hour at 17,000 rpm at 4° C. in a Sorvall SLC6000 Fixed-Angle Rotor. Protein was purified from the supernatant via nickel affinity chromatography followed by cation exchange chromatography and size exclusion chromatography. First, the recombinant His₆-tagged proteins were purified with Ni(II)-affinity chromatography (HisTrap FF, GE-Healthcare). The supernatant was run over the column, which was subsequently washed twice with buffer to remove nonspecific binding. Two mL fractions were eluted over twelve column volumes by increasing the imidazole concentration to 500 mM. The His₆-tag was cleaved with TEV overnight in pH 8.0 dialysis buffer containing 20 mM Tris base, 100 mM NaCl, 5 mM BME, 5% glycerol with a 3 kDa molecular weight cutoff filter. The cleaved protein was diluted to reduce NaCl concentration in pH 8.0 buffer containing 20 mM Tris, 1 mM DTT, and 5% glycerol and loaded onto a cation exchange column (HiTrap SP, GE Healthcare). The column was washed with six column volumes of buffer to remove nonspecific binding. Two mL fractions were eluted over twelve column volumes with increasing salt gradient up to 1 M NaCl. Pooled fractions containing protein were concentrated and loaded onto a size exclusion column (HiLoad 16/600 Superdex 200 prep grade, GE Healthcare). Protein was eluted in pH 7.3 buffer containing 50 mM NaH₂PO₄, 100 mM NaCl, and 2 mM EDTA. Protein purity was >95% based on SDS-PAGE gel electrophoresis and Coomassie staining. Pooled fractions were concentrated, flash-frozen in liquid nitrogen, and stored at −80° C. Protein was quantified using Pierce™ BCA Protein Assay Kit.

Recombinant expression and purification of CypA E81S/K82R, CypB E121S, CypE (131-301) K212A, K217A, and K218A mutants. CypA, CypB, and CypE mutants were purified similar to wild-type with modification as indicated. Following initial nickel affinity chromatography and overnight dialysis with thrombin-mediated His₆-tag cleavage, the remaining protein was again run over a Ni(II)-affinity chromatography column (HisTrap FF, GE-Healthcare) and flow-through and wash were collected to remove any unbound protein and His6 tags. Protein purity in flow-through and wash was >95% based on SDS-PAGE gel electrophoresis and Coomassie staining. Pooled flow-through and wash was concentrated, flash-frozen in liquid nitrogen, and stored at −80° C. Protein concentration was quantified using Pierce™ BCA Protein Assay Kit.

In vitro selection of a 256,000-member DNA-templated library with human His₆-CypD: Selection protocol utilized a 256,000-member library and recombinant N-His₆-CypD₄₅₋₂₀₇ adapted from previous work⁵². Resuspended magnetic Ni-NTA beads (Dynabeads™His-Tag Isolation and Pulldown, Invitrogen™) (25 L) were immobilized on a MagJET Separation Rack (ThermoFisher) with the supernatant subsequently removed. Beads were washed 2 times with 300 L 50 mM Sodium Phosphate pH 8.0, 300 mM NaCl, 0.01% Tween-20, 2 mM TCEP (PBST). 40 g of CypD protein was loaded onto solid support in 300 L PBST, incubated on rotary for 1 hour at 4° C., and washed twice with 200 L 50 mM Tris-HCl pH 8.0, 150 mM NaCl, 0.05% Tween-20, 2 mM TCEP (TBST). Immobilized protein was suspended in 100 L Blocking buffer (TBST+0.1 mg/mL BSA+0.6 mg/mL yeast total RNA) for 25 minutes at 4° C. After supernatant was removed, 20 μmol of second-generation 256,000-member DTS library was suspended with bead immobilized protein in 50 μL Blocking Buffer and incubated for 1 hour at 4° C. Supernatant was removed (FT fraction) and beads were washed three times with TBST, retaining the supernatants (W1, W2, W3). CypD was eluted off Ni-NTA beads by incubating with 50 μL PBST supplemented with 300 mM imidazole (Elution Buffer) for 5 minutes. The elution was PCR amplified and barcoded with Illumina Primers as previous reported⁴. PCR amplicons were purified by polyacrylamide gel electrophoresis (PAGE), extracted, and quantified with KAPA qPCR analysis and QuBit (Invitrogen™). High-throughput DNA sequencing were performed on an Illumina MiSeq using manufacturer's instructions for sample preparation. Reads generated were analyzed using Python scripts to quantify library barcodes and calculate change in % abundance for each library member compared to pre-selection library.

Chymotrypsin coupled prolyl-isomerase assay: Adapting a previously described protocol⁵³, an Agilent Bravo Velocity 11 with a 384ST head, 90 μL of assay buffer (25 mM HEPES pH 7.3, 100 mM NaCl, 0.01% Triton X-100) containing 5.28 nM cyclophilin was added into a flat, clear-bottom, black 384 well plate pre-chilled in a Corning CoolBox™ at 2-3° C. 5 μL of compound pre-dissolved in 5% DMSO/assay buffer was then added to appropriate wells and incubated at 2-3° C. for 5 minutes. 5 μL of 0.5 mM α-chymotrypsin from bovine pancreas in 20% 1 mM HCl/Assay Buffer (Type II, lyophilized powder, Millipore-Sigma) was then added to each well and incubated at 2-3° C. for 5 minutes. Plate was then quickly transferred to a FLIPR Tetra High-Throughput Cellular Screening System (Molecular Devices). Using the FLIPR's internal liquid handling system, 1 μL of 0.5 mM Suc-AAPF-AMC (Chymotrypsin Substrate II, Millipore-Sigma) dissolved in 0.55 M LiCl/2,2,2-trifluoroethanol was added to each well, which was then mixed for 5 secconds, followed by immediate fluorescence measurements every 1 sec for 330 seconds using a 360-380 nM excitation LED module and a 400-460 emission filter. Final concentrations of the plate include 5 nM cyclophilin, 0.25% DMSO, 25 μM α-chymotrypsin, 5 μM Suc-AAPF-AMC. Raw fluorescence data was analyzed by non-linear regression analysis with Prism 9 by fitting one-phase association curves to each well. Rate constants calculated for each well (s⁻¹) were then normalized to substrate only (no prolyl isomerase) and enzyme+substrate only controls. IC₅₀ values were calculated to be the value at which 50% inhibition was achieved on each compound's non-linear regression fitted curve.

Anisotropy binding assay: Adapted from previous work⁵⁴, into a flat-bottom black untreated 96 well plate (Corning), titrated cyclophilin in assay buffer (25 mM HEPES pH 7.3, 100 mM NaCl, 0.01% Triton X-100) was incubated with 0.5 nM fluorescein labeled macrocycle for 6 hours at room temperature. Final assay volume was 100 μL with 0.25% DMSO. Fluorescence anisotropy was then measured using a Tecan Spark® plate reader using 492 nm/523 nm excitation/emission settings. Raw fluorescence polarization data was analyzed by non-linear regression analysis with Prism 9 by fitting a one site-total binding equation, providing a dissociation constant (K_{d}) for each cyclophilin-compound pair.

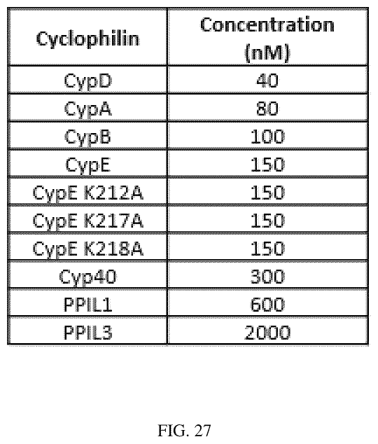

Competition anisotropy binding assay with A26-Fl: Adapted from previous work⁵⁴, into a flat-bottom black untreated 96 well plate (Corning), cyclophilin in assay buffer (25 mM HEPES pH 8.0, 100 mM NaCl, 0.01% Triton X-100) at a predetermined concentration from FIG. 27 was incubated with 0.5 nM A26-Fl for 10 minutes at room temperature. Competitor macrocycle was then added to each well and incubated for 24 hours. Final assay volume was 100 μL with 0.25% DMSO. Fluorescence anisotropy was then measured using a Tecan Spark® plate reader using 492 nm/523 nm excitation/emission settings. Raw fluorescence polarization data was normalized to protein+fluorescent probe and buffer+fluorescent probe and Kᵢ values were calculated using one site-competitive binding equation with Prism 9, importing K_{d} values from A26-Fl (FIGS. 12A-12C).

Surface Plasmon Resonance (SPR) analysis of CypD ligands: Adapting a previously used protocol⁵⁵, employing a Biacore T200 SPR and with a Sensor S NTA (Biacore), chip pre-loaded with 0.3 M NiCl₂, His₆-CypD (50.24 nM, 1 μg/mL) was flowed over the chip with a solution of 10 mM HEPES, 150 mM NaCl, 0.005% Tween-20 and 1 mM TCEP (SPR Buffer). Protein was covalently immobilized subsequently with injection of 200 mM 1-Ethyl-3-(3-dimethylaminopropyl)carbodiimide (EDC) 50 mM N-hydroxysuccinimide (NHS). EDC/NHS activated chip was quenched by flowing 1 M ethanolamine over the chip. Compounds dissolved in 1% DMSO/SPR Buffer and flowed over the SPR chip. Binding analysis was conducted from raw sensorgram RU values, where individual replicates represent average RU value during time of compound administration at each dose. RU values were normalized to DMSO treated and the the maximum RU value during compound administration at the highest dose. These values were evaluated using Prism 9 by fitting one site-specific binding to calculate K_{d} values.

Assessment of covalent modification of CypE and other cyclophilin proteins: Into PCR strip, CypE or other cyclophilin in assay buffer (25 mM HEPES pH 8.0, 100 mM NaCl, 0.01% Triton X-100) was incubated with compound for 1 hour at room temperature. Final concentrations were 20 μM CypE, 100 μM compound, 0.5% DMSO, at a final volume of 20 μL. For analysis of lysine-iminoboronate modification, samples were directly submitted to Harvard's Center for Mass Spectroscopy for LC-MS analysis. For amine-lysine modification, samples were treated with 5 μL of 125 mM sodium cyanoborohydride dissolved in 25 mM Ammonium bicarbonate solution (pH 8.0) and incubated for 4 hours at room temperature. For all other wild-type cyclophilins, protein was treated for 4 hours with C3A, followed by 16 hour treatment with NaCNBH₃. Final [NaCNBH₃]=25 mM. Samples were then submitted for LC-MS as described above.

Co-crystallization of CypD K175I PPI domain (45-207) with macrocycle compounds: Macrocycles were incubated with 15 mg ml⁻¹ CypD K175I (45-207) (JOMBt, B1, B2, B3—1:3, A26—1:1.5, B21, B23, B25, B52, B53—1:2) for 15 minutes on ice. Crystals were obtained by mixing 1 μL of the protein:drpg complex with 1 μL of mother liquor (JOMBt: 19% PEG 3350, 0.5M KH₂PO₄ pH 7.3, A26: 29% PEG 3350, 0.5M KH₂PO₄ pH 7.3, B1: 23% PEG 3350, 0.5M KH₂PO₄ pH 7.3, B2: 28% PEG 3350, 0.5M KH₂PO₄ pH 7.3, B3: 28% PEG 3350, 0.5M KH₂PO₄ pH 7.3, B21: 20% PEG 3350, 0.5M KH₂PO₄ pH 7.3, B23: 21% PEG 3350, 0.5M KH₂PO₄ pH 8.2, B25: 13% PEG 3350, 0.5M KH₂PO₄ pH 8.0, 7.5% glycerol, B52: 15% PEG 3350, 0.5M KH₂PO₄ pH 6.0, 1 mM NaCl, B53: 22.5% PEG 3350, 0.5M KH₂PO₄ pH 6.0, 1 mM NaCl) and equilibrating against 1 mL of the same reservoir solution at room temperature.

Large single rectangular crystals formed within 24 hours and were equilibrated in a cryoprotective crystallography buffer composed of mother liquor containing 30% glycerol then snap-frozen in liquid nitrogen. Diffraction data was collected from single crystals at 100 K at the FMX (CypD-JOMBt: λ=0.97933 Å, CypD-A26: λ=0.97933 Å, CypD-B1: λ=0.97933 Å, CypD-B2: λ=0.97933 Å, CypD-B3: λ=0.97933 Å, CypD-B21: λ=0.92016 Å, CypD-B23: λ=0.97931 Å) and the AMX (CypD-B25: λ=0.92011 Å, CypD-B52: λ=0.92011 Å, CypD-B53: λ=0.92011 Å) beamlines at the National Synchrotron Light Source II operated by Brookhaven National Laboratory at the indicated wavelengths.

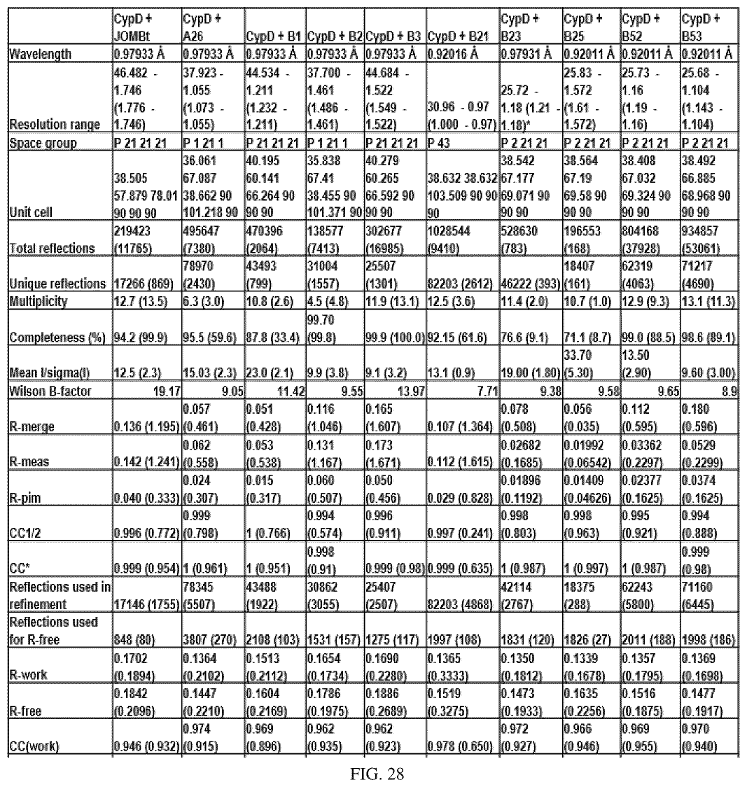

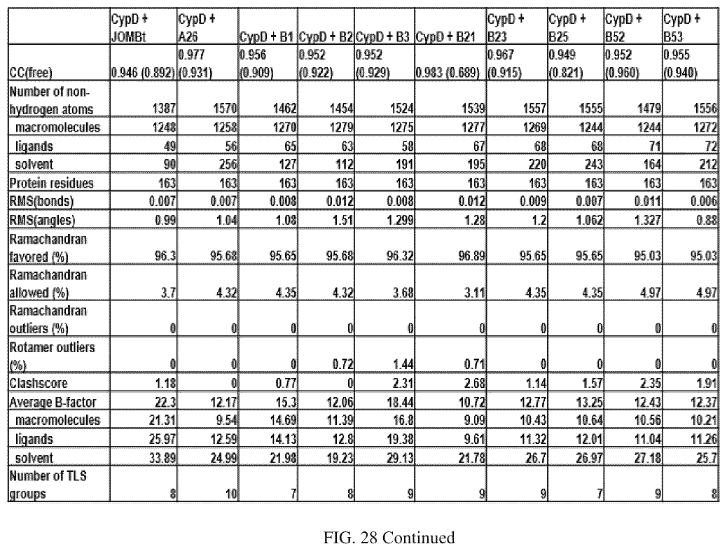

Crystal structure refinement: Diffraction data for JOMBt, A26, B1, B2, and B3 was indexed, integrated, and scaled with autoPROC⁵⁶, and diffraction data for B21, B23, B25, B52, and B53 was indexed, integrated, and scaled with FastDP⁵⁷. Both programs use additional functionality within XDS⁵⁸ and CCP4.⁵⁹ Phases were assigned via molecular replacement in Phaser⁶⁰ with the apo structure of CypD K175I (PDB: 2BIT) for CypD-JOMBt and subsequently with previously solved structures of CypD K175I complexed with macrocyclic inhibitors as the search model. All refinements to the model were made in PHENIX⁶¹. Model building was performed in Coot⁶² with ligands and waters fit into the initial |Fₒ|−|F_{c}|. Macrocycle restraints were generated using eLBOW⁶³ (JOMBt, A26, B1, B2, B3) and the ProDrg server⁶⁴ (B21, B23, B25, B52, B53). The coordinates of the holo-structures of CypD have been deposited in the RCSB Protein Data Bank. Additional crystallographic and data collection statistics are listed in FIG. 28.

Molecular Footprinting Analysis: Molecular footprints, defined as per-residue decomposition of the Van der Waals, electrostatic, and hydrogen bonding energies between the ligand and the receptor, were generated with the crystal structures in the DOCK6.9 molecular modeling software as described by Balius et al⁶⁵. Briefly, two crystal structures were structurally superimposed in UCSF Chimera⁶⁶ based on lowest pairwise root-mean square deviation using the Needleman-Wunsch alignment algorithm⁶⁷. Both the reference ligand and B52 were saved in relation to the CypD-B52 protein structure and were parameterized using the GAFF force field⁶⁸ and the Gasteiger charging method^{69,70}. The ligands were then rigidly docked into the receptor using DOCK6.9, and the pairwise interaction energies for the top fifty contributing residues were visualized using matplotlib⁷¹ in Python.

Isolation of mouse liver mitochondria: All procedures used in animal studies were approved by the Institutional Animal Care and Use Committee at Massachusetts General Hospital. Female C57B1/6 J mice (Jackson Labs) age 10-12 weeks were anesthetized by isoflurane. The liver from one mouse was rinsed in ice-cold PBS, minced in ice-cold isolation buffer containing 0.28 M sucrose, 10 mM Hepes-KOH pH 7.2, 0.2 mM EDTA and 1% (w/v) BSA, gently homogenized with 4 strokes of a tight-fitting Teflon pestle at 1000 rpm, and then centrifuged for 10 minutes at 600 g at 4° C. The supernatant was recovered and centrifuged for 10 minutes at 8000 g at 4° C. The loose outer buffy coat was rinsed off and the pellet resuspended gently in isolation buffer and the spins were repeated. The remaining buffer coat layer was rinsed off and the pellet resuspended in buffer containing 137 mM KCl, 10 mM Hepes-KOH pH 7.2, 2.5 mM MgCl₂ for a final concentration between 40-80 mg/ml as assessed by Bradford. The suspension was kept on ice and all assays performed within 4-6 hours following isolation. Quality control was done with every preparation by adding 250 mcg mitochondria to 500 μl of assay buffer containing 137 mM KCl, 10 mM Hepes-KOH pH 7.2, 2.5 mM MgCl₂, 5 mM each of glutamate and malate, and 3 mM KH₂PO₄. Sequential 150 μM ADP pulses were then delivered. Preparations with respiratory control ratio>6 (as assessed by the ratio of ADP-coupled state 3 respiration to state 4 respiration) were used for further experiments. Measurements were made in a custom-built fluorimeter using a cuvette with a Red Eye oxygen patch (Ocean Optics) and an optical probe that is connected to the fluorimeter.

Mitochondrial calcium retention capacity assays: 250 mcg of mouse liver mitochondria isolated as above were added to 500 ml of assay buffer containing 125 mM KCl, 20 mM Hepes-KOH pH 7.2, 1 mM MgCl₂, 5 mM each of glutamate and malate, and 3 mM KH₂PO₄. 0.5 μM Calcium Green-5N (Molecular Probes) was included to monitor extramitochondrial free Ca²⁺. Fluorescence was continuously monitored in the custom-built fluorimeter described above, with excitation 470 nm and emission 520-560 nM. Sequential 60 μM CaCl₂) pulses were delivered until Ca²⁺ uptake ceased and an abrupt release of previously taken up Ca²⁺ was observed, consistent with mPTP opening. The calcium retention capacity ratio (CRC) was obtained by normalizing the number of Ca²⁺ pulses that could be taken up in a given condition by that taken up under DMSO control conditions, as previously described⁷². Student's t test was used to determine statistical significance between predefined comparisons (DMSO vs CsA, inactive vs active enantiomers of B52-Cy5, and inactive vs active enantiomers of B53-Cy5).

Fluorescence microscopy on Hela cells. Into 96 well black, clear bottom TC treated plates, cells were seeded in Dulbecco's Modified Eagle Medium (DMEM) supplemented with 10% fetal bovine serum 24 hours prior at a density where wells were ˜80% confluent at time of experiment. Media was removed and cells were then stained with 50 μL of mixture containing Cy5 labeled compound (6 μM) in Kreb's ringer solution HEPES buffered pH 7.2 (KRB), for 1 hour at 37° C. Then, 50 μL Hoechst 33342 (8 μM) and Mitotracker Green FM (0.1 μM) in KRB was added directly to wells containing cells and incubated for 15 minutes at 37° C. Media was removed and cells were washed two times with 100 μL KRB. Cells were then kept adherent to the wells in 100 μL KRB for imaging. Fluorescence microscopy was conducted on an Opera Phenix Plus High-Content Screening System, using Alexa 488, Alexa 647, and Hoescht 33342 channels. Images were analyzed using Harmony 4.9.

In vitro esterase activity on ester pro-drug Cy5 derivatives of CypD inhibitors. Into PCR tubes, ester compound (6 μM) and either buffer only, CES1 (0.25 μM), or CES2 (0.25 μM) was diluted in 100 mM Tris-HCl buffer, pH 7.4, with a final DMSO concentration of 1%. Samples were maintained on a PCR block at 37° C. for 8 hours. After incubation, samples were diluted with 20 μL acetonitrile and analyzed by LC-MS at Harvard's Center for Mass Spectroscopy. Di-ester, mono-ester, and di-acid abundances were quantified by total ion count of the primary isotope, and the three compounds were summed and each one expressed as a fraction of the total sum.

Cellular esterase activity on ester pro-drug Cy5 derivatives of CypD inhibitors. Into 96-well clear, flat-bottom TC-treated plates, cells were seeded in DMEM supplemented with 10% FBS for HEK293T, HeLa, and MEFs, F-12K supplemented with 10% FBS for A549, or MEM supplemented with 10% FBS for HepG2, 24 hours prior at a density where wells were ˜70% confluent at time of experiment. Media was removed and cells were incubated with 100 μL of mixture containing each Cy5-conjugated compound (6 μM) in their respective media at 1% DMSO for 48 hours (36 hours for HepG2) at 37° C. at 5% CO2. Supernatant was removed, and cells were washed 2 times with 100 μL of PBS pH 7.4 (Gibco). Cells were then lysed for 15 minutes with 30 μL of 10 mM Tris-HCl pH 8.0 buffer containing 0.05% SDS. Lysates were diluted with 100 μL acetonitrile and filtered. Lysates were analyzed by LC-MS at Harvard's Center for Mass Spectroscopy. Di-ester, mono-ester, and di-acid abundances were quantified by total ion count of the primary isotope, and the three compounds were summed and each one expressed as a fraction of the total sum.

paxdb^{4.1} quantification of human cyclophilin abundance: Relative human cyclophilin abundances were calculated using paxdb^{4.1 47} filtered for Homo sapiens and each cyclophilin's gene identifier (FIG. 30). Ppm values were reported from calculated whole organism (integrated) abundance.

### Example 5. General Synthetic Methods

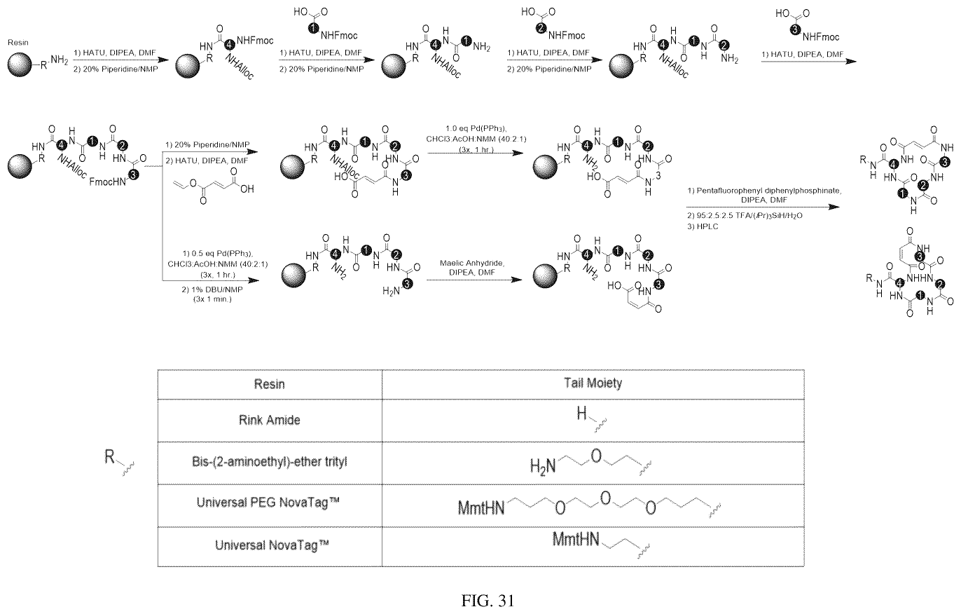

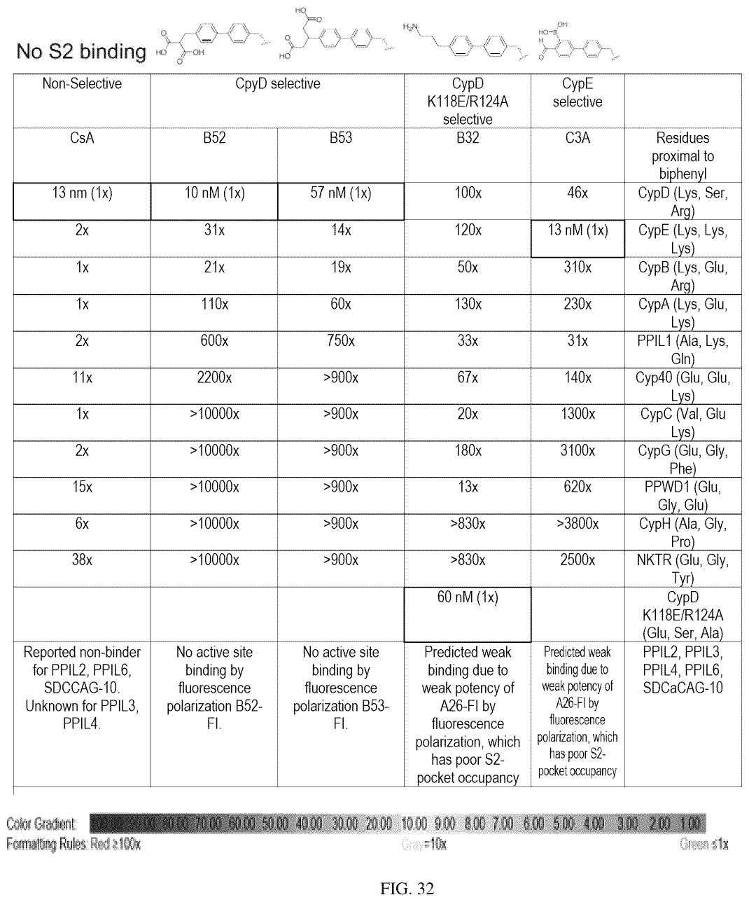

General procedure for solid-phase peptide synthesis of macrocycle inhibitors: Adapted from previous work⁵², appropriate resin shown in FIG. 31 (0.05-0.2 mmol) was swelled in ˜10-15 mL N,N′-dimethylformamide (DMF) for 1 hour in a peptide synthesis vessel with mixing provided by N₂ bubbling. In a separate flask, N-allyloxycarbonyl-N′-Fmoc-amino acid (4^{th} building block, 5 eq) and 1-[Bis(dimethylamino)methylene]-1H-1,2,3-triazolo[4,5-b]pyridinium 3-oxid hexafluorophosphate (HATU, 4.75 equiv.) was dissolved in ˜10-15 mL DMF then treated with N,N′-diisopropylethylamine (DIPEA, 10 eq) for 5 minutes, observing a color change. This mixture was added to the pre-swollen resin and mixed at RT for 1-2 hours. The supernatant was then eluted from the reaction vessel leaving isolated resin. Resin was washed three times with ˜5 mL DMF, then three times with ˜5 mL N-methyl-2-pyrrolidone (NMP). Fmoc cleavage of resin bound amino acid was facilitated by suspending the resin with three 5-minute treatments of ˜10 mL of 20% piperidine/NMP. After Fmoc cleavage, beads were washed three times with ˜5 mL NMP and three times with ˜5 mL DMF. The 1^{st}, 2^{nd} and 3^{rd} building blocks were then sequentially coupled onto resin bound 4^{th} building block utilizing the same described protocol for the 4^{th} building block. Macrocycles with Fmoc-2-aminomethyl-phenylacetic acid as a building block were instead treated three separate times with 2 eq/1.9 eq of this building block/HATU for 8 hours each. Upon coupling of the final 3^{rd} building block, terminal Fmoc group was left on the peptide. 3^{rd} building blocks with piperidine like structures were allowed to be coupled onto resin for 16 hours.

For cis-alkene isomer, resin was washed three times with CHCl₃ then suspended in degassed CHCl₃, acetic acid, and N-methylmorpholine (40:2:1 ratio). Allyloxy deprotection was then afforded by treating resin three times at RT for 1 hour each with tetrakis(triphenylphosphine)palladium(0) (0.5 eq total). Resin was then washed with ˜20 mL 5% DIPEA/DMF, ˜20 mL 5% (w/v) sodium diethyldithiocarbamate trihydrate in DMF, ˜20 mL of 5% (w/v) hydroxybenzotriazole monohydrate in DMF, then finally with ˜20 mL 1:1 CH₂Cl₂/DMF. In a separate flask, maleic anhydride (10 eq) was dissolved in DMF and treated with DIPEA (20 eq), turning a dark red/black color. Mixture was then added to resin and allowed to mix at RT for 1 hour. Resin was then washed five times with DMF to remove as much maleic anhydride as possible. Fmoc deprotection was then afforded by mixing resin with ˜10 mL of 1% 1,8-Diazabicyclo[5.4.0]undec-7-ene (DBU) in DMF, with three, 1 minute treatments. Resin was then washed three times with 20% DIPEA/DMF.

For trans-alkene isomer, the terminal, 3^{rd} building block Fmoc was cleaved using previously described deconditions with 20% piperidine/NMP. In a separate flask allyl-fumarate monoester⁵² (10 eq) was dissolved in DMF with HATU (9.5 eq) and treated with DIPEA (20 eq), observing a change of color to dark red/black. Mixture was then added to resin and allowed to mix at RT for 1 hour. Resin was then washed three times with DMF and three times with CHCl₃. Resin was then suspended in degassed CHCl₃, acetic acid, and N-methylmorpholine (40:2:1 ratio). Concurrent allyloxy and allyl deprotection was then afforded by treating resin three times at RT for 1 hour each with tetrakis(triphenylphosphine)palladium(0) (1.0 eq total). Resin was then washed with ˜20 mL 5% DIPEA/DMF, ˜20 mL 5% (w/v) sodium diethyldithiocarbamate trihydrate in DMF, ˜20 mL of 5% (w/v) hydroxybenzotriazole monohydrate in DMF, then finally with ˜20 mL CH₂Cl₂/DMF.

For both cis and trans isomer, macrocyclization was afforded by treating resin with pentafluorophenyl diphenylphosphinate (5 eq), anhydrous DIPEA (10 eq) dissolved in ˜10 mL anhydrous DMF for 3-16 hours. Resin was then washed three times with DMF, three times with CH₂Cl₂ and allowed to dry in the fume hood. Macrocycle product was then cleaved off resin with two treatments of ˜15 mL solution containing 95% trifluoroacetic acid (TFA), 2.5% triisopropylsilane (TIPS) and 2.5% water. Macrocycles sensitive to hydrosilane reduction were cleaved without TIPS. Resin was then washed two times with TFA. Combined supernatants were then dried to an oily residue through rotary evaporation. The peptide was the precipitated out of solution with Et₂O cooled to −78° C. Ether was then decanted and isolated precipitate was dissolved in a minimal amount of 3:1 DMF/water, and filtered through 0.22 L Ultrafree-MC Centrifugal Filters (Millipore Sigma). For hydrophobic macrocycles, no ether precipitation was conducted and sample was directly dissolved in 3:1 DMF/water. Sample was then purified by reverse phase HPLC, with a gradient of 10-60% acetonitrile/water containing 0.1% TFA over 40 minutes. Fractions that contained product was then freeze dried to produce a white powder. Yields for reported macrocycles varied from 1-10% of the initial resin loading.

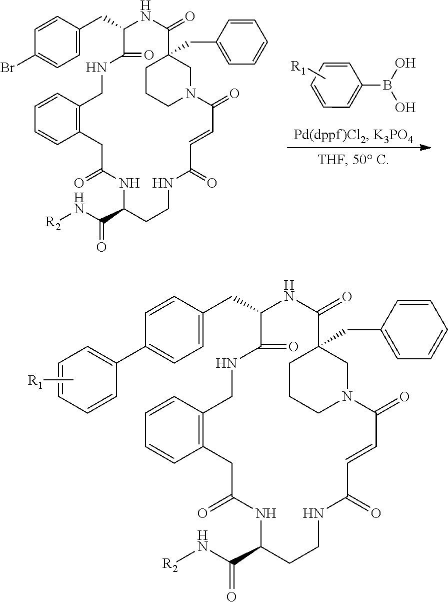

Generalized procedure A for Suzuki-Miyaura cross-coupling of macrocycles: Adapting conditions described previously⁷³, into a 10 mL screw-top vial, bromo-macrocycle (0.011 mmol, 1.0 eq), boronic acid (0.11 mmol, 10 eq), [1,1′-Bis(di-tert-butylphosphino)ferrocene]dichloropalladium (II) (0.0002 mmol, 0.2 eq), and potassium phosphate tribasic (0.23 mmol, 20 eq) was combined and sealed with a septum. Flask was evacuated and refilled with N₂ three times and left under N₂. Degassed THF (0.05 M), by cycling between vacuum and N₂ with concurrent sonication, was then added to the sealed flask and allowed to stir at 50° C. for 16 hours. After cooling, the reaction was cooled and solvent was removed under high-vac. Solid residue was dissolved in a minimal amount of 3:1 DMF/water with 5% TFA and filtered through 0.22 L Ultrafree®-MC Centrifugal Filters (Milipore-Sigma). Sample was then purified by reverse HPLC, with a 10-60% or 10-100% gradient of acetonitrile/water containing 0.1% TFA depending on hydrophobicity of compound. Fractions that contained product was then freeze dried to produce a white powder.

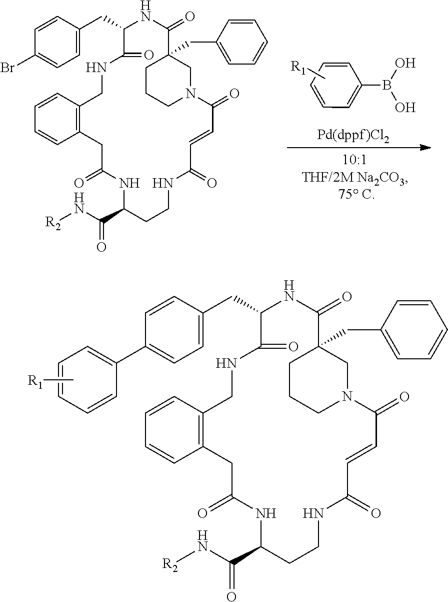

Generalized procedure B for Suzuki-Miyaura cross-coupling of macrocycles: Into a 10 mL screw-top vial, bromo-macrocycle (0.011 mmol, 1.0 eq), boronic acid (0.11 mmol, 10 eq), [1,1′-Bis(di-tert-butylphosphino)ferrocene]dichloropalladium (II) (0.0002 mmol, 0.2 eq), were combined and sealed with a septum. Flask was evacuated and refilled with N₂ three times and left under N₂. Degassed 10:1 THF/2M Na₂CO₃ (0.05 M) was added and reaction as heated to at 75° C. for 16 hours. After cooling, the reaction was cooled and solvent was removed under high-vac. Solid residue was dissolved in a minimal amount of 3:1 DMF/water with 5% TFA and filtered through 0.22 L Ultrafree®-MC Centrifugal Filters (Milipore-Sigma). Sample was then purified by reverse HPLC, with a 10-60% or 10-100% gradient of acetonitrile/water containing 0.1% TFA depending on hydrophobicity of compound. Fractions that contained product was then freeze dried to produce a white powder.

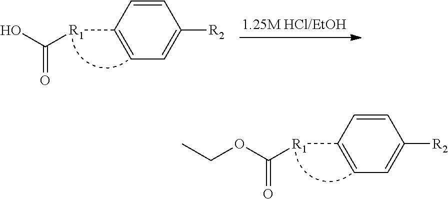

General conditions for ethyl ester protection of carboxylic acids: In a 50 mL roundbottom flask, ester (1 mmol, 1 eq) was suspended in 1.25 M HCl/EtOH at RT for 16 hours. Reaction was then diluted slowly with 100 mL saturated NaHCO₃, and aqueous layer was extracted three times with EtOAc. Combined organic layers were washed with saturated NaCl, dried over Na₂SO₄, and concentrated by rotary evaporation to afford ester product.

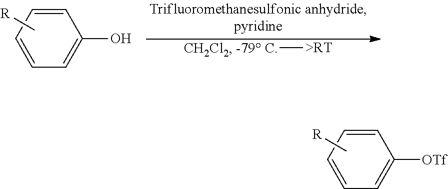

Generalized conditions for triflation of phenols: Into a N₂ flushed 100 mL roundbottom flask, phenol derivative (1.0 mmol, 1.0 eq), was dissolved in CH₂Cl₂ and cooled to −78° C. in a dry ice/acetone bath. After sequential dropwise addition of pyridine (3.0 mmol, 3.0 eq) and trifluoromethanesulfonic anhydride (1.1 mmol, 1.1 eq), reaction was allowed to stir for 16 hours, warming up to RT. Reaction was then diluted with 100 mL saturated NaHCO₃, and aqueous layer was extracted three times with EtOAc. Combined organic layers were washed with saturated NaCl, dried over Na₂SO₄, and concentrated by rotary evaporation to afford triflate product.

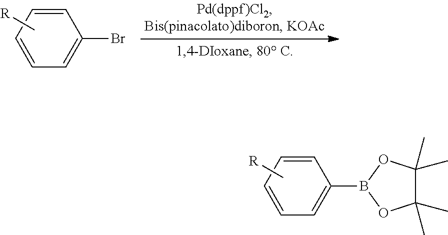

Generalized Miyaura borylation conditions: Into a 100 mL roundbottom flask, aryl bromide (1.0 mmol, 1.0 eq), bis(pinacolato)diboron (2.0 mmol, 2 eq), [1,1′-Bis(diphenylphosphino)ferrocene]dichloropalladium (II) (0.1 mmol, 0.1 eq), and potassium acetate (3.0 mmol, 3 eq) was combined and sealed with a septum. Flask was evacuated and refilled with N₂ three times and left under N₂. Degassed 1,4-dioxane (0.1 M), by cycling between vacuum and N₂ with concurrent sonication, was then added to the sealed flask and allowed to stir at 80° C. for 16 hours. After cooling, the reaction was cooled and diluted with 1 M HCl and transferred to a separatory funnel. Aqueous layer was extracted three times with EtOAc and combined organic layers were washed with saturated NaCl, dried over Na₂SO₄, and concentrated by rotary evaporation. Residue was purified by column chromatography to afford product.

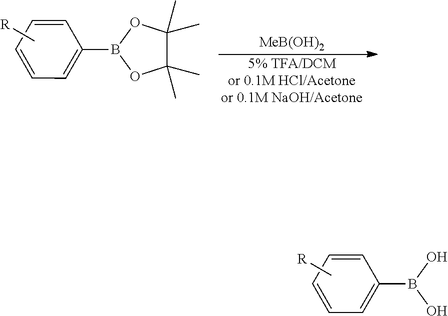

General pinacol deprotection conditions: Adapting conditions described previously⁷⁴, in a 50 mL roundbottom flask, pinacol boronic ester (1 mmol, 1 eq) and methylboronic acid (10 mmol, 10 eq) were suspended in 5% TFA/CH₂Cl₂, 0.1 M HCl/Acetone, or 0.1 M NaOH/Acetone depending on acid sensitivity of substrate and allowed to stir at RT for 16 hours. Solvent was removed by rotary evaporation and the residue was resuspended in 0.1 M HCl. HCl was removed by rotary evaporation, removing excess methylboronic acid and methylboronic acid pinacol ester. This cycle was repeated three times until no methylboronic acid was present by NMR analysis. For acid sensitive substrates, residue was resuspended in water during rotary evaporation steps. For substrates dissolved in 0.1 M NaOH/Acetone, solution was neutralized to ˜pH 3, filtered, and concentrated by rotary evaporation. Excess MeB(OH)₂ was removed as described above using water.

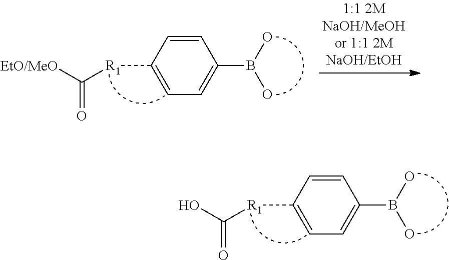

General conditions for saponification of methyl/ethyl esters: In a 50 mL round bottom flask, ester (1 mmol, 1 eq) was suspended in 1:1 2 M NaOH/MeOH (0.1 M) for methyl esters or 1:1 2 M NaOH/EtOH (0.1 M) for ethyl esters and stirred at RT for 15 hours. Reaction was neutralized by diluting reaction with 50 mL 1 M HCl, and aqueous layer was extracted three times with EtOAc. Combined organic layers were washed with saturated NaCl, dried over Na₂SO₄, and concentrated by rotary evaporation to isolated carboxylic acid.

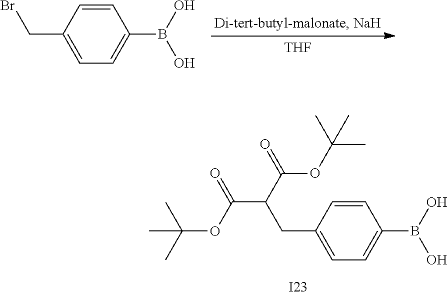

I23: Adapting a previously reported protocol⁷⁶, into a N₂ flushed 50 mL roundbottom flask, sodium hydride (60% dispersion in mineral oil) (1.47 mmol, 1.05 eq) was dissolved in anhydrous THF (0.1 M) followed by dropwise addition of di-tert-butyl-malonate. Once gas finished forming from the solution, 4-(Bromomethyl)phenylboronic acid dissolved in 1 mL THF was added to the flask, which was allowed to stir at room temperature for 16 hours. The reaction was then diluted with saturated NH₄Cl and extracted three times with EtOAc. Combined organic layers were washed with saturated NaCl, dried over Na₂SO₄, and concentrated by rotary evaporation. Residue was purified by silica gel chromatography (0-10% MeOH/CH₂Cl₂) to afford product as white solid. Yield: 84%

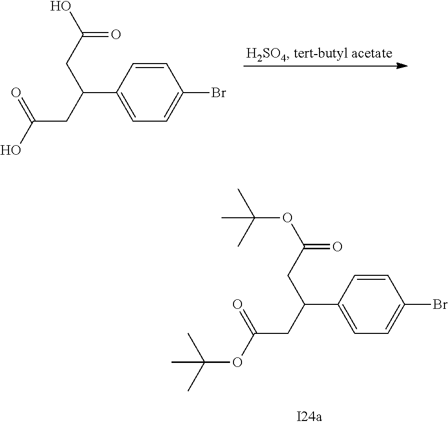 [SMILES: O=C(O)CC(CC(=O)O)c1ccc(Br)cc1 ; CC(C)(C)OC(=O)CC(CC(=O)OC(C)(C)C)c1ccc(Br)cc1]

I24a: Adopting a previously reported protocol⁷⁷, into a N₂ flushed 50 mL roundbottom flask, B-(4-Bromophenyl)glutaric acid (0.7 mmol, 1.0 eq) was suspended in tert-butyl-acetate (0.2 M), followed by addition of concentrated H₂SO₄ (0.7 mmol, 1 eq). Reaction was allowed to stir at room temperature for 16 hours. Reaction was diluted with sat. bicarbonate and extracted three times with EtOAc. Combined organic layers were washed with saturated NaCl, dried over Na₂SO₄, and concentrated by rotary evaporation. Residue was purified by column chromatography (0-50% EtOAc/Hex) to afford product as white solid. Yield: 47%

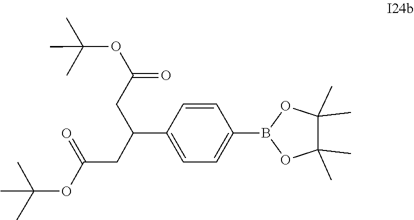 [SMILES: CC(C)(C)OC(=O)CC(CC(=O)OC(C)(C)C)c1ccc(B2OC(C)(C)C(C)(C)O2)cc1]

I24b: Compound was synthesized using generalized Miyaura borylation reaction procedure with with I24a and no acidic workup. Crude reaction was directly purified by column chromatography (0-50% EtOAc/Hex) to afford product as white powder with trace amounts of bis(pinacolato)diboron. Yield: 97%

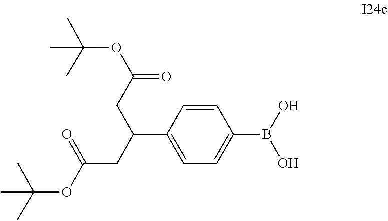 [SMILES: CC(C)(C)OC(=O)CC(CC(=O)OC(C)(C)C)c1ccc(B(O)O)cc1]

I24c: Compound was synthesized using general pinacol deprotection conditions with I24b and substituting 0.1 N NaOH/Acetone as solvent. After 16 hours, reaction was neutralized to ˜pH 2-3 with 1N HCl and filtered to remove salts. Solvent was removed by rotary evaporation and the residue was resuspended in H₂O and once again removed by rotary evaporation to remove excess MeB(OH)₂. This step was repeated until all MeB(OH)₂ was removed as seen on ¹H NMR. Product was isolated as a white solid with small impurities. Product was carried on to next step without further purification Yield: quantitative

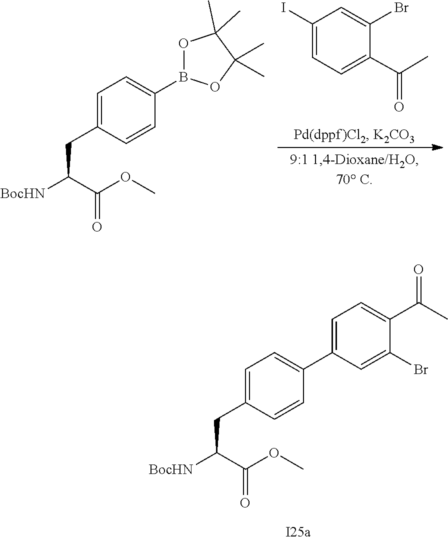

I25a: Adapting a previously reported protocol⁷⁸, a mixture of Boc-4-borono pinacol ester-L phenylalanine methyl ester (1.98 mmol, 1 eq.), 1-(2-Bromo-4-iodophenyl)ethanone (2.56 mmol, 1.3 eq.), [1,1-Bis(diphenylphosphino)ferrocene]dichloropalladium(II) (0.19 mmol, 0.1 eq.), and potassium carbonate (7.90 mmol, 4 eq.) was dissolved in 18 mL of degassed 1,4-dioxane and 2 mL of degassed water (0.1 M) and stirred at 70° C. in a sealed round bottom flask overnight. The reaction was then cooled down to room temperature, diluted with water, extracted three times with ethyl acetate (EtOAc), washed with brine, dried over sodium sulfate (Na₂SO₄), and evaporated under reduced pressure. The crude product was then dry loaded onto a 25G SNAP column, purified by flash column chromatography using 0-50% EtOAc/Hexanes, and yielded I25a as a yellow foam. Yield: 42% yield

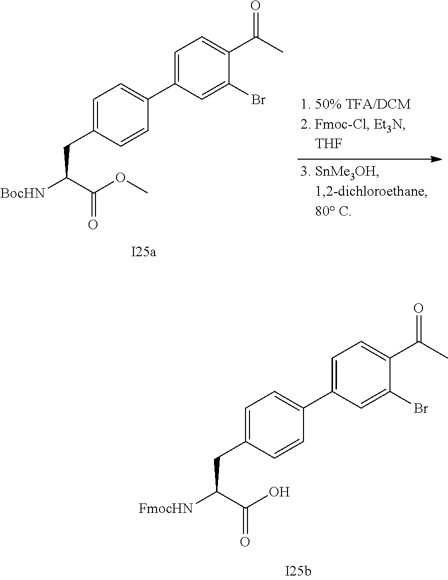

I25b: Compound I25a (0.84 mmol, 1 eq.) was dissolved in 4.5 mL trifluoroacetic acid and 4.5 mL dichloromethane (0.1 M solution) to remove the Boc protecting group. The reaction was stirred for one hour at room temperature and the solvent was evaporated under reduced pressure and dried on high vacuum overnight. Then, a mixture of the crude Boc deprotected product and sodium bicarbonate (4.21 mmol, 5 eq.) was dissolved in 2 mL of 1,4-dioxane and 4 mL of water and stirred. 9-Fluorenylmethoxycarbonyl chloride (1.05 mmol, 1.25 eq.) was separately dissolved in 2 mL of 1,4-dioxane and added to the reaction. The reaction was stirred overnight at room temperature, then suspended in water, extracted three times with EtOAc, dried over Na₂SO₄, and evaporated under reduced pressure. To deprotect the methyl ester, a mixture of the crude Fmoc protected product and trimethyltin hydroxide (1.26 mmol, 1.5 eq.) was dissolved in 8 mL of 1,2-dichloroethane (0.1 M) and stirred at 80° C. under reflux for three hours. The reaction was then cooled down to room temperature, suspended in ethyl acetate, washed two times with 1 M hydrochloric acid, and dried over Na₂SO₄. The crude product was then dry loaded onto a 10G SNAP column, purified by flash column chromatography using 0-100% EtOAc/Hexanes, and yielded I25b as a yellow solid. Yield: 76%

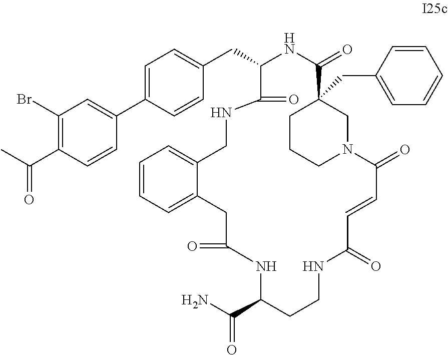 [SMILES: CC(=O)c1ccc(-c2ccc(C[C@@H]3NC(=O)[C@]4(Cc5ccccc5)CCCN(C4)C(=O)/C=C/C(=O)NCC[C@@H](C(N)=O)NC(=O)Cc4ccccc4CNC3=O)cc2)cc1Br]

I25c: Macrocycle was synthesized using general procedure for solid-phase peptide synthesis of macrocycles inhibitors. I25c was obtained as a white powder. Yield: 7%
Resin: Rink Amide
4^{th} Building Block: N^{α}-Fmoc-N^{γ}-allyloxycarbonyl-L-2,4-diaminobutyric acid
1^{st} Building Block: Fmoc-2-aminomethyl-phenylacetic acid
2^{nd} Building Block: I25b
3^{rd} Building Block: (S)-Fmoc-3-benzyl-piperidine-3-carboxylic acid

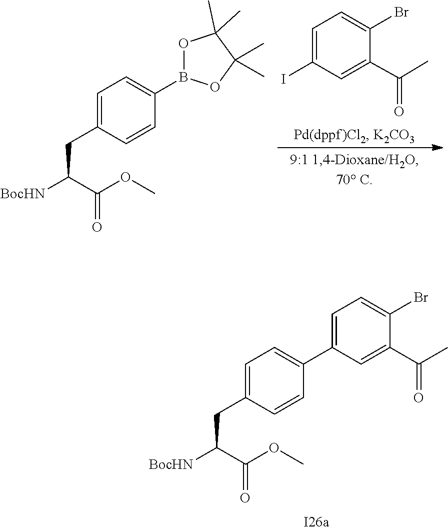

I26a: Compound was synthesized and purified as described in I25a with 1-(2-Bromo-5-iodophenyl)ethanone, affording I26a as a yellow foam. Yield: 37% yield

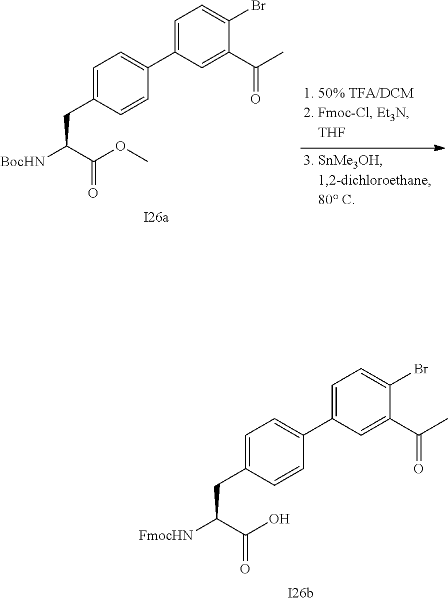

I26b: Compound was synthesized and purified as described in I25b, with I26a (0.73 mmol, 1 eq.), affording I26b as a yellow solid. Yield: 55%

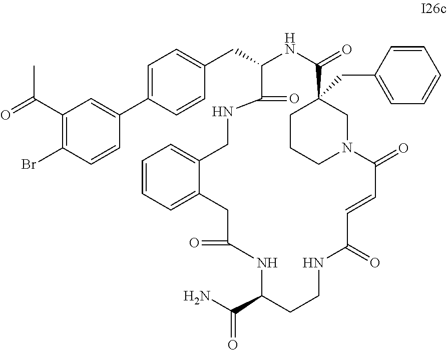 [SMILES: CC(=O)c1cc(-c2ccc(C[C@@H]3NC(=O)[C@]4(Cc5ccccc5)CCCN(C4)C(=O)/C=C/C(=O)NCC[C@@H](C(N)=O)NC(=O)Cc4ccccc4CNC3=O)cc2)ccc1Br]

I26c: Macrocycle was assembled using general procedure for solid-phase peptide synthesis of macrocycles inhibitors and was obtained as a white powder. Yield: 7%
Resin: Rink Amide
4^{th} Building Block: N^{α}-Fmoc-N^{γ}-allyloxycarbonyl-L-2,4-diaminobutyric acid
1^{st} Building Block: Fmoc-2-aminomethyl-phenylacetic acid
2^{nd} Building Block: I26b
3^{rd} Building Block: (S)-Fmoc-3-benzyl-piperidine-3-carboxylic acid

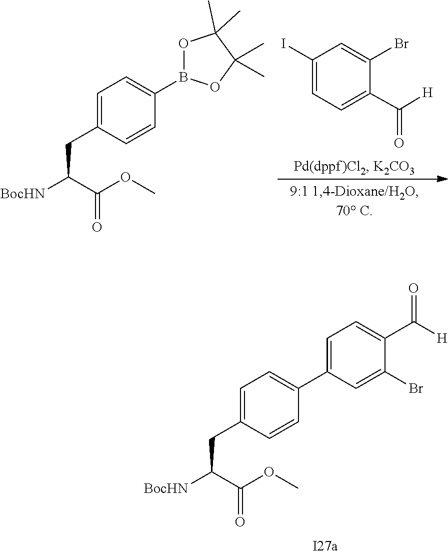

I27a: Compound was synthesized and purified as described in I25a with 2-Bromo-4-iodobenzaldehyde, affording I27a as a yellow foam. Yield: 46%

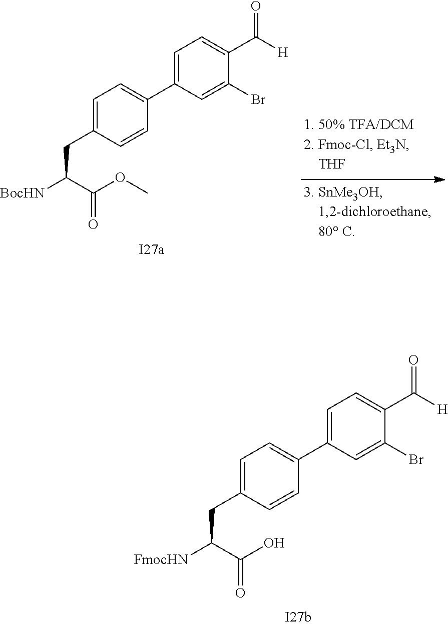

I27b: Compound was synthesized and purified as described in I25b, with I27a (0.91 mmol, 1 eq.), affording I27b as a yellow solid. Yield: 63%

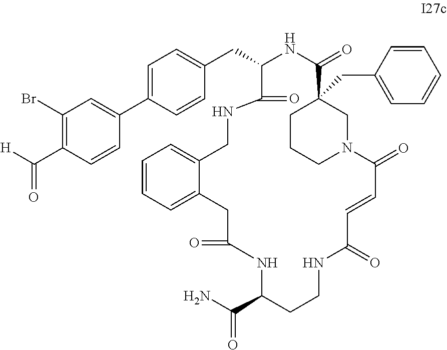 [SMILES: NC(=O)[C@@H]1CCNC(=O)/C=C/C(=O)N2CCC[C@](Cc3ccccc3)(C2)C(=O)N[C@@H](Cc2ccc(-c3ccc(C=O)c(Br)c3)cc2)C(=O)NCc2ccccc2CC(=O)N1]

I27c: Macrocycle was assembled using general procedure for solid-phase peptide synthesis of macrocycle inhibitors and was obtained as a white powder. Yield: 1%
Resin: Rink Amide
4^{th} Building Block: N^{α}-Fmoc-N^{γ}-allyloxycarbonyl-L-2,4-diaminobutyric acid
1^{st} Building Block: Fmoc-2-aminomethyl-phenylacetic acid
2^{nd} Building Block: I27b
3^{rd} Building Block: (S)-Fmoc-3-benzyl-piperidine-3-carboxylic acid

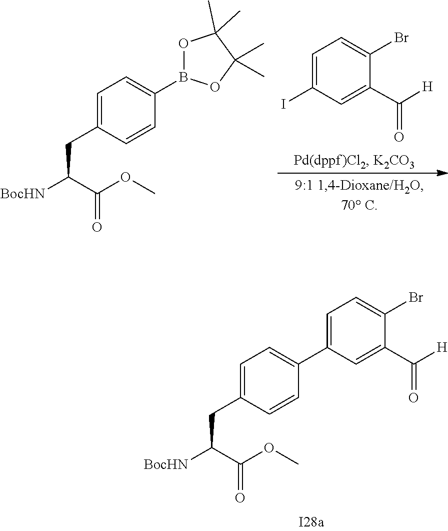

I28a: Compound was synthesized and purified as described in I25a with 2-Bromo-5-iodobenzaldehyde, affording I28a as a yellow foam. Yield: 42%

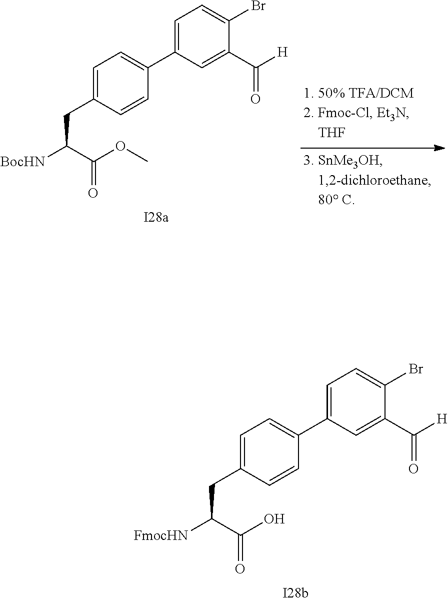

I28b: Compound was synthesized and purified as described in I25b, with I28a (0.87 mmol, 1 eq.), affording I28b as a yellow solid. Yield: 36%

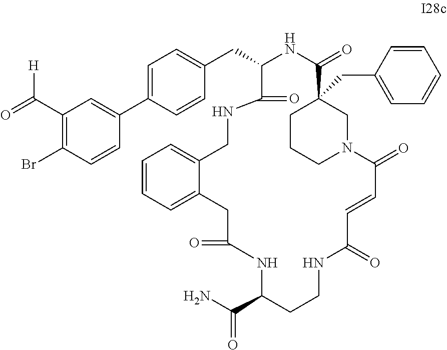 [SMILES: NC(=O)[C@@H]1CCNC(=O)/C=C/C(=O)N2CCC[C@](Cc3ccccc3)(C2)C(=O)N[C@@H](Cc2ccc(-c3ccc(Br)c(C=O)c3)cc2)C(=O)NCc2ccccc2CC(=O)N1]

I28c: Macrocycle was assembled using general procedure for solid-phase peptide synthesis of macrocycles inhibitors and was obtained as a white powder. Yield: 1%
Resin: Rink Amide
4^{th} Building Block: N^{α}-Fmoc-N^{γ}-allyloxycarbonyl-L-2,4-diaminobutyric acid
1^{st} Building Block: Fmoc-2-aminomethyl-phenylacetic acid
2^{nd} Building Block: I28b
3^{rd} Building Block: (S)-Fmoc-3-benzyl-piperidine-3-carboxylic acid

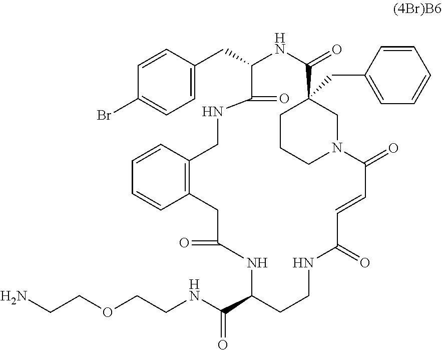 [SMILES: NCCOCCNC(=O)[C@@H]1CCNC(=O)/C=C/C(=O)N2CCC[C@](Cc3ccccc3)(C2)C(=O)N[C@@H](Cc2ccc(Br)cc2)C(=O)NCc2ccccc2CC(=O)N1]

(4Br)B6: Macrocycle was assembled using general procedure for solid-phase peptide synthesis of macrocycle inhibitors.
Resin: Bis-(2-aminoethyl)-ether trityl
4^{th} Building Block: N^{α}-Fmoc-N^{γ}-allyloxycarbonyl-L-2,4-diaminobutyric acid
1^{st} Building Block: Fmoc-2-aminomethyl-phenylacetic acid
2^{nd} Building Block: Fmoc-β-(2-furyl)-Ala-OH
3^{rd} Building Block: (S)-Fmoc-3-benzyl-piperidine-3-carboxylic acid

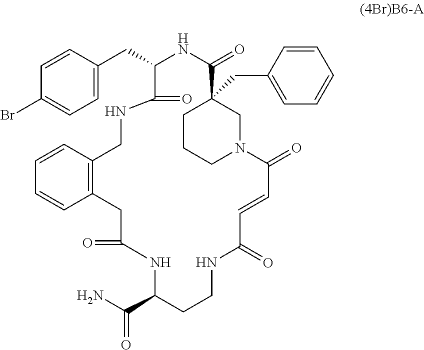 [SMILES: NC(=O)[C@@H]1CCNC(=O)/C=C/C(=O)N2CCC[C@](Cc3ccccc3)(C2)C(=O)N[C@@H](Cc2ccc(Br)cc2)C(=O)NCc2ccccc2CC(=O)N1]

(4Br)B6-A: Macrocycle was assembled using general procedure for solid-phase peptide synthesis of macrocycle inhibitors.
Resin: NovaPEG Rink Amide
4^{th} Building Block: N^{α}-Fmoc-N^{γ}-allyloxycarbonyl-L-2,4-diaminobutyric acid
1^{st} Building Block: Fmoc-2-aminomethyl-phenylacetic acid
2^{nd} Building Block: Fmoc-β-(2-furyl)-Ala-OH
3^{rd} Building Block: (S)-Fmoc-3-benzyl-piperidine-3-carboxylic acid

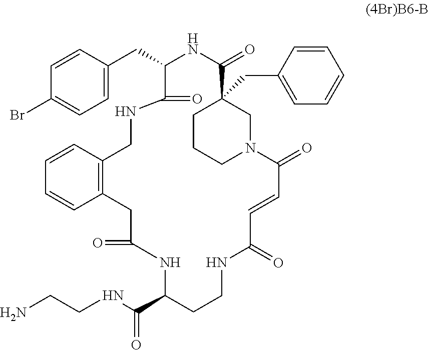 [SMILES: NCCNC(=O)[C@@H]1CCNC(=O)/C=C/C(=O)N2CCC[C@](Cc3ccccc3)(C2)C(=O)N[C@@H](Cc2ccc(Br)cc2)C(=O)NCc2ccccc2CC(=O)N1]

(4Br)B6-B: Macrocycle was assembled using general procedure for solid-phase peptide synthesis of macrocycle inhibitors.
Resin: Universal NovaTag
4^{th} Building Block: N^{α}-Fmoc-N^{γ}-allyloxycarbonyl-L-2,4-diaminobutyric acid
1^{st} Building Block: Fmoc-2-aminomethyl-phenylacetic acid
2^{nd} Building Block: Fmoc-β-(2-furyl)-Ala-OH
3^{rd} Building Block: (S)-Fmoc-3-benzyl-piperidine-3-carboxylic acid

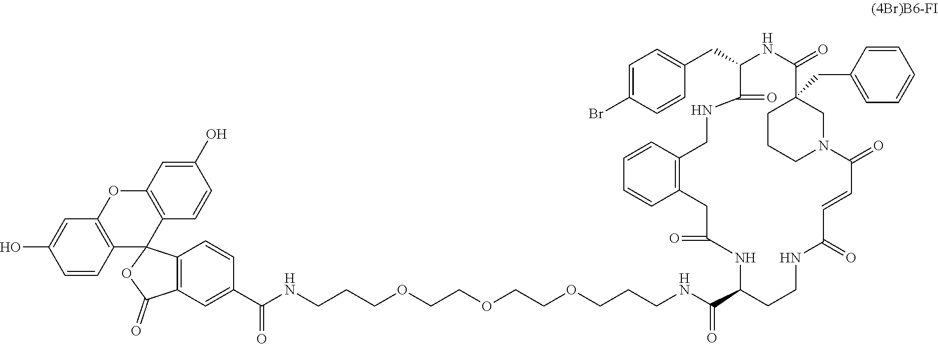 [SMILES: O=C1/C=C/C(=O)N2CCC[C@](Cc3ccccc3)(C2)C(=O)N[C@@H](Cc2ccc(Br)cc2)C(=O)NCc2ccccc2CC(=O)N[C@H](C(=O)NCCCOCCOCCOCCCNC(=O)c2ccc3c(c2)C(=O)OC32c3ccc(O)cc3Oc3cc(O)ccc32)CCN1]

(4Br)B6-Fl: Macrocycle was assembled using general procedure for solid-phase peptide synthesis of macrocycle inhibitors. Prior to acidic cleavage, resin was suspended with three 1-hour treatments of 1M hydroxybenzotriazole monohydrate dissolved in 1:1 CH₂Cl₂/trifluoroethanol to remove Mmt group. 5-carboxyfluorescein (5 eq) and (HATU, 4.75 equiv.) were dissolved in a separate flask in ˜10-15 mL DMF then treated with N,N′-diisopropylethylamine (DIPEA, 10 eq) for 5 minutes, observing a color change. This flask was then transferred to resin swollen in ˜10 mL DMF and allowed to mix for 24 hours. Fluorescein labeled peptide were then cleaved from resin and purified as described in general procedure.
Resin: Universal PEG NovaTag™_Resin
4^{th} Building Block: N^{α}-Fmoc-N^{γ}-allyloxycarbonyl-L-2,4-diaminobutyric acid
1^{st} Building Block: Fmoc-2-aminomethyl-phenylacetic acid
2^{nd} Building Block: Fmoc-β-(2-furyl)-Ala-OH
3^{rd} Building Block: (S)-Fmoc-3-benzyl-piperidine-3-carboxylic acid

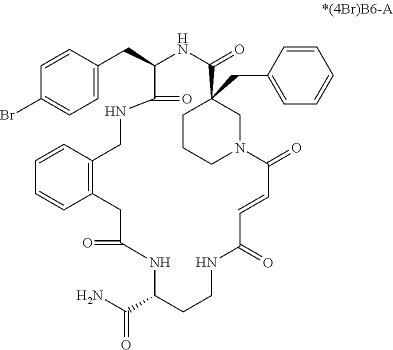 [SMILES: NC(=O)[C@H]1CCNC(=O)/C=C/C(=O)N2CCC[C@](Cc3ccccc3)(C2)C(=O)N[C@H](Cc2ccc(Br)cc2)C(=O)NCc2ccccc2CC(=O)N1]

*(4Br)B6-A: Macrocycle was assembled using general procedure for solid-phase peptide synthesis of macrocycle inhibitors.
Resin: NovaPEG Rink Amide
4^{th} Building Block: N^{α}-Fmoc-N^{γ}-allyloxycarbonyl-D-2,4-diaminobutyric acid
1^{st} Building Block: Fmoc-2-aminomethyl-phenylacetic acid
2^{nd} Building Block: Fmoc-β-(2-furyl)-D-Ala-OH
3^{rd} Building Block: (R)-Fmoc-3-benzyl-piperidine-3-carboxylic acid

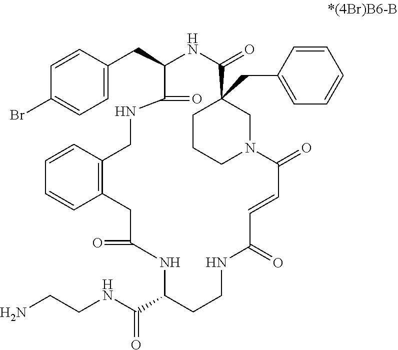 [SMILES: NCCNC(=O)[C@H]1CCNC(=O)/C=C/C(=O)N2CCC[C@](Cc3ccccc3)(C2)C(=O)N[C@H](Cc2ccc(Br)cc2)C(=O)NCc2ccccc2CC(=O)N1]

*(4Br)B6-B: Macrocycle was assembled using general procedure for solid-phase peptide synthesis of macrocycle inhibitors.
Resin: Universal NovaTag
4^{th} Building Block: N^{α}-Fmoc-N^{γ}-allyloxycarbonyl-D-2,4-diaminobutyric acid
1^{st} Building Block: Fmoc-2-aminomethyl-phenylacetic acid
2^{nd} Building Block: Fmoc-β-(2-furyl)-D-Ala-OH
3^{rd} Building Block: (R)-Fmoc-3-benzyl-piperidine-3-carboxylic acid

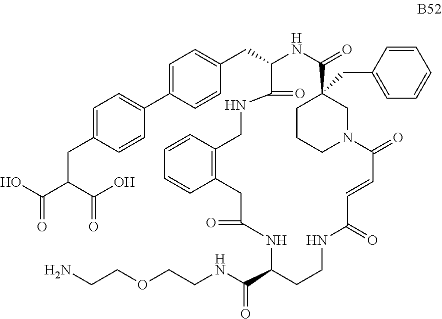 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccc(CC(C(=O)O)C(=O)O)cc3)cc2)C(=O)NCc2ccccc2CC(=O)N1]

B52: Compound was synthesized using Generalized procedure A for Suzuki-Miyaura cross-coupling of macrocycles using (4Br)B6 and 123. Tert-butyl protected product was treated with 1 mL TFA for 1 hour, and re-purified under same HPLC conditions to yield carboxylic acid product. Yield: 10%

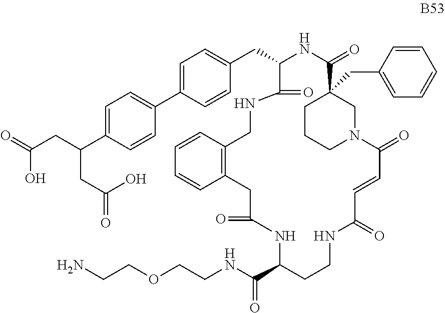 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccc(C(CC(=O)O)CC(=O)O)cc3)cc2)C(=O)NCc2ccccc2CC(=O)N1]

B53: Compound was synthesized using Generalized procedure A for Suzuki-Miyaura cross-coupling of macrocycles using (4Br)B6 and I24c. Tert-butyl protected product was treated with 1 mL TFA for 1 hour, and re-purified under same HPLC conditions to yield carboxylic acid product. Yield: 21%

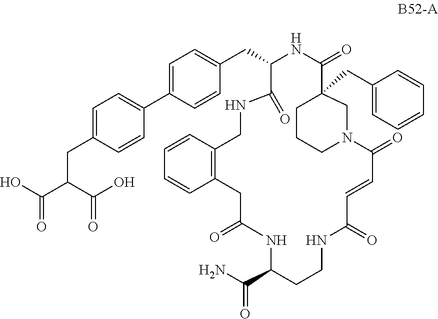 [SMILES: NC(=O)[C@@H]1CCNC(=O)/C=C/C(=O)N2CCC[C@](Cc3ccccc3)(C2)C(=O)N[C@@H](Cc2ccc(-c3ccc(CC(C(=O)O)C(=O)O)cc3)cc2)C(=O)NCc2ccccc2CC(=O)N1]

B52A: Compound was synthesized using Generalized procedure A for Suzuki-Miyaura cross-coupling of macrocycles using (4Br)B6-A and I23. Tert-butyl protected product was treated with 1 mL TFA for 1 hour, and re-purified under same HPLC conditions to yield carboxylic acid product. Yield: 40%

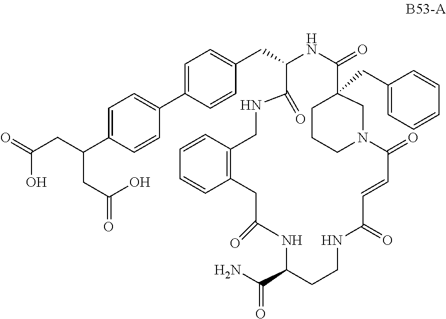 [SMILES: NC(=O)[C@@H]1CCNC(=O)/C=C/C(=O)N2CCC[C@](Cc3ccccc3)(C2)C(=O)N[C@@H](Cc2ccc(-c3ccc(C(CC(=O)O)CC(=O)O)cc3)cc2)C(=O)NCc2ccccc2CC(=O)N1]

B53-A: Compound was synthesized using Generalized procedure A for Suzuki-Miyaura cross-coupling of macrocycles using (4Br)B6-A and I24c. Tert-butyl protected product was treated with 1 mL TFA for 1 hour, and re-purified under same HPLC conditions to yield carboxylic acid product. Yield: 86%

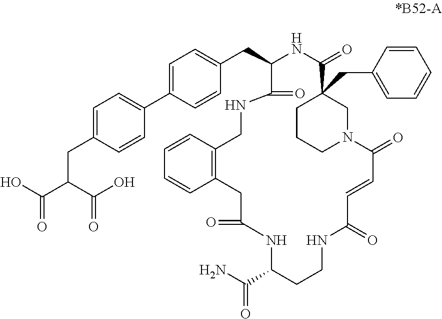 [SMILES: NC(=O)[C@H]1CCNC(=O)/C=C/C(=O)N2CCC[C@](Cc3ccccc3)(C2)C(=O)N[C@H](Cc2ccc(-c3ccc(CC(C(=O)O)C(=O)O)cc3)cc2)C(=O)NCc2ccccc2CC(=O)N1]

*B52-A: Compound was synthesized using Generalized procedure A for Suzuki-Miyaura cross-coupling of macrocycles using *(4Br)B6-A and 123. Tert-butyl protected product was treated with 1 mL TFA for 1 hour, and re-purified under same HPLC conditions to yield carboxylic acid product. Yield: 21%.

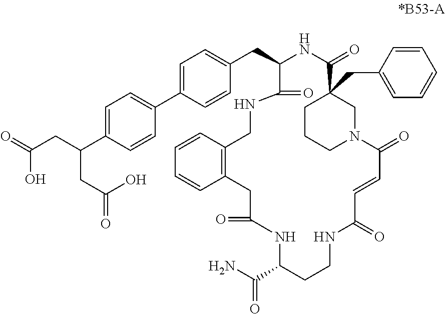 [SMILES: NC(=O)[C@H]1CCNC(=O)/C=C/C(=O)N2CCC[C@](Cc3ccccc3)(C2)C(=O)N[C@H](Cc2ccc(-c3ccc(C(CC(=O)O)CC(=O)O)cc3)cc2)C(=O)NCc2ccccc2CC(=O)N1]

*B53-A: Compound was synthesized using Generalized procedure A for Suzuki-Miyaura cross-coupling of macrocycles using *(4Br)B6-A and I24c. Tert-butyl protected product was treated with 1 mL TFA for 1 hour, and re-purified under same HPLC conditions to yield carboxylic acid product. Yield: 85%.

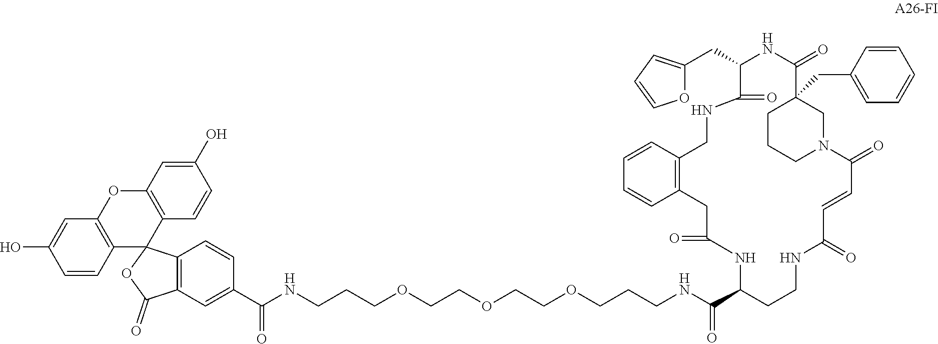 [SMILES: O=C1/C=C/C(=O)N2CCC[C@](Cc3ccccc3)(C2)C(=O)N[C@@H](Cc2ccco2)C(=O)NCc2ccccc2CC(=O)N[C@H](C(=O)NCCCOCCOCCOCCCNC(=O)c2ccc3c(c2)C(=O)OC32c3ccc(O)cc3Oc3cc(O)ccc32)CCN1]

A26-Fl: Macrocycle was assembled using general procedure for solid-phase peptide synthesis of macrocycle inhibitors. Prior to acidic cleavage, resin was suspended with three 1-hour treatments of 1 M hydroxybenzotriazole monohydrate dissolved in 1:1 CH₂Cl₂/trifluoroethanol to remove Mmt group. 5-carboxyfluorescein (5 eq) and (HATU, 4.75 equiv.) were dissolved in a separate flask in ˜10-15 mL DMF then treated with N,N″-diisopropylethylamine (DIPEA, 10 eq) for 5 minutes, observing a color change. This flask was then transferred to resin swollen in ˜10 mL DMF and allowed to mix for 24 hours. Fluorescein labeled peptide were then cleaved from resin and purified as described in general procedure.
Resin: Universal PEG NovaTag™_Resin
4^{th} Building Block: N^{α}-Fmoc-N^{γ}-allyloxycarbonyl-L-2,4-diaminobutyric acid
1^{st} Building Block: Fmoc-2-aminomethyl-phenylacetic acid
2^{nd} Building Block: Fmoc-β-(2-furyl)-Ala-OH
3^{rd} Building Block: (S)-Fmoc-3-benzyl-piperidine-3-carboxylic acid

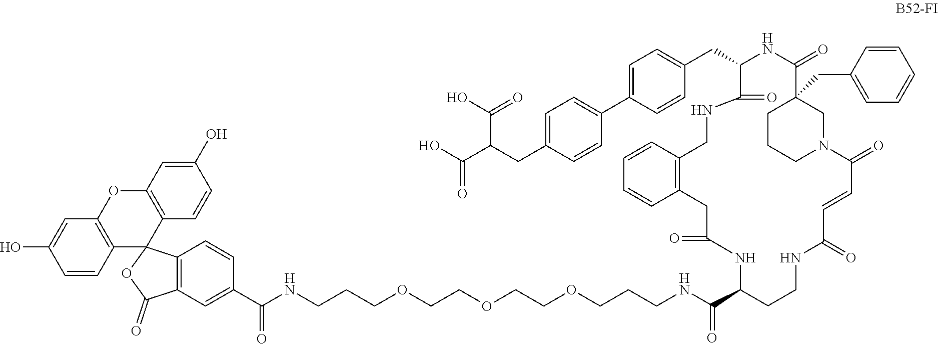 [SMILES: O=C1/C=C/C(=O)N2CCC[C@](Cc3ccccc3)(C2)C(=O)N[C@@H](Cc2ccc(-c3ccc(CC(C(=O)O)C(=O)O)cc3)cc2)C(=O)NCc2ccccc2CC(=O)N[C@H](C(=O)NCCCOCCOCCOCCCNC(=O)c2ccc3c(c2)C(=O)OC32c3ccc(O)cc3Oc3cc(O)ccc32)CCN1]

B52-Fl: Compound was synthesized using Generalized procedure A for Suzuki-Miyaura cross-coupling of macrocycles using (4Br)B6-Fl and 123. Tert-butyl protected product was treated with 1 mL TFA for 1 hour, and re-purified under same HPLC conditions to yield carboxylic acid product. Yield: 32%.

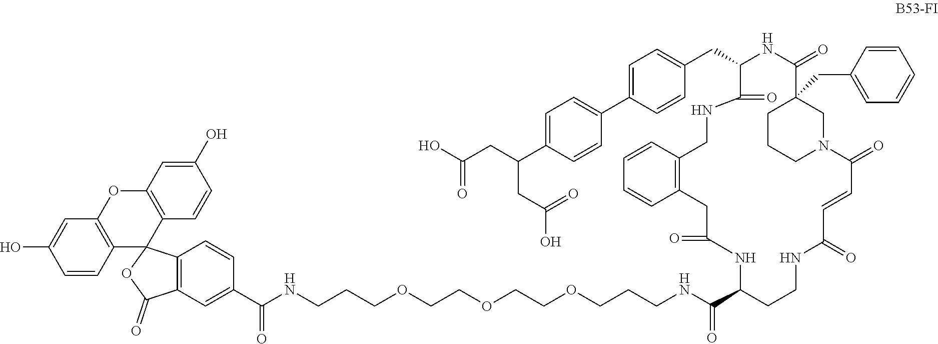 [SMILES: O=C(O)CC(CC(=O)O)c1ccc(-c2ccc(C[C@@H]3NC(=O)[C@]4(Cc5ccccc5)CCCN(C4)C(=O)/C=C/C(=O)NCC[C@@H](C(=O)NCCCOCCOCCOCCCNC(=O)c4ccc5c(c4)C(=O)OC54c5ccc(O)cc5Oc5cc(O)ccc54)NC(=O)Cc4ccccc4CNC3=O)cc2)cc1]

B53-Fl: Compound was synthesized using Generalized procedure A for Suzuki-Miyaura cross-coupling of macrocycles using (4Br)B6-Fl and I24c. Tert-butyl protected product was treated with 1 mL TFA for 1 hour, and re-purified under same HPLC conditions to yield carboxylic acid product. Yield: 33%

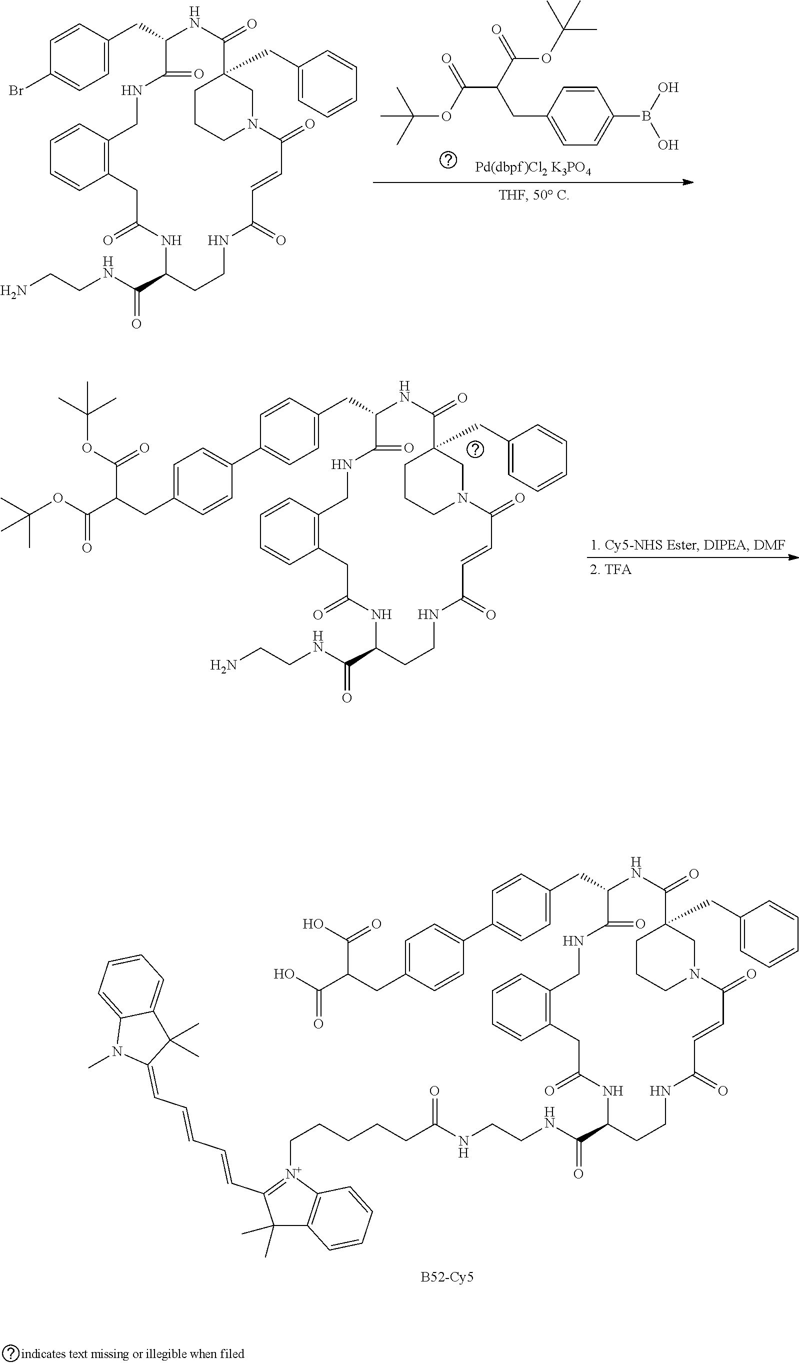

B52-Cy5: Compound was synthesized using Generalized procedure A for Suzuki-Miyaura cross-coupling of macrocycles using (4Br)B6-B and 123. Tert-butyl protected product was dissolved in a 1.5 mL Eppendorf tube with 100 μL DMF and mixed with DIPEA (5 eq.). Cy5-NHS ester (Lumiprobe) (2 eq.) dissolved in 50 μL DMF was then added to the flask allowed to stir for 1 hour. Resulting crude mixture was purified under same HPLC conditions to yield Cy5 coupled product. This product was then treated with 1 mL TFA for 1 hour, and re-purified under same HPLC conditions to yield carboxylic acid product. Yield: 25%.

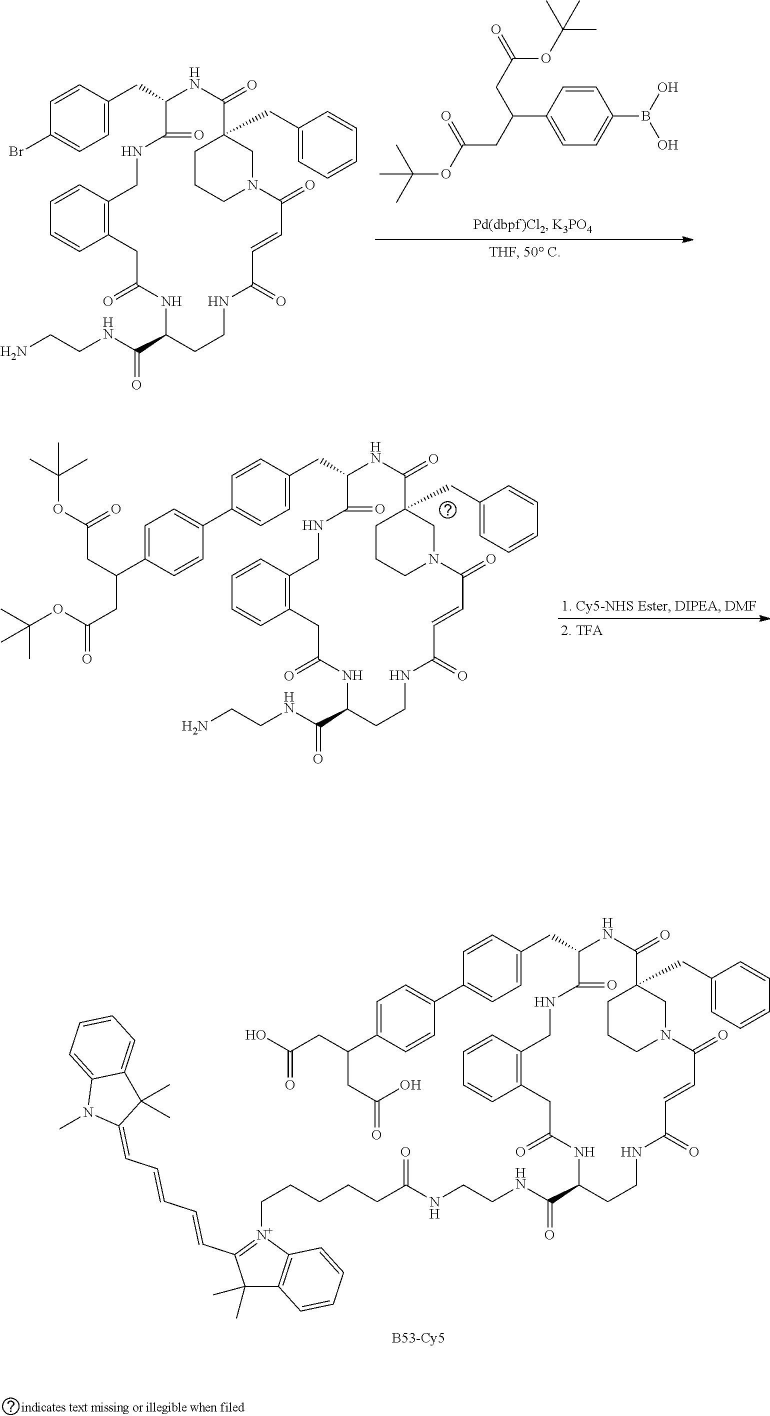

B53-Cy5: Compound was synthesized using Generalized procedure A for Suzuki-Miyaura cross-coupling of macrocycles using (4Br)B6-B and I24c. Tert-butyl protected product was dissolved in a 1.5 mL Eppendorf tube with 100 μL DMF and mixed with DIPEA (5 eq.). Cy5-NHS ester (Lumiprobe) (2 eq.) dissolved in 50 μL DMF was then added to the flask allowed to stir for 1 hour. Resulting crude mixture was purified under same HPLC conditions to yield Cy5 coupled product. This product was then treated with 1 mL TFA for 1 hour, and re-purified under same HPLC conditions to yield carboxylic acid product. Yield: 6%.

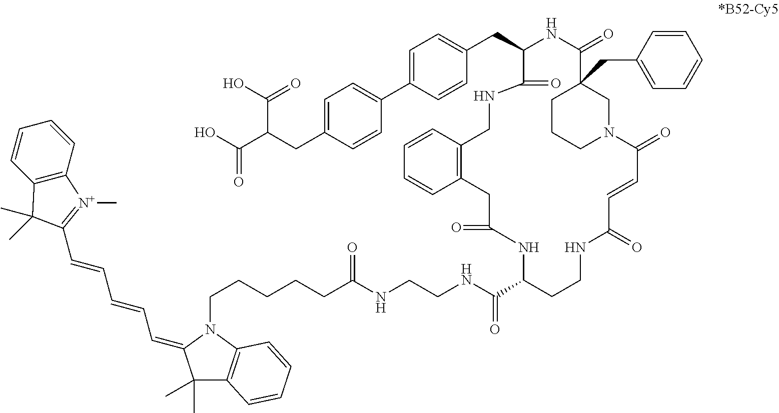 [SMILES: C[N+]1=C(/C=C/C=C/C=C2\N(CCCCCC(=O)NCCNC(=O)[C@H]3CCNC(=O)/C=C/C(=O)N4CCC[C@@](Cc5ccccc5)(C4)C(=O)N[C@H](Cc4ccc(-c5ccc(CC(C(=O)O)C(=O)O)cc5)cc4)C(=O)NCc4ccccc4CC(=O)N3)c3ccccc3C2(C)C)C(C)(C)c2ccccc21]

*B52-Cy5: Compound was synthesized as described in B52-Cy5 using *(4Br)B6-B as starting material. Yield: 6%

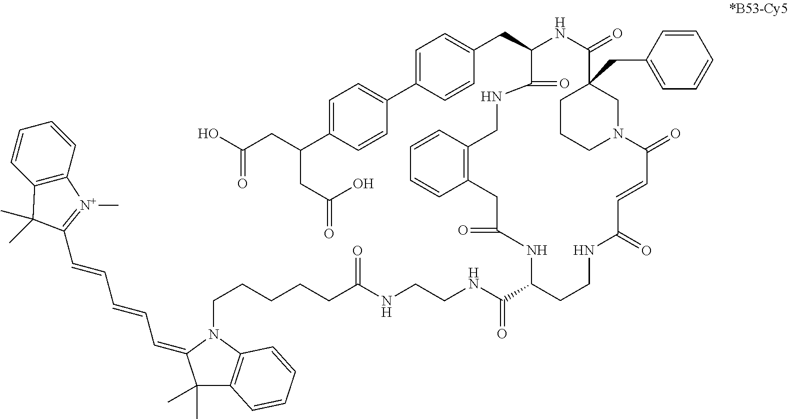 [SMILES: C[N+]1=C(/C=C/C=C/C=C2\N(CCCCCC(=O)NCCNC(=O)[C@H]3CCNC(=O)/C=C/C(=O)N4CCC[C@@](Cc5ccccc5)(C4)C(=O)N[C@H](Cc4ccc(-c5ccc(C(CC(=O)O)CC(=O)O)cc5)cc4)C(=O)NCc4ccccc4CC(=O)N3)c3ccccc3C2(C)C)C(C)(C)c2ccccc21]

B53-Cy5: Compound was synthesized as described in B53-Cy5 using *(4Br)B6-B as starting material. Yield: 8%.

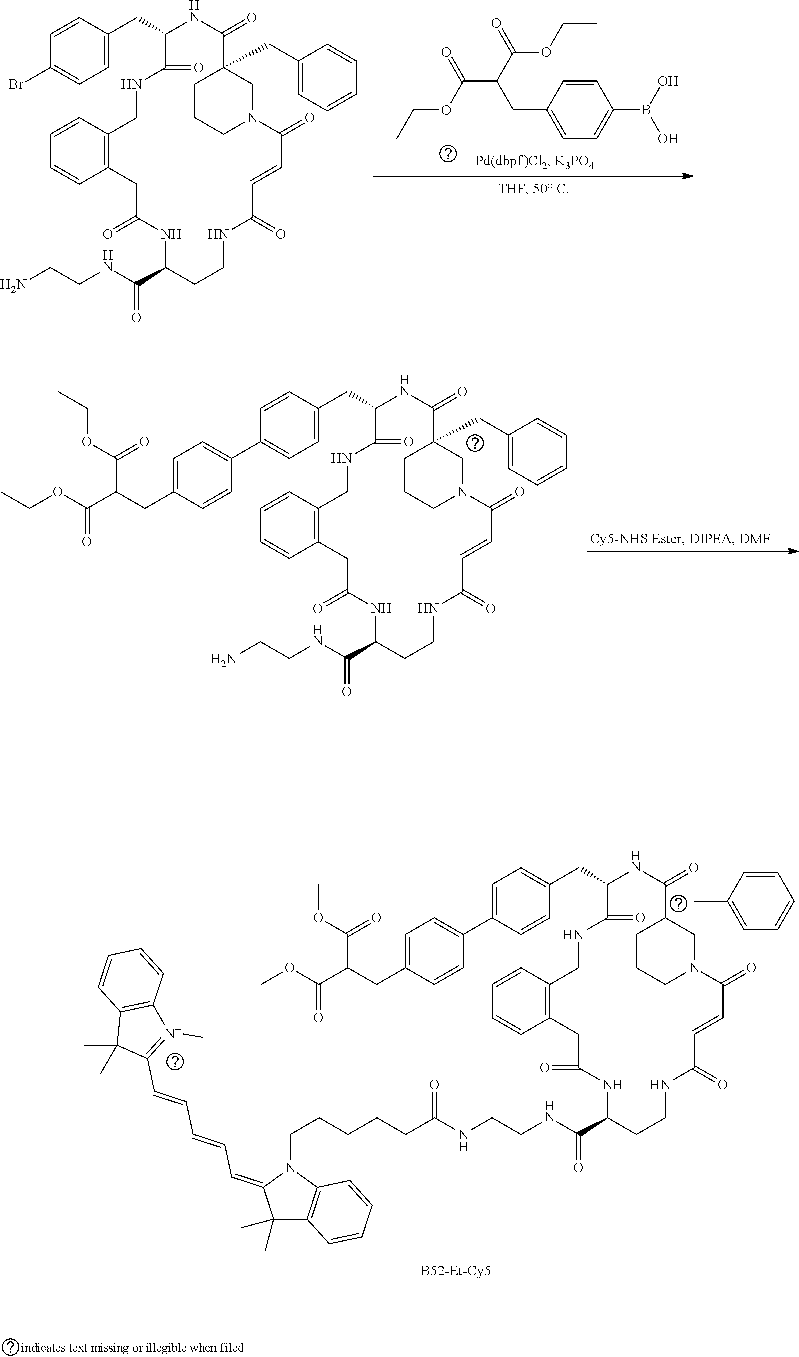

B52-Et-Cy5: Compound was synthesized using Generalized procedure A for Suzuki-Miyaura cross-coupling of macrocycles using (4Br)B6-B and I29b. Suzuki product was dissolved in a 1.5 mL Eppendorf tube with 100 μL DMF and mixed with DIPEA (5 eq.). Cy5-NHS ester (Lumiprobe) (2 eq.) dissolved in 50 μL DMF was then added to the flask allowed to stir for 1 hour. Excess Cy5-NHS ester was quenched with N-Acetylethylenediamine (10 eq.), to make Cy5-enAc. Resulting crude mixture was purified under same HPLC conditions to yield the two Cy5 coupled products. Yield: 20%

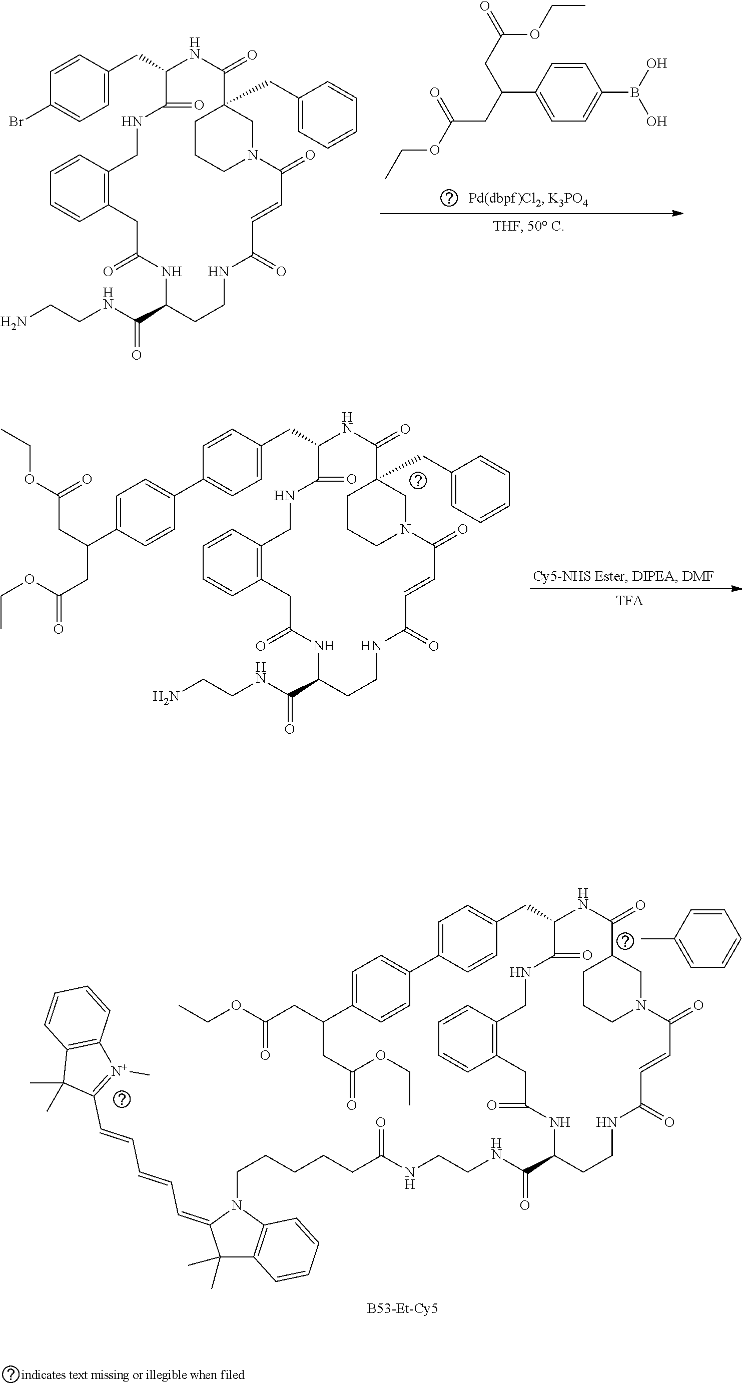

B53-Et-Cy5: Compound was synthesized using Generalized procedure A for Suzuki-Miyaura cross-coupling of macrocycles using (4Br)B6-B and I30c. Suzuki product was dissolved in a 1.5 mL Eppendorf tube with 100 uLDMF and mixed with DIPEA (5 eq.). Cy5-NHS ester (Lumiprobe) (2 eq.) dissolved in 50 μL DMF was then added to the flask allowed to stir for 1 hour. Excess Cy5-NHS ester was quenched with N-Acetylethylenediamine (10 eq.), to make Cy5-enAc. Resulting crude mixture was purified under same HPLC conditions to yield the two Cy5 coupled products. Yield: 21%

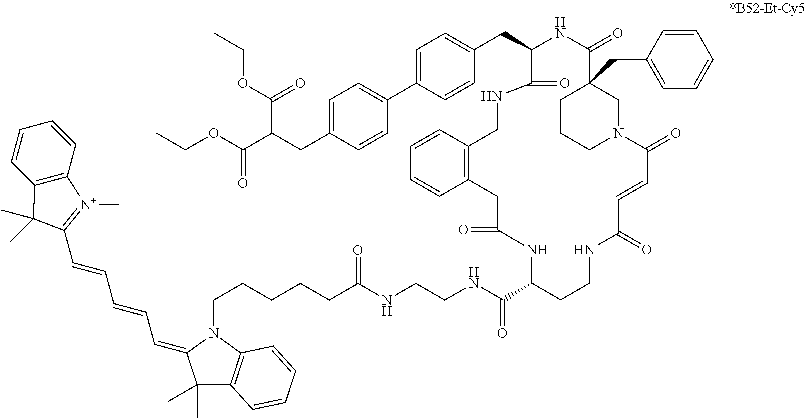 [SMILES: CCOC(=O)C(Cc1ccc(-c2ccc(C[C@H]3NC(=O)[C@@]4(Cc5ccccc5)CCCN(C4)C(=O)/C=C/C(=O)NCC[C@H](C(=O)NCCNC(=O)CCCCCN4\C(=C/C=C/C=C/C5=[N+](C)c6ccccc6C5(C)C)C(C)(C)c5ccccc54)NC(=O)Cc4ccccc4CNC3=O)cc2)cc1)C(=O)OCC]

*B52-Et-Cy5: Compound was synthesized as described in B52-Et-Cy5 using *(4Br)B6-B as starting material. Yield: 18%

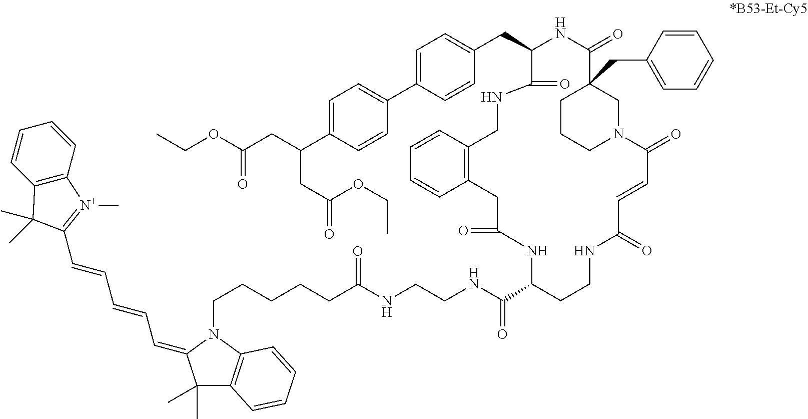 [SMILES: CCOC(=O)CC(CC(=O)OCC)c1ccc(-c2ccc(C[C@H]3NC(=O)[C@@]4(Cc5ccccc5)CCCN(C4)C(=O)/C=C/C(=O)NCC[C@H](C(=O)NCCNC(=O)CCCCCN4\C(=C/C=C/C=C/C5=[N+](C)c6ccccc6C5(C)C)C(C)(C)c5ccccc54)NC(=O)Cc4ccccc4CNC3=O)cc2)cc1]

*B53-Et-Cy5: Compound was synthesized as described in B53-Et-Cy5 using *(4Br)B6-B as starting material. Yield: 17%.

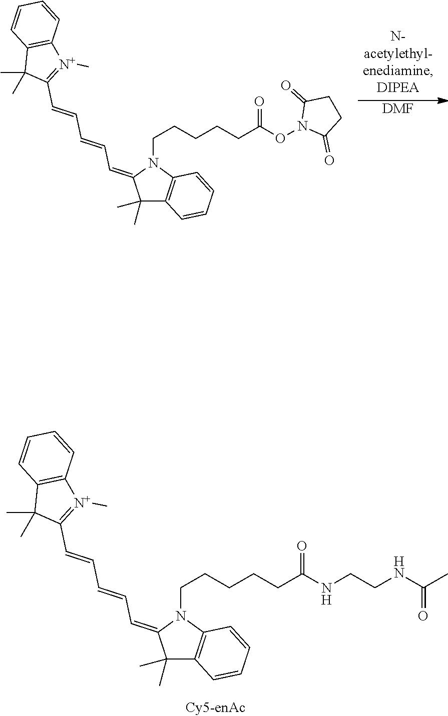

Cy5-enAc: Compound was isolated from excess Cy5-NHS ester that was quenched with N-Acetylethylenediamine (10 eq.) during syntheses of B52-Et-Cy5 and B53-Et-Cy5. HPLC purification was able to separate Cy5-enAc successfully from of B52-Et-Cy5 and B53-Et-Cy5.

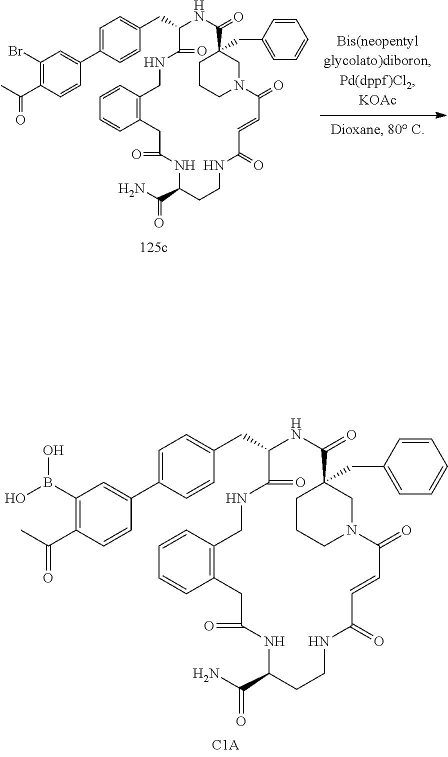

C1A: A mixture of I25c (0.008 mmol, 1 eq.), bis(neopentyl glycolato)diboron (0.08 mmol, 10 eq.), [1,1-Bis(diphenylphosphino)ferrocene]dichloropalladium(II) (0.001 mmol, 0.2 eq.), and KOAc (0.08 mmol, 10 eq.) was dissolved in 1 mL 1,4-dioxane (0.008 M). The reaction was stirred for thirty minutes at 80° C. The reaction was then cooled down to room temperature, solvent removed under vacuum, dissolved in DMF, and filtered. The crude product was purified by reverse-phase HPLC using 10-80% acetonitrile in water in 0.1% trifluoroacetic acid and lyophilized to afford C1A as a white powder. Yield: 20% yield

Bis(neopentyl glyolato) and the shorter 30-minute reaction time was due to significant protodeborylation observed with bis(pinacolato)diboron and longer reaction times.

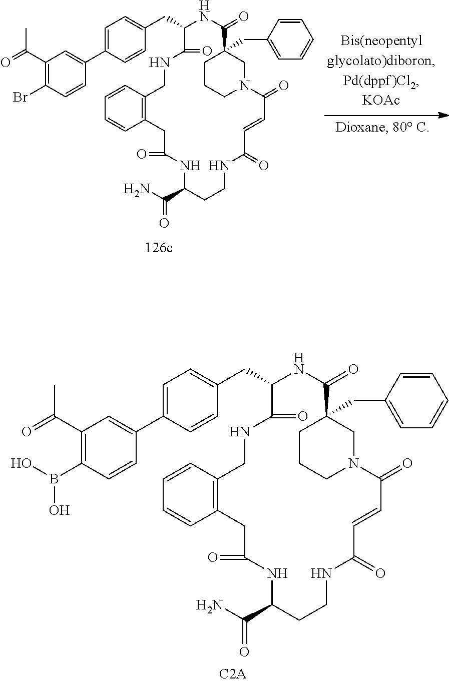

C2A: Compound was synthesized and purified as described in C1A with I26c, affording C2A as a white powder. Yield: 29%

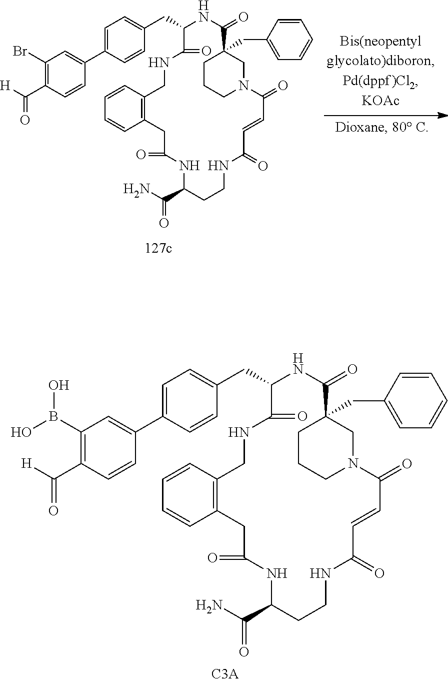

C3A: Compound was synthesized and purified as described in C1A with I27c, affording C3A as a white powder. Yield: 14%

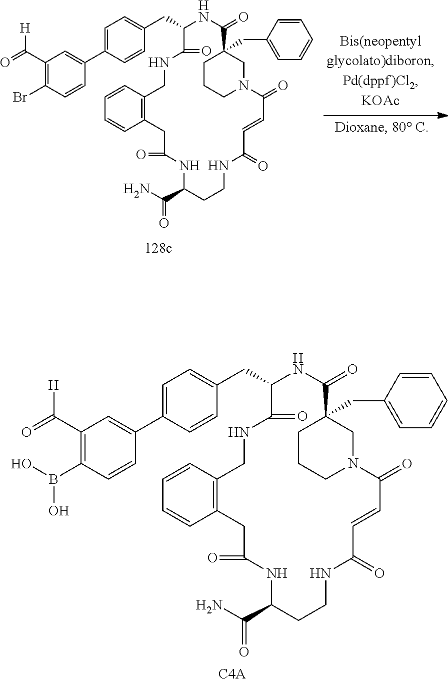

C4A: Compound was synthesized and purified as described in C1A with I28c, affording C4A as a white powder. Some impurities were observed by ¹H-NMR that were unable to be removed by HPLC, compound was tested without further purification. Yield: 4%

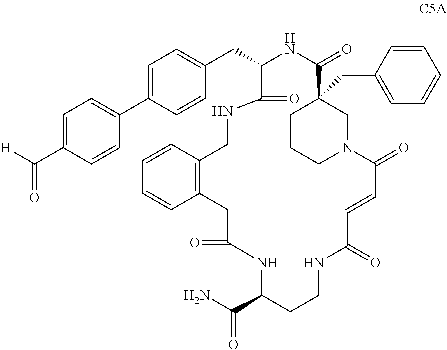 [SMILES: NC(=O)[C@@H]1CCNC(=O)/C=C/C(=O)N2CCC[C@](Cc3ccccc3)(C2)C(=O)N[C@@H](Cc2ccc(-c3ccc(C=O)cc3)cc2)C(=O)NCc2ccccc2CC(=O)N1]

C5A: Compound was synthesized according to Generalized procedure A for Suzuki-Miyaura cross-coupling of macrocycles with (4Br)B6-A and 4-formylphenylboronic acid, affording C5A as a white powder. Yield: 16%

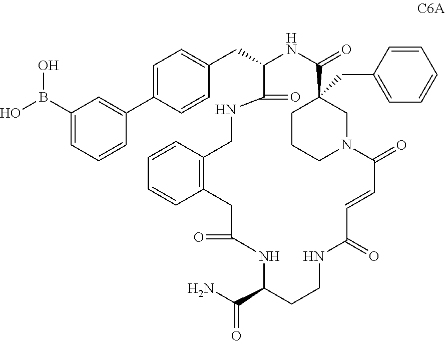 [SMILES: NC(=O)[C@@H]1CCNC(=O)/C=C/C(=O)N2CCC[C@](Cc3ccccc3)(C2)C(=O)N[C@@H](Cc2ccc(-c3cccc(B(O)O)c3)cc2)C(=O)NCc2ccccc2CC(=O)N1]

C6A: Compound was synthesized according to Generalized procedure A for Suzuki-Miyaura cross-coupling of macrocycles with (4Br)B6-A and benzene 1,3-diboronic acid, affording C6A as a white powder. Yield: 13%

### REFERENCES

1. Davis, T. L. et al. Structural and biochemical characterization of the human cyclophilin family of peptidyl-prolyl isomerases. PLoS Biol. 8, (2010).
2. Chen, J. et al. Biochemical Features of Recombinant Human Cyclophilin J. Anticancer Res. 36, 1175-1180 (2016).
3. Dunyak, B. M. & Gestwicki, J. E. Peptidyl-Proline Isomerases (PPIases): Targets for Natural Products and Natural Product-Inspired Compounds. J. Med. Chem. 59, 9622-9644 (2016).
4. Austin, C. et al. Prolyl Isomerases—Old Proteins as New Therapeutic Targets. 1-12 (2015).
5. Amanakis, G. & Murphy, E. Cyclophilin D: An Integrator of Mitochondrial Function. Front. Physiol. 11, 1-6 (2020).
6. Izzo, V., Bravo-San Pedro, J. M., Sica, V., Kroemer, G. & Galluzzi, L. Mitochondrial Permeability Transition: New Findings and Persisting Uncertainties. Trends Cell Biol. 26, 655-667 (2016).
7. Briston, T., Selwood, D. L., Szabadkai, G. & Duchen, M. R. Mitochondrial Permeability Transition: A Molecular Lesion with Multiple Drug Targets. Trends Pharmacol. Sci. 40, 50-70 (2018).
8. Baines, C. P. et al. Loss of cyclophilin D reveals a critical role for mitochondrial permeability transition in cell death. Nature 434, 658 (2005).
9. Nakagawa, T. et al. Cyclophilin D-dependent mitochondrial permeability transition regulates some necrotic but not apoptotic cell death. Nature 434, 652-658 (2005).
10. Mishra, J. et al. Cyclosporin A Increases Mitochondrial Buffering of Calcium: An Additional Mechanism in Delaying Mitochondrial Permeability Transition Pore Opening. Cells 8, 1052 (2019).
11. Kroemer, G., Galluzzi, L. & Brenner, C. Mitochondrial Membrane Permeabilization in Cell Death. Physiol. Rev. 87, 99-163 (2007).
12. Morciano, G. et al. Molecular identity of the mitochondrial permeability transition pore and its role in ischemia-reperfusion injury. J. Mol. Cell. Cardiol. 78, 142-153 (2015).
13. Panel, M. et al. Small-Molecule Inhibitors of Cyclophilins Block Opening of the Mitochondrial Permeability Transition Pore and Protect Mice From Hepatic Ischemia/Reperfusion Injury. Gastroenterology 157, 1368-1382 (2019).
14. Martin, L. J. et al. GNX-4728, a novel small molecule drug inhibitor of mitochondrial permeability transition, is therapeutic in a mouse model of amyotrophic lateral sclerosis. Front. Cell. Neurosci. 8, 1-7 (2014).
15. Du, H. et al. Cyclophilin D deficiency attenuates mitochondrial and neuronal perturbation and ameliorates learning and memory in Alzheimer's disease. Nat. Med. 14, 1097-1105 (2008).
16. Valasani, K. R. et al. Identification of a Small Molecule Cyclophilin D Inhibitor for Rescuing AP-Mediated Mitochondrial Dysfunction. ACS Med. Chem. Lett. (2016) doi:10.1021/acsmedchemlett.5b00451.
17. Quintanilla, R. A., Jin, Y. N., Bernhardi, R. Von & Johnson, G. V. W. Mitochondrial permeability transition pore induces mitochondria injury in Huntington disease. Mol. Neurodegener. 8, 1-19 (2013).
18. Rao, V. K., Carlson, E. A. & Yan, S. S. Mitochondrial permeability transition pore is a potential drug target for neurodegeneration. Biochim. Biophys. Acta—Mol. Basis Dis. 1842, 1267-1272 (2018).
19. Forte, M. et al. Cyclophilin D inactivation protects axons in experimental autoimmune encephalomyelitis, an animal model of multiple sclerosis. Proc. Natl. Acad. Sci. U.S.A 104, 7558-7563 (2007).
20. López-Erauskin, J., Ferrer, I., Galea, E. & Pujol, A. Cyclophilin D as a potential target for antioxidants in neurodegeneration: The X-ALD case. Biol. Chem. 394, 621-629 (2013).
21. Rasheed, M. Z., Tabassum, H. & Parvez, S. Mitochondrial permeability transition pore: a promising target for the treatment of Parkinson's disease. Protoplasma 254, 33-42 (2017).
22. Simón Serrano, S. et al. Evaluation of NV556, a Novel Cyclophilin Inhibitor, as a Potential Antifibrotic Compound for Liver Fibrosis. Cells 8, 1409 (2019).
23. Panel, M., Ghaleh, B. & Morin, D. Mitochondria and aging: A role for the mitochondrial transition pore?Aging Cell 17, 1-15 (2018).
24. Zhou, B. et al. Mitochondrial Permeability Uncouples Elevated Autophagy and Lifespan Extension. Cell 177, 1-16 (2019).
25. Taddeo, E. P. et al. Mitochondrial proton leak regulated by Cyclophilin D elevates insulin secretion in islets at nonstimulatory glucose levels. Diabetes 69, 131-145 (2020).
26. Yan, S. et al. FiFO ATP synthase-cyclophilin d interaction contributes to diabetes-induced synaptic dysfunction and cognitive decline. Diabetes 65, 3482-3494 (2016).
27. Sileikyte, J. & Forte, M. The Mitochondrial Permeability Transition in Mitochondrial Disorders. Oxid. Med. Cell. Longev. 2019, 1-11 (2019).
28. Carraro, M., Carrer, A., Urbani, A. & Bernardi, P. Molecular nature and regulation of the mitochondrial permeability transition pore(s), drug target(s) in cardioprotection. J. Mol. Cell. Cardiol. 144, 76-86 (2020).
29. Bround, M. J., Bers, D. M. & Molkentin, J. D. A 20/20 view of ANT function in mitochondrial biology and necrotic cell death. J. Mol. Cell. Cardiol. 0-1 (2020) doi:10.1016/j.yjmcc.2020.05.012.
30. Carraro, M., Checchetto, V., Szabo, I. & Bernardi, P. F-ATP synthase and the permeability transition pore: fewer doubts, more certainties. FEBS Lett. (2019) doi:10.1002/1873-3468.13485.
31. Thapar, R. Roles of prolyl isomerases in RNA-mediated gene expression. Biomolecules 5, 974-999 (2015).
32. Theuerkorn, M., Fischer, G. & Schiene-Fischer, C. Prolyl cis/trans isomerase signalling pathways in cancer. Curr. Opin. Pharmacol. 11, 281-287 (2011).
33. Schiene-Fischer, C. Multidomain Peptidyl Prolyl cis/trans Isomerases. Biochim. Biophys. Acta—Gen. Subj. 1850, 2005-2016 (2015).
34. Frausto, S. D., Lee, E. & Tang, H. Cyclophilins as modulators of viral replication. Viruses 5, 1684-1701 (2013).
35. Tanaka, Y., Sato, Y. & Sasaki, T. Suppression of coronavirus replication by cyclophilin inhibitors. Viruses 5, 1250-1260 (2013).
36. Ke, H. & Huai, Q. Crystal Structures of Cyclophilin and its Partners. Front. Biosci. 9, 2285-2296 (2004).
37. Kenji Kajitani, Masahiro Fujihashi, Yukiko Kobayashi, S. S. & Yoshihide Tsujimoto, and K. M. Crystal structure of human cyclophilin D in complex with its inhibitor, cyclosporin A at 0.96-A resolution. Proteins 70, 1635-1639 (2007).
38. De Simone, A. et al. A computationally designed binding mode flip leads to a novel class of potent tri-vector cyclophilin inhibitors. Chem. Sci. 542-547 (2019) doi:10.1039/C8SC03831G.
39. Ahmed-Belkacem, A. et al. Fragment-based discovery of a new family of non-peptidic small-molecule cyclophilin inhibitors with potent antiviral activities. Nat. Commun. 7, (2016).
40. Elkamhawy, A. et al. Novel quinazoline-urea analogues as modulators for Ap-induced mitochondrial dysfunction: Design, synthesis, and molecular docking study. Eur. J. Med. Chem. 84, 466-475 (2014).
41. Park, I. et al. Discovery of non-peptidic small molecule inhibitors of cyclophilin D as neuroprotective agents in Ap-induced mitochondrial dysfunction. J. Comput. Aided. Mol. Des. 31, 929-941 (2017).
42. Gradler, U. et al. Discovery of novel Cyclophilin D inhibitors starting from three dimensional fragments with millimolar potencies. Bioorganic Med. Chem. Lett. 29, 126717 (2019).
43. Steadman, V. A. et al. Discovery of Potent Cyclophilin Inhibitors Based on the Structural Simplification of Sanglifehrin A. J. Med. Chem. 60, 1000-1017 (2017).
44. Kuo, J. et al. A pan-cyclophilin inhibitor, CRV431, decreases fibrosis and tumor development in chronic liver disease models. J. Pharmacol. Exp. Ther. 371, 231-241 (2019).
45. Hyun, S. et al. One-bead-one-compound screening approach to the identification of cyclic peptoid inhibitors of cyclophilin D as neuroprotective agents from mitochondrial dysfunction. Chem. Commun. 57, 2388-2391 (2021).
46. Usanov, D. L., Chan, A. I., Maianti, J. P. & Liu, D. R. Second-generation DNA-templated macrocycle libraries for the discovery of bioactive small molecules. Nat. Chem. 1-11 (2018) doi:10.1038/s41557-018-0033-8.
47. Wang, M., Herrmann, C. J., Simonovic, M., Szklarczyk, D. & von Mering, C. Version 4.0 of PaxDb: Protein abundance data, integrated across model organisms, tissues, and cell-lines. Proteomics 15, 3163-3168 (2015).
48. Zhou, L. et al. Mitochondria-Localized Self-Reporting Small-Molecule-Decorated Theranostic Agents for Cancer-Organelle Transporting and Imaging. ACS Appl. Bio Mater. 2, 5164-5173 (2019).
49. Akgay, G. et al. Inhibition of Mcl-1 through covalent modification of a noncatalytic lysine side chain. Nat. Chem. Biol. 12, 931-936 (2016).
50. Quach, D. et al. Strategic Design of Catalytic Lysine-Targeting Reversible Covalent BCR-ABL Inhibitors. Angew. Chemie—Int. Ed. 60, 17131-17137 (2021)
51. Studier, F. W. Protein production by auto-induction in high density shaking cultures. Protein Expr. Purif 41, 207-234 (2005).
52. Usanov, D. L., Chan, A. I., Maianti, J. P. & Liu, D. R. Second-generation DNA-templated macrocycle libraries for the discovery of bioactive small molecules. Nat. Chem. 10, 1-11 (2018).
53. Mori, T. et al. Use of a real-time fluorescence monitoring system for high-throughput screening for Prolyl isomerase inhibitors. J. Biomol. Screen. 14, 419-424 (2009).
54. Liu, Y. et al. A fluorescence polarization-based assay for peptidyl prolyl cis/trans isomerase cyclophilin A. Anal. Biochem. 356, 100-107 (2006).
55. Wear, M. A. et al. A surface plasmon resonance-based assay for small molecule inhibitors of human cyclophilin A. Anal. Biochem. 345, 214-226 (2005).
56. Vonrhein, C. et al. Data processing and analysis with the autoPROC toolbox. Acta Crystallogr. D. Biol. Crystallogr. 67, 293-302 (2011).
57. Winter, G. & McAuley, K. E. Automated data collection for macromolecular crystallography. Methods 55, 81-93 (2011).
58. Kabsch, W. Xds. Acta Crystallogr D Biol Crystallogr 66, 125-132 (2010).
59. Winn, M. D. et al. Overview of the CCP4 suite and current developments. Acta Crystallogr. D. Biol. Crystallogr. 67, 235-242 (2011).
60. McCoy, A. J. et al. Phaser crystallographic software. J. Appl. Crystallogr. 40, 658-674 (2007).
61. Liebschner, D. et al. Macromolecular structure determination using X-rays, neutrons and electrons: recent developments in Phenix. Acta Crystallogr. Sect. D, Struct. Biol. 75, 861-877 (2019).
62. Emsley, P., Lohkamp, B., Scott, W. G. & Cowtan, K. Features and development of Coot. Acta Crystallogr. D. Biol. Crystallogr. 66, 486-501 (2010).
63. Moriarty, N. W., Grosse-Kunstleve, R. W. & Adams, P. D. electronic Ligand Builder and Optimization Workbench (eLBOW): a tool for ligand coordinate and restraint generation. Acta Crystallogr. D. Biol. Crystallogr. 65, 1074-1080 (2009).
64. SchUttelkopf, A. W. & van Aalten, D. M. F. PRODRG: a tool for high-throughput crystallography of protein-ligand complexes. Acta Crystallogr. D. Biol. Crystallogr. 60, 1355-1363 (2004).
65. Balius, T. E., Mukherjee, S. & Rizzo, R. C. Implementation and evaluation of a docking-rescoring method using molecular footprint comparisons. J. Comput. Chem. 32, 2273-2289 (2011).
66. Pettersen, E. F. et al. UCSF Chimera—a visualization system for exploratory research and analysis. J. Comput. Chem. 25, 1605-1612 (2004).
67. Needleman, S. B. & Wunsch, C. D. A general method applicable to the search for similarities in the amino acid sequence of two proteins. J. Mol. Biol. 48, 443-453 (1970).
68. Wang, J., Wolf, R. M., Caldwell, J. W., Kollman, P. A. & Case, D. A. Development and testing of a general amber force field. J. Comput. Chem. 25, 1157-1174 (2004).
69. Gasteiger, J. & Marsili, M. A new model for calculating atomic charges in molecules. Tetrahedron Lett. 19, 3181-3184 (1978).
70. Gasteiger, J. & Marsili, M. Iterative partial equalization of orbital electronegativity—a rapid access to atomic charges. Tetrahedron 36, 3219-3228 (1980).
71. Hunter, J. D. Matplotlib: A 2D Graphics Environment. Comput. Sci. Eng. 9, 90-95 (2007).
72. Carroll, J., He, J., Ding, S., Fearnley, I. M. & Walker, J. E. Persistence of the permeability transition pore in human mitochondria devoid of an assembled ATP synthase. Proc. Natl. Acad. Sci. 201904005 (2019) doi:10.1073/pnas.1904005116.
73. Qiao, J. X. et al. Synthesis of Fmoc-Protected Arylphenylalanines (Bip Derivatives) via Nonaqueous Suzuki-Miyaura Cross-Coupling Reactions. J. Org. Chem. 81, 9499-9506 (2016).
74. Hinkes, S. P. A. & Klein, C. D. P. Virtues of Volatility: A Facile Transesterification Approach to Boronic Acids. Org. Lett. 21, 3048-3052 (2019).
75. Schlatter, D. et al. Crystal engineering yields crystals of cyclophilin D diffracting to 1.7 A resolution. Acta Crystallogr. D. Biol. Crystallogr. 61, 513-519 (2005).
76. Wilent, J. & Petersen, K. S. Enantioselective desymmetrization of diesters. J. Org. Chem. 79, 2303-2307 (2014).
77. Lunn, D. J. et al. Scalable synthesis of an architectural library of well-defined poly(acrylic acid) derivatives: Role of structure on dispersant performance. J. Polym. Sci. Part A Polym. Chem. 57, 716-725 (2019).
78. Quach, D. et al. Strategic Design of Catalytic Lysine-Targeting Reversible Covalent BCR-ABL Inhibitors. Angew. Chemie Int. Ed. 60, 17131-17137 (2021).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I-A): 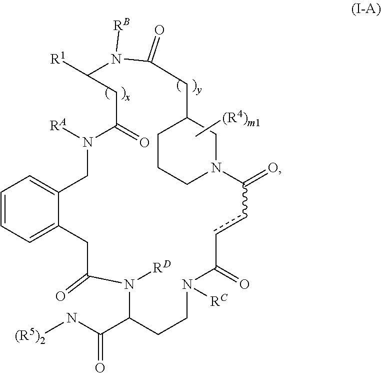
or a pharmaceutically acceptable salt, solvate, hydrate, polymorph, co-crystal, tautomer, stereoisomer, isotopically enriched form, prodrug, or mixture thereof, wherein:
each instance of [symbol] is independently a single or double C—C bond, as valency permits, wherein when [symbol] is a double C—C bond adjacent to [symbol] , then [symbol] indicates that the adjacent C—C double bond may be in a cis or trans configuration;
R¹ is 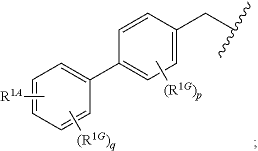
R^{1A} is 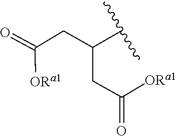
or —(CH₂)ₙN(R^{a1})₂;
each instance of R^{a1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a1} are joined together with the intervening atoms to form a substituted or unsubstituted heterocyclyl ring;
each instance of R^{1G} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{g1}, —NO₂, —N(R^{g2})₂, —SR^{g1}, —SO₂R^{g1}, —CN, or —SCN; R^{g1} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —N(R^{g2})₂, or —O(R^{g3});
each instance of R^{g2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two instances of R^{g2} are taken together to form a ring when attached to nitrogen;
each instance of R^{g3} is independently hydrogen, substituted or unsubstituted alkyl, substituted or unsubstituted acyl, or an oxygen protecting group when attached to an oxygen atom;
R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl;
each instance of R⁵ is independently hydrogen, substituted or unsubstituted alkyl, substituted or unsubstituted alkenyl, substituted or unsubstituted alkynyl, substituted or unsubstituted acyl, or a nitrogen protecting group;
each of R^{A}, R^{B}, R^{C}, and R^{D} is independently hydrogen, substituted or unsubstituted alkyl, substituted or unsubstituted acyl, or a nitrogen protecting group;
x is 0 or 1;
y is 0 or 1;
m1 is 0, 1, 2, 3, 4, 5, or 6;
n is 3, 4, 5, 6, 7, 8, 9, or 10;
p is 0, 1, 2, 3, or 4; and
q is 0, 1, 2, 3, or 4.

2. A compound of Formula (I-B): 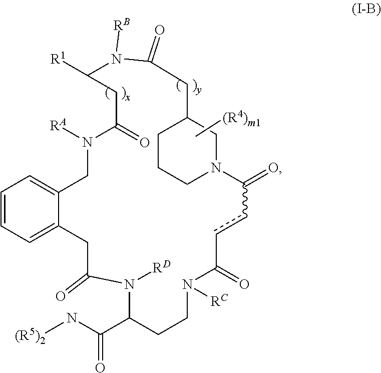
or a pharmaceutically acceptable salt, solvate, hydrate, polymorph, co-crystal, tautomer, stereoisomer, isotopically enriched form, prodrug, or mixture thereof, wherein:
each instance of [symbol] is independently a single or double C—C bond, as valency permits, wherein when [symbol] is a double C—C bond adjacent to [symbol] , then [symbol] indicates that the adjacent C—C double bond may be in a cis or trans configuration;
R¹ is 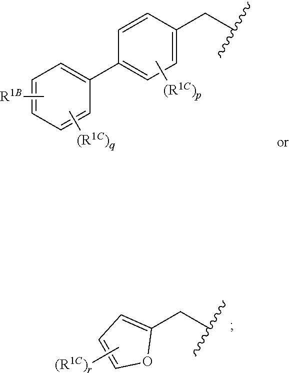
R^{1B} is 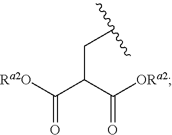
each instance of R^{a2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a2} are joined together with the intervening atoms to form a substituted or unsubstituted heterocyclyl ring;
each instance of R^{1C} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —SO₂R^{c1}, —CN, or —SCN;
R^{c1} is halogen, hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —N(R^{c2})₂, or —O(R^{c3});
each instance of R^{c2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two instances of R^{c2} are taken together to form a ring when attached to nitrogen;
each instance of R^{c3} is independently hydrogen, substituted or unsubstituted alkyl, substituted or unsubstituted acyl, or an oxygen protecting group when attached to an oxygen atom;
R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl;
each instance of R⁵ is independently hydrogen or 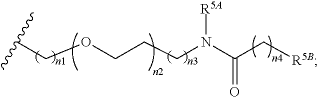
R^{5A} is independently hydrogen, substituted or unsubstituted alkyl, substituted or unsubstituted acyl, or a nitrogen protecting group;
R^{5B} is substituted or unsubstituted carbocyclyl, substituted or unsubstituted aryl, substituted or unsubstituted heterocyclyl, or substituted or unsubstituted heteroaryl;
each of R^{A}, R^{B}, R^{C}, and R^{D} is independently hydrogen, substituted or unsubstituted alkyl, substituted or unsubstituted acyl, or a nitrogen protecting group;
x is 0 or 1;
y is 0 or 1;
m1 is 0, 1, 2, 3, 4, 5, or 6;
p is 0, 1, 2, 3, or 4;
q is 0, 1, 2, 3, or 4;
r is 0, 1, 2, or 3;
n1 is 0, 1, 2, 3, 4, 5, 6, 7, 8, 9, or 10;
n2 is 0, 1, 2, 3, 4, 5, 6, 7, 8, 9, or 10;
n3 is 0, 1, 2, 3, 4, 5, 6, 7, 8, 9, or 10; and
n4 is 0, 1, 2, 3, 4, 5, 6, 7, 8, 9, or 10.

3. A compound of Formula (I-C): 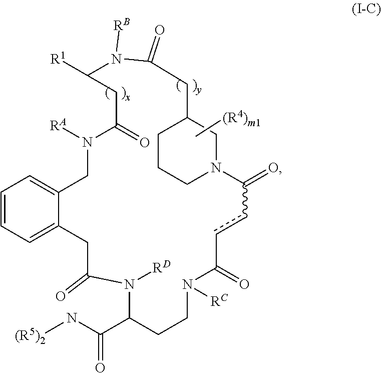
or a pharmaceutically acceptable salt, solvate, hydrate, polymorph, co-crystal, tautomer, stereoisomer, isotopically enriched form, prodrug, or mixture thereof, wherein:
each instance of [symbol] is independently a single or double C—C bond, as valency permits, wherein when [symbol] is a double C—C bond adjacent to [symbol] , then [symbol] indicates that the adjacent C—C double bond may be in a cis or trans configuration;
R¹ is 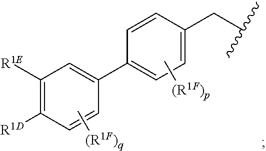
R^{1D} is hydrogen, —B(OR^{a3})₂, or —C(O)R^{a3};
R^{1E} is hydrogen, —B(OR^{a3})₂, or —C(O)R^{a3};
each instance of R^{a3} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl, or optionally wherein two instances of R^{a3} are joined together with the intervening atoms to form a substituted or unsubstituted heterocyclyl or heteroaryl ring;
each instance of R^{1F} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{f1}, —NO₂, —N(R^{f2})₂, —SR^{f1}, —SO₂R^{f1}, —CN, or —SCN;
R^{f1} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, —N(R^{f2})₂, or —O(R^{f3});
each instance of R^{f2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two instances of R^{f2} are taken together to form a ring when attached to nitrogen;
each instance of R^{f3} is independently hydrogen, substituted or unsubstituted alkyl, substituted or unsubstituted acyl, or an oxygen protecting group when attached to an oxygen atom;
R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl;
each instance of R⁵ is independently hydrogen, substituted or unsubstituted alkyl, substituted or unsubstituted alkenyl, substituted or unsubstituted alkynyl, substituted or unsubstituted acyl, or a nitrogen protecting group;
each of R^{A}, R^{B}, R^{C}, and R^{D} is independently hydrogen, substituted or unsubstituted alkyl, substituted or unsubstituted acyl, or a nitrogen protecting group;
x is 0 or 1;
y is 0 or 1;
m1 is 0, 1, 2, 3, 4, 5, or 6;
p is 0, 1, 2, 3, or 4; and
q is 0, 1, 2, or 3;
provided that R¹ is not

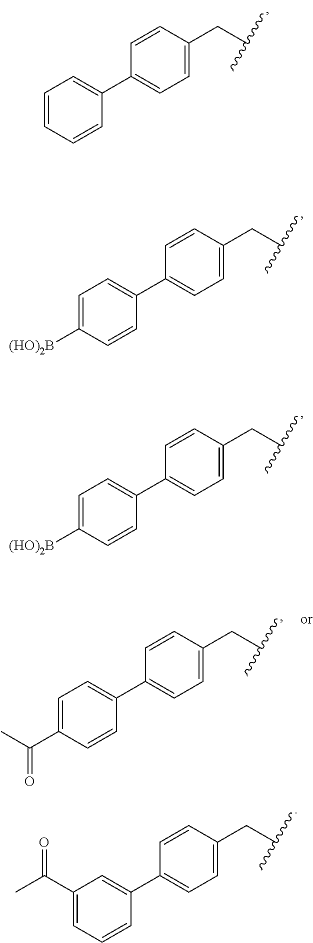 [SMILES: *Cc1ccc(-c2ccccc2)cc1 ; *Cc1ccc(-c2ccc(B(O)O)cc2)cc1 ; *Cc1ccc(-c2ccc(B(O)O)cc2)cc1 ; *Cc1ccc(-c2ccc(C(C)=O)cc2)cc1 ; *Cc1ccc(-c2cccc(C(C)=O)c2)cc1]

4. The compound of any one of claims 1-3, wherein the compound is of formula: 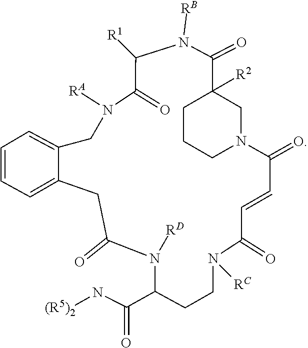
or a pharmaceutically acceptable salt, solvate, hydrate, polymorph, co-crystal, tautomer, stereoisomer, isotopically enriched form, prodrug, or mixture thereof, wherein:
R² is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, or substituted or unsubstituted heteroaryl.

5. The compound of any one of claims 1-4, wherein the compound is of formula: 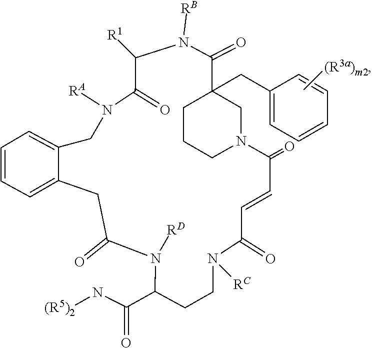
or a pharmaceutically acceptable salt, solvate, hydrate, polymorph, co-crystal, tautomer, stereoisomer, isotopically enriched form, prodrug, or mixture thereof, wherein:
each instance of R^{3a} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN; and
m2 is 0, 1, 2, 3, 4, or 5.

6. The compound of any one of claim 1, 4, or 5, wherein the compound is of formula: 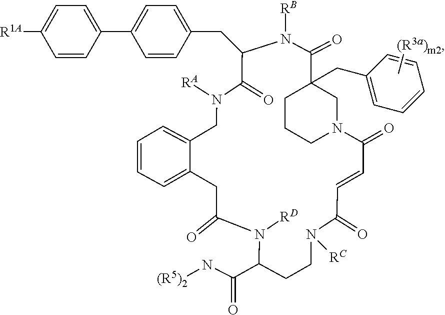
or a pharmaceutically acceptable salt, solvate, hydrate, polymorph, co-crystal, tautomer, stereoisomer, isotopically enriched form, prodrug, or mixture thereof.

7. The compound of any one of claim 1 or 4-6, wherein the compound is of formula: 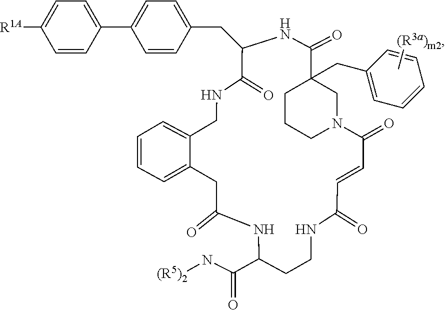
or a pharmaceutically acceptable salt, solvate, hydrate, polymorph, co-crystal, tautomer, stereoisomer, isotopically enriched form, prodrug, or mixture thereof.

8. The compound of any one of claim 1 or 4-7, wherein the compound is of formula: 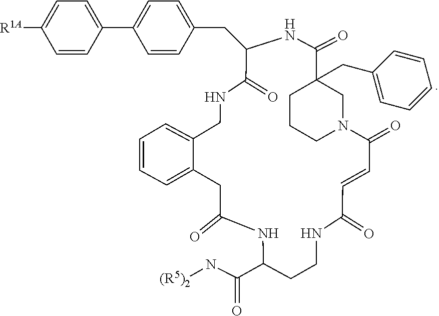
or a pharmaceutically acceptable salt, solvate, hydrate, polymorph, co-crystal, tautomer, stereoisomer, isotopically enriched form, prodrug, or mixture thereof.

9. The compound of any one of claim 1 or 4-8, wherein the compound is of formula: 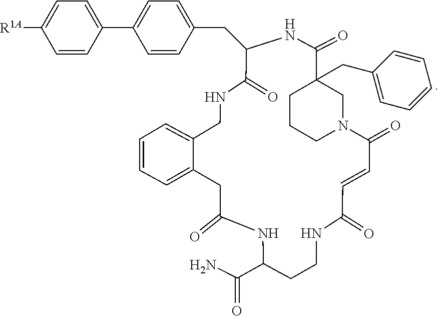
or a pharmaceutically acceptable salt, solvate, hydrate, polymorph, co-crystal, tautomer, stereoisomer, isotopically enriched form, prodrug, or mixture thereof.

10. The compound of any one of claim 1 or 4-6, wherein the compound is of formula: 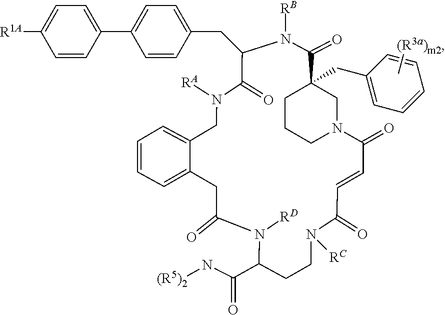
or a pharmaceutically acceptable salt, solvate, hydrate, polymorph, co-crystal, tautomer, stereoisomer, isotopically enriched form, prodrug, or mixture thereof.

11. The compound of any one of claim 1, 4-6, or 10, wherein the compound is of formula: 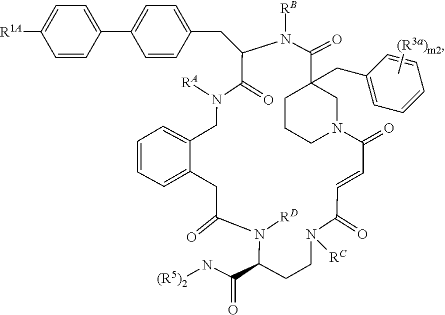
or a pharmaceutically acceptable salt, solvate, hydrate, polymorph, co-crystal, tautomer, stereoisomer, isotopically enriched form, prodrug, or mixture thereof.

12. The compound of any one of claim 1, 4-6, 10, or 11, wherein the compound is of formula: 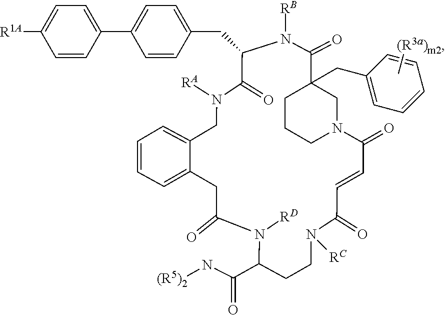
or a pharmaceutically acceptable salt, solvate, hydrate, polymorph, co-crystal, tautomer, stereoisomer, isotopically enriched form, prodrug, or mixture thereof.

13. The compound of any one of claim 1, 4-6, or 10-12, wherein the compound is of formula: 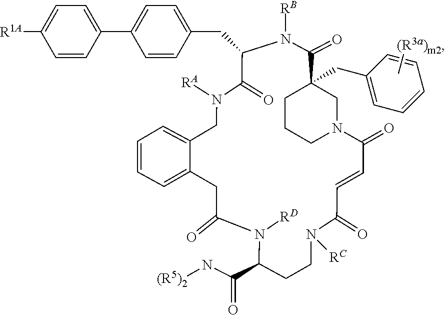
or a pharmaceutically acceptable salt, solvate, hydrate, polymorph, co-crystal, tautomer, stereoisomer, isotopically enriched form, prodrug, or mixture thereof.

14. The compound of any one of claim 1 or 4-7, wherein the compound is of formula: 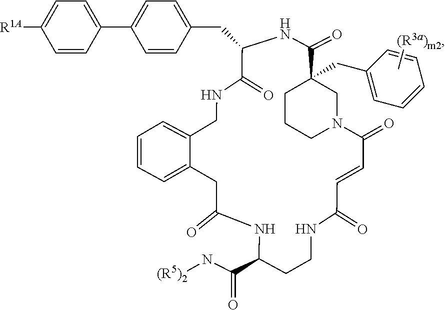
or a pharmaceutically acceptable salt, solvate, hydrate, polymorph, co-crystal, tautomer, stereoisomer, isotopically enriched form, prodrug, or mixture thereof.

15. The compound of any one of claim 1, 4-8, or 14, wherein the compound is of formula: 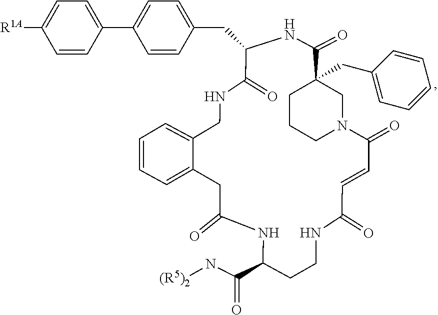
or a pharmaceutically acceptable salt, solvate, hydrate, polymorph, co-crystal, tautomer, stereoisomer, isotopically enriched form, prodrug, or mixture thereof.

16. The compound of any one of claim 1 or 4-15, wherein the compound is of formula: 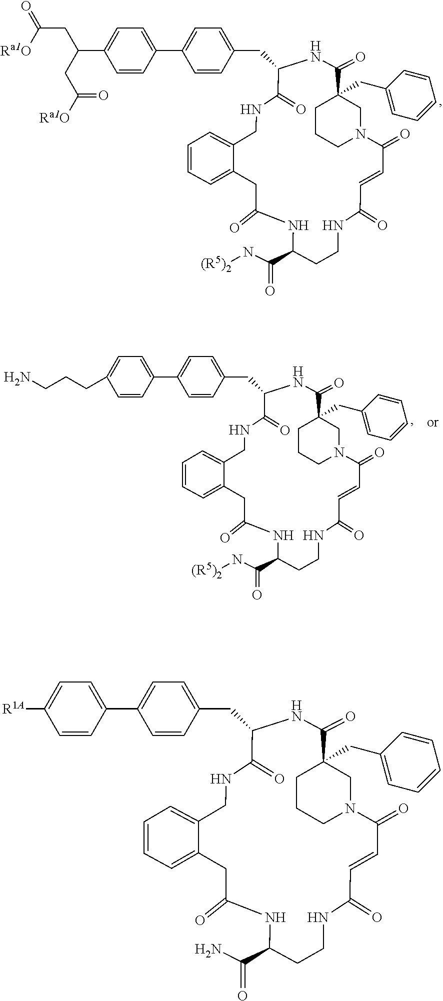
or a pharmaceutically acceptable salt, solvate, hydrate, polymorph, co-crystal, tautomer, stereoisomer, isotopically enriched form, prodrug, or mixture thereof.

17. The compound of any one of claim 2, 4, or 5, wherein the compound is of formula: 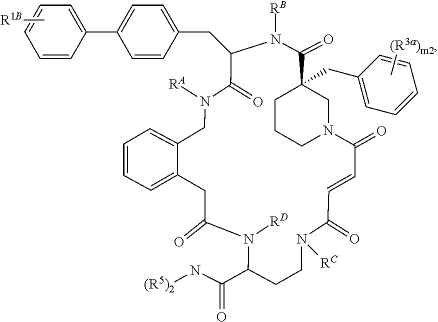
or a pharmaceutically acceptable salt, solvate, hydrate, polymorph, co-crystal, tautomer, stereoisomer, isotopically enriched form, prodrug, or mixture thereof.

18. The compound of any one of claim 2, 4, 5, or 17, wherein the compound is of formula: 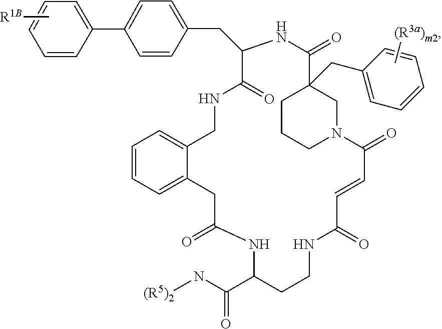
or a pharmaceutically acceptable salt, solvate, hydrate, polymorph, co-crystal, tautomer, stereoisomer, isotopically enriched form, prodrug, or mixture thereof.

19. The compound of any one of claim 2, 4, 5, 17, or 18, wherein the compound is of formula: 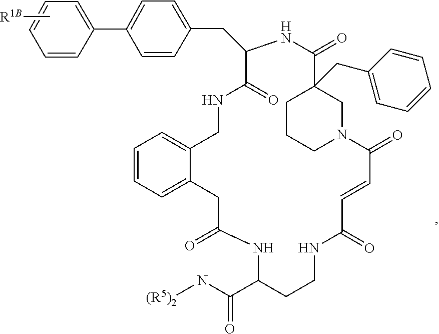
or a pharmaceutically acceptable salt, solvate, hydrate, polymorph, co-crystal, tautomer, stereoisomer, isotopically enriched form, prodrug, or mixture thereof.

20. The compound of any one of claim 2, 4, 5, or 17-19, wherein the compound is of formula: 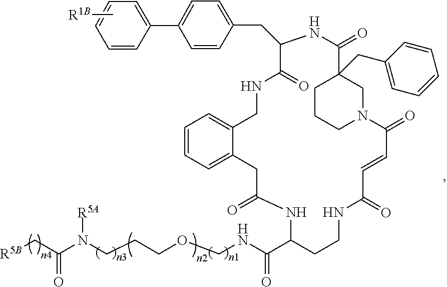
or a pharmaceutically acceptable salt, solvate, hydrate, polymorph, co-crystal, tautomer, stereoisomer, isotopically enriched form, prodrug, or mixture thereof.

21. The compound of any one of claim 2, 4, 5, or 17, wherein the compound is of formula: 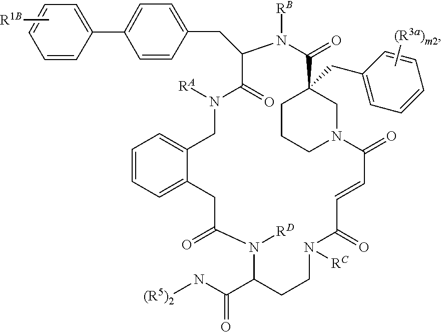
or a pharmaceutically acceptable salt, solvate, hydrate, polymorph, co-crystal, tautomer, stereoisomer, isotopically enriched form, prodrug, or mixture thereof.

22. The compound of any one of claim 2, 4, 5, 17, or 21, wherein the compound is of formula: 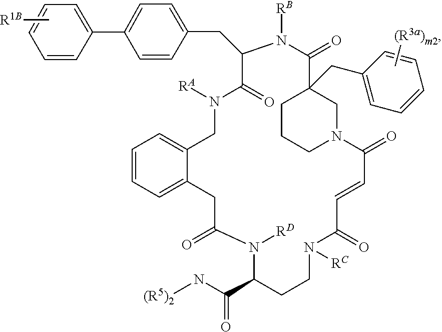
or a pharmaceutically acceptable salt, solvate, hydrate, polymorph, co-crystal, tautomer, stereoisomer, isotopically enriched form, prodrug, or mixture thereof.

23. The compound of any one of claim 2, 4, 5, 17, 21, or 22, wherein the compound is of formula: 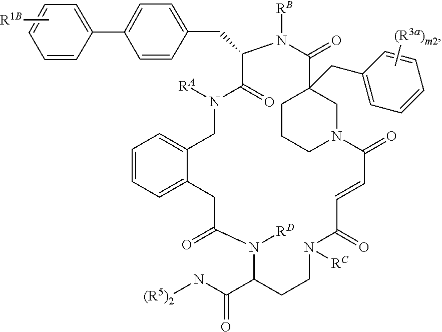
or a pharmaceutically acceptable salt, solvate, hydrate, polymorph, co-crystal, tautomer, stereoisomer, isotopically enriched form, prodrug, or mixture thereof.

24. The compound of any one of claim 2, 4, 5, 17, or 21-23, wherein the compound is of formula: 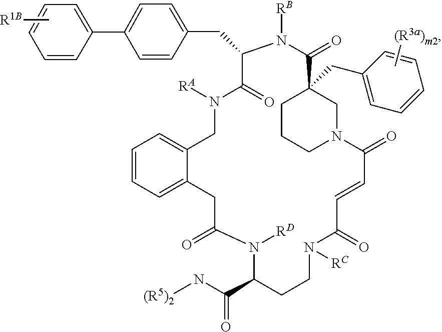
or a pharmaceutically acceptable salt, solvate, hydrate, polymorph, co-crystal, tautomer, stereoisomer, isotopically enriched form, prodrug, or mixture thereof.

25. The compound of any one of claim 2, 4, 5, 17, 18, or 21-24, wherein the compound is of formula: 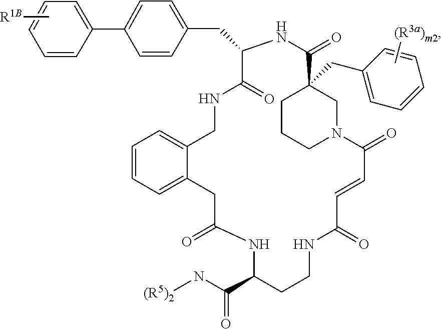
or a pharmaceutically acceptable salt, solvate, hydrate, polymorph, co-crystal, tautomer, stereoisomer, isotopically enriched form, prodrug, or mixture thereof.

26. The compound of any one of claim 2, 4, 5, 17-19, or 21-25, wherein the compound is of formula: 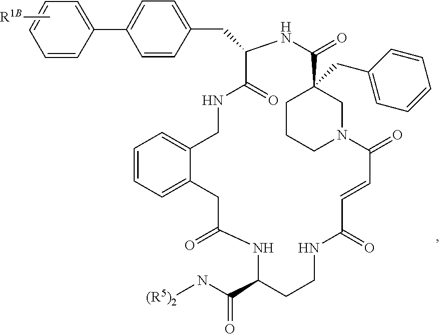
or a pharmaceutically acceptable salt, solvate, hydrate, polymorph, co-crystal, tautomer, stereoisomer, isotopically enriched form, prodrug, or mixture thereof.

27. The compound of any one of claim 2, 4, 5, or 17-26, wherein the compound is of formula: 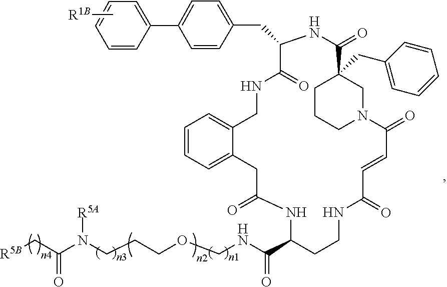
or a pharmaceutically acceptable salt, solvate, hydrate, polymorph, co-crystal, tautomer, stereoisomer, isotopically enriched form, prodrug, or mixture thereof.

28. The compound of any one of claim 2, 4, or 5, wherein the compound is of formula: 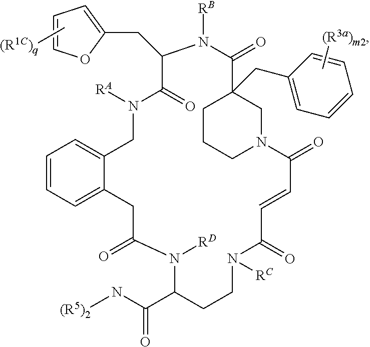
or a pharmaceutically acceptable salt, solvate, hydrate, polymorph, co-crystal, tautomer, stereoisomer, isotopically enriched form, prodrug, or mixture thereof.

29. The compound of any one of claim 2, 4, 5, or 28, wherein the compound is of formula: 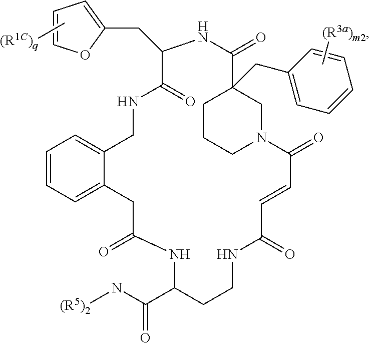
or a pharmaceutically acceptable salt, solvate, hydrate, polymorph, co-crystal, tautomer, stereoisomer, isotopically enriched form, prodrug, or mixture thereof.

30. The compound of any one of claim 2, 4, 5, 28, or 29, wherein the compound is of formula: 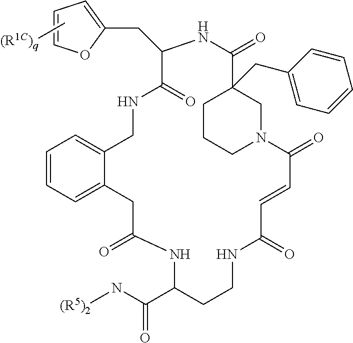
or a pharmaceutically acceptable salt, solvate, hydrate, polymorph, co-crystal, tautomer, stereoisomer, isotopically enriched form, prodrug, or mixture thereof.

31. The compound of any one of claim 2, 4, 5, or 28, wherein the compound is of formula: 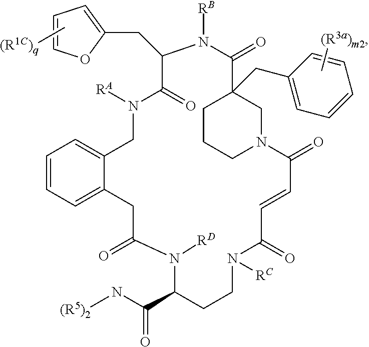
or a pharmaceutically acceptable salt, solvate, hydrate, polymorph, co-crystal, tautomer, stereoisomer, isotopically enriched form, prodrug, or mixture thereof.

32. The compound of any one of claim 2, 4, 5, 28, or 31, wherein the compound is of formula: 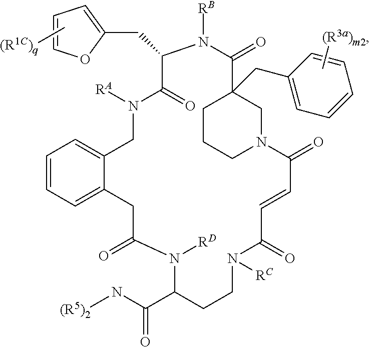
or a pharmaceutically acceptable salt, solvate, hydrate, polymorph, co-crystal, tautomer, stereoisomer, isotopically enriched form, prodrug, or mixture thereof.

33. The compound of any one of claim 2, 4, 5, 28, 31, or 32, wherein the compound is of 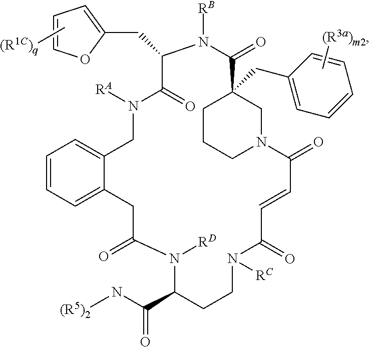
or a pharmaceutically acceptable salt, solvate, hydrate, polymorph, co-crystal, tautomer, stereoisomer, isotopically enriched form, prodrug, or mixture thereof.

34. The compound of any one of claim 2, 4, 5, 28, 29, or 31-33, wherein the compound is of formula: 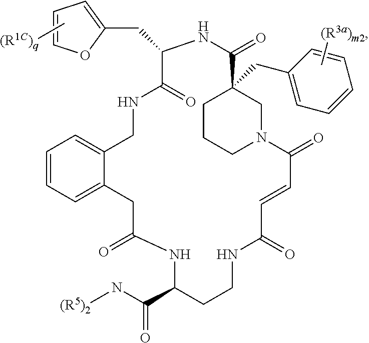
or a pharmaceutically acceptable salt, solvate, hydrate, polymorph, co-crystal, tautomer, stereoisomer, isotopically enriched form, prodrug, or mixture thereof.

35. The compound of any one of claim 2, 4, 5, or 28-34, wherein the compound is of formula: 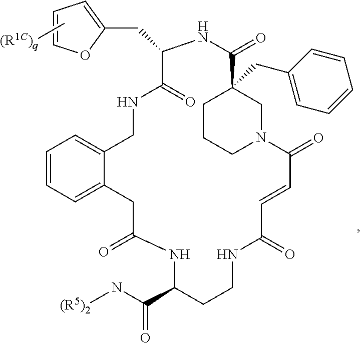
or a pharmaceutically acceptable salt, solvate, hydrate, polymorph, co-crystal, tautomer, stereoisomer, isotopically enriched form, prodrug, or mixture thereof.

36. The compound of any one of claim 2, 4, 5, or 28-35, wherein the compound is of formula: 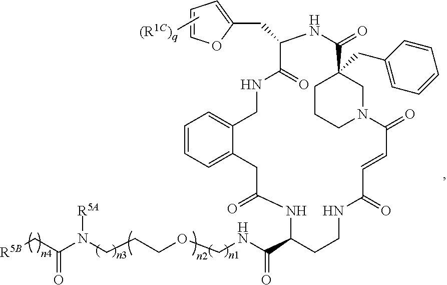
or a pharmaceutically acceptable salt, solvate, hydrate, polymorph, co-crystal, tautomer, stereoisomer, isotopically enriched form, prodrug, or mixture thereof.

37. The compound of any one of claims 3-5, wherein the compound is of formula: 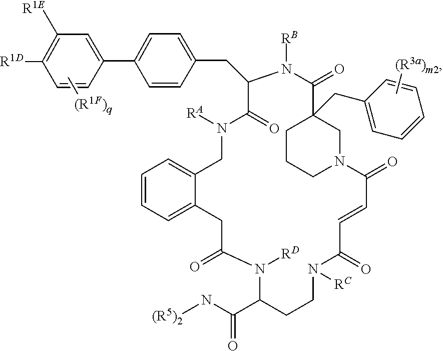
or a pharmaceutically acceptable salt, solvate, hydrate, polymorph, co-crystal, tautomer, stereoisomer, isotopically enriched form, prodrug, or mixture thereof.

38. The compound of any one of claims 3-5 or 37, wherein the compound is of formula: 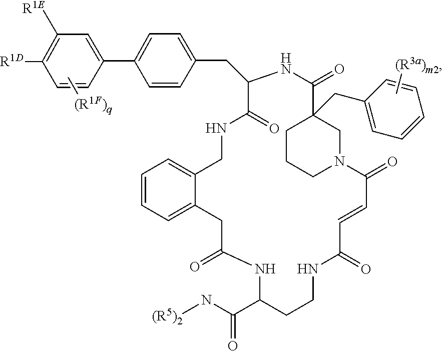
or a pharmaceutically acceptable salt, solvate, hydrate, polymorph, co-crystal, tautomer, stereoisomer, isotopically enriched form, prodrug, or mixture thereof.

39. The compound of any one of claims 3-5, 37, or 38, wherein the compound is of formula: 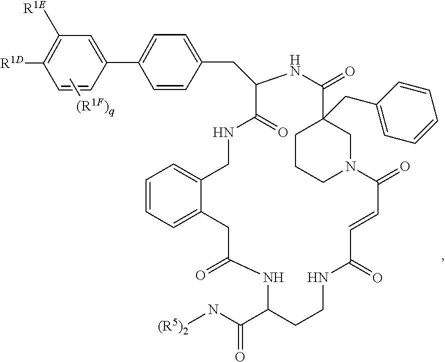
or a pharmaceutically acceptable salt, solvate, hydrate, polymorph, co-crystal, tautomer, stereoisomer, isotopically enriched form, prodrug, or mixture thereof.

40. The compound of any one of claims 3-5 or 37-39, wherein the compound is of formula: 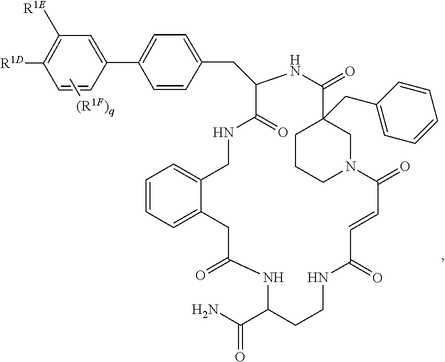
or a pharmaceutically acceptable salt, solvate, hydrate, polymorph, co-crystal, tautomer, stereoisomer, isotopically enriched form, prodrug, or mixture thereof.

41. The compound of any one of claims 3-5 or 37, wherein the compound is of formula: 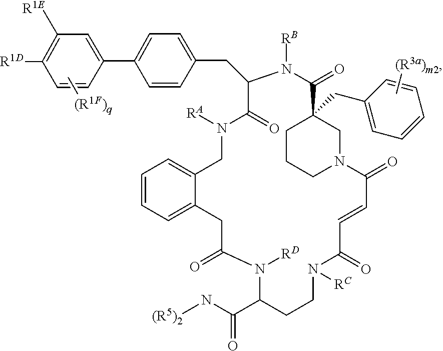
or a pharmaceutically acceptable salt, solvate, hydrate, polymorph, co-crystal, tautomer, stereoisomer, isotopically enriched form, prodrug, or mixture thereof.

42. The compound of any one of claims 3-5, 37, or 41, wherein the compound is of formula: 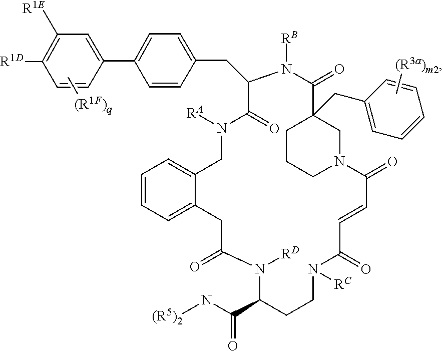
or a pharmaceutically acceptable salt, solvate, hydrate, polymorph, co-crystal, tautomer, stereoisomer, isotopically enriched form, prodrug, or mixture thereof.

43. The compound of any one of claims 3-5, 37, 41, or 42, wherein the compound is of 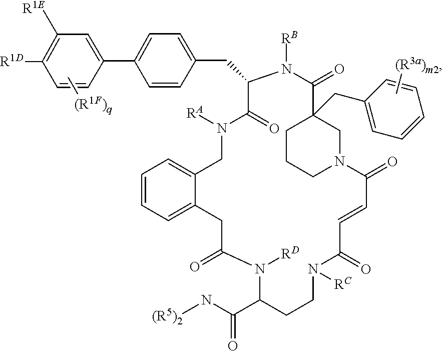
or a pharmaceutically acceptable salt, solvate, hydrate, polymorph, co-crystal, tautomer, stereoisomer, isotopically enriched form, prodrug, or mixture thereof.

44. The compound of any one of claims 3-5, 37, or 41-43, wherein the compound is of formula: 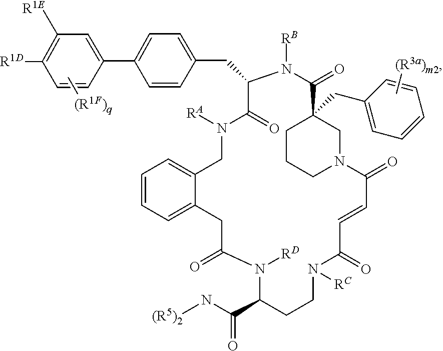
or a pharmaceutically acceptable salt, solvate, hydrate, polymorph, co-crystal, tautomer, stereoisomer, isotopically enriched form, prodrug, or mixture thereof.

45. The compound of any one of claims 3-5, 37, 38, or 41-44, wherein the compound is of 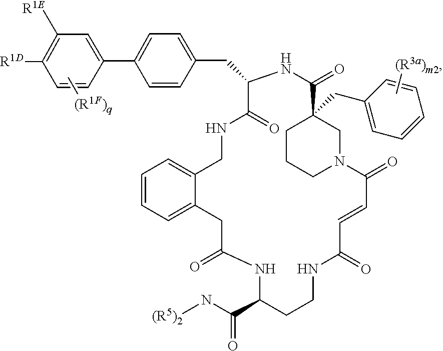
or a pharmaceutically acceptable salt, solvate, hydrate, polymorph, co-crystal, tautomer, stereoisomer, isotopically enriched form, prodrug, or mixture thereof.

46. The compound of any one of claims 3-5, 37-39, or 41-45, wherein the compound is of formula: 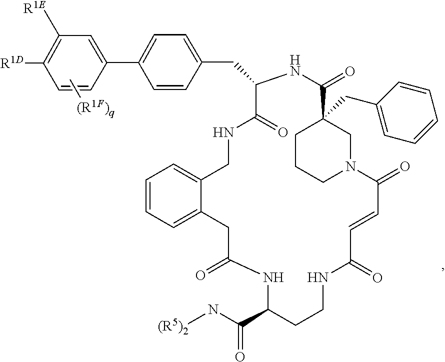
or a pharmaceutically acceptable salt, solvate, hydrate, polymorph, co-crystal, tautomer, stereoisomer, isotopically enriched form, prodrug, or mixture thereof.

47. The compound of any one of claims 3-5 or 37-46, wherein the compound is of formula: 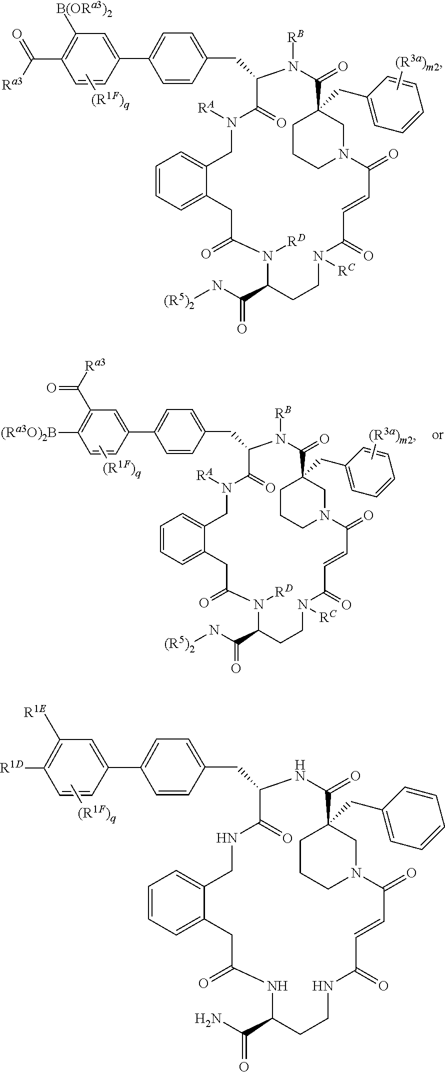
or a pharmaceutically acceptable salt, solvate, hydrate, polymorph, co-crystal, tautomer, stereoisomer, isotopically enriched form, prodrug, or mixture thereof.

48. The compound of any one of claims 1-47, wherein the compound is of formula: 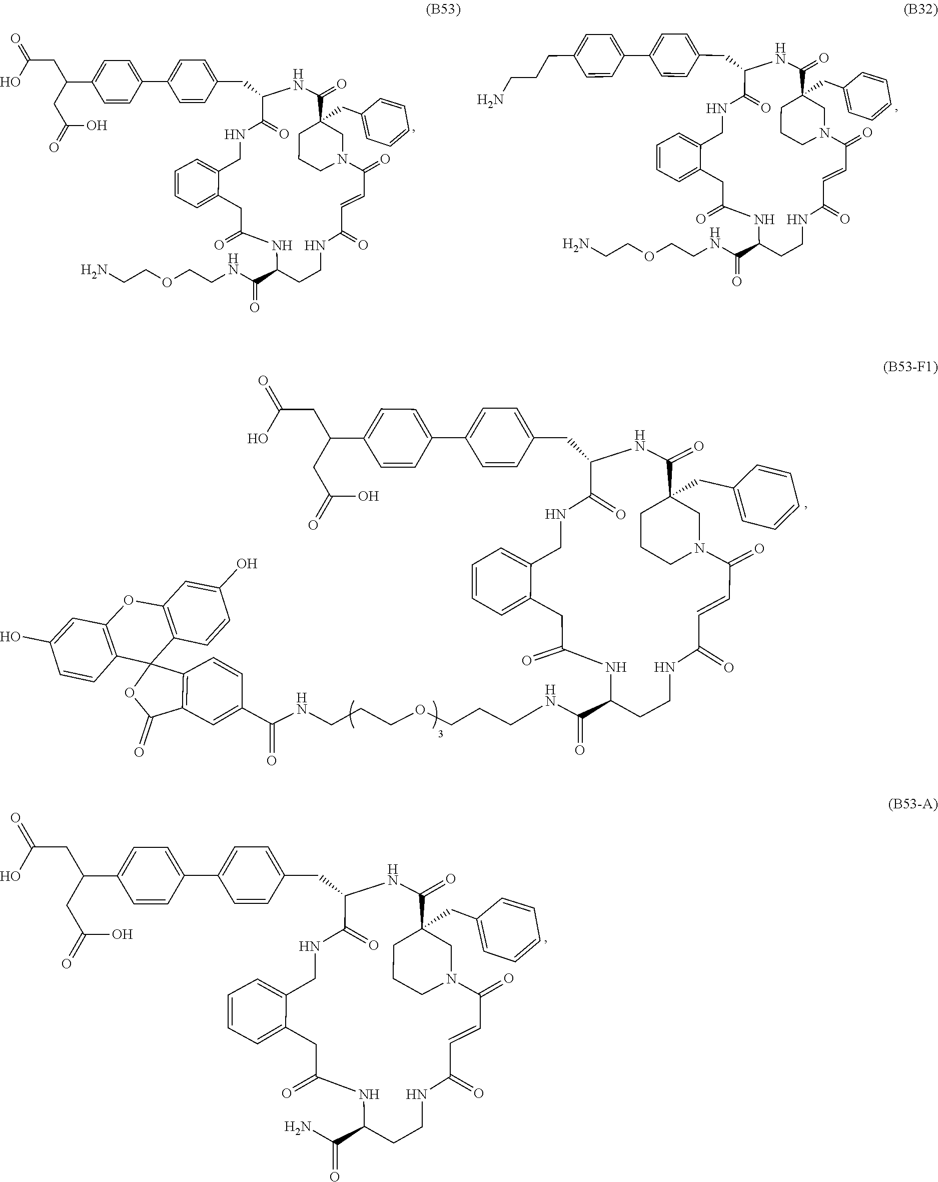 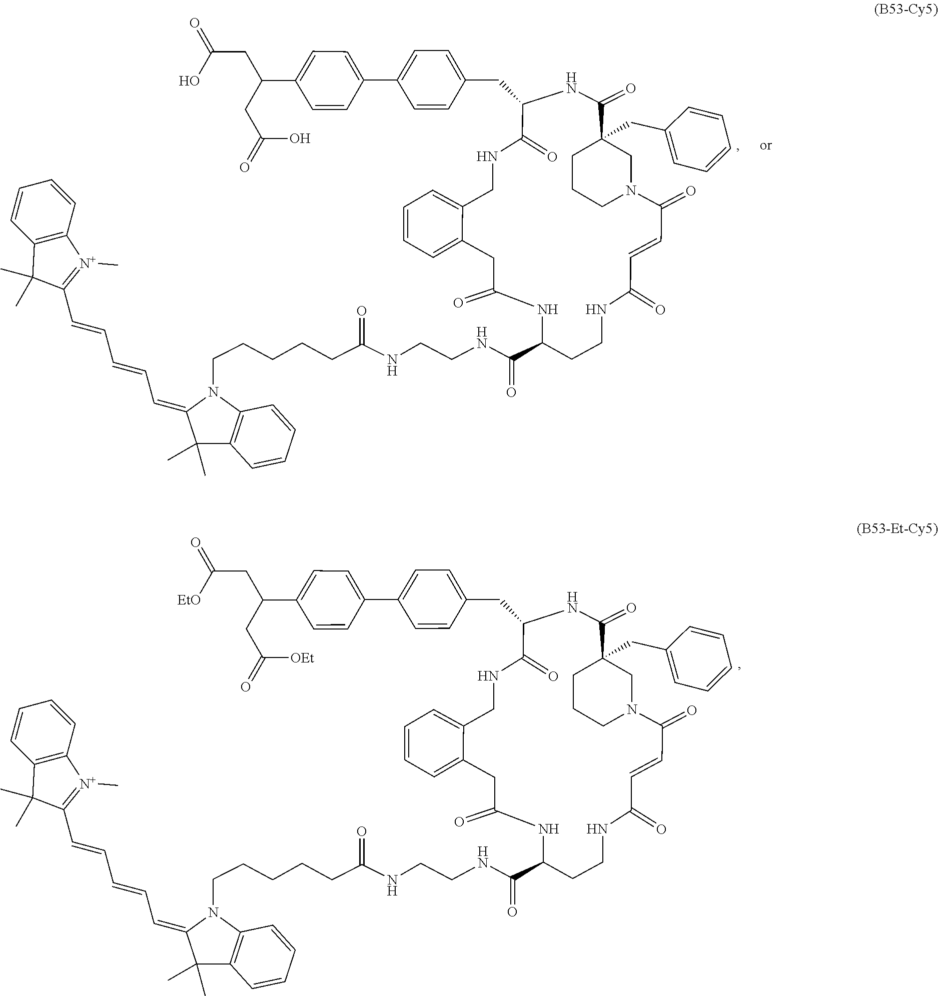 [SMILES: C[N+]1=C(/C=C/C=C/C=C2\N(CCCCCC(=O)NCCNC(=O)[C@@H]3CCNC(=O)/C=C/C(=O)N4CCC[C@](Cc5ccccc5)(C4)C(=O)N[C@@H](Cc4ccc(-c5ccc(C(CC(=O)O)CC(=O)O)cc5)cc4)C(=O)NCc4ccccc4CC(=O)N3)c3ccccc3C2(C)C)C(C)(C)c2ccccc21 ; CCOC(=O)CC(CC(=O)OCC)c1ccc(-c2ccc(C[C@@H]3NC(=O)[C@]4(Cc5ccccc5)CCCN(C4)C(=O)/C=C/C(=O)NCC[C@@H](C(=O)NCCNC(=O)CCCCCN4\C(=C/C=C/C=C/C5=[N+](C)c6ccccc6C5(C)C)C(C)(C)c5ccccc54)NC(=O)Cc4ccccc4CNC3=O)cc2)cc1]
or a pharmaceutically acceptable salt, solvate, hydrate, polymorph, co-crystal, tautomer, stereoisomer, isotopically enriched form, prodrug, or mixture thereof.

49. The compound of any one of claims 1-47, wherein the compound is of formula: 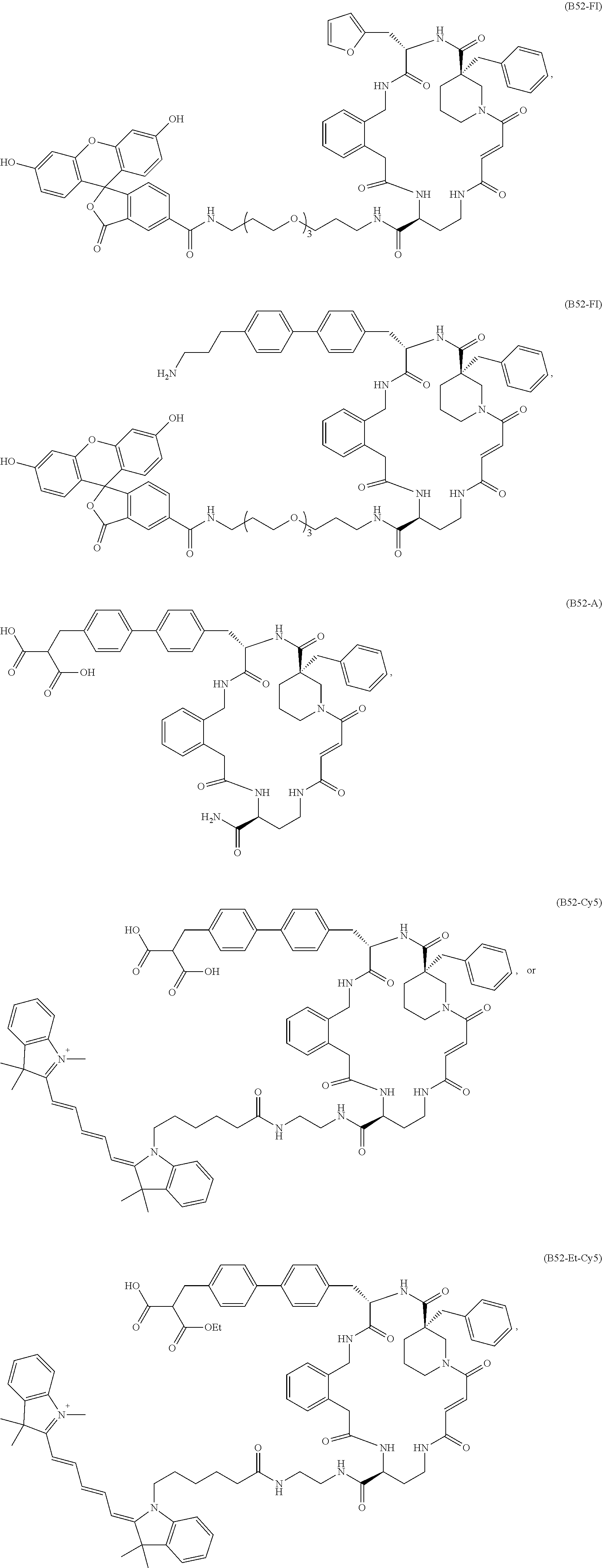
or a pharmaceutically acceptable salt, solvate, hydrate, polymorph, co-crystal, tautomer, stereoisomer, isotopically enriched form, prodrug, or mixture thereof.

50. The compound of any one of claims 1-47, wherein the compound is of formula: 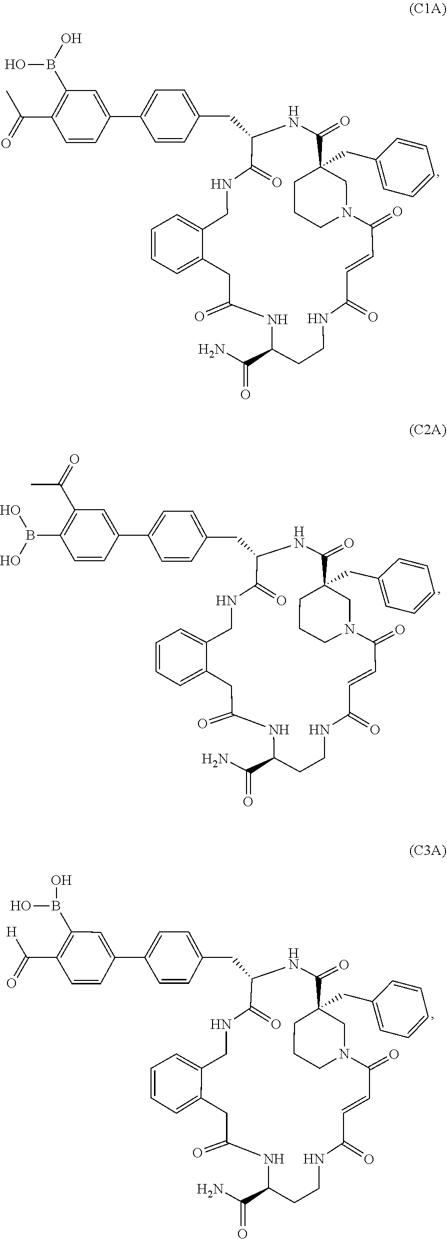 [SMILES: CC(=O)c1ccc(-c2ccc(C[C@@H]3NC(=O)[C@]4(Cc5ccccc5)CCCN(C4)C(=O)/C=C/C(=O)NCC[C@@H](C(N)=O)NC(=O)Cc4ccccc4CNC3=O)cc2)cc1B(O)O ; CC(=O)c1cc(-c2ccc(C[C@@H]3NC(=O)[C@]4(Cc5ccccc5)CCCN(C4)C(=O)/C=C/C(=O)NCC[C@@H](C(N)=O)NC(=O)Cc4ccccc4CNC3=O)cc2)ccc1B(O)O ; NC(=O)[C@@H]1CCNC(=O)/C=C/C(=O)N2CCC[C@](Cc3ccccc3)(C2)C(=O)N[C@@H](Cc2ccc(-c3ccc(C=O)c(B(O)O)c3)cc2)C(=O)NCc2ccccc2CC(=O)N1] 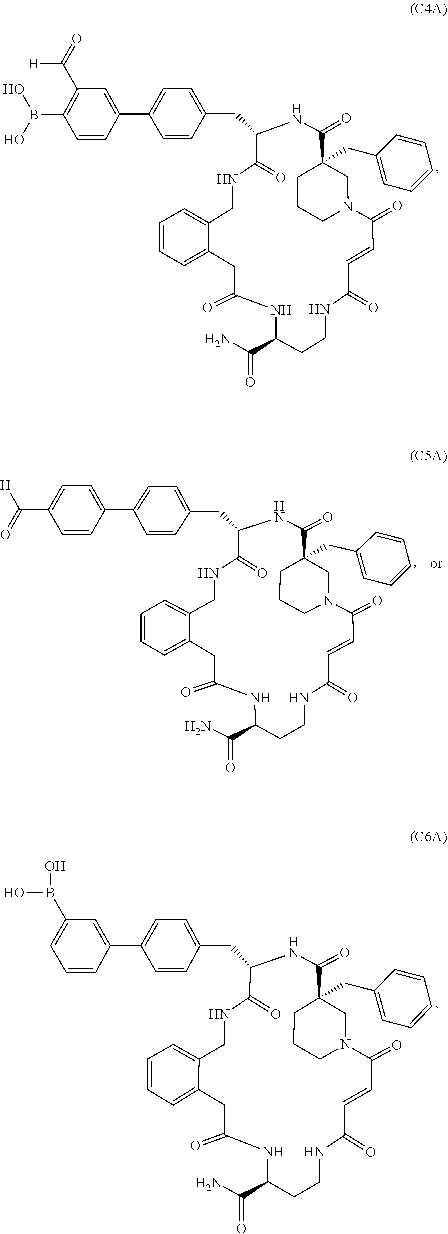 [SMILES: NC(=O)[C@@H]1CCNC(=O)/C=C/C(=O)N2CCC[C@](Cc3ccccc3)(C2)C(=O)N[C@@H](Cc2ccc(-c3ccc(B(O)O)c(C=O)c3)cc2)C(=O)NCc2ccccc2CC(=O)N1 ; NC(=O)[C@@H]1CCNC(=O)/C=C/C(=O)N2CCC[C@](Cc3ccccc3)(C2)C(=O)N[C@@H](Cc2ccc(-c3ccc(C=O)cc3)cc2)C(=O)NCc2ccccc2CC(=O)N1 ; NC(=O)[C@@H]1CCNC(=O)/C=C/C(=O)N2CCC[C@](Cc3ccccc3)(C2)C(=O)N[C@@H](Cc2ccc(-c3cccc(B(O)O)c3)cc2)C(=O)NCc2ccccc2CC(=O)N1]
or a pharmaceutically acceptable salt, solvate, hydrate, polymorph, co-crystal, tautomer, stereoisomer, isotopically enriched form, prodrug, or mixture thereof.

51. The compound of any one of claims 1-48, wherein the compound is of formula: 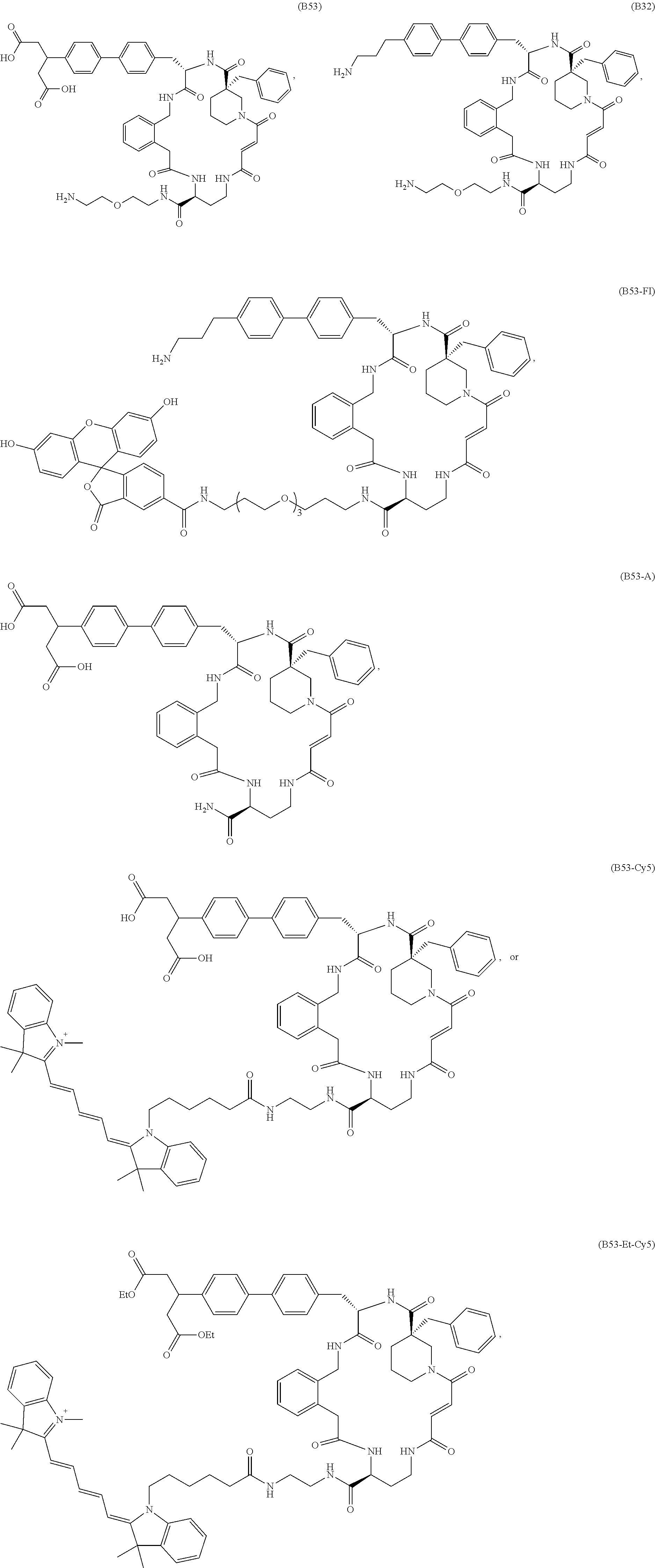
or a pharmaceutically acceptable salt thereof.

52. The compound of any one of claims 1-47 and 49, wherein the compound is of formula: 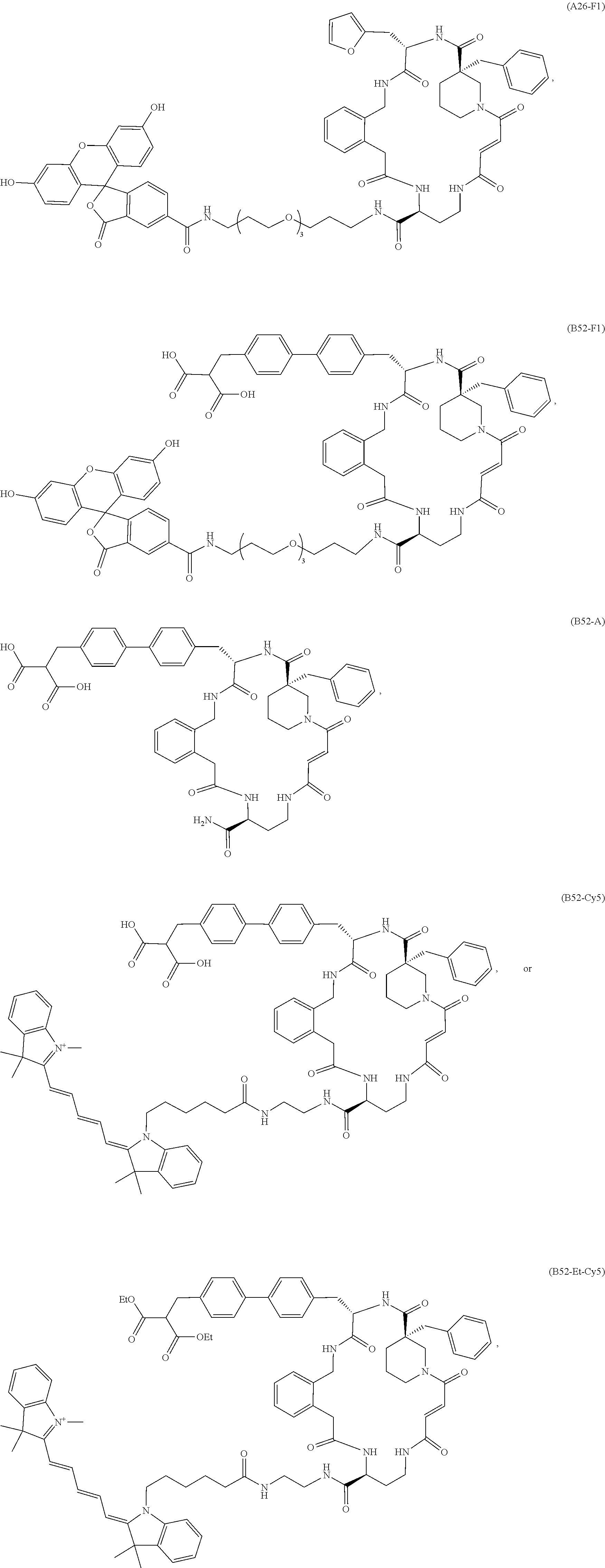
or a pharmaceutically acceptable salt thereof.

53. The compound of any one of claims 1-47 and 50, wherein the compound is of formula: 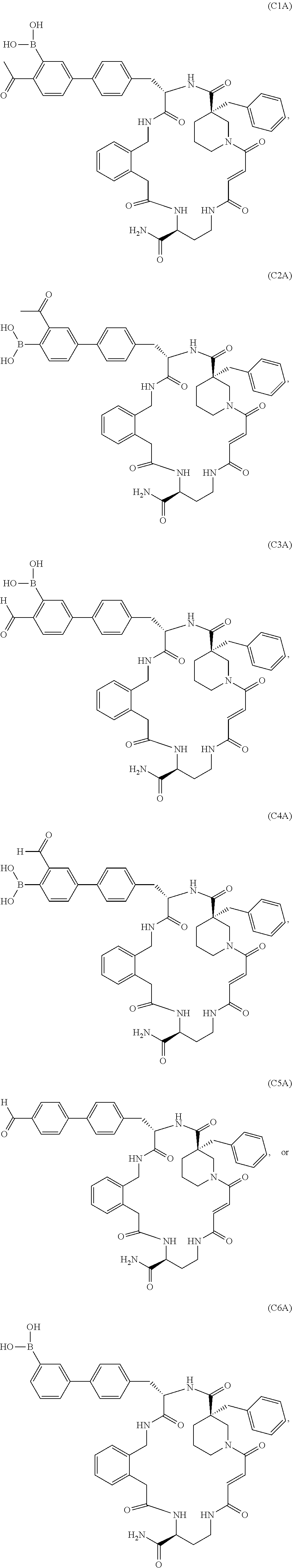
or a pharmaceutically acceptable salt thereof.

54. The compound of any one of claims 1-53, wherein the compound is capable of selectively binding cyclophilin D over other cyclophilins.

55. The compound of any one of claims 1-53, wherein the compound is capable of selectively binding cyclophilin E over other cyclophilins.

56. A pharmaceutical composition comprising a compound of any one of claims 1-55, or a pharmaceutically acceptable salt, solvate, hydrate, polymorph, co-crystal, tautomer, stereoisomer, isotopically enriched form, prodrug, or mixture thereof, and a pharmaceutically acceptable excipient.

57. The pharmaceutical composition of claim 56, wherein the pharmaceutical composition comprises a therapeutically effective amount of the compound for use in treating a disease in a subject in need thereof.

58. A method of treating a disease and/or condition in a subject in need thereof, the method comprising administering to the subject a therapeutically effective amount of a compound of any one of claims 1-55, or a pharmaceutically acceptable salt, solvate, hydrate, polymorph, co-crystal, tautomer, stereoisomer, isotopically enriched form, prodrug, or mixture thereof, or a pharmaceutical composition of claim 56 or 57.

59. The method of claim 58, wherein the disease and/or condition is associated with cyclophilin D.

60. The method of claim 58 or 59, wherein the disease and/or condition is ischemia-reperfusion injury (IRI), Alzheimer's disease, Huntington's disease, multiple sclerosis (MS), Parkinson's disease, amyotrophic lateral sclerosis (ALS), X-linked adrenoleukodystrophy (X-ALD), liver cirrhosis, or diabetes.

61. A method of reducing oxidative stress in a subject in need thereof, the method comprising administering to the subject a therapeutically effective amount of a compound of any one of claims 1-55, or a pharmaceutically acceptable salt, solvate, hydrate, polymorph, co-crystal, tautomer, stereoisomer, isotopically enriched form, prodrug, or mixture thereof, or a pharmaceutical composition of claim 56 or 57.

62. A method of reducing oxidative stress in a cell, tissue, or biological sample, the method comprising:
contacting the cell, tissue, or biological sample with an effective amount of a compound of any one of claims 1-55, or a pharmaceutically acceptable salt, solvate, hydrate, polymorph, co-crystal, tautomer, stereoisomer, isotopically enriched form, prodrug, or mixture thereof, or a pharmaceutical composition of claim 56 or 57.

63. A method of binding a cyclophilin in a subject in need thereof, the method comprising: administering to the subject a therapeutically effective amount of a compound of any one of claims 1-55, or a pharmaceutically acceptable salt, solvate, hydrate, polymorph, co-crystal, tautomer, stereoisomer, isotopically enriched form, prodrug, or mixture thereof, or a pharmaceutical composition of claim 56 or 57.

64. A method of binding a cyclophilin in a cell, tissue, or biological sample, the method comprising:
contacting the cell, tissue, or biological sample with an effective amount of a compound of any one of claims 1-55, or a pharmaceutically acceptable salt, solvate, hydrate, polymorph, co-crystal, tautomer, stereoisomer, isotopically labeled derivative, prodrug, mixture thereof, or a pharmaceutical composition of claim 56 or 57.

65. The method of claim 63 or 64, wherein the cyclophilin is cyclophilin D.

66. The method of any one of claims 63-65, wherein the compound is capable of selectively binding cyclophilin D over other cyclophilins.

67. The method of any one of claims 63-66, wherein the compound is capable of binding the S2 pocket of cyclophilin D.

68. The method of any one of claims 63-67, wherein the compound is capable of binding gatekeeper residues of cyclophilin D.

69. The method of any one of claims 63-68, wherein the compound is capable of binding the gatekeeper residues serine 123 and/or arginine 124 of cyclophilin D.

70. The method of any one of claims 63-69, wherein the compound is capable of preventing mPTP opening.

71. The method of claim 63 or 64, wherein the cyclophilin is cyclophilin E.

72. The method of any one of claims 63, 64, or 71, wherein the compound is capable of selectively binding cyclophilin E over other cyclophilins.

73. The method of any one of claims 63, 64, 71, or 72, wherein the compound is capable of binding the S2 pocket of cyclophilin E.

74. The method of any one of claims 63, 64, or 71-73, wherein the compound is capable of binding gatekeeper residues of cyclophilin E.

75. The method of any one of claims 63, 64, or 71-74, wherein the compound is capable of binding the lysine gatekeeper residues lysine 217 and/or lysine 218 of cyclophilin E.

76. The method of any one of claims 63-75, comprising inhibiting the activity of the cyclophilin.

77. The method of any one of claims 58-61 and 63 further comprising administering to the subject a therapeutically effective amount of an additional pharmaceutical agent in combination with the compound of any one of claims 1-55, or a pharmaceutically acceptable salt, solvate, hydrate, polymorph, co-crystal, tautomer, stereoisomer, isotopically labeled derivative, prodrug, or mixture thereof, or the pharmaceutical composition of any one of claim 56 or 57.

78. Use of a compound of any one of claims 1-55, or a pharmaceutically acceptable salt, solvate, hydrate, polymorph, co-crystal, tautomer, stereoisomer, isotopically enriched form, prodrug, or mixture thereof, or a pharmaceutical composition of claim 56 or 57, to treat a disease and/or condition in a subject in need thereof.

79. A kit comprising:
a compound of any one of claims 1-55, or a pharmaceutically acceptable salt, solvate, hydrate, polymorph, co-crystal, tautomer, stereoisomer, isotopically enriched form, prodrug, or mixture thereof, or a pharmaceutical composition of claim 56 or 57; and
instructions for administering to a subject or contacting a cell, tissue, or biological sample with the compound, or pharmaceutically acceptable salt, solvate, hydrate, polymorph, co-crystal, tautomer, stereoisomer, isotopically labeled derivative, prodrug, or mixture thereof, or pharmaceutical composition.
